(19) United States
(12) Patent Application Publication
(10) Pub. No.: US 2024/0336588 A1
(43) Pub. Date: Oct. 10, 2024

(54) COMPOSITIONS AND METHODS FOR ANTIOXIDANT AND ANTI-INFLAMMATORY THERAPEUTICS

(71) Applicant: AVANTI BIOSCIENCES INC., Stony Brook, NY (US)

(72) Inventor: Gian Luca ARALDI, East Setauket, NY (US)

(21) Appl. No.: 18/576,176

(22) PCT Filed: Jul. 14, 2022
(86) PCT No.: PCT/US2022/037208

§ 371 Date: Jan. 3, 2024

Related U.S. Application Data
(60) Provisional application No. 63/224,202, filed on Jul. 21, 2021.
(60) Provisional application No. 63/222,673, filed on Jul. 16, 2021.

(51) Int. Cl. C07D 311/74; A61K 9/00; A61K 31/353; A61P 25/28

(52) U.S. Cl. CPC C07D 311/74; A61K 9/0043; A61K 31/353; A61P 25/28

(57) ABSTRACT

The present invention relates to compositions and methods for antioxidant and anti-inflammatory therapeutics.

### FEDERAL FUNDING STATEMENT

This invention was made with government support under Grant No. 1R43AG063560-01 and Grant No. R44AG056181, each awarded by the National Institutes on Aging. The government has certain rights in the invention.

### BACKGROUND

DYRK1A is a member of the DYRK family, and it affects tau phosphorylation and the formation of tau neurofibrillary tangles. In addition, DYRK1A alters APP phosphorylation and induces amyloid beta (AB) production, and DYRK1A expression in the hippocampus is increased in neurodegenerative diseases. Moreover, DYRK1A is strongly associated with neuroinflammation. These findings support DYRK1A as a potential target for preventing or treating a variety of diseases.

### SUMMARY

In one aspect, the disclosure provides a compound of Formula I

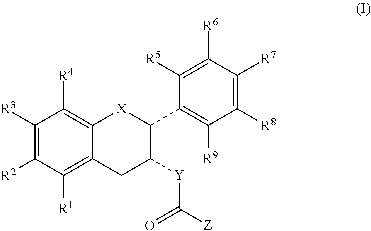

or a pharmaceutically acceptable salt thereof, wherein
R¹, R², R³, and R⁴ are each independently hydrogen, halogen, —NO₂, —CN, C₁-C₁₀ alkyl, C₁-C₁₀ haloalkyl, —NH₂, —NH(C₁-C₁₀ alkyl), —N(C₁-C₁₀ alkyl)₂, —OH, C₁-C₁₀ alkoxy, C₁-C₁₀ haloalkoxy, —SH, hydroxy(C₁-C₁₀ alkyl), alkoxy(C₁-C₁₀ alkyl), amino(C₁-C₁₀ alkyl), —CONH₂, —CONH(C₁-C₁₀ alkyl), —CON(C₁-C₁₀ alkyl)₂, —OC(O)NH₂, —OC(O)NH(C₁-C₁₀ alkyl), —OC(O)N(C₁-C₁₀ alkyl)₂, —CO₂H, —CO₂(C₁-C₁₀ alkyl), —CHO, —CO(C₁-C₁₀ alkyl), —OC(O)(C₁-C₁₀ alkyl), —S(O)₀₋₂(C₁-C₁₀ alkyl), or —NH(S(O)₀₋₂(C₁-C₁₀ alkyl));
R⁵ and R⁹ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or —SH;
R⁷ is hydrogen, halogen, —NO₂, —CN, C₁-C₁₀ alkyl, C₁-C₁₀ haloalkyl, —NH₂, —NH(C₁-C₁₀ alkyl), —N(C₁-C₁₀ alkyl)₂, —OH, C₁-C₁₀ alkoxy, C₁-C₁₀ haloalkoxy, —SH, hydroxy(C₁-C₁₀ alkyl), alkoxy(C₁-C₁₀ alkyl), amino(C₁-C₁₀ alkyl), —CONH₂, —CONH(C₁-C₁₀ alkyl), —CON(C₁-C₁₀ alkyl)₂, —CO₂H, —CO₂(C₁-C₁₀ alkyl), —CHO, —CO(C₁-C₁₀ alkyl), —S(O)₀₋₂(C₁-C₁₀ alkyl), or —NH(S(O)₀₋₂(C₁-C₁₀ alkyl));
R⁶ and R⁸ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or —SH;
X is O or C;
Y is O or NH; and
Z is

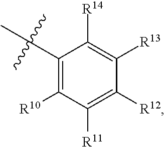

wherein
   R¹⁰ and R¹⁴ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or —SH;
   R¹² is hydrogen, halogen, —NO₂, —CN, C₁-C₁₀ alkyl, C₁-C₁₀ haloalkyl, —NH₂, —NH(C₁-C₁₀ alkyl), —N(C₁-C₁₀ alkyl)₂, —OH, C₁-C₁₀ alkoxy, C₁-C₁₀ haloalkoxy, —SH, hydroxy(C₁-C₁₀ alkyl), alkoxy(C₁-C₁₀ alkyl), amino(C₁-C₁₀ alkyl), —CONH₂, —CONH(C₁-C₁₀ alkyl), —CON(C₁-C₁₀ alkyl)₂, —CO₂H, —CO₂(C₁-C₁₀ alkyl), —CHO, —CO(C₁-C₁₀ alkyl), —S(O)₀₋₂(C₁-C₁₀ alkyl), or —NH(S(O)₀₋₂(C₁-C₁₀ alkyl));
   R¹¹ and R¹³ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or —SH;
or Z is

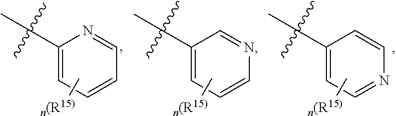

wherein
   n is 0-4; and
   each R¹⁵ is independently C₁-C₆ alkyl, C₁-C₆ haloalkyl, —NH₂, —NH(C₁-C₆ alkyl), —N(C₁-C₆ alkyl)₂, —OH, C₁-C₆ alkoxy, or C₁-C₆ haloalkoxy;
or Z is

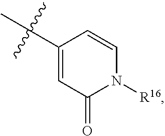

wherein
   R¹⁶ is hydrogen, C₁-C₆ alkyl, C₁-C₆ haloalkyl, —OH, C₁-C₆ alkoxy, or C₁-C₆ haloalkoxy;
or Z is

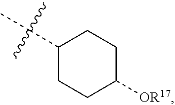

wherein
   R¹⁷ is hydrogen, C₁-C₆ alkyl, or C₁-C₆ haloalkyl.

In one embodiment, the compound of Formula I is not (2R,3R)-2-(3,5-dihydroxy-4-methoxyphenyl)-5,7-dihydroxychroman-3-yl 3,4,5-trihydroxybenzoate, (2R,3R)-5,7-dihydroxy-2-(3,4,5-trihydroxyphenyl)chroman-3-yl 3,5-dihydroxy-4-methoxybenzoate, (2R,3R)-5,7-dihydroxy-2-(3,4,5-trihydroxyphenyl)chroman-3-yl 3,4,5-trihydroxybenzoate, (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2R,3R)-2-(3,4-dihydroxyphenyl)-5,7-dihydroxychroman-3-yl 3,4,5-trihydroxybenzoate, (2S,3R)-2-(3,4-dihydroxyphenyl)-5,7-dihydroxychroman-3-yl 3,4,5-trihydroxybenzoate, (2R,3R)-5,7-dihydroxy-2-(3,4,5-trihydroxyphenyl)chroman-3-yl 3,4-dihydroxy-5-methoxybenzoate; (2R,3S)-5,7-dihydroxy-2-(3,4,5-trihydroxyphenyl)chroman-3-yl 3,4-difluorobenzoate; or (2S,3R)-5,7-dihydroxy-2-(3,4,5-trihydroxyphenyl)chroman-3-yl 2,3,4-trihydroxybenzoate.

In another aspect, the disclosure provides a pharmaceutical composition comprising one or more compounds of the disclosure as described herein and a pharmaceutically acceptable carrier, excipient, adjuvant, and/or diluent.

In another aspect, the disclosure provides an intranasal pharmaceutical composition comprising one or more compounds of the disclosure as described herein, present in a combined amount of 1-40 wt. %, and one or more of
a permeation enhancer, present in an amount of 0.1-20 wt. %;
a chelator/anti-oxidant, present in an amount of 0.1-20 wt. %;
a humectant; present in an amount of 1-30 wt. %; and
a preservative, present in an amount of 0.03-2 wt. %;
   wherein the pH of the composition is 4.0-6.5.

In another aspect, the disclosure provides methods of treating or limiting oxidative stress and/or inflammation, including but not limited to oxidative stress and/or inflammation related to a neurological disorder or a viral infection. Such methods comprise administering to a subject in need thereof one or more compounds of the disclosure as described herein or a pharmaceutical composition of the disclosure as described herein.

In another aspect, the disclosure provides a method for inhibiting DYRK1A, comprising administering to a subject in need thereof one or more compounds of the disclosure as described herein or a pharmaceutical composition of the disclosure as described herein.

Additional aspects of the disclosure will be evident from the disclosure herein.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1 is a graph showing the efficacy of compound 40 described herein in a MOG₃₅₋₅₅-induced murine model of chronic progressive Experimental Autoimmune Encephalomyelitis (EAE).

FIG. 2 is a graph comparing the effect of compound 40 described herein on histological markers of the chronic progressive Experimental Autoimmune Encephalomyelitis (EAE) therapeutic model.

FIG. 3 is a graph showing the effect of compound 68 described herein on hippocampus levels of TNF-α levels in a lipopolysaccharide-induced TNF-α inflammation model. (*P<0.05; **P<0.01; ***P<0.001; VS. G2 Vehicle)

FIG. 4 is a graph showing the effect of compound 68 described herein on plasma levels of TNF-α in a lipopolysaccharide-induced TNF-α inflammation model. (*P<0.05; **P<0.01; ***P<0.001; VS. G2 Vehicle)

FIG. 5 is a graph showing the effect of compound 68 described herein on pTau levels in hippocampus tissue in a lipopolysaccharide-induced TNF-α inflammation model.

### DETAILED DESCRIPTION

Before the disclosed processes and materials are described, it is to be understood that the aspects described herein are not limited to specific embodiments. As such, they can, of course, vary. It is also to be understood that the terminology used herein is for the purpose of describing particular aspects only and, unless specifically defined herein, is not intended to be limiting.

As disclosed herein, compounds of Formula I effectively treat or limit oxidative stress and/or inflammation in vivo (e.g., at significantly lower therapeutic doses, increasing overall safety and significantly reducing toxicity). Additionally, the present inventors note that the highly bioavailable compounds described herein can advantageously be useful for indications outside of the central nervous system. Thus, the compounds of the disclosure are particularly useful in treating or limiting diseases related to oxidative stress and/or inflammation, including but not limited to neurological disorders and viral infections.

### Therapeutic Applications

Accordingly, one aspect of the disclosure provides a method for treating or limiting oxidative stress and/or inflammation, comprising administering to a subject in need thereof a therapeutically effective amount of one or more compounds of Formula I or a pharmaceutically acceptable salt thereof (i.e., as otherwise described herein); or a pharmaceutical composition comprising one or more compounds of Formula I or a pharmaceutically acceptable salt thereof, and a pharmaceutically acceptable carrier, excipient, adjuvant, and/or diluent (i.e., as otherwise described herein); or an intranasal pharmaceutical composition comprising one or more compounds of Formula I (i.e., as otherwise described herein).

One aspect of the disclosure provides a method of treating or limiting a central nervous system disorder, a tumor, diabetes, obesity, or a systemic disorder, comprising administering to a subject in need thereof a therapeutically effective amount of one or more compounds of Formula I or a pharmaceutically acceptable salt thereof (i.e., as otherwise described herein); or a pharmaceutical composition comprising one or more compounds of Formula I or a pharmaceutically acceptable salt thereof, and a pharmaceutically acceptable carrier, excipient, adjuvant, and/or diluent (i.e., as otherwise described herein); or an intranasal pharmaceutical composition comprising one or more compounds of Formula I (i.e., as otherwise described herein).

One aspect of the disclosure provides a method of treating or limiting a neurological disorder, comprising administering to a subject in need thereof a therapeutically effective amount of one or more compounds of Formula I or a pharmaceutically acceptable salt thereof (i.e., as otherwise described herein); or a pharmaceutical composition comprising one or more compounds of Formula I or a pharmaceutically acceptable salt thereof, and a pharmaceutically acceptable carrier, excipient, adjuvant, and/or diluent (i.e., as otherwise described herein); or an intranasal pharmaceutical composition comprising one or more compounds of Formula I (i.e., as otherwise described herein). In certain embodiments as otherwise described herein, the neurological disorder is selected from multiple sclerosis, central pontine myelinolysis, acute disseminated encephalomyelitis, progressive multifocal leukoencephalopathy, subacute sclerosing panencephalitis, post-infectious encephalomyelitis, chronic inflammatory demyelinating polyneuropathy, Devic's disease, Balo's concentric sclerosis, the leukodystrophies, optic neuritis, transverse myelitis, cerebral palsy, spinal cord injury, age-associated myelin deficiency, Down syndrome, Alzheimer's Disease, Parkinson disorders, ataxia of Charlevoix-Saguenay (ARSACS), and acquired and inherited neuropathies in the peripheral nervous system.

One aspect of the disclosure provides a method for treating or limiting uveitis, comprising administering to a subject in need thereof a therapeutically effective amount of one or more compounds of Formula I or a pharmaceutically acceptable salt thereof (i.e., as otherwise described herein); or a pharmaceutical composition comprising one or more compounds of Formula I or a pharmaceutically acceptable salt thereof, and a pharmaceutically acceptable carrier, excipient, adjuvant, and/or diluent (i.e., as otherwise described herein); or an intranasal pharmaceutical composition comprising one or more compounds of Formula I (i.e., as otherwise described herein). Uveitis refers to various intraocular inflammatory diseases occurred in the uvea (i.e., iris, ciliary body, and choroid) and its adjacent structures (including cornea, vitreous body, retina, and optic nerve). Without timely diagnosis and treatment on chronic inflammation in the eye, it will lead to cataracts, glaucoma, corneal lesion, macular edema, or even permanent vision loss. Based on inflammatory involvement of the anatomic framework, the International Uveitis Study Group (IUSG) classified uveitis into anterior, intermediate, posterior, or pan-uveitis. Despite the effort in finding treatment for uveitis, none of the new agents are able to represent the perfect sole treatment with each owning particular side effects. Research is still needed to improve the efficiency and safety of treatments against uveitis. Because of its etiology, the use of anti-inflammatory agents has been seen as a viable route to cure uveitis. Anti-inflammatory agents that target novel pathways will provide another much needed treatment option for uveitis patients.

DYRK1A is an enzyme that has been implicated as an important drug target in various therapeutic areas, including neurological disorders (e.g., Down syndrome, Alzheimer's disease), oncology, and type 2 diabetes (e.g., pancreatic β-cell expansion). A sizeable and increasing body of evidence points to a role for DYRK1A in inflammation. Recently, it has been shown that DYRK1A phosphorylates Cyclin D1 leading to a decrease of p21 in the cells and ultimately to lower expression of the Nuclear factor erythroid 2-related factor 2 (Nrf2), a transcription factor that induces the expression of genes involved in antioxidant pathways, which reduce ROS levels. DYRK1A inhibitors can potentiate the neuroprotective p21-Nrf2 pathway and contribute to neuronal survival by suppressing pro-inflammatory cytokine production caused by neuroinflammation. DYRK1A inhibitors can also reduce inflammation by targeting other essential proteins like GFAP and STAT. Without wishing to be bound by theory, it is presently hypothesized that by reducing DYRK1A activity, it will be possible to reduce ocular inflammation and cure uveitis. Advantageously, the presently disclosed compounds possess enhanced efficacy relative to conventional therapies through multiple mechanisms of action combined with a superior safety profile.

One aspect of the disclosure provides a method for treating or limiting nonalcoholic fatty liver disease (NAFLD) (e.g., treating or limiting nonalcoholic steatohepatitis), comprising administering to a subject in need thereof a therapeutically effective amount of one or more compounds of Formula I or a pharmaceutically acceptable salt thereof (i.e., as otherwise described herein); or a pharmaceutical composition comprising one or more compounds of Formula I or a pharmaceutically acceptable salt thereof, and a pharmaceutically acceptable carrier, excipient, adjuvant, and/or diluent (i.e., as otherwise described herein); or an intranasal pharmaceutical composition comprising one or more compounds of Formula I (i.e., as otherwise described herein). Nonalcoholic fatty liver disease (NAFLD) is the most prevalent liver disease worldwide and there is no approved pharmacotherapy for it. Vitamin E, an antioxidant agent, and Pioglitazone, a Type II antidiabetic drug, have been shown to confer benefit in nonalcoholic steatohepatitis (NASH), a progressive form of NAFLD, but have compounding issues that limit their utility. GLP-1RA and SGLT2 inhibitors, which are approved for use in Type II diabetes (T2D), have similarly shown some efficacy in NASH. Without wishing to be bound by theory, it is presently believed that the compounds described herein can function as a potent and selective negative allosteric modulator of Dual-specificity tyrosine-(Y)-phosphorylation Regulated Kinase (DYRK).

DYRK1A has been implicated as an important drug target in various therapeutic areas including neurological disorders (e.g., Down syndrome, Alzheimer's disease), oncology, and T2D (e.g., pancreatic β-cell expansion). A sizeable and increasing body of evidence points to a role for DYRK1A and close family member DYRK1B in NAFLD pathogenesis. Notably, because DYRK1A phosphorylates the nuclear factors of activated T-cells (NFAT), DYRK1A inhibitors can induce B-cell proliferation and reduce blood glucose levels. Furthermore, DYRK1A phosphorylates Cyclin D1, which decreases p21 and ultimately lowers the expression of Nuclear factor erythroid 2-related factor 2 (Nrf2), a transcription factor that induces the expression of genes involved in antioxidant pathways and consequently reduces ROS levels. DYRK1A inhibitors potentiate the neuroprotective p21-Nrf2 pathway and contribute to neuronal survival by suppressing pro-inflammatory cytokine production caused by neuroinflammation. DYRK1A inhibitors also reduce inflammation by targeting other essential proteins like GFAP and STAT. Diabetes, oxidative stress and inflammation are all pathological hallmarks of NAFLD. Very recently, DYRK1B has also emerged as an important target for liver disease. DYRK1B is highly expressed in NASH, activates mTORC2, and causes hypertriglyceridemia, fatty liver, and hepatic insulin resistance (IR). Furthermore, DYRK1B is a potential therapeutic target for liver fibrosis by suppressing collagen production in hepatic stellate cells (HSCs). Avanti Biosciences claims that by reducing DYRK1A/B activity, it will be possible to intervene in NAFLD pathogenesis and slow or stop the disease progression. The projected advantage of a DYRK1A/B inhibitor over current therapies is based on its multiple mechanisms of action combined with a superior safety profile.

One aspect of the disclosure provides a method of limiting or treating a viral infection, comprising administering to a subject in need thereof a therapeutically effective amount of one or more compounds of Formula I or a pharmaceutically acceptable salt thereof (i.e., as otherwise described herein); or a pharmaceutical composition comprising one or more compounds of Formula I or a pharmaceutically acceptable salt thereof, and a pharmaceutically acceptable carrier, excipient, adjuvant, and/or diluent (i.e., as otherwise described herein); or an intranasal pharmaceutical composition comprising one or more compounds of Formula I (i.e., as otherwise described herein). In certain embodiments as otherwise described herein, the viral infection is a coronavirus infection. In other embodiments, the viral infection is a beta-coronavirus infection. In one such embodiment, the beta-coronavirus is selected from the group consisting of Human coronavirus HKU1, SARS-COV (including but not limited to SARS-COV-2), and MERS-COV In another embodiment, the viral infection is a severe acute respiratory syndrome coronavirus 2 (SARS-COV-2) infection. In certain embodiments as otherwise described herein, the subject has a viral infection or is “at risk” for a viral infection. In certain embodiments, the “at risk” subject is immunodeficient (e.g., due to conditions including malnutrition, drug addiction, alcoholism, and certain diseases states such as diabetes and AIDS) or at increased risk of exposure to a virus (e.g., health care workers, first responders to emergencies, subjects otherwise exposed to the virus, etc.).

In certain embodiments, the methods described herein can treat or limit oxidative stress and/or inflammation via specific receptor antagonism. For example, in certain embodiments, the methods described herein can inhibit COX (e.g., COX-2). The present inventors note that COX-2 has broad anti-inflammatory activity in the brain. In another example, in certain embodiments, the methods described herein can inhibit DYRK1A. The present inventors note that inhibition of DYRK1A can lead to up-regulation of nuclear factor erythroid 2-rated factor 2 (Nrf2), which modulates virus-induced oxidative stress, ROS generation and disease pathogenesis, which are vital in the viral life cycle. Moreover, Nrf2 can reduce expression of angiotensin-converting enzyme 2 (ACE2) receptors, which can function as receptors for coronavirus surface spike glycoproteins.

Accordingly, one aspect of the disclosure provides a method for inhibiting COX-2, comprising administering to a subject in need thereof a therapeutically effective amount of one or more compounds of Formula I or a pharmaceutically acceptable salt thereof (i.e., as otherwise described herein); or a pharmaceutical composition comprising one or more compounds of Formula I or a pharmaceutically acceptable salt thereof, and a pharmaceutically acceptable carrier, excipient, adjuvant, and/or diluent (i.e., as otherwise described herein); or an intranasal pharmaceutical composition comprising one or more compounds of Formula I (i.e., as otherwise described herein).

Another aspect of the disclosure provides a method for inhibiting DYRK1A, comprising administering to a subject in need thereof a therapeutically effective amount of one or more compounds of Formula I or a pharmaceutically acceptable salt thereof (i.e., as otherwise described herein); or a pharmaceutical composition comprising one or more compounds of Formula I or a pharmaceutically acceptable salt thereof, and a pharmaceutically acceptable carrier, excipient, adjuvant, and/or diluent (i.e., as otherwise described herein); or an intranasal pharmaceutical composition comprising one or more compounds of Formula I (i.e., as otherwise described herein).

Advantageously, the present inventors note that the methods described herein can inhibit overexpressed DYRK1A in a subject with Down syndrome. The methods can improve synaptic plasticity and/or delay the onset of Alzheimer's disease pathology, including tau hyperphosphorylation. Accordingly, in certain embodiments, the methods described herein can treat or limit Down syndrome and/or Alzheimer's disease. In certain desirable embodiments, the methods described herein can treat or limit Alzheimer's disease in a subject with Down syndrome (e.g., Down syndrome-related Alzheimer's disease).

In certain embodiments as otherwise described herein, the administration comprises oral administration or intranasal administration (e.g., of a pharmaceutical composition as otherwise described herein). For example, in certain embodiments as otherwise described herein, the administration is an intranasal pharmaceutical composition comprising one or more compounds of Formula I (i.e., as otherwise described herein).

In certain embodiments as otherwise described herein, the compound of Formula I is a compound of Table 1, below. In certain embodiments as otherwise described herein, the compound of Formula I is selected from compound 9-23, 25-37, and 39-81 of Table 1, below. In certain embodiments as otherwise described herein, the compound of Formula I is (2S,3R)-5,7-dihydroxy-2-(3,4,5-trihydroxyphenyl)chroman-3-yl 2,3,4-trihydroxybenzoate; (2S,3R)-5,7-dihydroxy-2-(3,4,5-trihydroxyphenyl)chroman-3-yl 2-fluoro-3,4,5-trihydroxybenzoate; (2S,3R)-5,7-dihydroxy-2-(3,4,5-trihydroxyphenyl)chroman-3-yl 2-fluoro-4,5-dihydroxybenzoate; (2S,3R)-2-(2-fluoro-3,4,5-trihydroxyphenyl)-5,7-dihydroxychroman-3-yl 3,4,5-trihydroxybenzoate; (2S,3R)-2-(2-fluoro-3,4,5-trihydroxyphenyl)-5,7-dihydroxychroman-3-yl 2-fluoro-3,4,5-trihydroxybenzoate; (2S,3R)-2-(2-fluoro-4,5-dihydroxyphenyl)-5,7-dihydroxychroman-3-yl 2-fluoro-3,4,5-trihydroxybenzoate; (2S,3R)-5,7-dihydroxy-2-(3,4,5-trihydroxyphenyl)chroman-3-yl 2-fluoro-4,5-dihydroxy-3-methoxybenzoate; (2S,3R)-5,7-dihydroxy-2-(3,4,5-trihydroxyphenyl)chroman-3-yl 2,6-difluoro-3,4,5-trihydroxybenzoate; (2S,3R)-2-(3,4-dihydroxyphenyl)-5,7-dihydroxychroman-3-yl 2-fluoro-3,4,5-trihydroxybenzoate; (2S,3R)-5,7-dihydroxy-2-(3,4,5-trihydroxyphenyl)chroman-3-yl 2,6-difluoro-3,4-dihydroxy-5-methoxybenzoate; (2S,3R)-5,7-dihydroxy-2-(3,4,5-trihydroxyphenyl)chroman-3-yl 2-fluoro-3,4-dihydroxy-5-methoxybenzoate; or (2R,3R)-5,7-dihydroxy-2-(3,4,5-trihydroxyphenyl)chroman-3-yl 2-fluoro-3,4,5-trihydroxybenzoate. In certain desirable embodiments as otherwise described herein, the compound of Formula I is (2S,3R)-5,7-dihydroxy-2-(3,4,5-trihydroxyphenyl)chroman-3-yl 2-fluoro-3,4-dihydroxy-5-methoxybenzoate. In certain desirable embodiments as otherwise described herein, the compound of Formula I is (2R,3R)-5,7-dihydroxy-2-(3,4,5-trihydroxyphenyl)chroman-3-yl 2-fluoro-3,4,5-trihydroxybenzoate.

### Compounds

As provided above, one aspect of the disclosure provides compounds of Formula I.

In certain embodiments as otherwise described herein, the compound of Formula I is not (2R,3R)-2-(3,5-dihydroxy-4-methoxyphenyl)-5,7-dihydroxychroman-3-yl 3,4,5-trihydroxybenzoate, (2R,3R)-5,7-dihydroxy-2-(3,4,5-trihydroxyphenyl)chroman-3-yl 3,5-dihydroxy-4-methoxybenzoate, (2R,3R)-5,7-dihydroxy-2-(3,4,5-trihydroxyphenyl)chroman-3-yl 3,4,5-trihydroxybenzoate, (2S,3R)-5,7-dihydroxy-2-(3,4,5-trihydroxyphenyl)chroman-3-yl 3,4,5-trihydroxybenzoate, (2R,3S)-5,7-dihydroxy-2-(3,4,5-trihydroxyphenyl)chroman-3-yl 3,4,5-trihydroxybenzoate, (2R,3R)-2-(3,4-dihydroxyphenyl)-5,7-dihydroxychroman-3-yl 3,4,5-trihydroxybenzoate, (2S,3R)-2-(3,4-dihydroxyphenyl)-5,7-dihydroxychroman-3-yl 3,4,5-trihydroxybenzoate, (2R,3R)-5,7-dihydroxy-2-(3,4,5-trihydroxyphenyl)chroman-3-yl 3,4-dihydroxy-5-methoxybenzoate; (2R,3S)-5,7-dihydroxy-2-(3,4,5-trihydroxyphenyl)chroman-3-yl 3,4-difluorobenzoate; (2S,3R)-5,7-dihydroxy-2-(3,4,5-trihydroxyphenyl)chroman-3-yl 2,3,4-trihydroxybenzoate.

In certain embodiments as otherwise described herein, the compound is of Formula I-A or Formula I-B

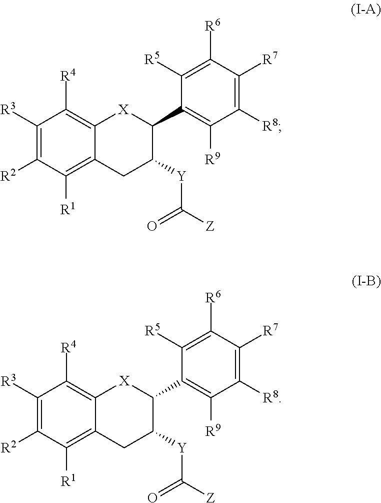

In certain embodiments as otherwise described herein, X is O. In certain embodiments as otherwise described herein, Y is O. For example, in certain desirable embodiments as otherwise described herein, X and Y are each O.

In certain embodiments as otherwise described herein, R¹, R², R³, and R⁴ are each independently hydrogen, —OH, C₁-C₁₀ alkoxy, —OC(O)(C₁-C₁₀ alkyl), or —OC(O)NH(C₁-C₁₀ alkyl). For example, in certain embodiments as otherwise described herein, R¹ and R³ are each independently —OH, C₁-C₁₀ alkoxy, —OC(O)(C₁-C₁₀ alkyl) (e.g., —OC(O)(C₁-C₆ alkyl)), or —OC(O)NH(C₁-C₁₀ alkyl) (e.g., —OC(O)NH(C₁-C₆ alkyl)); and R² and R⁴ are each hydrogen. In certain such embodiments, R¹ is —OH, R³ is —OC(O)(C₁-C₁₀ alkyl) (e.g., —OC(O)(C₁-C₆ alkyl)) or —OC(O)NH(C₁-C₁₀ alkyl) (e.g., —OC(O)NH(C₁-C₆ alkyl)), and R² and R⁴ are each hydrogen. In certain desirable embodiments as otherwise described herein, R¹ and R³ are each independently —OH; and R² and R⁴ are each hydrogen. In certain such embodiments, the compound is of Formula I-A or Formula I-B.

In certain embodiments as otherwise described herein, R⁵ and R⁹ are each independently hydrogen, —F, or —OH. For example, in certain embodiments as otherwise described herein, R⁵ and R⁹ are each independently hydrogen or —F; and at least one of R⁵ and R⁹ is hydrogen. In certain such embodiments, R⁵ is —F and R⁹ is hydrogen. In certain such embodiments, R⁵ and R⁹ are each hydrogen.

In certain embodiments as otherwise described herein, R⁷ is —OH, C₁-C₁₀ alkoxy, —CONH₂, —CONH(C₁-C₁₀ alkyl), —CO(C₁-C₁₀ alkyl), or —NH(S(O)₀₋₂(C₁-C₁₀ alkyl)). For example, in certain embodiments as otherwise described herein, R⁷ is —OH, C₁-C₁₀ alkoxy (e.g., C₁-C₄ alkoxy), or —CONH(C₁-C₁₀ alkyl) (e.g., —CONH(C₁-C₄ alkyl)). In certain such embodiments, R⁷ is C₁-C₄ alkoxy (e.g., methoxy) or —CONH(C₁-C₄ alkyl). In certain desirable embodiments as otherwise described herein, R⁷ is —OH.

In certain embodiments as otherwise described herein, R⁶ and R⁸ are each independently hydrogen, —OH, C₁-C₆ alkoxy, or C₁-C₆ haloalkoxy. For example, in certain embodiments as otherwise described herein, R⁶ and R⁸ are each independently hydrogen or —OH; and at least one of R⁶ and R⁸ is —OH. In certain desirable embodiments as otherwise described herein, R⁶ is hydrogen and R⁸ is —OH. In certain desirable embodiments as otherwise described herein, R⁶ and R⁸ are each —OH. In certain such embodiments, R⁷ is —OH.

For example, in certain embodiments as otherwise described herein, R⁵ and R⁹ are each independently hydrogen or —F; R⁷ is —OH; R⁶ and R⁸ are each independently hydrogen or —OH; at least one of R⁵ and R⁹ is hydrogen; and at least one of R⁶ and R⁸ is —OH. In certain such embodiments, R⁵ is —F and R⁹ is hydrogen. In certain such embodiments, R⁵ and R⁹ are each hydrogen. In certain such embodiments, R⁶ is hydrogen and R⁸ is —OH. In certain such embodiments, R⁶ and R⁸ are each —OH. In certain such embodiments, R¹ and R³ are each independently —OH; and R² and R⁴ are each hydrogen. In certain such embodiments, the compound is of Formula I-A.

In certain embodiments as otherwise described herein, Z is

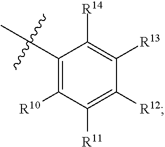

R¹⁰ and R¹⁴ are each independently hydrogen, —F, or —OH; R¹² is —OH, C₁-C₁₀ alkoxy, —CONH₂, —CONH(C₁-C₁₀ alkyl), —CO(C₁-C₁₀ alkyl), or —NH(S(O)₀₋₂(C₁-C₁₀ alkyl)); and R¹¹ and R¹³ are each independently hydrogen, —OH, C₁-C₆ alkoxy, or C₁-C₆ haloalkoxy. In certain embodiments as otherwise described herein, at least one of R¹⁰ and R¹⁴ is —F. In certain embodiments as otherwise described herein, R¹² is —OH. In certain embodiments as otherwise described herein, at least one of R¹¹ and R¹³ is —OH, C₁-C₆ alkoxy, or C₁-C₆ haloalkoxy. In certain embodiments as otherwise described herein, R¹⁰, R¹¹, and R¹² are each —OH. In certain embodiments as otherwise described herein, R¹³ and R¹⁴ are each hydrogen. In certain embodiments as otherwise described herein, R¹³ and R¹⁴ are each —OH.

For example, in certain embodiments as otherwise described herein, the compound is of Formula II

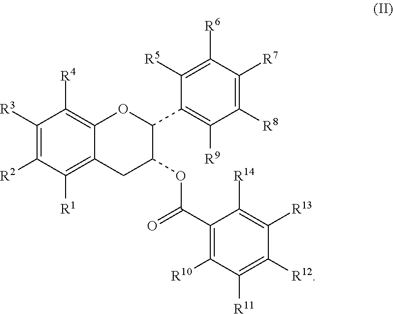

In certain desirable embodiments as otherwise described herein, the compound is of Formula II-A or Formula II-B

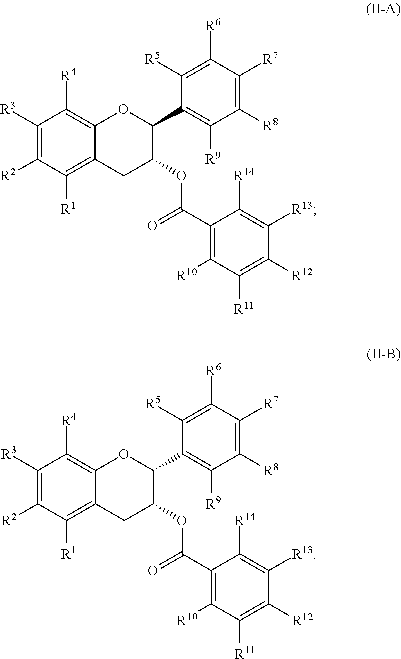

In certain embodiments as otherwise described herein, R¹ and R³ are each —OH; and R² and R⁴ are each hydrogen. Accordingly, in certain embodiments as otherwise described herein, the compound is of Formula III, Formula III-A, or Formula III-B

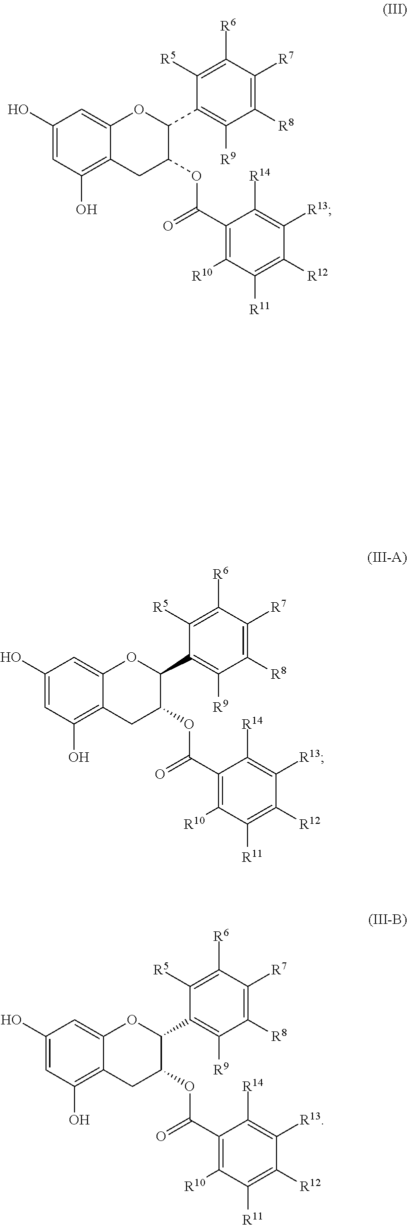

Surprisingly, the present inventors have determined that in certain such embodiments (e.g., compounds of Formula II, II-A, II-B, III, III-A, and III-B), the identities of substituents R⁵-R⁹ and R¹⁰-R¹⁴ can be interchangeable with respect to the inhibitory properties of the compounds towards DYRK1A. That is, the present inventors note that certain desirable configurations of R¹⁰-R¹⁴ identified below could, in certain embodiments, be similarly useful as a corresponding configuration of R⁵-R¹⁰.

Accordingly, in certain embodiments as otherwise described herein, at least one (e.g., one or two) of R⁵, R⁹, R¹⁰, and R¹⁴ is not hydrogen. In certain embodiments as otherwise described herein, R⁵, R⁹, R¹⁰, and R¹⁴ are each independently hydrogen, halogen, or —OH.

Advantageously, the present inventors have determined that a compound of Formula II (e.g., Formula II-A, Formula III, Formula III-A) substituted with a fluorine atom at one or more of R⁵, R⁹, R¹⁰, and R¹⁴, can have significantly improved inhibitory properties (e.g., a 2-3 fold improvement in activity towards DYRK1A, relative a corresponding compound lacking the fluorine substitution). Accordingly, in certain embodiments as otherwise described herein, at least one (e.g., one or two) of R⁵, R⁹, R¹⁰, and R¹⁴ is —F. In certain desirable embodiments as otherwise described herein, R⁵, R⁹, R¹⁰, and R¹⁴ are each independently hydrogen or —F, and one or two of R⁵, R⁹, R¹⁰, and R¹⁴ are —F.

In certain embodiments as otherwise described herein, R⁷ and R¹² are each independently —OH, C₁-C₁₀ alkoxy, —CONH₂, —CONH(C₁-C₁₀ alkyl), —CO(C₁-C₁₀ alkyl), or —NH(S(O)₀₋₂(C₁-C₁₀ alkyl)). For example, in certain embodiments as otherwise described herein, R⁷ is —OH or C₁-C₁₀ alkoxy (e.g., C₁-C₄ alkoxy). In certain such embodiments, R⁷ is —OH. In other such embodiments, R⁷ is C₁-C₄ alkoxy (e.g., methoxy). In certain desirable embodiments as otherwise described herein, R⁷ and R¹² are each independently —OH or C₁-C₁₀ alkoxy (e.g., C₁-C₄ alkoxy).

In certain embodiments as otherwise described herein, R⁶, R⁸, R¹¹, and R¹³ are each independently hydrogen, —OH, C₁-C₆ alkoxy, or C₁-C₆ haloalkoxy. For example, in certain embodiments as otherwise described herein, at least one (e.g., one or two) of R⁶, R⁸, R¹¹, and R¹³ is C₁-C₆ alkoxy (e.g., C₁-C₄ alkoxy) or C₁-C₆ haloalkoxy (e.g., C₁-C₄ haloalkoxy). In certain such embodiments, one or two of R⁶, R⁸, R¹¹, and R¹³ is C₁-C₄ alkoxy (e.g., methoxy).

In certain embodiments as otherwise described herein, R⁷, R⁸, R¹¹, and R¹² are each —OH; and R¹³ is —OH, C₁-C₆ alkoxy (e.g., C₁-C₄ alkoxy), or C₁-C₆ haloalkoxy (e.g., C₁-C₄ haloalkoxy). In certain such embodiments, R¹³ is —OH. In other such embodiments, R¹³ is C₁-C₄ alkoxy (e.g., methoxy).

Advantageously, the present inventors have determined that the inhibitory potency of compounds described herein (e.g., compounds of Formula II, II-A, III, or III-A) towards inhibition of DYRK1A is surprisingly high where at least one of R⁵-R⁹ and R¹⁰-R¹⁴ include a fluorine substituent positioned para to an oxy substituent such as, for example, —OH, alkoxy, or haloalkoxy. Accordingly, in certain desirable embodiments as otherwise described herein, at least one of R⁵, R⁹, R¹⁰, and R¹⁴ is —F, positioned para to a substituent selected from —OH, C₁-C₆ alkoxy, or C₁-C₆ haloalkoxy. In certain such embodiments, at least one of R⁵, R⁹, R¹⁰, and R¹⁴ is —F, positioned para to a substituent selected from —OH or C₁-C₆ alkoxy (e.g., C₁-C₄ alkoxy). For example, in certain embodiments as otherwise described herein, R¹³ is —OH, C₁-C₆ alkoxy (e.g., C₁-C₄ alkoxy), or C₁-C₆ haloalkoxy (e.g., C₁-C₄ haloalkoxy); and R¹⁰ is —F. In certain such embodiments, R¹³ is —OH. In other such embodiments, R¹³ is C₁-C₄ alkoxy (e.g., methoxy).

In certain embodiments as otherwise described herein, R¹, R³, R⁷, R⁸, R¹¹, and R¹² are each —OH; R², R⁴, and R⁹ are each hydrogen; R⁶ and R¹³ are each —OH, C₁-C₆ alkoxy (e.g., C₁-C₄ alkoxy), or C₁-C₆ haloalkoxy (e.g., C₁-C₄ haloalkoxy); and at least one of R⁵, R¹⁰, and R¹⁴ is —F. In certain such embodiments, R⁶ and R¹³ are each —OH or C₁-C₄ alkoxy (e.g., methoxy). In certain such embodiments, R⁶ is —OH and R¹³ is C₁-C₄ alkoxy (e.g., methoxy). In certain embodiments as otherwise described herein, R⁵ and R¹⁴ are each hydrogen, and R¹⁰ is —F. In certain such embodiments, the compound is of Formula III-A or Formula III-B.

In certain embodiments as otherwise described herein, R¹, R³, R⁷, and R¹² are each —OH; R², R⁴, and R⁹ are each hydrogen; R⁵, R¹³, and R¹⁴ are each independently hydrogen or —F; R⁶ and R⁸ are each independently hydrogen or —OH; and R¹⁰ and R¹¹ are each independently hydrogen, —OH, C₁-C₆ alkoxy (e.g., C₁-C₄ alkoxy), or C₁-C₆ haloalkoxy (e.g., C₁-C₄ haloalkoxy). In certain such embodiments, R⁵ is hydrogen and R¹⁴ is —F. In other such embodiments, R⁵ and R¹⁴ are each hydrogen. In certain such embodiments, R⁶ is hydrogen and R⁸ is —OH. In other such embodiments, R⁶ and R⁸ are each —OH. In certain such embodiments, R¹³ is hydrogen, and R¹⁰ and R¹¹ are each independently —OH or C₁-C₄ alkoxy (e.g., methoxy). In certain such embodiments, the compound is of Formula III-A or Formula III-B.

In certain embodiments as otherwise described herein, R¹, R³, R⁶, R⁷, R⁸, R¹⁰, R¹¹, and R¹² are each —OH; and R², R⁴, R⁵, R⁹, R¹³, and R¹⁴ are each individually hydrogen or —F. In certain such embodiments, R², R⁴, R⁵, R⁹, R¹³, and R¹⁴ are each hydrogen.

In certain embodiments as otherwise described herein, Z is

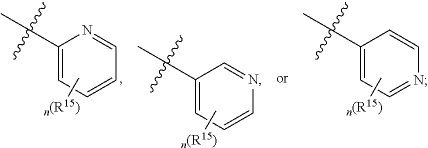

n is 0-2; and each R¹⁵ is independently —NH₂, —OH, or C₁-C₆ alkoxy. For example, in certain embodiments as otherwise described herein, Z is

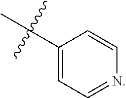 [SMILES: *c1ccncc1]

In another example, in certain embodiments as otherwise described herein, Z is

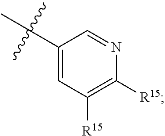

and each R¹⁵ is independently —NH₂ or —OH. In another example, in certain embodiments as otherwise described herein, Z is

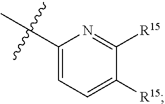

and each R¹⁵ is independently —NH₂ or —OH. In certain such embodiments, R¹ and R³ are each —OH; and R² and R⁴ are each hydrogen. In certain such embodiments, R⁵ and R⁹ are each independently hydrogen or —F; R⁷ is —OH; R⁶ and R⁸ are each independently hydrogen or —OH; at least one of R⁵ and R⁹ is hydrogen; and at least one of R⁶ and R⁸ is —OH. In certain such embodiments, the compound is of Formula I-A.

In certain embodiments as otherwise described herein, Z is

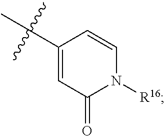

and R¹⁶ is hydrogen or —OH. In certain such embodiments, R¹ and R³ are each —OH; and R² and R⁴ are each hydrogen. In certain such embodiments, R⁵ and R⁹ are each independently hydrogen or —F; R⁷ is —OH; R⁶ and R⁸ are each independently hydrogen or —OH; at least one of R⁵ and R⁹ is hydrogen; and at least one of R⁶ and R⁸ is —OH. In certain such embodiments, the compound is of Formula I-A.

In certain embodiments as otherwise described herein, Z is

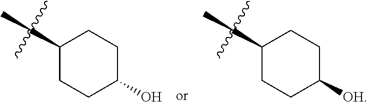 [SMILES: *[C@H]1CC[C@H](O)CC1 ; "or" ; *[C@H]1CC[C@@H](O)CC1]

In certain such embodiments, R¹ and R³ are each —OH; and R² and R⁴ are each hydrogen. In certain such embodiments, R⁵ and R⁹ are each independently hydrogen or —F; R⁷ is —OH; R⁶ and R⁸ are each independently hydrogen or —OH; at least one of R⁵ and R⁹ is hydrogen; and at least one of R⁶ and R⁸ is —OH. In certain such embodiments, the compound is of Formula I-A.

Certain compounds of Formula I are provided in Table 1.

TABLE 1

| No. | Structure | Compound |
|---|---|---|
| 1 | 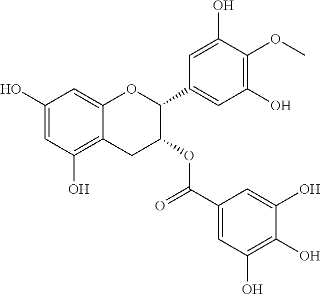 [SMILES: COc1c(O)cc([C@H]2Oc3cc(O)cc(O)c3C[C@H]2OC(=O)c2cc(O)c(O)c(O)c2)cc1O] | (2R,3R)-2-(3,5-dihydroxy-4- methoxyphenyl)-5,7- dihydroxychroman-3-yl 3,4,5- trihydroxybenzoate |
| 2 | 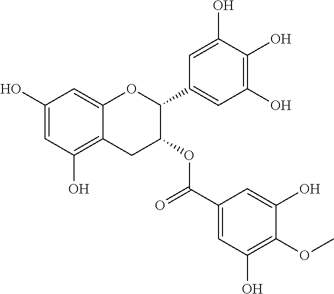 [SMILES: COc1c(O)cc(C(=O)O[C@@H]2Cc3c(O)cc(O)cc3O[C@@H]2c2cc(O)c(O)c(O)c2)cc1O] | (2R,3R)-5,7-dihydroxy-2-(3,4,5- trihydroxyphenyl)chroman-3-yl 3,5-dihydroxy-4- methoxybenzoate |
| 3 | 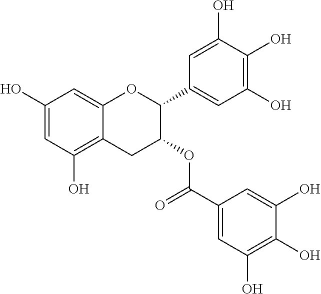 [SMILES: O=C(O[C@@H]1Cc2c(O)cc(O)cc2O[C@@H]1c1cc(O)c(O)c(O)c1)c1cc(O)c(O)c(O)c1] | (2R,3R)-5,7-dihydroxy-2-(3,4,5- trihydroxyphenyl)chroman-3-yl 3,4,5-trihydroxybenzoate |
| 4 | 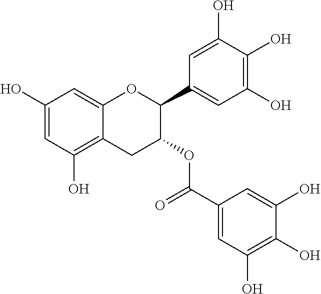 [SMILES: O=C(O[C@@H]1Cc2c(O)cc(O)cc2O[C@H]1c1cc(O)c(O)c(O)c1)c1cc(O)c(O)c(O)c1] | (2S,3R)-5,7-dihydroxy-2-(3,4,5- trihydroxyphenyl)chroman-3-yl 3,4,5-trihydroxybenzoate |
| 5 | 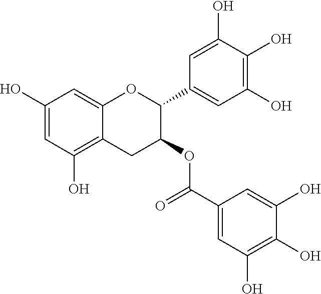 [SMILES: O=C(O[C@H]1Cc2c(O)cc(O)cc2O[C@@H]1c1cc(O)c(O)c(O)c1)c1cc(O)c(O)c(O)c1] | (2R,3S)-5,7-dihydroxy-2-(3,4,5- trihydroxyphenyl)chroman-3-yl 3,4,5-trihydroxybenzoate |
| 6 | 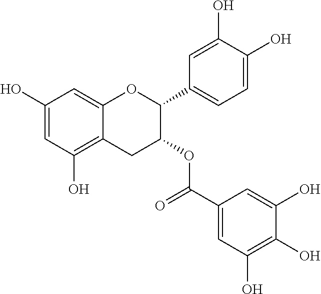 [SMILES: O=C(O[C@@H]1Cc2c(O)cc(O)cc2O[C@@H]1c1ccc(O)c(O)c1)c1cc(O)c(O)c(O)c1] | (2R,3R)-2-(3,4-dihydroxyphenyl)- 5,7-dihydroxychroman-3-yl 3,4,5- trihydroxybenzoate |
| 7 | 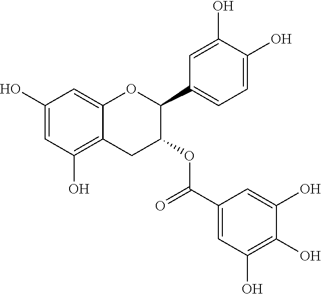 [SMILES: O=C(O[C@@H]1Cc2c(O)cc(O)cc2O[C@H]1c1ccc(O)c(O)c1)c1cc(O)c(O)c(O)c1] | (2S,3R)-2-(3,4-dihydroxyphenyl)- 5,7-dihydroxychroman-3-yl 3,4,5- trihydroxybenzoate |
| 8 | 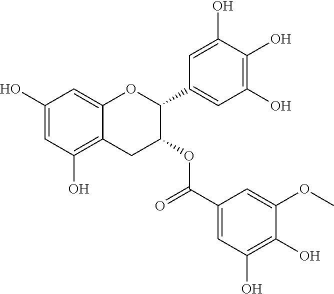 [SMILES: COc1cc(C(=O)O[C@@H]2Cc3c(O)cc(O)cc3O[C@@H]2c2cc(O)c(O)c(O)c2)cc(O)c1O] | (2R,3R)-5,7-dihydroxy-2-(3,4,5- trihydroxyphenyl)chroman-3-yl 3,4-dihydroxy-5- methoxybenzoate |
| 9 | 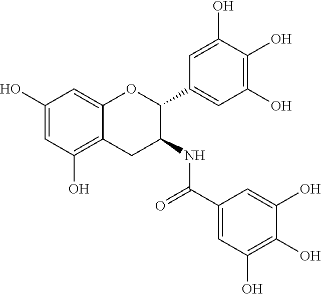 [SMILES: O=C(N[C@H]1Cc2c(O)cc(O)cc2O[C@@H]1c1cc(O)c(O)c(O)c1)c1cc(O)c(O)c(O)c1] | N-((2R,3S)-5,7-dihydroxy-2- (3,4,5-trihydroxyphenyl)chroman- 3-yl)-3,4,5-trihydroxybenzamide |
| 10 | 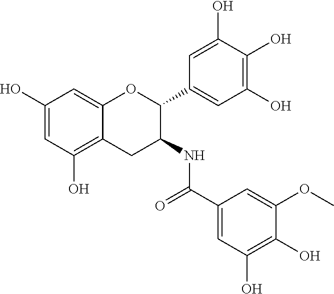 [SMILES: COc1cc(C(=O)N[C@H]2Cc3c(O)cc(O)cc3O[C@@H]2c2cc(O)c(O)c(O)c2)cc(O)c1O] | N-((2R,3S)-5,7-dihydroxy-2- (3,4,5-trihydroxyphenyl)chroman- 3-yl)-3,4-dihydroxy-5- methoxybenzamide |
| 11 | 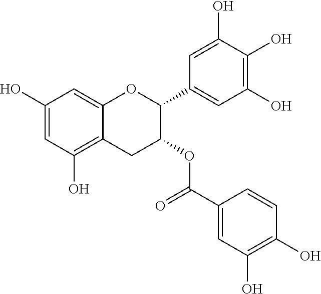 [SMILES: O=C(O[C@@H]1Cc2c(O)cc(O)cc2O[C@@H]1c1cc(O)c(O)c(O)c1)c1ccc(O)c(O)c1] | (2R,3R)-5,7-dihydroxy-2-(3,4,5- trihydroxyphenyl)chroman-3-yl 3,4-dihydroxybenzoate |
| 12 | 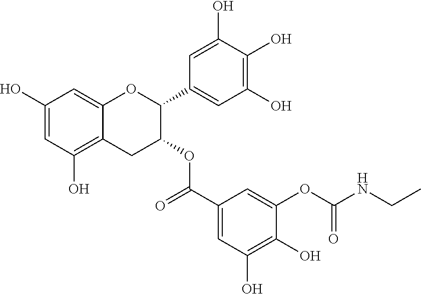 [SMILES: CCNC(=O)Oc1cc(C(=O)O[C@@H]2Cc3c(O)cc(O)cc3O[C@@H]2c2cc(O)c(O)c(O)c2)cc(O)c1O] | (2R,3R)-5,7-dihydroxy-2-(3,4,5- trihydroxyphenyl)chroman-3-yl 3- ((ethylcarbamoyl)oxy)-4,5- dihydroxybenzoate |
| 13 | 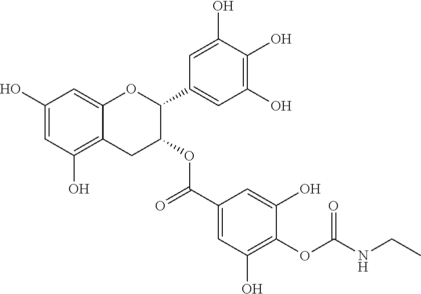 [SMILES: CCNC(=O)Oc1c(O)cc(C(=O)O[C@@H]2Cc3c(O)cc(O)cc3O[C@@H]2c2cc(O)c(O)c(O)c2)cc1O] | (2R,3R)-5,7-dihydroxy-2-(3,4,5- trihydroxyphenyl)chroman-3-yl 4- ((ethylcarbamoyl)oxy)-3,5- dihydroxybenzoate |
| 14 | 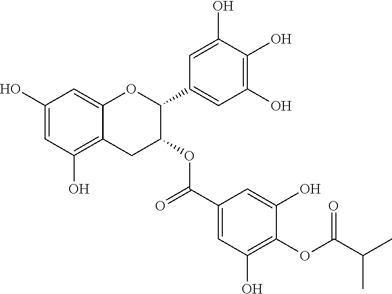 [SMILES: CC(C)C(=O)Oc1c(O)cc(C(=O)O[C@@H]2Cc3c(O)cc(O)cc3O[C@@H]2c2cc(O)c(O)c(O)c2)cc1O] | (2R,3R)-5,7-dihydroxy-2-(3,4,5- trihydroxyphenyl)chroman-3-yl 3,5-dihydroxy-4- (isobutyryloxy)benzoate |
| 15 | 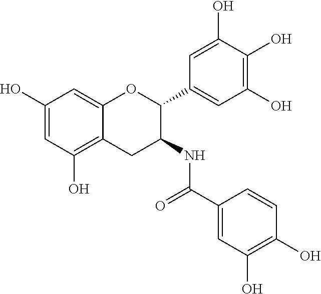 [SMILES: O=C(N[C@H]1Cc2c(O)cc(O)cc2O[C@@H]1c1cc(O)c(O)c(O)c1)c1ccc(O)c(O)c1] | N-((2R,3S)-5,7-dihydroxy-2- (3,4,5-trihydroxyphenyl)chroman- 3-yl)-3,4-dihydroxybenzamide |
| 16 | 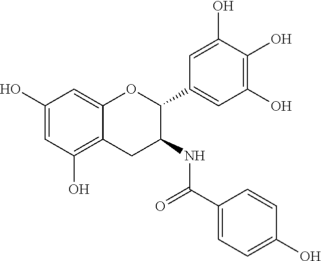 [SMILES: O=C(N[C@H]1Cc2c(O)cc(O)cc2O[C@@H]1c1cc(O)c(O)c(O)c1)c1ccc(O)cc1] | N-((2R,3S)-5,7-dihydroxy-2- (3,4,5-trihydroxyphenyl)chroman- 3-yl)-4-hydroxybenzamide |
| 17 | 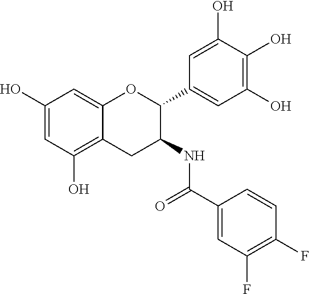 [SMILES: O=C(N[C@H]1Cc2c(O)cc(O)cc2O[C@@H]1c1cc(O)c(O)c(O)c1)c1ccc(F)c(F)c1] | N-((2R,3S)-5,7-dihydroxy-2- (3,4,5-trihydroxyphenyl)chroman- 3-yl)-3,4-difluorobenzamide |
| 18 | 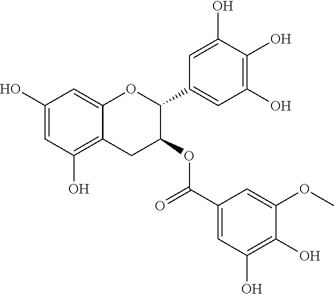 [SMILES: COc1cc(C(=O)O[C@H]2Cc3c(O)cc(O)cc3O[C@@H]2c2cc(O)c(O)c(O)c2)cc(O)c1O] | (2R,3S)-5,7-dihydroxy-2-(3,4,5- trihydroxyphenyl)chroman-3-yl 3,4-dihydroxy-5- methoxybenzoate |
| 19 | 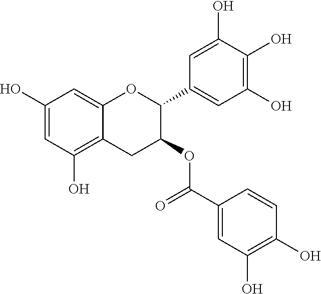 [SMILES: O=C(O[C@H]1Cc2c(O)cc(O)cc2O[C@@H]1c1cc(O)c(O)c(O)c1)c1ccc(O)c(O)c1] | (2R,3S)-5,7-dihydroxy-2-(3,4,5- trihydroxyphenyl)chroman-3-yl 3,4-dihydroxybenzoate |
| 20 | 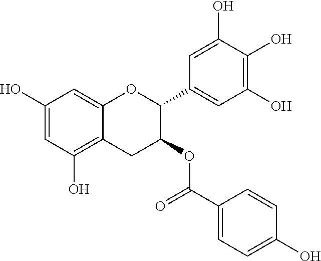 [SMILES: O=C(O[C@H]1Cc2c(O)cc(O)cc2O[C@@H]1c1cc(O)c(O)c(O)c1)c1ccc(O)cc1] | (2R,3S)-5,7-dihydroxy-2-(3,4,5- trihydroxyphenyl)chroman-3-yl 4- hydroxybenzoate |
| 21 | 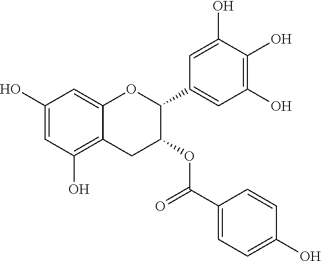 [SMILES: O=C(O[C@@H]1Cc2c(O)cc(O)cc2O[C@@H]1c1cc(O)c(O)c(O)c1)c1ccc(O)cc1] | (2R,3R)-5,7-dihydroxy-2-(3,4,5- trihydroxyphenyl)chroman-3-yl 4- hydroxybenzoate |
| 22 | 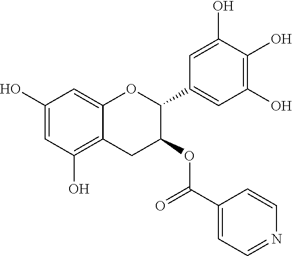 [SMILES: O=C(O[C@H]1Cc2c(O)cc(O)cc2O[C@@H]1c1cc(O)c(O)c(O)c1)c1ccncc1] | (2R,3S)-5,7-dihydroxy-2-(3,4,5- trihydroxyphenyl)chroman-3-yl isonicotinate |
| 23 | 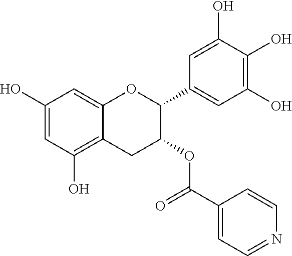 [SMILES: O=C(O[C@@H]1Cc2c(O)cc(O)cc2O[C@@H]1c1cc(O)c(O)c(O)c1)c1ccncc1] | (2R,3R)-5,7-dihydroxy-2-(3,4,5- trihydroxyphenyl)chroman-3-yl isonicotinate |
| 24 | 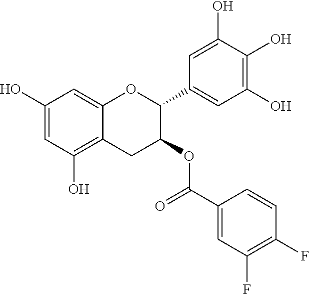 [SMILES: O=C(O[C@H]1Cc2c(O)cc(O)cc2O[C@@H]1c1cc(O)c(O)c(O)c1)c1ccc(F)c(F)c1] | (2R,3S)-5,7-dihydroxy-2-(3,4,5- trihydroxyphenyl)chroman-3-yl 3,4-difluorobenzoate |
| 25 | 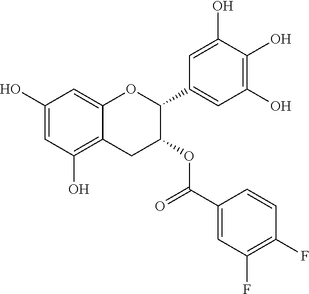 [SMILES: O=C(O[C@@H]1Cc2c(O)cc(O)cc2O[C@@H]1c1cc(O)c(O)c(O)c1)c1ccc(F)c(F)c1] | (2R,3R)-5,7-dihydroxy-2-(3,4,5- trihydroxyphenyl)chroman-3-yl 3,4-difluorobenzoate |
| 26 | 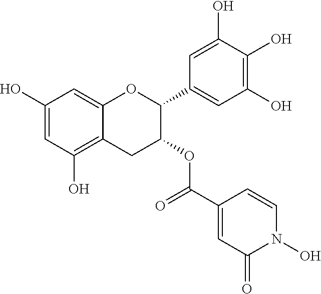 [SMILES: O=C(O[C@@H]1Cc2c(O)cc(O)cc2O[C@@H]1c1cc(O)c(O)c(O)c1)c1ccn(O)c(=O)c1] | (2R,3R)-5,7-dihydroxy-2-(3,4,5- trihydroxyphenyl)chroman-3-yl 1- hydroxy-2-oxo-1,2- dihydropyridine-4-carboxylate |
| 27 | 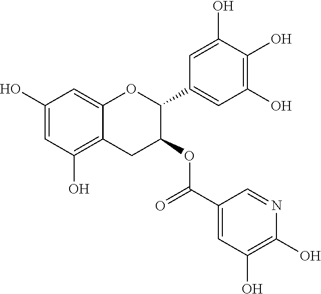 [SMILES: O=C(O[C@H]1Cc2c(O)cc(O)cc2O[C@@H]1c1cc(O)c(O)c(O)c1)c1cnc(O)c(O)c1] | (2R,3S)-5,7-dihydroxy-2-(3,4,5- trihydroxyphenyl)chroman-3-yl 5,6-dihydroxynicotinate |
| 28 | 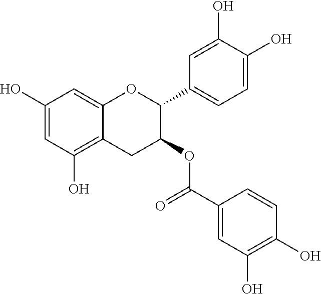 [SMILES: O=C(O[C@H]1Cc2c(O)cc(O)cc2O[C@@H]1c1ccc(O)c(O)c1)c1ccc(O)c(O)c1] | (2R,3S)-2-(3,4-dihydroxyphenyl)- 5,7-dihydroxychroman-3-yl 3,4- dihydroxybenzoate |
| 29 | 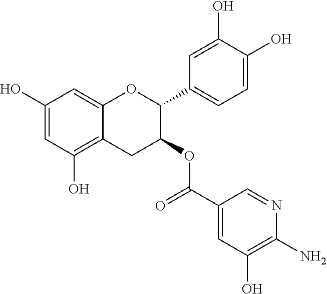 [SMILES: Nc1ncc(C(=O)O[C@H]2Cc3c(O)cc(O)cc3O[C@@H]2c2ccc(O)c(O)c2)cc1O] | (2R,3S)-2-(3,4-dihydroxyphenyl)- 5,7-dihydroxychroman-3-yl 6- amino-5-hydroxynicotinate |
| 30 | 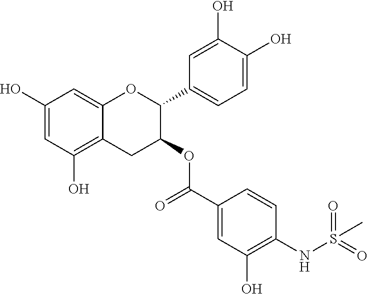 [SMILES: CS(=O)(=O)Nc1ccc(C(=O)O[C@H]2Cc3c(O)cc(O)cc3O[C@@H]2c2ccc(O)c(O)c2)cc1O] | (2R,3S)-2-(3,4-dihydroxyphenyl)- 5,7-dihydroxychroman-3-yl 3- hydroxy-4- (methylsulfonamido)benzoate |
| 31 | 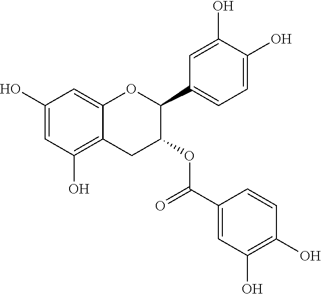 [SMILES: O=C(O[C@@H]1Cc2c(O)cc(O)cc2O[C@H]1c1ccc(O)c(O)c1)c1ccc(O)c(O)c1] | (2S,3R)-2-(3,4-dihydroxyphenyl)- 5,7-dihydroxychroman-3-yl 3,4- dihydroxybenzoate |
| 32 | 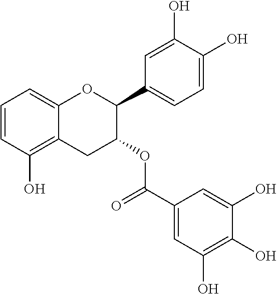 [SMILES: O=C(O[C@@H]1Cc2c(O)cccc2O[C@H]1c1ccc(O)c(O)c1)c1cc(O)c(O)c(O)c1] | (2S,3R)-5-hydroxy-2-(3,4,5- trihydroxyphenyl)chroman-3-yl 3,4,5-trihydroxybenzoate |
| 33 | 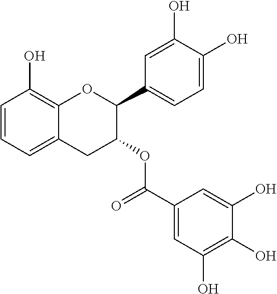 [SMILES: O=C(O[C@@H]1Cc2cccc(O)c2O[C@H]1c1ccc(O)c(O)c1)c1cc(O)c(O)c(O)c1] | (2R,3S)-5-hydroxy-3-(3,4,5- trihydroxyphenyl)-1,2,3,4- tetrahydronaphthalen-2-yl 3,4,5- trihydroxybenzoate |
| 34 | 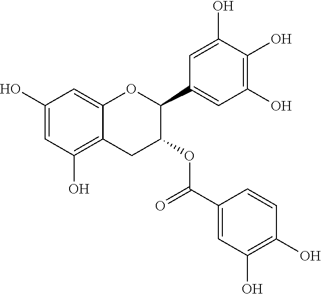 [SMILES: O=C(O[C@@H]1Cc2c(O)cc(O)cc2O[C@H]1c1cc(O)c(O)c(O)c1)c1ccc(O)c(O)c1] | (2S,3R)-5,7-dihydroxy-2-(3,4,5- trihydroxyphenyl)chroman-3-yl 3,4-dihydroxybenzoate |
| 35 | 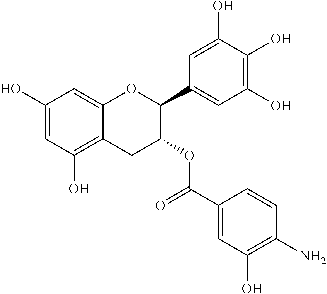 [SMILES: Nc1ccc(C(=O)O[C@@H]2Cc3c(O)cc(O)cc3O[C@H]2c2cc(O)c(O)c(O)c2)cc1O] | (2S,3R)-5,7-dihydroxy-2-(3,4,5- trihydroxyphenyl)chroman-3-yl 4- amino-3-hydroxybenzoate |
| 36 | 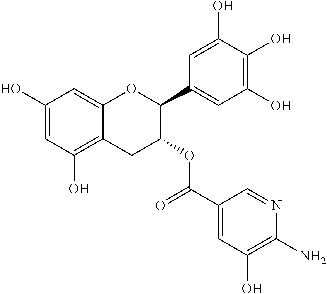 [SMILES: Nc1ncc(C(=O)O[C@@H]2Cc3c(O)cc(O)cc3O[C@H]2c2cc(O)c(O)c(O)c2)cc1O] | (2S,3R)-5,7-dihydroxy-2-(3,4,5- trihydroxyphenyl)chroman-3-yl 6- amino-5-hydroxynicotinate |
| 37 | 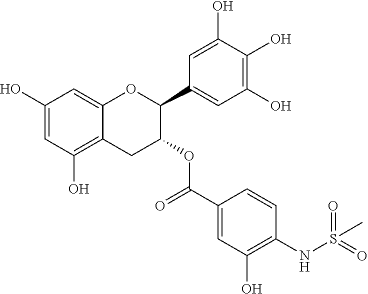 [SMILES: CS(=O)(=O)Nc1ccc(C(=O)O[C@@H]2Cc3c(O)cc(O)cc3O[C@H]2c2cc(O)c(O)c(O)c2)cc1O] | (2S,3R)-5,7-dihydroxy-2-(3,4,5- trihydroxyphenyl)chroman-3-yl 3- hydroxy-4- (methylsulfonamido)benzoate |
| 38 | 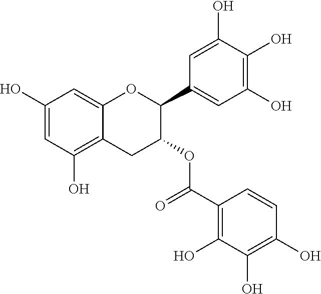 [SMILES: O=C(O[C@@H]1Cc2c(O)cc(O)cc2O[C@H]1c1cc(O)c(O)c(O)c1)c1ccc(O)c(O)c1O] | (2S,3R)-5,7-dihydroxy-2-(3,4,5- trihydroxyphenyl)chroman-3-yl 2,3,4-trihydroxybenzoate |
| 39 | 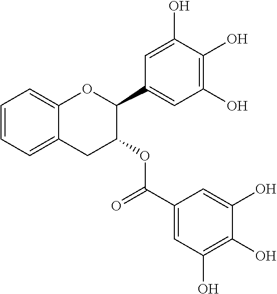 [SMILES: O=C(O[C@@H]1Cc2ccccc2O[C@H]1c1cc(O)c(O)c(O)c1)c1cc(O)c(O)c(O)c1] | (2S,3R)-2-(3,4,5- trihydroxyphenyl)chroman-3-yl 3,4,5-trihydroxybenzoate |
| 40 | 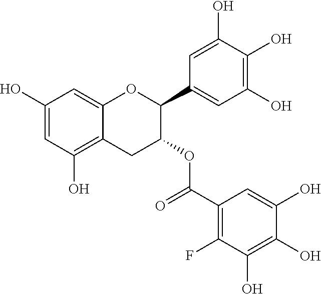 [SMILES: O=C(O[C@@H]1Cc2c(O)cc(O)cc2O[C@H]1c1cc(O)c(O)c(O)c1)c1cc(O)c(O)c(O)c1F] | (2S,3R)-5,7-dihydroxy-2-(3,4,5- trihydroxyphenyl)chroman-3-yl 2- fluoro-3,4,5-trihydroxybenzoate |
| 41 | 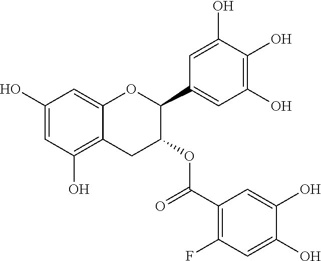 [SMILES: O=C(O[C@@H]1Cc2c(O)cc(O)cc2O[C@H]1c1cc(O)c(O)c(O)c1)c1cc(O)c(O)cc1F] | (2S,3R)-5,7-dihydroxy-2-(3,4,5- trihydroxyphenyl)chroman-3-yl 2- fluoro-4,5-dihydroxybenzoate |
| 42 | 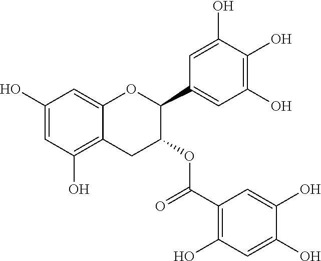 [SMILES: O=C(O[C@@H]1Cc2c(O)cc(O)cc2O[C@H]1c1cc(O)c(O)c(O)c1)c1cc(O)c(O)cc1O] | (2S,3R)-5,7-dihydroxy-2-(3,4,5- trihydroxyphenyl)chroman-3-yl 2,4,5-trihydroxybenzoate |
| 43 | 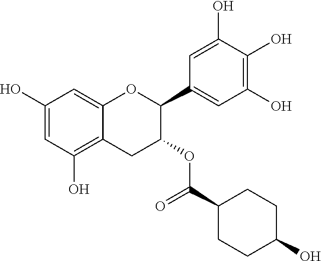 [SMILES: O=C(O[C@@H]1Cc2c(O)cc(O)cc2O[C@H]1c1cc(O)c(O)c(O)c1)[C@H]1CC[C@@H](O)CC1] | (2S,3R)-5,7-dihydroxy-2-(3,4,5- trihydroxyphenyl)chroman-3-yl (1s,4S)-4-hydroxycyclohexane-1- carboxylate |
| 44 | 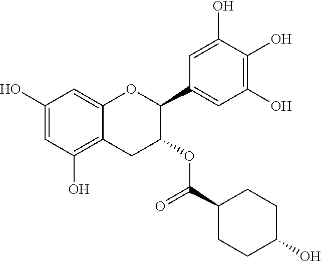 [SMILES: O=C(O[C@@H]1Cc2c(O)cc(O)cc2O[C@H]1c1cc(O)c(O)c(O)c1)[C@H]1CC[C@H](O)CC1] | (2S,3R)-5,7-dihydroxy-2-(3,4,5- trihydroxyphenyl)chroman-3-yl (1r,4R)-4-hydroxycyclohexane-1- carboxylate |
| 45 | 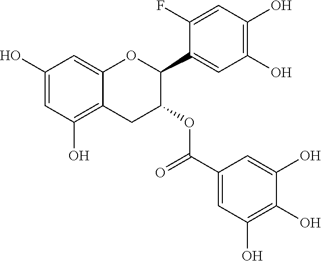 [SMILES: O=C(O[C@@H]1Cc2c(O)cc(O)cc2O[C@H]1c1cc(O)c(O)cc1F)c1cc(O)c(O)c(O)c1] | (2S,3R)-2-(2-fluoro-4,5- dihydroxyphenyl)-5,7- dihydroxychroman-3-yl 3,4,5- trihydroxybenzoate |
| 46 | 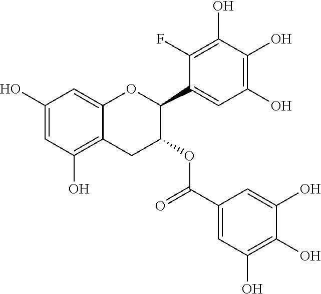 [SMILES: O=C(O[C@@H]1Cc2c(O)cc(O)cc2O[C@H]1c1cc(O)c(O)c(O)c1F)c1cc(O)c(O)c(O)c1] | (2S,3R)-2-(2-fluoro-3,4,5- trihydroxyphenyl)-5,7- dihydroxychroman-3-yl 3,4,5- trihydroxybenzoate |
| 47 | 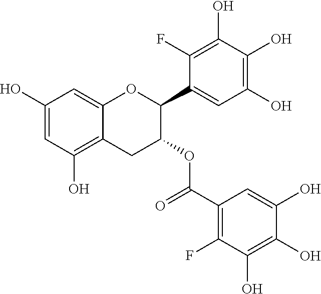 [SMILES: O=C(O[C@@H]1Cc2c(O)cc(O)cc2O[C@H]1c1cc(O)c(O)c(O)c1F)c1cc(O)c(O)c(O)c1F] | (2S,3R)-2-(2-fluoro-3,4,5- trihydroxyphenyl)-5,7- dihydroxychroman-3-yl 2-fluoro- 3,4,5-trihydroxybenzoate |
| 48 | 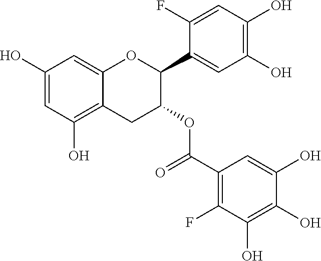 [SMILES: O=C(O[C@@H]1Cc2c(O)cc(O)cc2O[C@H]1c1cc(O)c(O)cc1F)c1cc(O)c(O)c(O)c1F] | (2S,3R)-2-(2-fluoro-4,5- dihydroxyphenyl)-5,7- dihydroxychroman-3-yl 2-fluoro- 3,4,5-trihydroxybenzoate |
| 49 | 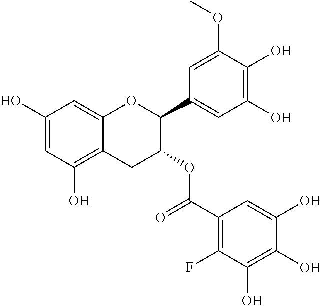 [SMILES: COc1cc([C@@H]2Oc3cc(O)cc(O)c3C[C@H]2OC(=O)c2cc(O)c(O)c(O)c2F)cc(O)c1O] | (2S,3R)-2-(3,4-dihydroxy-5- methoxyphenyl)-5,7- dihydroxychroman-3-yl 2-fluoro- 3,4,5-trihydroxybenzoate |
| 50 | 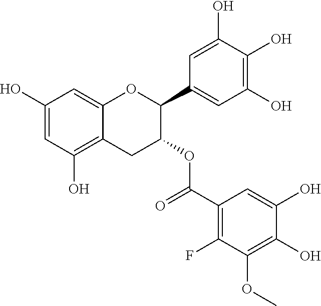 [SMILES: COc1c(O)c(O)cc(C(=O)O[C@@H]2Cc3c(O)cc(O)cc3O[C@H]2c2cc(O)c(O)c(O)c2)c1F] | (2S,3R)-5,7-dihydroxy-2-(3,4,5- trihydroxyphenyl)chroman-3-yl 2- fluoro-4,5-dihydroxy-3- methoxybenzoate |
| 51 | 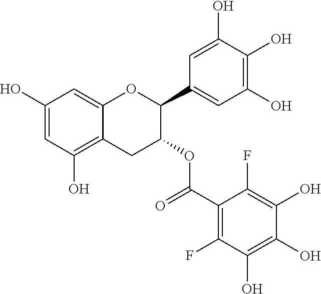 [SMILES: O=C(O[C@@H]1Cc2c(O)cc(O)cc2O[C@H]1c1cc(O)c(O)c(O)c1)c1c(F)c(O)c(O)c(O)c1F] | (2S,3R)-5,7-dihydroxy-2-(3,4,5- trihydroxyphenyl)chroman-3-yl 2,6-difluoro-3,4,5- trihydroxybenzoate |
| 52 | 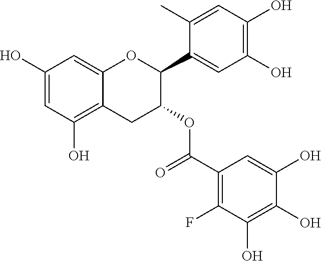 [SMILES: Cc1cc(O)c(O)cc1[C@@H]1Oc2cc(O)cc(O)c2C[C@H]1OC(=O)c1cc(O)c(O)c(O)c1F] | (2S,3R)-2-(4,5-dihydroxy-2- methylphenyl)-5,7- dihydroxychroman-3-yl 2-fluoro- 3,4,5-trihydroxybenzoate |
| 53 | 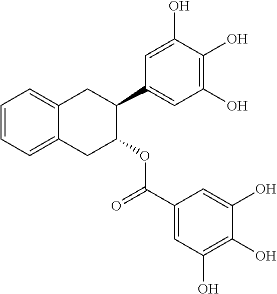 [SMILES: O=C(O[C@@H]1Cc2ccccc2C[C@H]1c1cc(O)c(O)c(O)c1)c1cc(O)c(O)c(O)c1] | (2R,3S)-3-(3,4,5- trihydroxyphenyl)-1,2,3,4- tetrahydronaphthalen-2-yl 3,4,5- trihydroxybenzoate |
| 54 | 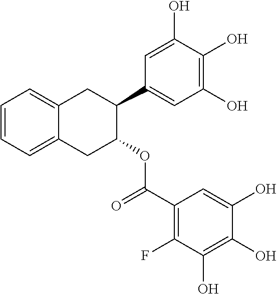 [SMILES: O=C(O[C@@H]1Cc2ccccc2C[C@H]1c1cc(O)c(O)c(O)c1)c1cc(O)c(O)c(O)c1F] | (2R,3S)-3-(3,4,5- trihydroxyphenyl)-1,2,3,4- tetrahydronaphthalen-2-yl 2- fluoro-3,4,5-trihydroxybenzoate |
| 55 | 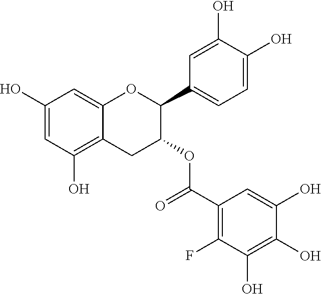 [SMILES: O=C(O[C@@H]1Cc2c(O)cc(O)cc2O[C@H]1c1ccc(O)c(O)c1)c1cc(O)c(O)c(O)c1F] | (2S,3R)-2-(3,4-dihydroxyphenyl)- 5,7-dihydroxychroman-3-yl 2- fluoro-3,4,5-trihydroxybenzoate |
| 56 | 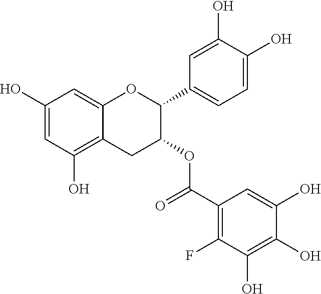 [SMILES: O=C(O[C@@H]1Cc2c(O)cc(O)cc2O[C@@H]1c1ccc(O)c(O)c1)c1cc(O)c(O)c(O)c1F] | (2R,3R)-2-(3,4-dihydroxyphenyl)- 5,7-dihydroxychroman-3-yl 2- fluoro-3,4,5-trihydroxybenzoate |
| 57 | 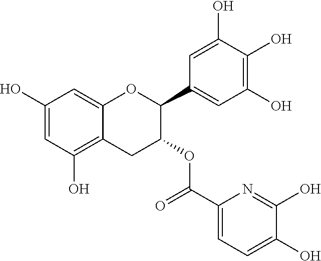 [SMILES: O=C(O[C@@H]1Cc2c(O)cc(O)cc2O[C@H]1c1cc(O)c(O)c(O)c1)c1ccc(O)c(O)n1] | (2S,3R)-5,7-dihydroxy-2-(3,4,5- trihydroxyphenyl)chroman-3-yl 5,6-dihydroxypicolinate |
| 58 | 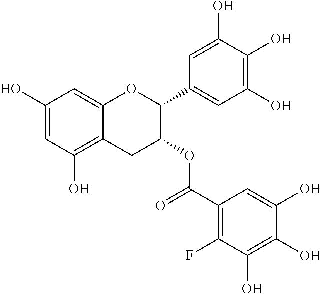 [SMILES: O=C(O[C@@H]1Cc2c(O)cc(O)cc2O[C@@H]1c1cc(O)c(O)c(O)c1)c1cc(O)c(O)c(O)c1F] | (2R,3R)-5,7-dihydroxy-2-(3,4,5- trihydroxyphenyl)chroman-3-yl 2- fluoro-3,4,5-trihydroxybenzoate |
| 59 | 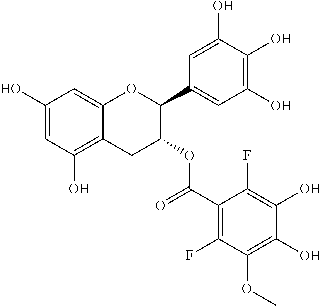 [SMILES: COc1c(O)c(O)c(F)c(C(=O)O[C@@H]2Cc3c(O)cc(O)cc3O[C@H]2c2cc(O)c(O)c(O)c2)c1F] | (2S,3R)-5,7-dihydroxy-2-(3,4,5- trihydroxyphenyl)chroman-3-yl 2,6-difluoro-3,4-dihydroxy-5- methoxybenzoate |
| 60 | 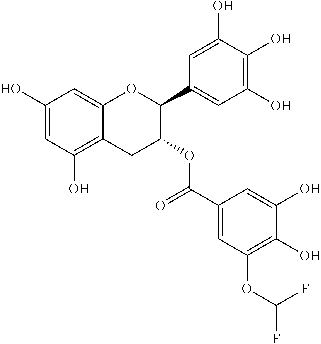 [SMILES: O=C(O[C@@H]1Cc2c(O)cc(O)cc2O[C@H]1c1cc(O)c(O)c(O)c1)c1cc(O)c(O)c(OC(F)F)c1] | (2S,3R)-5,7-dihydroxy-2-(3,4,5- trihydroxyphenyl)chroman-3-yl 3- (difluoromethoxy)-4,5- dihydroxybenzoate |
| 61 | 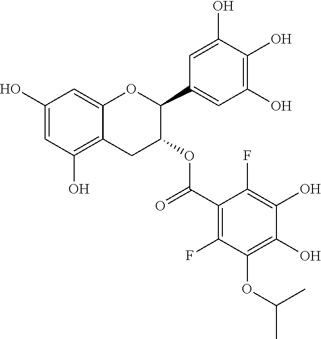 [SMILES: CC(C)Oc1c(O)c(O)c(F)c(C(=O)O[C@@H]2Cc3c(O)cc(O)cc3O[C@H]2c2cc(O)c(O)c(O)c2)c1F] | (2S,3R)-5,7-dihydroxy-2-(3,4,5- trihydroxyphenyl)chroman-3-yl 2,6-difluoro-3,4-dihydroxy-5- isopropoxybenzoate |
| 62 | 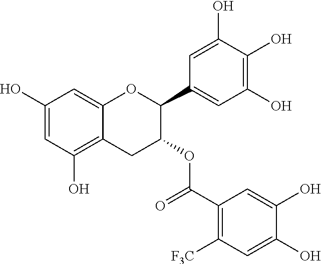 [SMILES: O=C(O[C@@H]1Cc2c(O)cc(O)cc2O[C@H]1c1cc(O)c(O)c(O)c1)c1cc(O)c(O)cc1C(F)(F)F] | (2S,3R)-5,7-dihydroxy-2-(3,4,5- trihydroxyphenyl)chroman-3-yl 4,5-dihydroxy-2- (trifluoromethyl)benzoate |
| 63 | 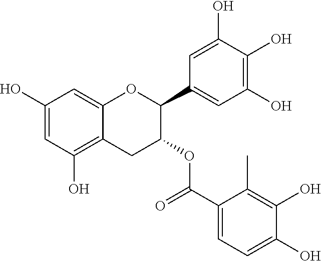 [SMILES: Cc1c(C(=O)O[C@@H]2Cc3c(O)cc(O)cc3O[C@H]2c2cc(O)c(O)c(O)c2)ccc(O)c1O] | (2S,3R)-5,7-dihydroxy-2-(3,4,5- trihydroxyphenyl)chroman-3-yl 3,4-dihydroxy-2-methylbenzoate |
| 64 | 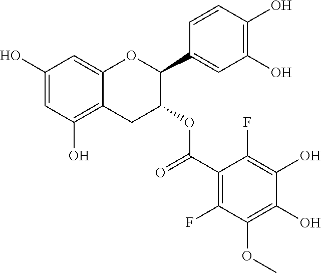 [SMILES: COc1c(O)c(O)c(F)c(C(=O)O[C@@H]2Cc3c(O)cc(O)cc3O[C@H]2c2ccc(O)c(O)c2)c1F] | (2S,3R)-2-(3,4-dihydroxyphenyl)- 5,7-dihydroxychroman-3-yl 2,6- difluoro-3,4-dihydroxy-5- methoxybenzoate |
| 65 | 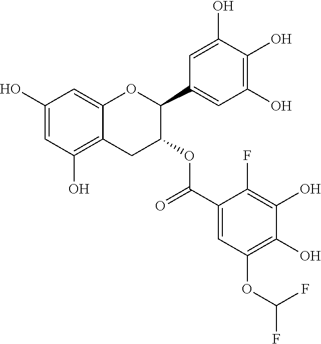 [SMILES: O=C(O[C@@H]1Cc2c(O)cc(O)cc2O[C@H]1c1cc(O)c(O)c(O)c1)c1cc(OC(F)F)c(O)c(O)c1F] | (2S,3R)-5,7-dihydroxy-2-(3,4,5- trihydroxyphenyl)chroman-3-yl 5- (difluoromethoxy)-2-fluoro-3,4- dihydroxybenzoate |
| 66 | 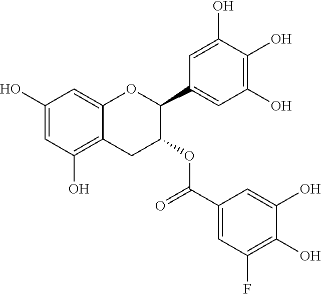 [SMILES: O=C(O[C@@H]1Cc2c(O)cc(O)cc2O[C@H]1c1cc(O)c(O)c(O)c1)c1cc(O)c(O)c(F)c1] | (2S,3R)-5,7-dihydroxy-2-(3,4,5- trihydroxyphenyl)chroman-3-yl 3- fluoro-4,5-dihydroxybenzoate |
| 67 | 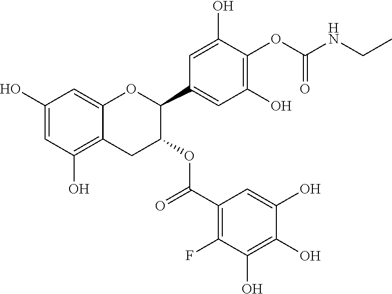 [SMILES: CCNC(=O)Oc1c(O)cc([C@@H]2Oc3cc(O)cc(O)c3C[C@H]2OC(=O)c2cc(O)c(O)c(O)c2F)cc1O] | (2S,3R)-2-(4- ((ethylcarbamoyl)oxy)-3,5- dihydroxyphenyl)-5,7- dihydroxychroman-3-yl 2-fluoro- 3,4,5-trihydroxybenzoate |
| 68 | 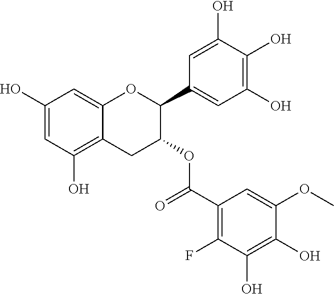 [SMILES: COc1cc(C(=O)O[C@@H]2Cc3c(O)cc(O)cc3O[C@H]2c2cc(O)c(O)c(O)c2)c(F)c(O)c1O] | (2S,3R)-5,7-dihydroxy-2-(3,4,5- trihydroxyphenyl)chroman-3-yl 2- fluoro-3,4-dihydroxy-5- methoxybenzoate |
| 69 | 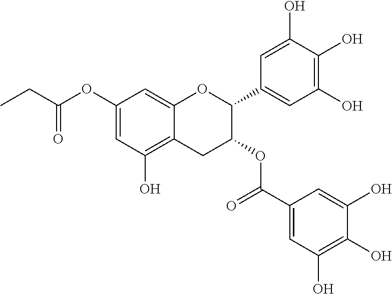 [SMILES: CCC(=O)Oc1cc(O)c2c(c1)O[C@H](c1cc(O)c(O)c(O)c1)[C@H](OC(=O)c1cc(O)c(O)c(O)c1)C2] | (2R,3R)-5-hydroxy-7- (propionyloxy)-2-(3,4,5- trihydroxyphenyl)chroman-3-yl 3,4,5-trihydroxybenzoate |
| 70 | 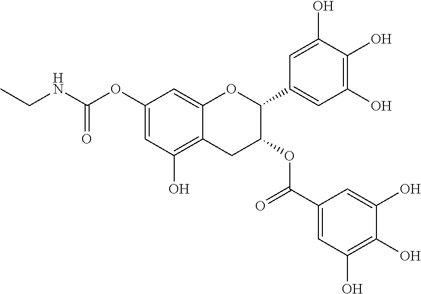 [SMILES: CCNC(=O)Oc1cc(O)c2c(c1)O[C@H](c1cc(O)c(O)c(O)c1)[C@H](OC(=O)c1cc(O)c(O)c(O)c1)C2] | (2R,3R)-7-((ethylcarbamoyl)oxy)- 5-hydroxy-2-(3,4,5- trihydroxyphenyl)chroman-3-yl 3,4,5-trihydroxybenzoate |
| 71 | 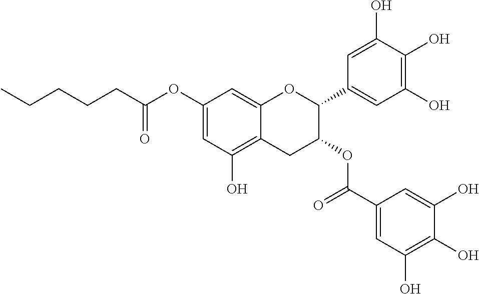 [SMILES: CCCCCC(=O)Oc1cc(O)c2c(c1)O[C@H](c1cc(O)c(O)c(O)c1)[C@H](OC(=O)c1cc(O)c(O)c(O)c1)C2] | (2R,3R)-7-(hexanoyloxy)-5- hydroxy-2-(3,4,5- trihydroxyphenyl)chroman-3-yl 3,4,5-trihydroxybenzoate |
| 72 | 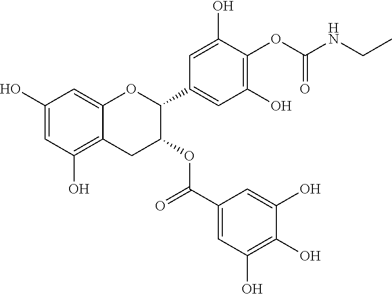 [SMILES: CCNC(=O)Oc1c(O)cc([C@H]2Oc3cc(O)cc(O)c3C[C@H]2OC(=O)c2cc(O)c(O)c(O)c2)cc1O] | (2R,3R)-2-(4- ((ethylcarbamoyl)oxy)-3,5- dihydroxyphenyl)-5,7- dihydroxychroman-3-yl 3,4,5- trihydroxybenzoate |
| 73 | 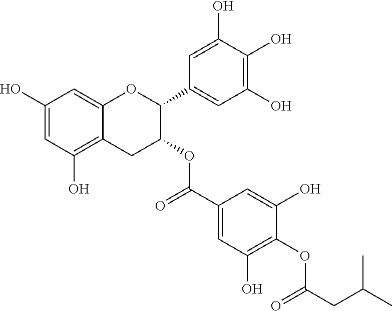 [SMILES: CC(C)CC(=O)Oc1c(O)cc(C(=O)O[C@@H]2Cc3c(O)cc(O)cc3O[C@@H]2c2cc(O)c(O)c(O)c2)cc1O] | (2R,3R)-5,7-dihydroxy-2-(3,4,5- trihydroxyphenyl)chroman-3-yl 3,5-dihydroxy-4-((3- methylbutanoyl)oxy)benzoate |
| 74 | 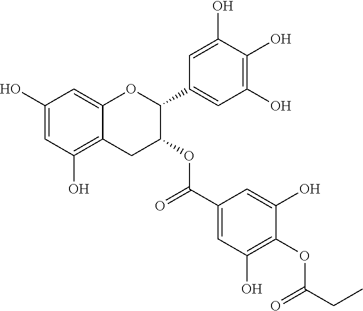 [SMILES: CCC(=O)Oc1c(O)cc(C(=O)O[C@@H]2Cc3c(O)cc(O)cc3O[C@@H]2c2cc(O)c(O)c(O)c2)cc1O] | (2R,3R)-5,7-dihydroxy-2-(3,4,5- trihydroxyphenyl)chroman-3-yl 3,5-dihydroxy-4- (propionyloxy)benzoate |
| 75 | 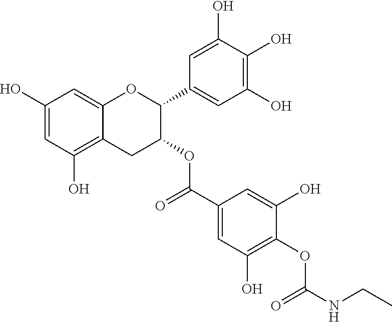 [SMILES: CCNC(=O)Oc1c(O)cc(C(=O)O[C@@H]2Cc3c(O)cc(O)cc3O[C@@H]2c2cc(O)c(O)c(O)c2)cc1O] | (2R,3R)-5,7-dihydroxy-2-(3,4,5- trihydroxyphenyl)chroman-3-yl 4- ((ethylcarbamoyl)oxy)-3,5- dihydroxybenzoate |
| 76 | 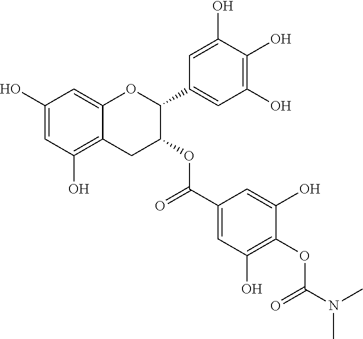 [SMILES: CN(C)C(=O)Oc1c(O)cc(C(=O)O[C@@H]2Cc3c(O)cc(O)cc3O[C@@H]2c2cc(O)c(O)c(O)c2)cc1O] | (2R,3R)-5,7-dihydroxy-2-(3,4,5- trihydroxyphenyl)chroman-3-yl 4- ((dimethylcarbamoyl)oxy)-3,5- dihydroxybenzoate |
| 77 | 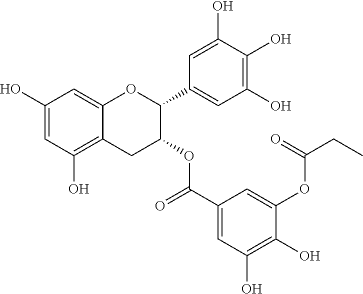 [SMILES: CCC(=O)Oc1cc(C(=O)O[C@@H]2Cc3c(O)cc(O)cc3O[C@@H]2c2cc(O)c(O)c(O)c2)cc(O)c1O] | (2R,3R)-5,7-dihydroxy-2-(3,4,5- trihydroxyphenyl)chroman-3-yl 3,4-dihydroxy-5- (propionyloxy)benzoate |
| 78 | 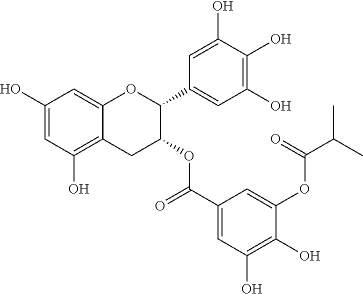 [SMILES: CC(C)C(=O)Oc1cc(C(=O)O[C@@H]2Cc3c(O)cc(O)cc3O[C@@H]2c2cc(O)c(O)c(O)c2)cc(O)c1O] | (2R,3R)-5,7-dihydroxy-2-(3,4,5- trihydroxyphenyl)chroman-3-yl 3,4-dihydroxy-5- (isobutyryloxy)benzoate |
| 79 | 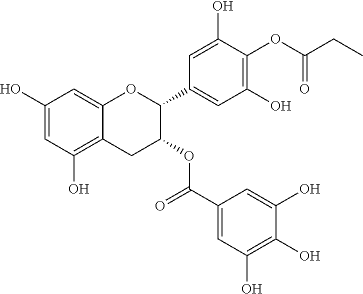 [SMILES: CCC(=O)Oc1c(O)cc([C@H]2Oc3cc(O)cc(O)c3C[C@H]2OC(=O)c2cc(O)c(O)c(O)c2)cc1O] | (2R,3R)-2-(3,5-dihydroxy-4- (propionyloxy)phenyl)-5,7- dihydroxychroman-3-yl 3,4,5- trihydroxybenzoate |
| 80 | 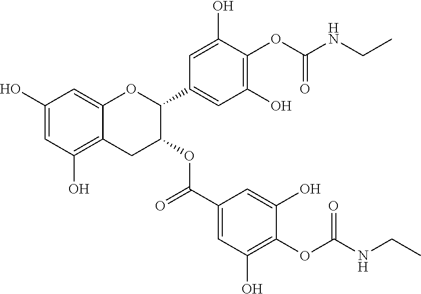 [SMILES: CCNC(=O)Oc1c(O)cc(C(=O)O[C@@H]2Cc3c(O)cc(O)cc3O[C@@H]2c2cc(O)c(OC(=O)NCC)c(O)c2)cc1O] | (2R,3R)-2-(4- ((ethylcarbamoyl)oxy)-3,5- dihydroxyphenyl)-5,7- dihydroxychroman-3-yl 4- ((ethylcarbamoyl)oxy)-3,5- dihydroxybenzoate |
| 81 | 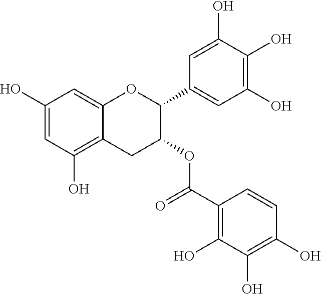 [SMILES: O=C(O[C@@H]1Cc2c(O)cc(O)cc2O[C@@H]1c1cc(O)c(O)c(O)c1)c1ccc(O)c(O)c1O] | (2R,3R)-5,7-dihydroxy-2-(3,4,5- trihydroxyphenyl)chroman-3-yl 2- fluoro-3,4,5-trihydroxybenzoate |

In certain embodiments as otherwise described herein, the compound of Formula I is selected from compounds 9-23, 25-37, and 39-81 of Table 1.

In certain embodiments as otherwise described herein, the compound of Formula I is compound 40, 41, 46, 47, 48, 50, 51, 55, 59, 68, or 81. For example, in certain embodiments as otherwise described herein, the compound of Formula I is compound 68. In another example, in certain embodiments as otherwise described herein, the compound of Formula I is compound 81.

### Pharmaceutical Compositions

In another aspect, the present disclosure provides pharmaceutical compositions comprising one or more of compounds as described herein, and a pharmaceutically acceptable carrier, excipient, adjuvant, and/or diluent. The exact nature of the carrier, excipient, adjuvant, and/or diluent will depend upon the desired use for the composition.

In certain embodiments, the pharmaceutical composition comprises one or more compounds of Formula I or a pharmaceutically acceptable salt thereof (i.e., as otherwise described herein), and a pharmaceutically acceptable carrier, excipient, adjuvant, and/or diluent. For example, in certain such embodiments, the pharmaceutical composition comprises one or more compounds of Formula II (e.g., Formula II-A or Formula II-B) or Formula III (e.g., Formula III-A or Formula III-B) or a pharmaceutically acceptable salt thereof (i.e., as otherwise described herein), and a pharmaceutically acceptable carrier, excipient, adjuvant, and/or diluent.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in a conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal (e.g., as described in more detail below), injection, transdermal, rectal, vaginal, etc., or a form suitable for administration by inhalation or insufflation.

For topical administration, the compound(s) may be formulated as solutions, gels, ointments, creams, suspensions, etc., as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agents. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen-free water, buffer, dextrose solution, etc., before use. To this end, the active compound(s) may be dried by any art-known technique, such as lyophilization, and reconstituted prior to use.

For oral administration, the pharmaceutical compositions may take the form of, for example, lozenges, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicles before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichloro-fluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example, capsules and cartridges comprised of gelatin) may be formulated containing a powder mix of the compound and a suitable powder base such as lactose or starch.

For ocular administration, the compound(s) may be formulated as a solution, emulsion, suspension, etc., suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of more significant toxicity.

The pharmaceutical compositions may, if desired, be presented in a pack or dispenser device, which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example, in an amount effective to treat or limit the particular disease being treated.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or limit the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

In some embodiments, the pharmaceutical composition is formulated for oral administration once a day or QD, and in some such formulations is a unit where the effective amount of the active ingredient ranges from 50 mg to 5000 mg. Alternatively, an oral solution may be provided, ranging from a concentration of 1 mg/ml to 50 mg/ml or higher.

One embodiment of the disclosure includes administering a compound of the disclosure to provide a serum concentration ranging from 0.1 μM to 50 μM. One embodiment of the disclosure includes administering a compound of the disclosure to provide a serum concentration ranging from 1 μM to 20 μM. One embodiment of the disclosure includes administering a compound of the disclosure to provide a serum concentration ranging from 5 μM to 20 μM. One embodiment of the disclosure includes administering a compound of the disclosure to provide a serum concentration of 10 μM, 20 UM, 5 UM, 1 μM, 15 UM, or 40 μM.

One embodiment of the disclosure includes administering a compound of the disclosure at a dose of 1 to 100 mg/kg/day, 5-40 mg/kg/day, 10-20 mg/kg/day, 1-2 mg/kg/day, 20-40 mg/kg/day, 45-50 mg/kg/day, 50-60 mg/kg/day, 55-65 mg/kg/day, 60-70 mg/kg/day or 65-75 mg/kg/day.

The compositions described herein may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of the dose. Where there is more than one administration in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The disclosure is not limited to dosing intervals that are spaced equally in time, but encompass doses at non-equal intervals, such as a priming schedule consisting of administration at 1 day, 4 days, 7 days, and 25 days, just to provide a non-limiting example.

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

As one aspect of the present disclosure contemplates the treatment of the disease/conditions with the compounds of the disclosure, the disclosure further relates to pharmaceutical compositions in kit form. When the composition of the disclosure is a part of a combination therapy with a secondary therapeutic agent, the kit may comprise two separate pharmaceutical compositions: one of compound of the present disclosure, and another of a second therapeutic agent. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

The compounds and compositions of the disclosure as described herein may also be administered in combination with one or more secondary therapeutic agents. Thus, in certain embodiments, the method also includes administering to a subject in need of such treatment an effective amount of one or more compounds of the disclosure as described herein (e.g., compounds of Formula I or Formula II, or those provided in Tables 1 and 2) or a pharmaceutical composition of the disclosure as described herein and one or more secondary therapeutic agents.

Combination therapy, in defining use of a compound of the present disclosure and the secondary therapeutic agent, is intended to embrace administration of each agent in a sequential manner in a regimen that will provide beneficial effects of the drug combination (e.g., the compounds and compositions of the disclosure as described herein and the secondary therapeutic agents can be formulated as separate compositions that are given sequentially), and is intended as well to embrace co-administration of these agents in a substantially simultaneous manner, such as in a single pharmaceutical composition having a fixed ratio of these active agents or in multiple or a separate pharmaceutical compositions for each agent. The disclosure is not limited in the sequence of administration: the compounds of and compositions of the disclosure may be administered either prior to or after (i.e., sequentially), or at the same time (i.e., simultaneously) as administration of the secondary therapeutic agent.

In certain embodiments, the secondary therapeutic agent may be administered in a previously established clinical dose when dosed for therapy in humans. In certain embodiments, the secondary therapeutic agent may be administered in an amount below its established human clinical dose when dosed for therapy. For example, the secondary therapeutic agent may be administered in an amount less than 1% of, e.g., less than 10%, or less than 25%, or less than 50%, or less than 75%, or even less than 90% of the established human clinical dose.

Examples of secondary therapeutic agents include, but are not limited to, steroids (such as, but are not limited to, dexamethasone, cortisone, hydrocortisone, hydrocortisone acetate, cortisone acetate, prednisolone, methylprednisolone, prednisone, betamethasone, betamethasone dipropionate, betamethasone valerate, clobetasol propionate, clobetasone, fluprednidene acetate, hydrocortisone aceponate, hydrocortisone buteprate, hydrocortisone butyrate, hydrocortisone valerate, fluocortolone, halometasone, mometasone, and prednicarbate), nonsteroidal anti-inflammatory drugs (NSAIDs) (such as, but not limited to, indomethacin, sulindac, ibuprofen, aspirin, naproxen, and tolmetin), immunomodulating agents (such as, but not limited to, azathioprine, cyclosporine, cyclophosphamide, deoxyspergualin, bredinin, rituximab, tocilizumab, sirolimus, methotrexate, anti CD3 antibodies, anti CD19 antibody, anti CD22 antibody, folinic acid, cyclosphosphamide, mycophenolate mofetil, and a B-cell targeting agent), chemotherapy agents (such as, but not limited to, didemnin B, dehydrodidemnin B, and bortezomib), intravenous gamma globulin (IVIG), thalidomide, inebilizumab, vascular health agents (such as, but not limited to, anticoagulants, antiplatelet agents, angiotensin-converting enzyme inhibitors, angiotensin II receptor blockers, angiotensin-receptor neprilysin inhibitors, beta blockers, calcium channel blockers, cholesterol-lowering medications, diuretics, and vasodilators), and convalescent plasma.

### Pharmaceutical Compositions

The present inventors have determined that the compounds described herein, when administered intranasally, can be more rapidly absorbed following intranasal administration (e.g., in the brain) relative to a corresponding dose administered orally. The present inventors note that rapid absorption can lead to a more rapid onset of action and efficacy at lower doses.

Accordingly, another aspect of the disclosure is an intranasal pharmaceutical composition comprising one or more compounds of Formula I or a pharmaceutically acceptable salt thereof (i.e., as otherwise described herein), present in a combined amount of 1-40 wt. %. The intranasal composition further includes one or more of a permeation enhancer, present in an amount of 0.1-20 wt. %; a chelator/anti-oxidant, present in an amount of 0.1-20 wt. %; a humectant; present in an amount of 1-30 wt. %; and a preservative, present in an amount of 0.03-2 wt. %. And the pH of the intranasal composition is 4.0-6.5.

In certain embodiments as otherwise described herein, the intranasal composition comprises one or more compounds of Formula II (e.g., Formula II-A or Formula II-B) or Formula III (e.g., Formula III-A or Formula III-B) or a pharmaceutically acceptable salt thereof (i.e., as otherwise described herein). For example, in certain such embodiments, the intranasal composition comprises one or more compounds of Table 1 (e.g., one or more of compounds 40, 41, 46, 47, 48, 50, 51, 55, 59, 68, and 81).

In certain embodiments as otherwise described herein, the intranasal composition includes one or more compounds of Formula I (e.g., Formula I-A, II, II-A, III, or III-A, as otherwise described herein) or a pharmaceutically acceptable salt thereof, present in a combined amount of 1-30 w/w %, e.g., 10-25 w/w %, or 1-12 w/w %

In certain embodiments as otherwise described herein, the intranasal composition includes a permeation enhancer, present in an amount of 1-20 wt. %. The present inventors note that by using a permeation enhancer, it is possible to further improve the aqueous solubility of the polyphenolic compounds such as catechin. Examples of suitable permeation enhancers include HP-β-CD, glycerin, and chitosan, transmucosal delivery enhancement agents including but not limited to alkylsaccharide transmucosal delivery enhancement agents (including but not limited to tetradecyl maltoside (TDM)), or combinations thereof. In various embodiments the permeation enhancer comprises HP-β-CD (such as at a concentration of from about 1.0% to about 20% w/w or any of the alternative embodiments listed for permeation enhancers in general), chitosan (such as at a concentration of from about 0.1% to about 2% w/w or any of the relevant alternative embodiments listed for permeation enhancers in general), glycerin (such as at a concentration of from about 1% to about 10% w/w or any of the relevant alternative embodiments listed for permeation enhancers in general), PEG 300 (such as at a concentration of from about 1% to about 20% w/w or any of the relevant alternative embodiments listed for permeation enhancers in general), PEG 400 (such as at a concentration of from about 1% to about 20% w/w or any of the relevant alternative embodiments listed for permeation enhancers in general), PEG 600 (such as at a concentration of from about 1% to about 20% w/w or any of the relevant alternative embodiments listed for permeation enhancers in general), and/or transmucosal delivery enhancement agents including but not limited to alkylsaccharide, including but not limited to tetradecyl maltoside (TDM) (such as at a concentration of from about 0.1% to about 2% w/w or any of the relevant alternative embodiments listed for permeation enhancers in general). The present inventors note that such permeation enhances can increase the solubility of the one or more compounds of Formula I or a pharmaceutically acceptable salt thereof (i.e., as otherwise described herein) in water to more than 10% w/w, desirably allowing for a more concentrated solution to be administered, facilitating a rapid onset of action and reducing irritancy).

In certain embodiments as otherwise described herein, the permeation enhancer is present in an amount of 1-20%, e.g., 1-18%, 2-18%, 3-17%, 4-16%, 5-15%, 6-14%, 7-13%, 8-12%, 9-11%, 2.3-10%, or 0.1% to 2% w/w. In certain such embodiments, the permeation enhancer comprises one or more compounds selected from cyclodextrin or analogs thereof, glycerin, PEG 400, sucrose monolaurate, chitosan, transmucosal delivery enhancement agents including but not limited to alkylsaccharide transmucosal delivery enhancement agents (including but not limited to tetradecyl maltoside (TDM)), pharmaceutically acceptable salts thereof, and any combination thereof. In certain such embodiments, the permeation enhancer comprises one or more compounds selected from (2-Hydroxypropyl)-β-cyclodextrin (HP-β-cyclodextrin); also referred to as HP-β-CD, or Hydroxypropyl betadex), randomly methylated cyclodextrin (also referred to as RM-β-CD), sulfobutylether-β-cyclodextrin (also referred to as SBE-β-CD), sucrose monolaurate, pharmaceutically acceptable salts thereof, and any combination thereof.

In certain embodiments as otherwise described herein, the intranasal composition includes an anti-oxidant/chelator, present in an amount of 0.1-20 wt. %. The present inventors note that the anti-oxidant/chelator can help to stabilize the one or more compounds of Formula I or a pharmaceutically acceptable salt thereof (i.e., as otherwise described herein) from auto-oxidation. In certain such embodiments, the anti-oxidant/chelator is present in an amount of 0.05-15%, 0.8-15%, 0.1-15%, 0.1-10%, 0.1-9%, or 0.1-6% w/w. In certain such embodiments, the anti-oxidant comprises one or more compounds selected from ascorbic acid, sodium metabisulfite, sodium bisulfite, tocopherol, and pharmaceutically acceptable salts thereof. In another embodiment, the anti-oxidant comprises ascorbic acid or a pharmaceutically acceptable salt thereof. The present inventors note that additional stabilizers may be used to improve chemical stability of the formulations, e.g., anti-oxidants such as ascorbic acid, sodium metabisulfite, sodium bisulfite or tocopherol, or metal chelators such as ethylenedaminetetraacetic acid (EDTA).

In certain embodiments as otherwise described herein, the intranasal composition includes a humectant, present in an amount of 1-30 wt. %. The present inventors note that the humectant can help to increase solubility of the one or more compounds of Formula I or a pharmaceutically acceptable salt thereof (i.e., as otherwise described herein) (e.g., improving patient acceptability of the nasal formulation). In certain such embodiments, the humectant is present in an amount of 1-25%, 1-20%, 1-15%, 1-10%, 1-9%, 2-8%, 3-7%, or 4-6% w/w. In certain such embodiments, the humectant comprises one or more compounds selected from glycerin, PEG (including but not limited to PEG 300, PEG400, and PEG 600), pharmaceutically acceptable salts thereof, and any combination thereof.

In certain embodiments as otherwise described herein, the intranasal composition includes a preservative, present in an amount of 0.03-2 wt. %. The present inventors note that the preservative can extend the shelf-life of the intranasal composition. In certain such embodiments, the preservative is present in an amount of 0.03-2%, e.g., 0.03-1%, or 0.03-0.5%, or 0.03-0.1 wt. %. In certain such embodiments, the preservative comprises one or more compounds selected from benzyl alcohol, parabens, thimerosal, chlorobutanol and benzalkonium chloride, and any combination thereof.

In certain embodiments as otherwise described herein, the intranasal composition includes a pH modifier such as, for example, a citrate, lactate, sodium hydroxide, or phosphate buffer. The present inventors note that the pH modifier can help to make the pH of the intranasal composition physiological and non-irritating (e.g., pH 5.0-6.5 for nasal mucosa). In certain embodiments as otherwise described herein, the intranasal composition comprises a pH modifier, present in an amount of 0.1-2% (e.g., 0.5-1.5%) w/w. In certain such embodiments, the pH modifier is sodium hydroxide or a pharmaceutically acceptable salt thereof. The present inventors additionally note that the intranasal composition can include one or more osmogens (e.g., sodium chloride, mannitol, glucose), e.g., to provide an isotonic formulation. The present inventors note that an osmolarity of 300-700 mOsmol/kg can desirably increase the viscosity and, accordingly, increase the residence time and improve absorption of the one or more compounds of Formula I or a pharmaceutically acceptable salt thereof (i.e., as otherwise described herein).

The present inventors note that the intranasal pharmaceutical composition may comprise any suitable form for intranasal administration. In certain embodiments as otherwise described herein, the intranasal composition is in the form of a liquid, a powder, a spray, a nose drop, a gel, an ointment, or any combination thereof. The intranasal composition can be formulated, for example, as a nasal emulsion, ointment, gel, (which offer advantages for local application because of their viscosity) or can be, for example powder formulations or nasal sprays. Such sprays typically comprise a solution of the active drug in physiological saline or other pharmaceutically suitable carrier liquids. Various nasal spray compression pumps can be used and calibrated to deliver a predetermined dose of the one or more compounds of Formula I or a pharmaceutically acceptable salt thereof (i.e., as otherwise described herein).

For example, the nasal formulations may be capable of delivering a dose of a compound of Formula I (e.g., one or more of compounds of Table 1; e.g., compound 40, 41, 46, 47, 48, 50, 51, 55, 59, 68, and/or 81) between about 1 mg to about 100 mg, or between about 5 mg to 20 mgs per shot (i.e.: per pump of a nasal spray) which can be given as one or more shots per nostril.

For solution formulations typical volumes used to deliver between about 1 mg to about 100 mg, or between about 5 mg to 20 mgs in man are 25 to 200 μL, or 75 to 150 μL per dose in each nostril. The intranasal solution formulations can be administered as drops from a nasal dropper bottle or as aerosols after being applied from squeeze bottles, single unit dose or metered-dose pump sprays.

The dose of a compound of Formula I (e.g., one or more of compounds of Table 1; e.g., compound 40, 41, 46, 47, 48, 50, 51, 55, 59, 68, and/or 81) can be combined with a mucoadhesive to enhance its contact with the olfactory mucosa. In some embodiments, the mucoadhesive is selected from the group consisting of a hydrophilic polymer, a hydrogel and a thermoplastic polymer. Preferred hydrophilic polymers include cellulose-based polymers (such as methylcellulose, hydroxyethyl cellulose, hydroxy propyl methyl cellulose, sodium carboxy methyl cellulose), a carbomer chitosan and plant gum. In some embodiments, the mucoadhesive is selected from the group consisting of poly(lactic acid) (“PLA”) and poly(glycolic acid) (“PGA”), and copolymers thereof. In some embodiments, the mucoadhesive formulation includes a penetration enhancer such as sodium glycocholate, sodium taurocholate, L-lysophosphotidyl choline, DMSO and a protease inhibitor. In some embodiments, the pharmaceutical composition includes a pharmaceutically acceptable carrier, a lipophilic micelle, a liposome, or a combination thereof. For example, the lipophilic micelle or liposome may comprise a ganglioside, a phosphatidylcholine, a phosphatidylserine, or a combination thereof.

According to some embodiments of intranasal delivery, it can be desirable to prolong the residence time of the pharmaceutical composition in the nasal cavity (e.g., in the olfactory region and/or in the sinus region), for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl), which are agents that enhance residence time in the nasal cavity. As a further approach, increasing the viscosity of the dosage formulation can also provide a means of prolonging contact of agent with olfactory epithelium.

The pharmaceutical formulation can also optionally include an absorption enhancer, such as an agent that inhibits enzyme activity, reduces mucous viscosity or elasticity, decreases mucociliary clearance effects, opens tight junctions, and/or solubilizes the active compound. Chemical enhancers are known in the art and include chelating agents (e.g., EDTA), fatty acids, bile acid salts, surfactants, and/or preservatives. Enhancers for penetration can be particularly useful when formulating compounds that exhibit poor membrane permeability, lack of lipophilicity, and/or are degraded by aminopeptidases. The concentration of the absorption enhancer in the pharmaceutical composition will vary depending upon the agent selected and the formulation.

The pharmaceutical formulation can optionally contain an odorant to provide a sensation of odor, to aid in inhalation of the composition so as to promote delivery to the olfactory epithelium and/or to trigger transport by the olfactory neurons. The pharmaceutical formulations may also optionally include a thickener, which may be present in an amount of 1%, 0.5%, 0.2%, 0.1% by weight or less (or not present at all).

Single unit-dose spray can be prepared aseptically or terminally sterilized to produce a sterile final product.

In the formulations, effective concentrations of one or more compounds or pharmaceutically acceptable derivatives is (are) mixed with a suitable pharmaceutical carrier or vehicle. The compounds may be derivatized as the corresponding salts, esters, enol ethers or esters, acids, bases, solvates, hydrates or prodrugs prior to formulation. Any suitable carrier or diluent may be used, including but not limited to a solvent of dispersion medium containing, for example, water, ethanol, polyol (e.g., glycerol, propylene glycol, and liquid polyethylene glycol, and the like), suitable mixtures thereof, and vegetable oils.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

Terms used herein may be preceded and/or followed by a single dash, “−”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” (i.e., the attachment is via the last portion of the name) unless a dash indicates otherwise. For example, C₁-C₆alkoxycarbonyloxy and —OC(O)C₁-C₆alkyl indicate the same functionality; similarly arylalkyl and -alkylaryl indicate the same functionality.

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

The term “alkylene” refers to a bivalent alkyl group. An “alkylene chain” is a polymethylene group, i.e., —(CH₂)ₙ—, wherein n is a positive integer, preferably from one to six, from one to four, from one to three, from one to two, or from two to three. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia.

The terms “cyano” and “nitrile” as used herein, mean a —CN group.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thia. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia.

The term “halo” or “halogen” as used herein, means —Cl, —Br, —I or —F.

The terms “haloalkyl” and “haloalkoxy” refer to an alkyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a benzo ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, 2,3-dihydrothieno[3,4-b][1,4]dioxan-5-yl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

The terms “heterocyclyl” and “heterocycloalkyl” as used herein, mean a monocyclic heterocycle or a bicyclic heterocycle. The monocyclic heterocycle is a 3, 4, 5, 6, 7, or 8 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a bridged monocyclic ring or a monocyclic heterocycle fused to either a phenyl, a monocyclic cycloalkyl, a monocyclic cycloalkenyl, a monocyclic heterocycle, or a monocyclic heteroaryl. Bridged monocyclic rings contain a monocyclic heterocycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thia. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia.

The term “oxo” as used herein means a ═O group.

The term “saturated” as used herein means the referenced chemical structure does not contain any multiple carbon-carbon bonds. For example, a saturated cycloalkyl group as defined herein includes cyclohexyl, cyclopropyl, and the like.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

The term “thia” as used herein means a ═S group.

The term “unsaturated” as used herein means the referenced chemical structure contains at least one multiple carbon-carbon bond, but is not aromatic. For example, an unsaturated cycloalkyl group as defined herein includes cyclohexenyl, cyclopentenyl, cyclohexadienyl, and the like.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. Both the R and the S stereochemical isomers, as well as all mixtures thereof, are included within the scope of the disclosure.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutically acceptable salt” refers to both acid and base addition salts.

“Therapeutically effective amount” or “effective amount” refers to that amount of a compound which, when administered to a subject, is sufficient to effect treatment for a disease or disorder described herein. The amount of a compound which constitutes a “therapeutically effective amount” will vary depending on the compound, the disorder and its severity, and the age of the subject to be treated, but can be determined routinely by one of ordinary skill in the art. An effective amount is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

“Treating” or “treatment” as used herein covers the treatment of a disease or disorder described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, ameliorating, or slowing progression of one or more symptoms of the disease or disorder.

As used herein, “limiting” or “limiting development of” a disease or disorder refers to reducing onset of the disease or disorder in a subject that does not have the disease or disorder. For example, “limiting” or “limiting development of” a viral infection includes:
i. limiting development of infection;
ii. reducing the severity a subsequent infection; and/or
iii. limiting development of symptoms after a subsequent infection.

“Subject” refers to a warm-blooded animal such as a mammal, preferably a human, or a human child, which is afflicted with, or has the potential to be afflicted with a disease as described herein.

### Methods of Preparation

### Gallocatechin

Another aspect of the disclosure is method for preparing gallocatechin. The present inventors note that gallocatechin:

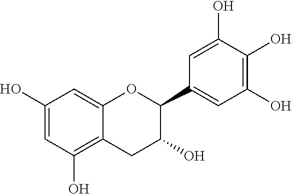 [SMILES: Oc1cc(O)c2c(c1)O[C@@H](c1cc(O)c(O)c(O)c1)[C@H](O)C2]

is a key precursor of certain desirable (2S,3R)-compounds described herein, such as, for example, compounds 40, 41, 50, 51, 59, and 68 of Table 1, above. Surprisingly, the present inventors have determined that gallocatechin can be prepared in desirably high yields, and in desirably high purity, by treatment of epigallocatechin:

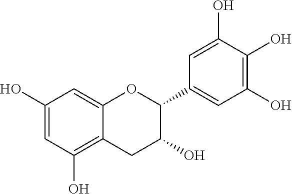 [SMILES: Oc1cc(O)c2c(c1)O[C@H](c1cc(O)c(O)c(O)c1)[C@H](O)C2]

with aqueous buffer at elevated temperatures. Moreover, the present inventors note that the crude reaction product, obtained by precipitation and filtration, can desirably be purified by simple recrystallization.

Accordingly, in certain embodiments as otherwise described herein, the method comprises contacting epigallocatechin with an aqueous buffer system at a first temperature, for a first period of time to provide a crude reaction mixture including gallocatechin. In certain embodiments as otherwise described herein, the buffer system has a pH of 7-8. For example, in certain such embodiments, the buffer system has a pH of 7-7.5, e.g., a pH of about 7.2. In certain embodiments as otherwise described herein, the buffer system is a phosphate buffer solution. In certain embodiments as otherwise described herein, the buffer system is a HEPES buffer solution.

In certain embodiments as otherwise described herein, the first temperature is greater than 50° C., e.g., greater than 60° C., or greater than 70° C., or greater than 80° C. In certain embodiments as otherwise described herein, the first temperature is reflux. In certain embodiments as otherwise described herein, the first period of time is at least 1 hour. For example, in certain such embodiments, the first period of time is 1-8 hours, e.g., 1-5 hours, or 1-3 hours.

In certain embodiments as otherwise described herein, the method comprises, after the first period of time, cooling the crude reaction mixture to a second temperature lower than the first temperature to provide a precipitated crude material comprising gallocatechin, and then separating the precipitated crude material (e.g., by filtration). In certain embodiments as otherwise described herein, the second temperature is less than 40° C., e.g., less than 30° C. In certain embodiments as otherwise described herein, the second temperature is room temperature.

In certain embodiments as otherwise described herein, the method comprises recrystallizing the filtered crude product in an aqueous solvent system to provide a purified material comprising gallocatechin. In certain embodiments as otherwise described herein, the aqueous solvent system is deionized water. In certain embodiments as otherwise described herein, the purified material comprises at least 90 wt. %, e.g., at least 95 wt. %, or at least 97.5 wt. % gallocatechin.

### General Methods

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/1, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

The compounds disclosed herein can be made using procedures familiar to the person of ordinary skill in the art and as described herein. For example, compounds of Formula I can be prepared according to Schemes 1-19, general procedures (below), and/or analogous synthetic procedures. One of skill in the art can adapt the reaction sequences of Schemes 1-19, general procedures, and Example 1 to fit the desired target molecule. Of course, in certain situations one of skill in the art will use different reagents to affect one or more of the individual steps or to use protected versions of certain of the substituents. Additionally, one skilled in the art would recognize that compounds of the disclosure can be synthesized using different routes altogether.

### General Procedures

Representative synthetic procedures for the preparation of compounds of the invention are outlined below in Schemes 1-19.

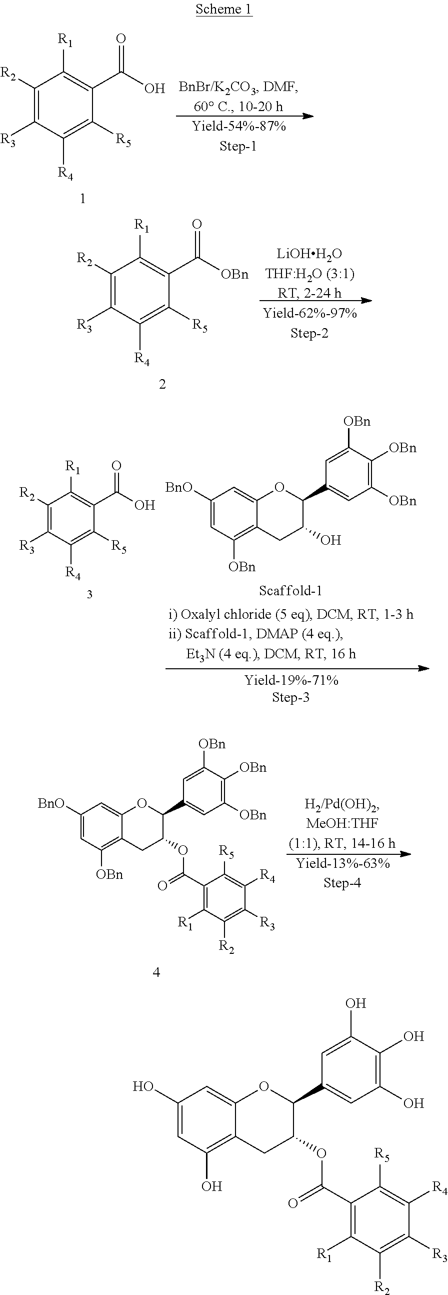

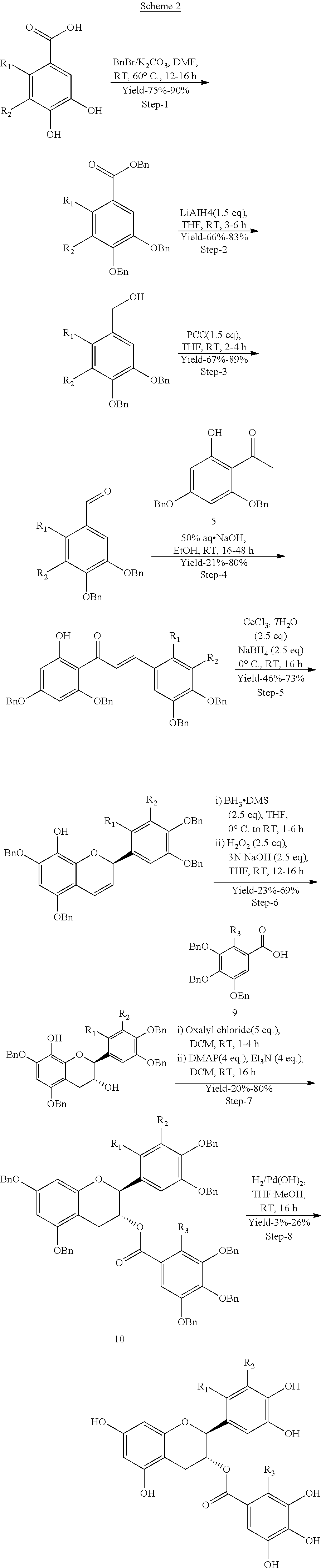

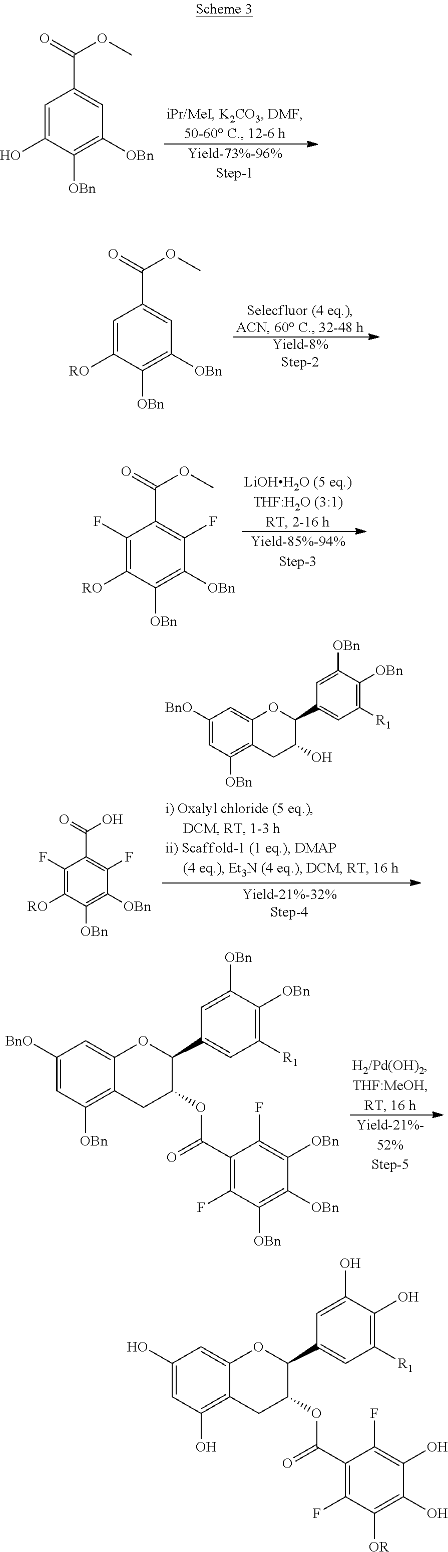

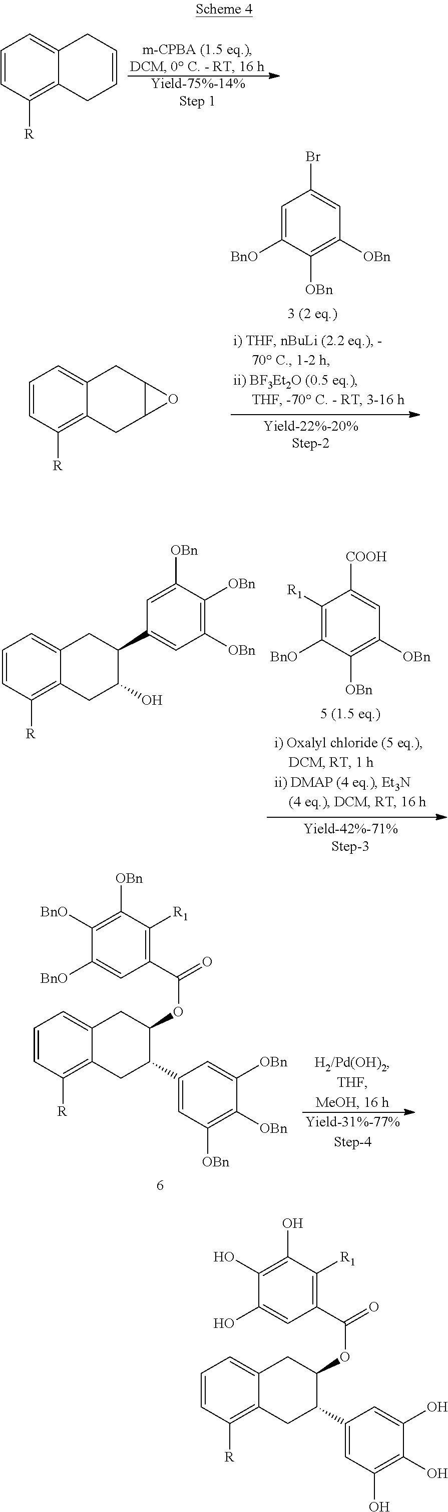

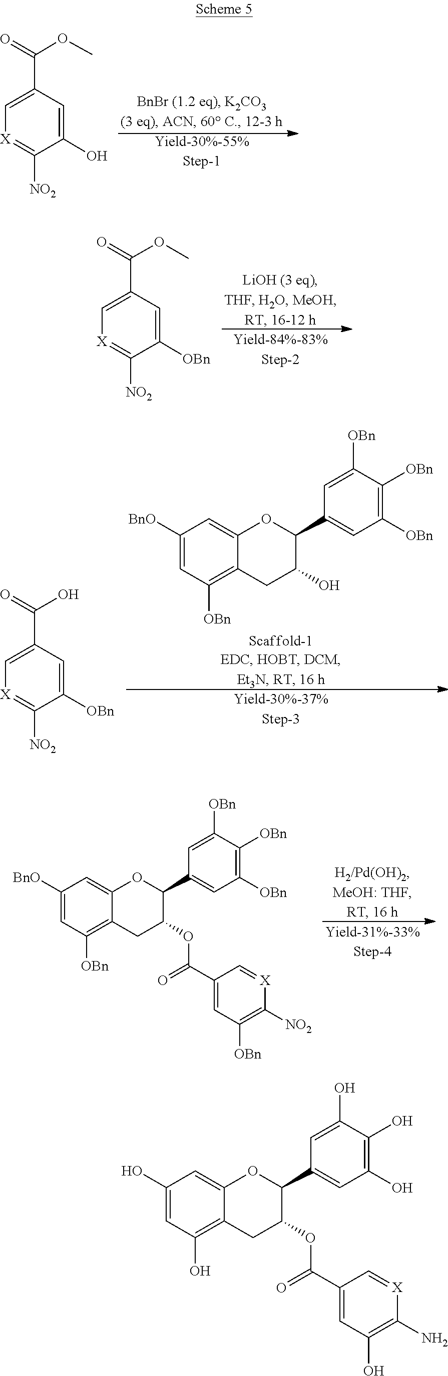

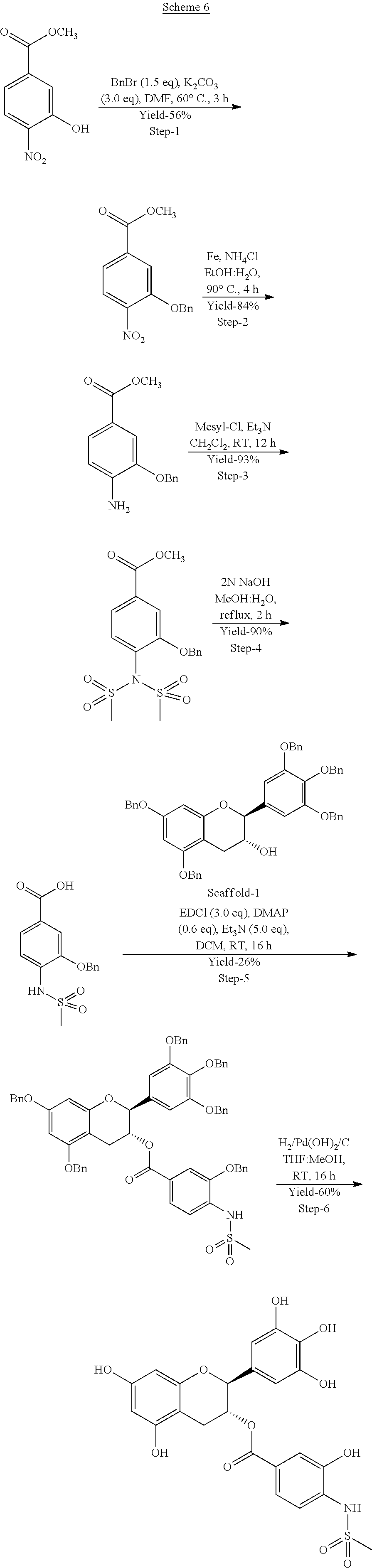

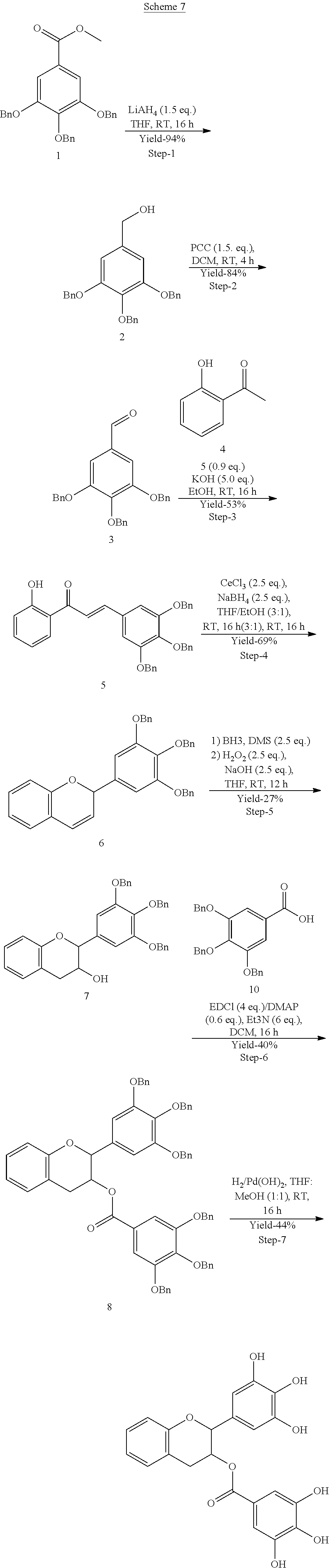

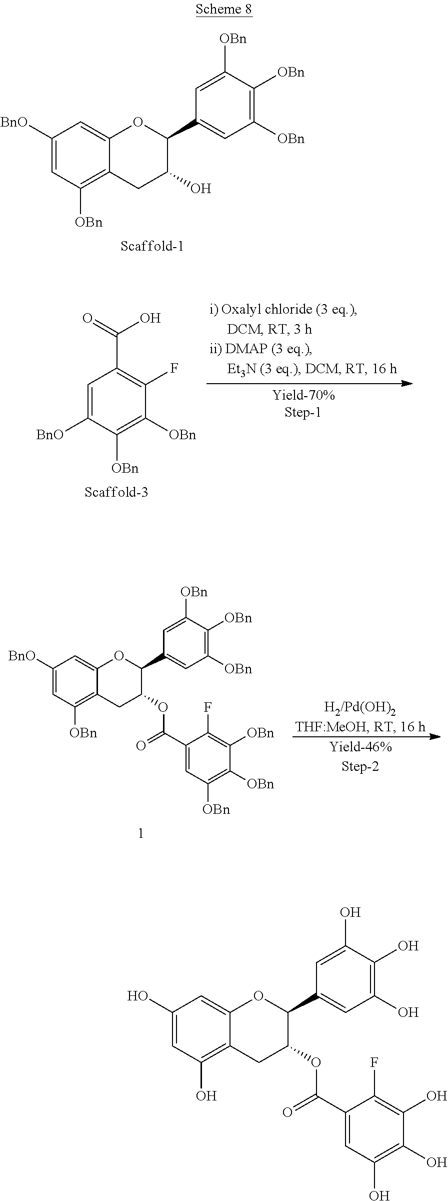 [SMILES: O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1cc(OCc2ccccc2)c(OCc2ccccc2)c(OCc2ccccc2)c1F ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1cc(OCc2ccccc2)c(OCc2ccccc2)c(OCc2ccccc2)c1F ; O=C(O[C@@H]1Cc2c(O)cc(O)cc2O[C@H]1c1cc(O)c(O)c(O)c1)c1cc(O)c(O)c(O)c1F]

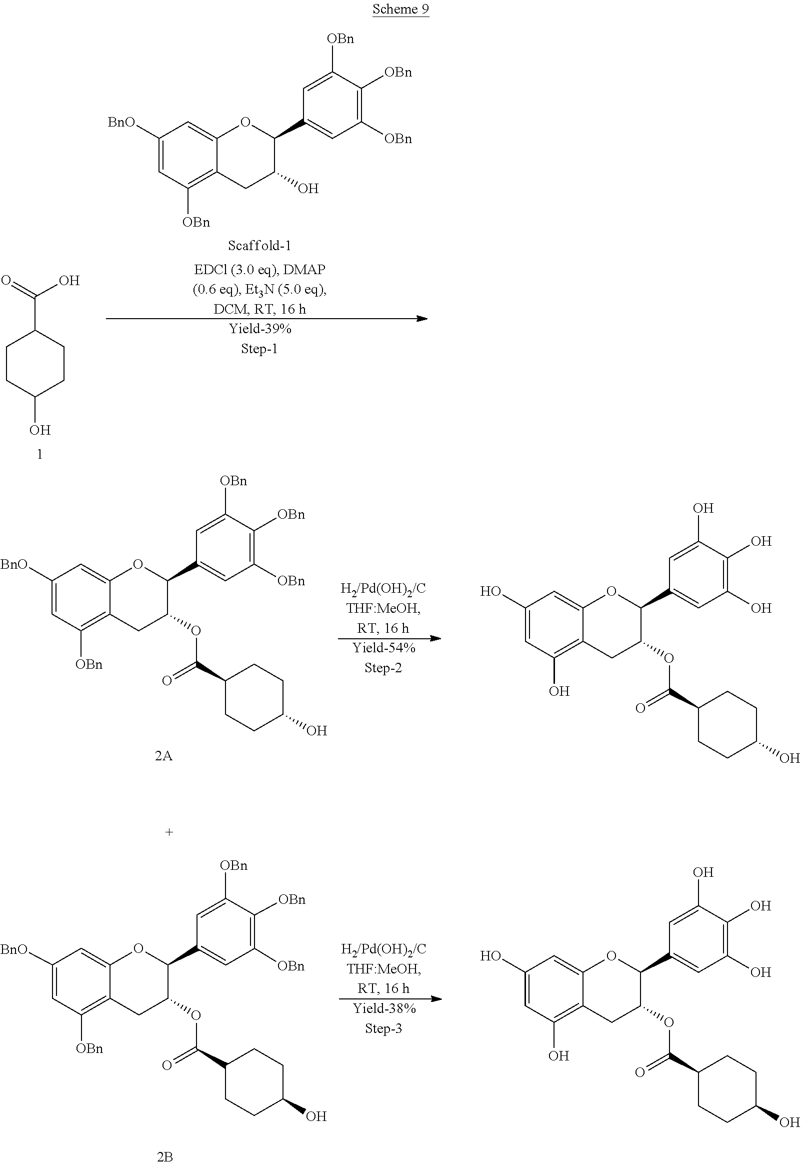

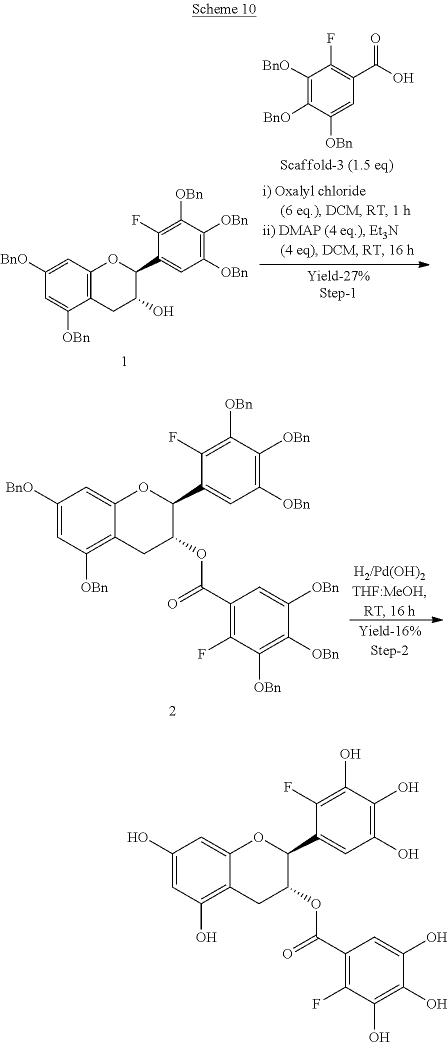 [SMILES: O=C(O)c1cc(OCc2ccccc2)c(OCc2ccccc2)c(OCc2ccccc2)c1F ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1F ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1F)c1cc(OCc2ccccc2)c(OCc2ccccc2)c(OCc2ccccc2)c1F ; O=C(O[C@@H]1Cc2c(O)cc(O)cc2O[C@H]1c1cc(O)c(O)c(O)c1F)c1cc(O)c(O)c(O)c1F]

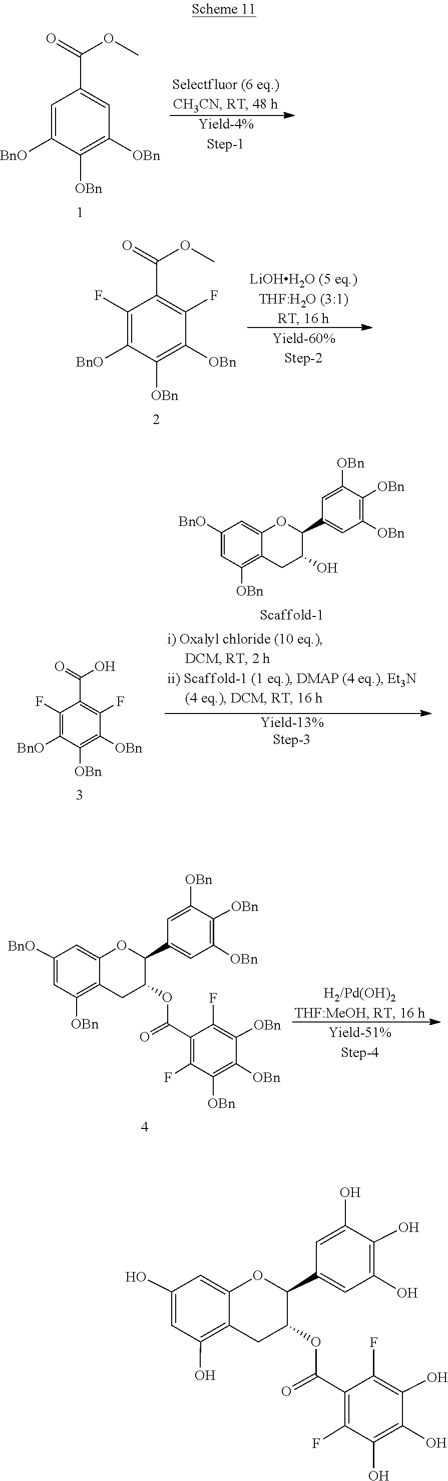 [SMILES: COC(=O)c1cc(OCc2ccccc2)c(OCc2ccccc2)c(OCc2ccccc2)c1 ; COC(=O)c1c(F)c(OCc2ccccc2)c(OCc2ccccc2)c(OCc2ccccc2)c1F ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1c(F)c(OCc2ccccc2)c(OCc2ccccc2)c(OCc2ccccc2)c1F ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1c(F)c(OCc2ccccc2)c(OCc2ccccc2)c(OCc2ccccc2)c1F ; O=C(O[C@@H]1Cc2c(O)cc(O)cc2O[C@H]1c1cc(O)c(O)c(O)c1)c1c(F)c(O)c(O)c(O)c1F]

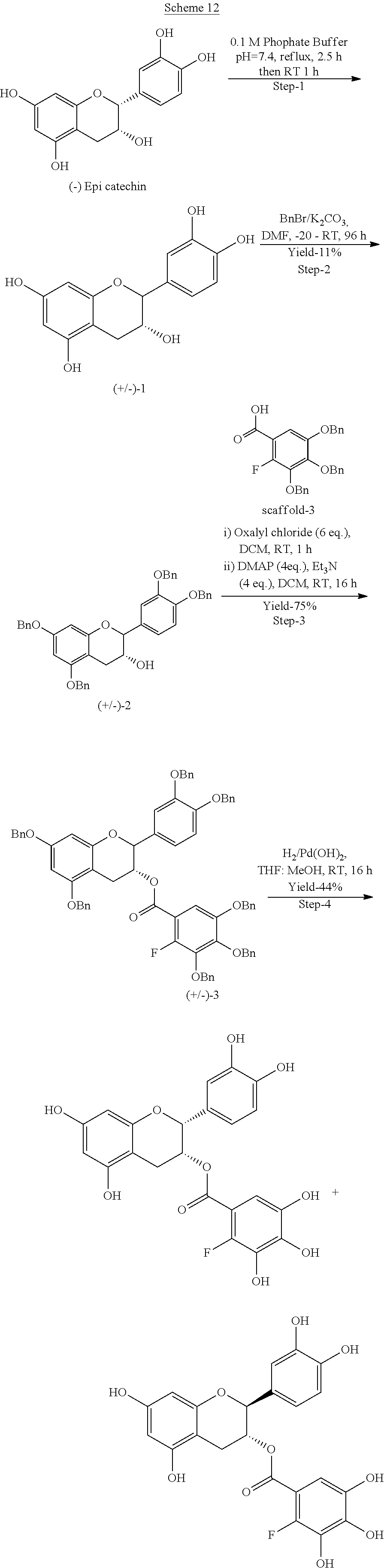

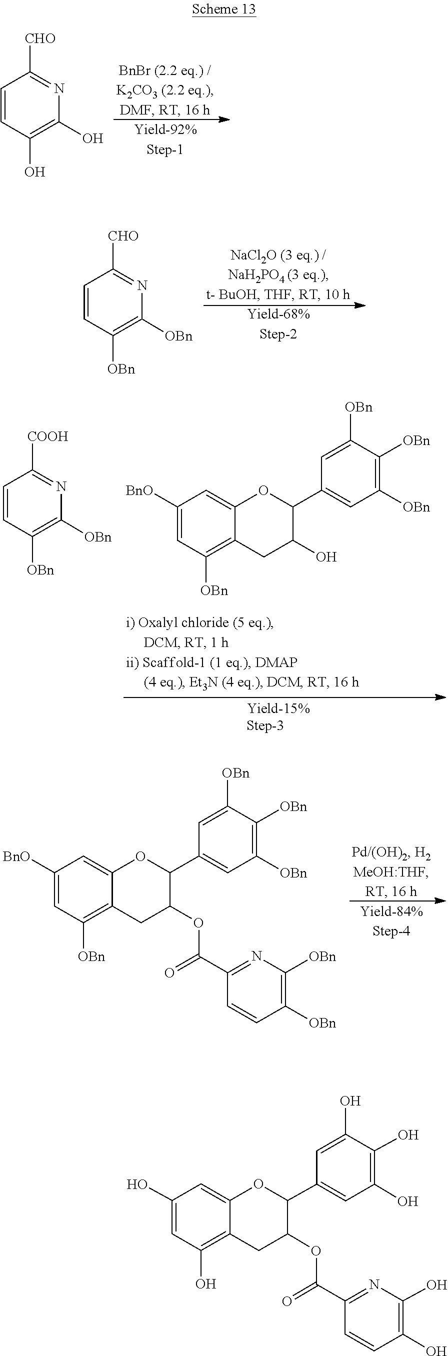 [SMILES: O=Cc1ccc(O)c(O)n1 ; O=Cc1ccc(OCc2ccccc2)c(OCc2ccccc2)n1 ; O=C(O)c1ccc(OCc2ccccc2)c(OCc2ccccc2)n1 ; OC1Cc2c(OCc3ccccc3)cc(OCc3ccccc3)cc2OC1c1cc(OCc2ccccc2)c(OCc2ccccc2)c(OCc2ccccc2)c1 ; O=C(OC1Cc2c(OCc3ccccc3)cc(OCc3ccccc3)cc2OC1c1cc(OCc2ccccc2)c(OCc2ccccc2)c(OCc2ccccc2)c1)c1ccc(OCc2ccccc2)c(OCc2ccccc2)n1 ; O=C(OC1Cc2c(O)cc(O)cc2OC1c1cc(O)c(O)c(O)c1)c1ccc(O)c(O)n1]

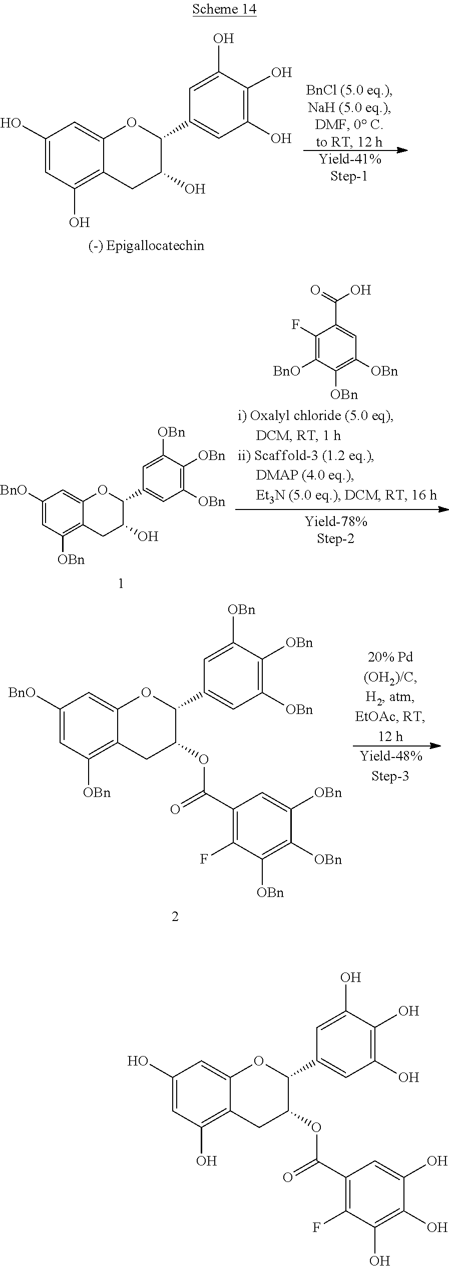 [SMILES: Oc1cc(O)c2c(c1)O[C@H](c1cc(O)c(O)c(O)c1)[C@H](O)C2 ; O=C(O)c1cc(OCc2ccccc2)c(OCc2ccccc2)c(OCc2ccccc2)c1F ; O[C@@H]1Cc2c(OCc3ccccc3)cc(OCc3ccccc3)cc2O[C@@H]1c1cc(OCc2ccccc2)c(OCc2ccccc2)c(OCc2ccccc2)c1 ; O=C(O[C@@H]1Cc2c(OCc3ccccc3)cc(OCc3ccccc3)cc2O[C@@H]1c1cc(OCc2ccccc2)c(OCc2ccccc2)c(OCc2ccccc2)c1)c1cc(OCc2ccccc2)c(OCc2ccccc2)c(OCc2ccccc2)c1F ; O=C(O[C@@H]1Cc2c(O)cc(O)cc2O[C@@H]1c1cc(O)c(O)c(O)c1)c1cc(O)c(O)c(O)c1F]

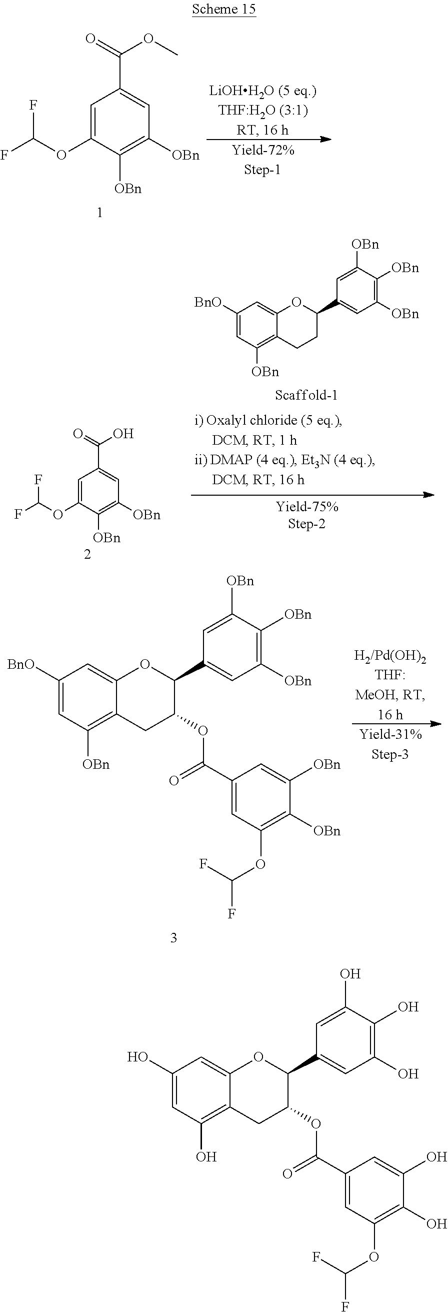 [SMILES: COC(=O)c1cc(OCc2ccccc2)c(OCc2ccccc2)c(OC(F)F)c1 ; c1ccc(COc2cc(OCc3ccccc3)c3c(c2)O[C@@H](c2cc(OCc4ccccc4)c(OCc4ccccc4)c(OCc4ccccc4)c2)CC3)cc1 ; O=C(O)c1cc(OCc2ccccc2)c(OCc2ccccc2)c(OC(F)F)c1 ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1cc(OCc2ccccc2)c(OCc2ccccc2)c(OC(F)F)c1 ; O=C(O[C@@H]1Cc2c(O)cc(O)cc2O[C@H]1c1cc(O)c(O)c(O)c1)c1cc(O)c(O)c(OC(F)F)c1]

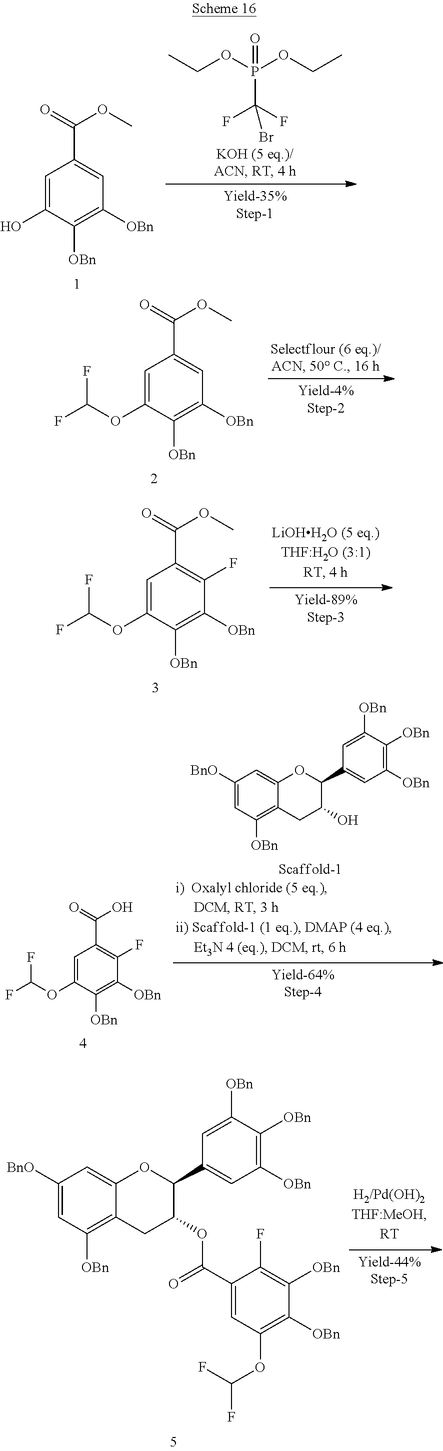

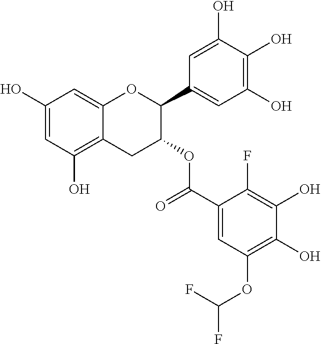 [SMILES: O=C(O[C@@H]1Cc2c(O)cc(O)cc2O[C@H]1c1cc(O)c(O)c(O)c1)c1cc(OC(F)F)c(O)c(O)c1F]

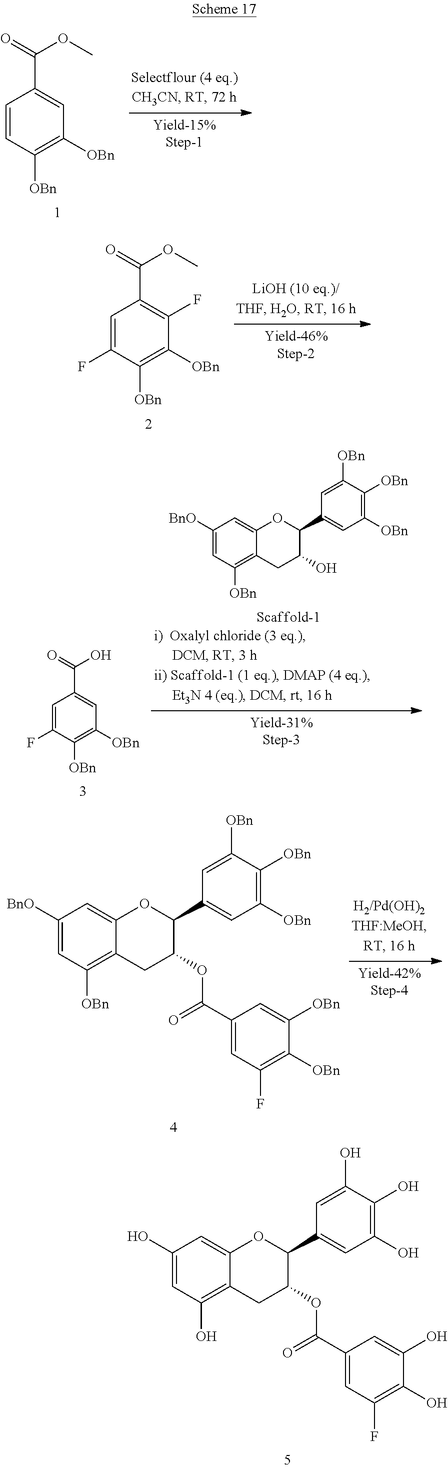 [SMILES: COC(=O)c1ccc(OCc2ccccc2)c(OCc2ccccc2)c1 ; COC(=O)c1cc(F)c(OCc2ccccc2)c(OCc2ccccc2)c1F ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1cc(F)c(OCc2ccccc2)c(OCc2ccccc2)c1 ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1cc(F)c(OCc2ccccc2)c(OCc2ccccc2)c1 ; O=C(O[C@@H]1Cc2c(O)cc(O)cc2O[C@H]1c1cc(O)c(O)c(O)c1)c1cc(O)c(O)c(F)c1]

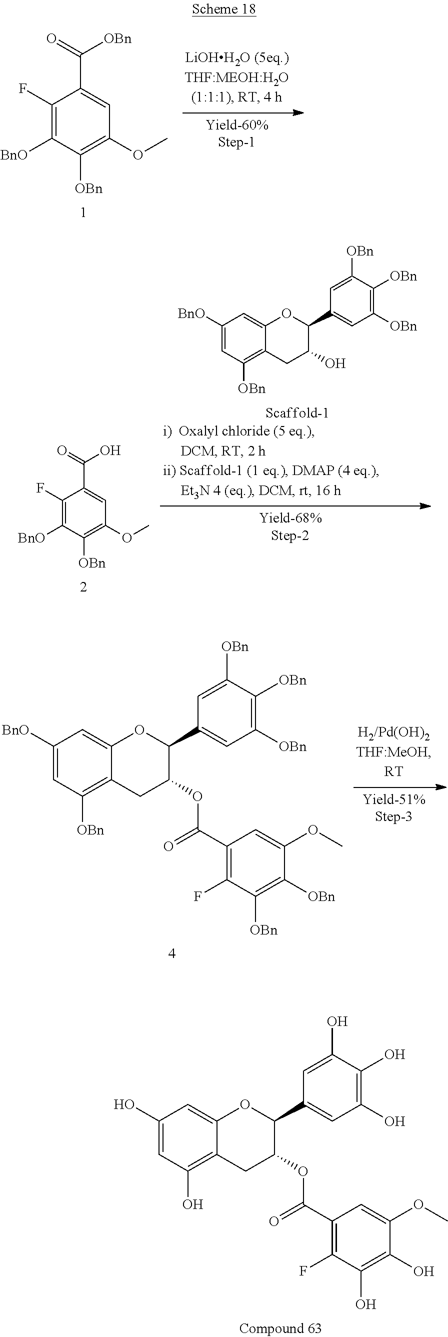 [SMILES: COc1cc(C(=O)OCc2ccccc2)c(F)c(OCc2ccccc2)c1OCc1ccccc1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; COc1cc(C(=O)O)c(F)c(OCc2ccccc2)c1OCc1ccccc1 ; COc1cc(C(=O)O[C@@H]2Cc3c(OCc4ccccc4)cc(OCc4ccccc4)cc3O[C@H]2c2cc(OCc3ccccc3)c(OCc3ccccc3)c(OCc3ccccc3)c2)c(F)c(OCc2ccccc2)c1OCc1ccccc1 ; COc1cc(C(=O)O[C@@H]2Cc3c(O)cc(O)cc3O[C@H]2c2cc(O)c(O)c(O)c2)c(F)c(O)c1O]

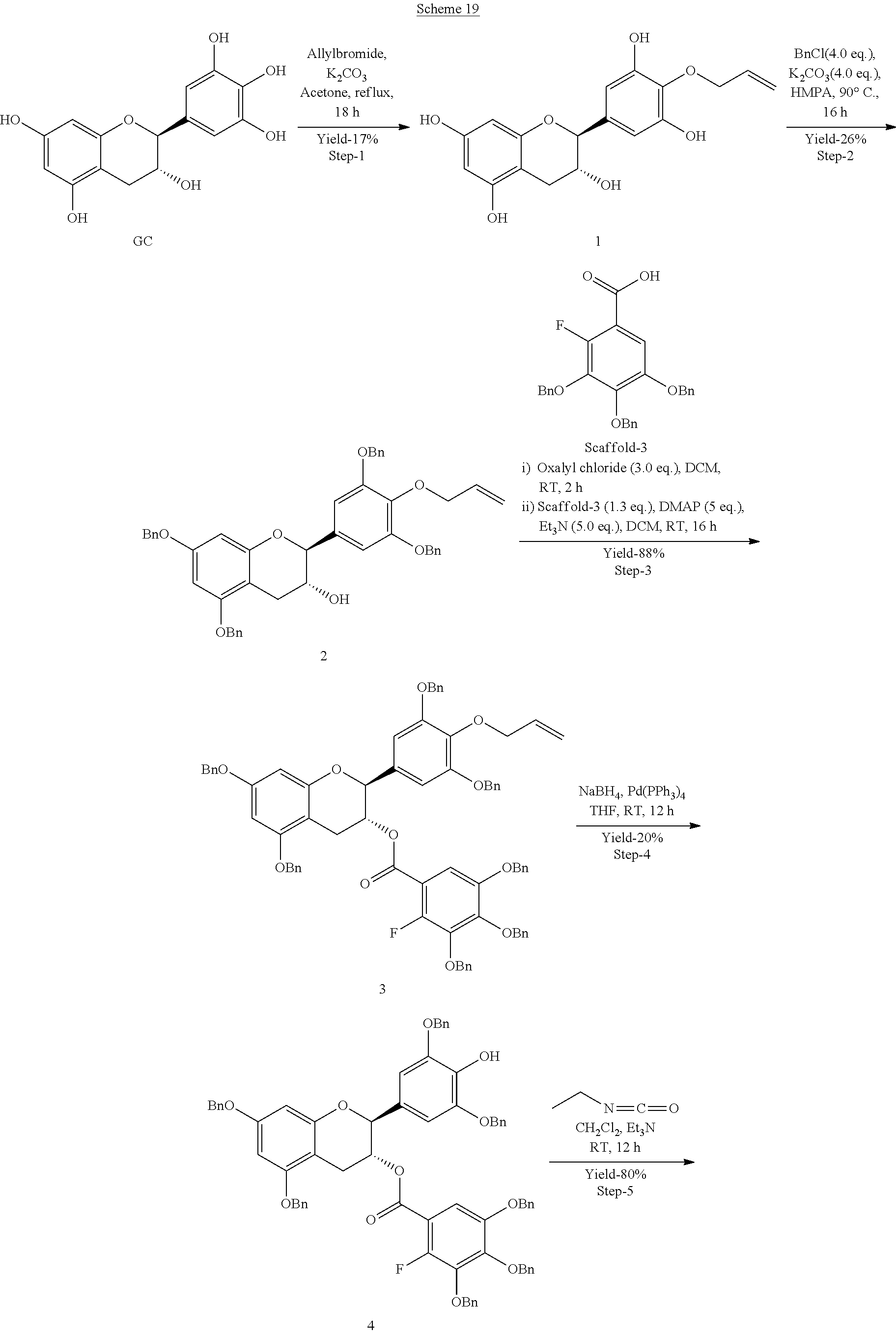

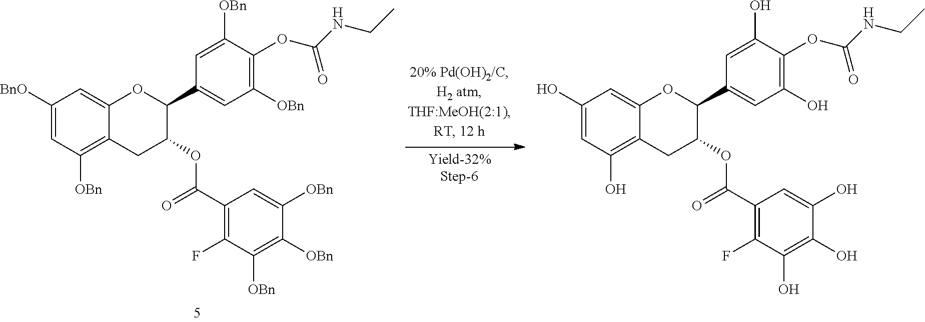 [SMILES: CCNC(=O)Oc1c(OCc2ccccc2)cc([C@@H]2Oc3cc(OCc4ccccc4)cc(OCc4ccccc4)c3C[C@H]2OC(=O)c2cc(OCc3ccccc3)c(OCc3ccccc3)c(OCc3ccccc3)c2F)cc1OCc1ccccc1 ; CCNC(=O)Oc1c(O)cc([C@@H]2Oc3cc(O)cc(O)c3C[C@H]2OC(=O)c2cc(O)c(O)c(O)c2F)cc1O]

### EXAMPLES

The compositions and methods of the disclosure are illustrated further by the following examples, which are not to be construed as limiting the disclosure in scope or spirit to the specific procedures and compounds described in them.

### Example 1. Preparation of Compounds of Table 1

### Compound 9

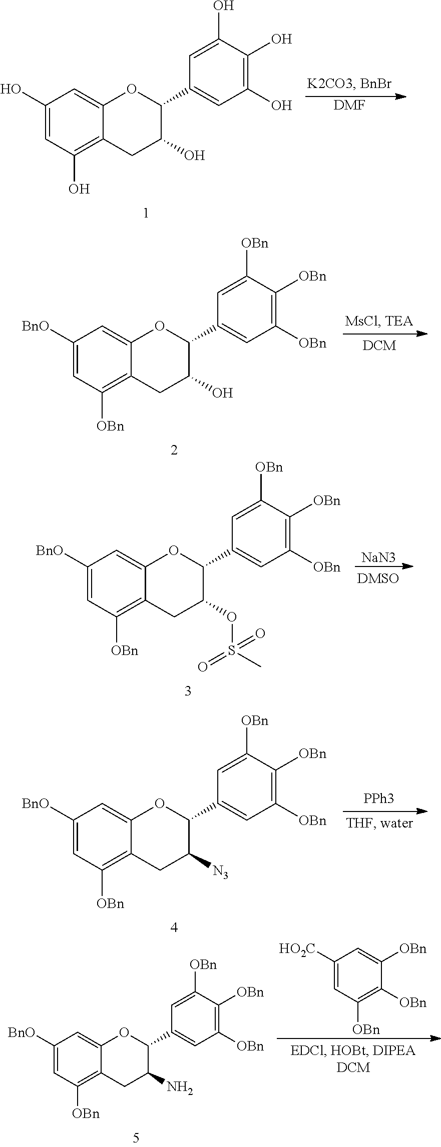

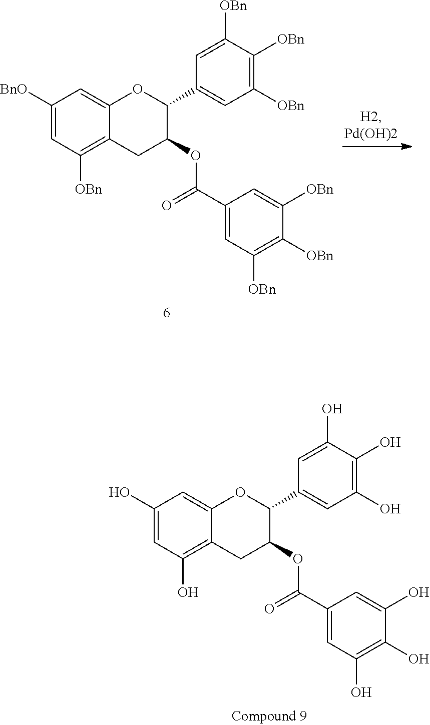 [SMILES: O=C(O[C@H]1Cc2c(OCc3ccccc3)cc(OCc3ccccc3)cc2O[C@@H]1c1cc(OCc2ccccc2)c(OCc2ccccc2)c(OCc2ccccc2)c1)c1cc(OCc2ccccc2)c(OCc2ccccc2)c(OCc2ccccc2)c1 ; O=C(O[C@H]1Cc2c(O)cc(O)cc2O[C@@H]1c1cc(O)c(O)c(O)c1)c1cc(O)c(O)c(O)c1]

Step 1: Synthesis of (2). To solution of (−)-EGC 1 (45.0 g, 0.147 mol) and K2CO3 (203.1 g, 1.469 mol, 10 eq) in DMF (400 mL) was added benzyl bromide (130.7 g, 0.764 mol, 5.2 eq) at −20° C. The mixture was stirred at r.t. for 48h and then poured into water (1500 mL). The resulting mixture was extracted with ethyl acetate and the extract was dried over Na2SO4. After evaporation of the solvent the residue was recrystallized several times from Et2O to 95+ % purity by NMR to afford compound 2 as white solid (33.2 g, 30% yield).

Step 2: Synthesis of (3). Triethylamine (3.0 g, 29.7 mmol, 1.5 eq) was added to a solution of compound 2 (15.0 g, 19.8 mmol) and methanesulfonyl chloride (2.95 g, 25.7 mmol, 1.3 eq) in EtOAc (800 mL) at 0° C. under nitrogen. The reaction mixture was stirred at r.t. for 4 hours. The reaction mixture was washed with saturated aqueous NaHCO3, brine, dried over Na2SO4, filtered and concentrated in vacuo at 40° C. to provide the title compound 3 as yellow oil (15.9 g, 96% yield). The mesylate 3 should be used in the next step immediately after preparation.

Step 3: Synthesis of (4). To a solution of compound 3 (15.9 g, 19.0 mmol) in 250 ml anhydrous DMSO was added sodium azide (2.47 g, 38.1 mmol, 2.0 eq), and the reaction mixture was stirred for 12 h at 100° C. The mixture was poured into cold water (1000 mL). The resulting mixture was extracted with ethyl acetate and the extract was washed with water, brine, dried over Na2SO4. The residue was purified by chromatography on silica gel with CH2Cl2. Yield 14.0 g, 94%.

Step 4: Synthesis of (5). Compound 5 (14.0 g, 17.9 mmol) and PPh3 (9.39 g, 35.8 mmol, 2.0 eq) in a mixture of THF and water (800 ml and 20 ml) was heated under reflux for 12h in inert atmosphere. Solvents were evaporated and the residue was purified by chromatography on silica gel with CHCl3: MeOH=80:1. Yield 10.9 g, 81%.

Step 5: Synthesis of (6). The mixture of compound 5 (700 mg, 0.926 mmol), acid 8 (1000 mg, 0.926 mmol, 1 eq), EDC (195 mg, 1.019 mmol, 1.1 eq), HOBT (125 mg, 0.926 mmol, 1 eq) and DIPEA (239 mg, 1.852 mmol, 2 eq) in 100 ml CH2Cl2 was stirred overnight. After the reaction was completed (TLC control) the mixture was washed with water, 5% citric acid and dried over Na2SO4. The residue was purified by chromatography on silica gel with CHCl3:MeOH=180:1. Yield 610 mg, 55%.

Step 6: Synthesis of Compound 9. To a solution of compound 6 (600 mg, 0.51 mmol) in THF:MeOH=1:1 (160 ml total) was added 300 mg 20% Pd(OH)2 on carbon and the mixture was stirred at atmosphere of H2 for 12-24 h (LCMS monitoring). (2R,3S)-5,7-dihydroxy-2-(3,4,5-trihydroxyphenyl)chroman-3-yl 3,4,5-trihydroxybenzoate was obtained after filtration, evaporation and purification by HPLC on YMC-Pack ODS-AQ column in neutral conditions with gradient H2O-acetonitrile. Yield 46 mg, 20%.

### Compound 28

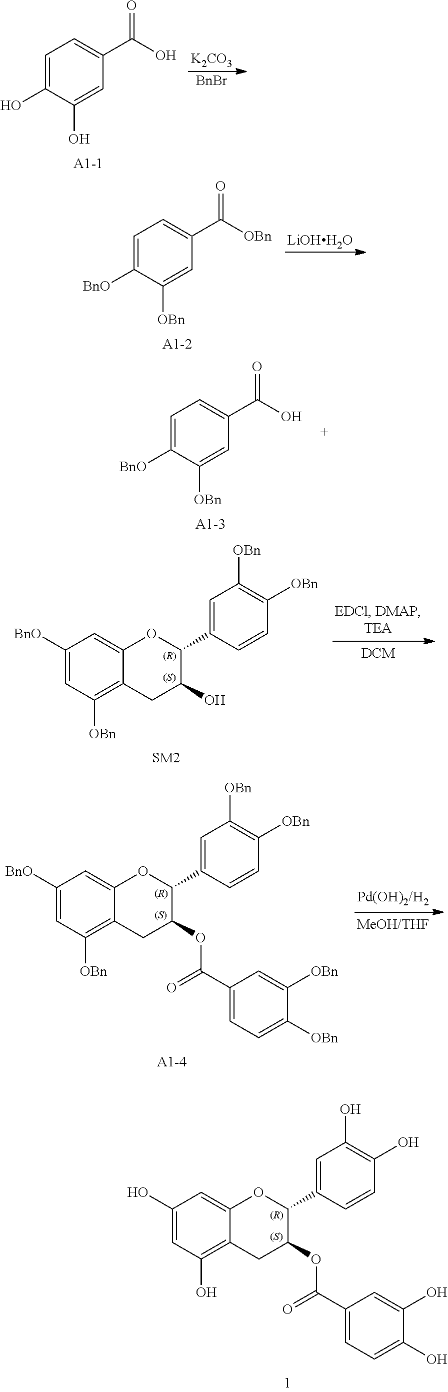

Step 1: Synthesis of benzyl 3,4-bis(benzyloxy)benzoate (SM2). To a solution of compound SM1 (1 g, 3.45 mmol) and K2CO3 (3.57 g, 25.87 mmol) in DMF (20 mL) was added BnBr (2.48 g, 14.49 mmol) at 0° C. The mixture was stirred at room temperature overnight. The reaction mixture was diluted with H2O (50 mL) and extracted with EA (50 mL×2). The combined organic layers were washed with brine (30 mL×2), dried over Na2SO4, filtered and concentrated. The residue was purified by flash chromatography on silica gel (PE/EA=3/1) to give compound SM2 (1.68 g, 75% yield) as a yellow solid. MS Calcd.: 650.3. MS Found: 651.0 [M+H]+.

Step 2: Synthesis of benzyl 3,4-bis(benzyloxy)benzoate (A1-2). To a solution of compound A1-1 (1 g, 36.5 mmol) and K2CO3 (3.13 g, 22.7 mmol) in DMF (20 mL) was added BnBr (4.13 g, 22.7 mmol) at 0° C. The mixture was stirred at room temperature overnight. The reaction mixture was diluted with H2O (50 mL) and extracted with EA (50 mL×2). The combined organic layers were washed with brine (30 mL×2), dried over Na2SO4, filtered and concentrated. The residue was purified by flash chromatography on silica gel (PE/EA=2/1) to give compound A1-2 (2.04 g, 74% yield) as a yellow solid. MS Calcd.: 424. MS Found: 425 [M+H]+.

Step 3: Synthesis of 3,4-bis(benzyloxy)benzoic acid (A1-3). A mixture of compound A1-2 (8.5 g, 0.02 mol) in MeOH (60 mL) was added LiOH·H2O (0.962 g, 0.04 mol) in H2O (20 mL). The solution was stirred at 50° C. overnight. The reaction mixture was concentrated to remove MeOH. Then the mixture was diluted with H2O (30 mL) and extracted with EA (80 mL×2). The aqueous phase was adjusted to pH<3 with 1 N HCl. Then the mixture was filtered and the filter cake was dried to give the compound A1-3 (5.3 g, 79% yield) as a white solid. MS Calcd.: 334.1. MS Found: 333.0 [M−H]−.

Step 4: Synthesis of (2R,3S)-5,7-bis(benzyloxy)-2-(3,4-bis(benzyloxy)phenyl)chroman-3-yl 3,4-bis(benzyloxy) benzoate (A1-4). To a mixture of compound SM2 (1 g, 1.54 mmol) and A1-3 (617 mg, 1.85 mmol) in DCM (20 mL) was added EDCI (589 mg, 308 mmol), DMAP (56.4 mg, 0.462 mmol) and TEA (311 mg, 3.08 mmol) under ice-water bath. The reaction mixture was stirred at room temperature overnight. The reaction mixture was diluted with H2O (50 mL) and the phases were separated. The organic layer was washed with brine (30 mL×2), dried over Na2SO4, filtered and concentrated. The residue was purified by flash chromatography on silica gel (PE/EA=5/1) to give compound A1-4 (780 mg, 52% yield) as yellow oil. MS Calcd.: 966.1. MS Found: 967.0 [M+H]+.

Step 5: Synthesis of Compound 28. To a mixture of compound A1-4 (500 mg, 0.517 mmol) in THF (10 mL) and MeOH (10 mL) was added Pd(OH)2 (10% wt., 50 mg). The mixture was stirred at room temperature under H2 atmosphere (15 psi) overnight. The reaction mixture was filtered and the filtrate was concentrated. The residue was purified by prep-HPLC to give (2R,3S)-5,7-bis(benzyloxy)-2-(3,4-bis(benzyloxy)phenyl)chroman-3-yl 3,4-bis(benzyloxy) benzoate (60 mg, 27.2% yield) as a white solid. 1H NMR (400 MHZ, DMSO-d6) δ: 9.76 (s, 1H), 9.32 (s, 2H), 9.09 (s, 1H), 8.89 (s, 2H), 7.24 (d, J=2.0 Hz, 1H), 7.29-7.18 (m, 1H), 6.76 (d, J=8.4 Hz, 2H), 6.68-6.61 (m, 2H), 5.94 (d, J=2.0 Hz, 1H), 5.81 (d, J=2.0 Hz, 1H), 5.27-5.22 (m, 1H), 5.05 (d, J=6.4 Hz, 1H), 2.73-2.58 (m, 2H). MS Calcd.: 426.1. MS Found: 427.0 [M+H]+.

### Compound 29

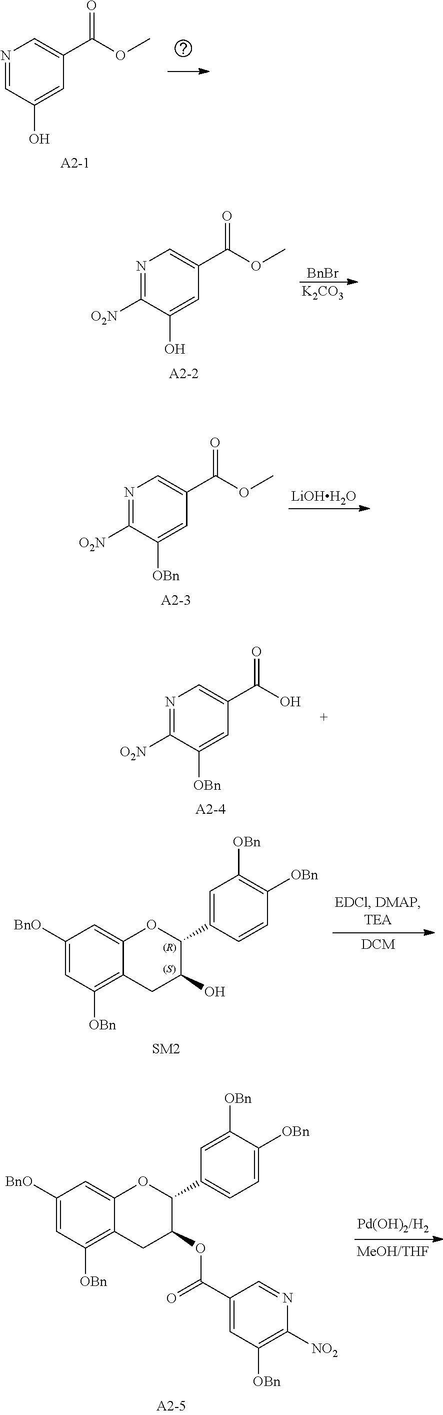

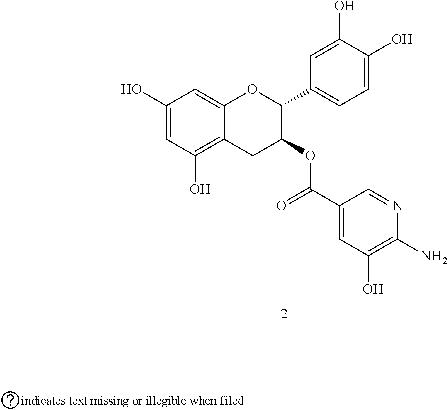 [SMILES: Nc1ncc(C(=O)O[C@H]2Cc3c(O)cc(O)cc3O[C@@H]2c2ccc(O)c(O)c2)cc1O]

Step 1: Synthesis of methyl 5-hydroxy-6-nitronicotinate (A2-2). To a solution of compound A2-1 (5 g, 32.67 mmol) in H2SO4 (50 mL) was added HNO3 (4.12 g, 65.34 mmol) at 0° C. The reaction mixture was stirred at room temperature overnight. The reaction mixture was poured into 100 mL of ice-water and stirred for 20 minutes. The mixture was filtered to give compound A2-2 (3.5 g, 54% yield) as a yellow solid. MS Calcd.: 198.03. MS Found: 199 [M+H]+.

Step 2: Synthesis of methyl 5-(benzyloxy)-6-nitronicotinate (A2-3). To a solution of compound A2-2 (2.64 g, 13.33 mmol) and K2CO3 (3.69 g, 26.67 mmol) in DMF (25 mL) was added BnBr (4.56 g, 26.67 mmol) at 0° C. The reaction mixture was stirred at room temperature overnight. The reaction mixture was diluted with H2O (50 mL) and extracted with EA (50 mL×2). The combined organic layers were washed with brine (30 mL×2), dried over Na2SO4, filtered and concentrated. The residue was purified by flash chromatography on silica gel (PE/EA=3/1) to give compound A2-3 (2.1 g, 54.7% yield) as a yellow solid. MS Calcd.: 288.1; MS Found: 289 [M+H]+.

Step 3: Synthesis of 5-(benzyloxy)-6-nitronicotinic acid (A2-4). To a mixture of compound A1-2 (2.1 g, 7.29 mmol) in THF (60 mL) was added LiOH·H2O (0.35 g, 14.58 mmol) in H2O (8 mL). The solution was stirred at room temperature overnight. The reaction mixture was concentrated in vacuo to remove the THF. The mixture was diluted with H2O (30 mL) and extracted with EtOAc (50 mL×2). The aqueous phase was adjusted to pH<3 with 1 N HCl and extracted with DCM (50 mL×2) to give compound A2-4 (1.7 g, 85% yield) as a white solid. MS Calcd.: 274.1. MS Found: 273.0 [M−H]−.

Step 4: Synthesis of (2R,3S)-5,7-bis(benzyloxy)-2-(3,4-bis(benzyloxy)phenyl)chroman-3-yl5-(benzyloxy)-6-nitronicotinate (A2-5). A mixture of compound SM2 (700 mg, 1.077 mmol) and compound A2-4 (354 mg, 1.29 mmol) in DCM (20 mL) was added EDCI (412 mg, 2.15 mmol), DMAP (39.4 mg, 0.32 mmol) and TEA (218 mg, 2.15 mmol) under ice-water bath. The mixture was stirred at room temperature overnight. The reaction mixture was diluted with H2O (50 mL) and the phases were separated. The organic layer was washed with brine (50 mL×2), dried over Na2SO4, filtered and concentrated. The residue was purified by flash chromatography on silica gel (PE/EA=5/1) to give compound A2-5 (550 mg, 56% yield) as yellow oil. MS Calcd.: 906.3. MS Found: 907.0 [M+H]+.

Step 5: Synthesis of Compound 29. To a mixture of compound A2-5 (500 mg, 0.517 mmol) in THF (10 mL) and MeOH (10 mL) was added Pd(OH)2 (10% wt., 50 mg). The reaction mixture was stirred at room temperature under H2 atmosphere (15 psi) overnight. The reaction mixture was filtered and the filtrate was concentrated. The residue was purified by prep-HPLC to give (2R,3S)-2-(3,4-dihydroxyphenyl)-5,7-dihydroxychroman-3-yl 6-amino-5-hydroxynicotinate (90 mg, 29.8% yield) as a white solid. 1H NMR (400 MHZ, DMSO-d6) δ: 9.90 (s, 1H), 9.34 (s, 1H), 9.09 (s, 1H), 8.91 (d, J=4.4 Hz, 2H), 7.97 (d, J=2.0 Hz, 1H), 7.14 (d, J=2.0 Hz, 1H), 6.76 (d, J=2.0 Hz, 1H), 6.69-6.62 (m, 2H), 6.41 (s, 2H), 5.95 (d, J=2.4 Hz, 1H), 5.81 (d, J=2.0 Hz, 1H), 5.28-5.21 (m, 1H), 5.05 (d, J=1 Hz, 1H), 2.77-2.57 (m, 2H). MS Calcd.: 426.1. MS Found: 427.1 [M+H]+.

### Compound 30

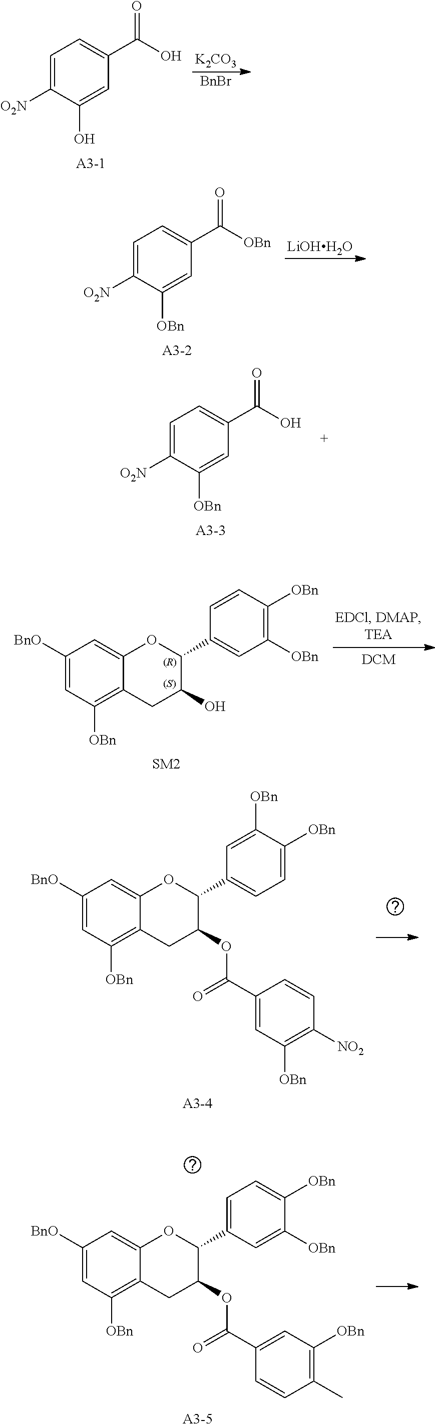

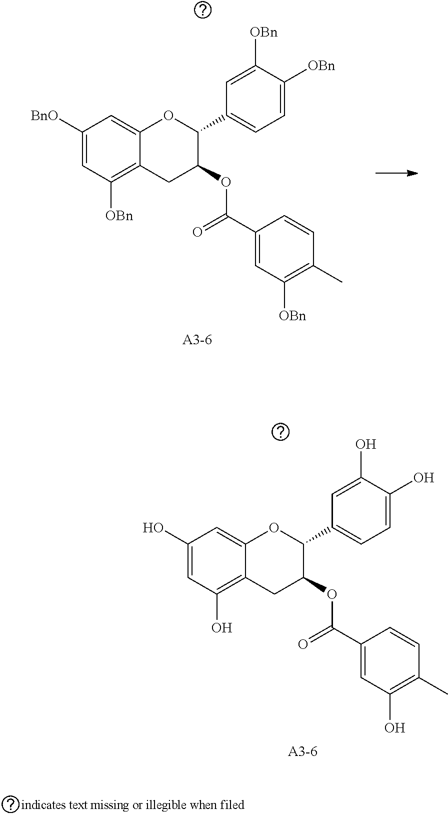 [SMILES: Cc1ccc(C(=O)O[C@H]2Cc3c(OCc4ccccc4)cc(OCc4ccccc4)cc3O[C@@H]2c2ccc(OCc3ccccc3)c(OCc3ccccc3)c2)cc1OCc1ccccc1 ; Cc1ccc(C(=O)O[C@H]2Cc3c(O)cc(O)cc3O[C@@H]2c2ccc(O)c(O)c2)cc1O]

Step 1: Synthesis of benzyl 3-(benzyloxy)-4-nitrobenzoate (A3-2). To a solution of compound A3-1 (1 g, 5.46 mmol) and K2CO3 (1.51 g, 10.92 mmol) in DMF (20 mL) was added BnBr (2.8 g, 16.38 mmol) at 0° C. The reaction mixture was stirred at room temperature overnight. The reaction mixture was diluted with H2O (40 mL) and extracted with EA (30 mL×2). The combined organic layers were washed with brine (30 mL×2), dried over Na2SO4, filtered and concentrated. The residue was purified by flash chromatography on silica gel (PE/EA=3/1) to give compound A3-2 (1.8 g, 90.9% yield) as a white solid. MS Calcd.: 363.1. MS Found: 364.0 [M+H]+.

Step 2: Synthesis of benzyl 3,4-bis(benzyloxy)benzoate (A3-3). To a mixture of compound A3-2 (1 g, 2.75 mmol) in THF (10 mL) was added LiOH·H2O (132 mg, 5.51 mmol) in H2O (4 mL). The solution was stirred at room temperature overnight. The reaction mixture was concentrated in vacuo to remove THF. The mixture was diluted with H2O (10 mL) and extracted with EA (30 mL×2). The aqueous phase was adjusted to pH<3 with 1 N HCl and extracted with DCM (30 mL×2) to give the compound A3-3 (700 mg, 93.1% yield) as a white solid. MS Calcd.: 273.1. MS Found: 272.0 [M−H]−.

Step 3: Synthesis of (2R,3S)-5,7-bis(benzyloxy)-2-(3,4-bis(benzyloxy)phenyl)chroman-3-yl3-(benzyloxy)-4-nitrobenzoate (A3-4). A mixture of compound SM2 (1 g, 1.54 mmol) and A3-3 (420 mg, 1.85 mmol) in DCM (20 mL) was added EDCI (589 mg, 308 mmol), DMAP (56.4 mg, 0.462 mmol) and TEA (311 mg, 3.08 mmol) under ice-water bath. The reaction mixture was stirred at room temperature overnight. The reaction mixture was diluted with H2O (50 mL) and the phases were separated. The organic layer was washed with brine (30 mL×2), dried over Na2SO4, filtered and concentrated. The residue was purified by flash chromatography on silica gel (PE/EA=5/1) to give compound A1-4 (780 mg, 52% yield) as yellow oil. MS Calcd.: 905.3. MS Found: 906.6 [M+H]+.

Step 4: Synthesis of (2R,3S)-5,7-bis(benzyloxy)-2-(3,4-bis(benzyloxy)phenyl)chroman-3-yl4-amino-3-(benzyloxy)benzoate (A3-5). A mixture of NH4Cl (45.6 mg, 0.85 mmol) and Fe (241.2 mg, 4.2 mmol) in 50 mL of EtOH and 10 mL of H2O was stirred at 90° C. for 1 hour. Then compound A3-4 (780 mg, 0.86 mmol) in ACN (5 mL) was slowly added and the mixture was stirred at 90° C. for 3 hours. The reaction mixture was cooled down to room temperature and filtered. The filtrate was concentrated to get a crude product, which was diluted with H2O (30 mL) and extracted with DCM (30 mL×2). The combined organic layers were washed with brine (30 mL×2) and dried over Na2SO4, filtered and concentrated. The residue was purified by flash chromatography on silica gel (PE/EA=1/1) to give compound A3-5 (550 mg, 66.2% yield) as yellow oil. MS Calcd.: 875.3. MS Found: 876.0 [M+H]+.

Step 5: Synthesis of (2R,3S)-5,7-bis(benzyloxy)-2-(3,4-bis(benzyloxy)phenyl)chroman-3-yl3-(benzyloxy)-4-(methylsulfonamido)benzoate (A3-6). To a solution of compound A3-5 (500 mg, 0.801 mmol) and TEA (485.4 mg, 4.806 mmol) in DCM (20 mL) was added MsCl (182.6 mg, 1.602 mmol) at 0° C. The reaction mixture was stirred at room temperature overnight. The reaction mixture was diluted with H2O (30 mL) and the phases were separated. The organic layer was washed with brine (30 mL×2), dried over Na2SO4, filtered and concentrated. The residue was purified by flash chromatography on silica gel (PE/EA=3/1) to give compound A3-6 (180 mg, 33% yield) as a yellow solid. MS Calcd.: 953.3. MS Found: 954.0 [M+H]+.

Step 6: Synthesis of Compound 30. To a mixture of compound A3-6 (180 mg, 0.14 mmol) in THF (10 mL) and MeOH (10 mL) was added Pd(OH)2 (10% wt., 18 mg). The mixture was stirred at room temperature under H2 atmosphere (15 PSI) overnight. The reaction mixture was filtered and concentrated. The residue was purified by prep-HPLC to give (2S,3R)-2-(3,4-dihydroxyphenyl)-5,7-dihydroxychroman-3-yl3-hydroxy-4-(methylsulfonamido)benzoate (96 mg, 8.7% yield) as a white solid. 1H NMR (400 MHZ, DMSO-d6) δ: 9.35 (s, 1H), 9.04 (s, 1H), 8.88 (s, 2H), 7.36-7.27 (m, 3H), 6.77 (d, J=1.6 Hz, 1H), 6.69-6.62 (m, 2H), 5.94 (d, J=2.0 Hz, 1H), 5.81 (d, J=2.4 Hz, 1H), 5.31-5.27 (m, 1H), 5.08 (d, J=6.0 Hz, 1H), 2.75 (s, 3H), 2.77-2.50 (m, 3H). MS Calcd.: 503.9. MS Found: 502.0 [M−H]−.

### Compound 31

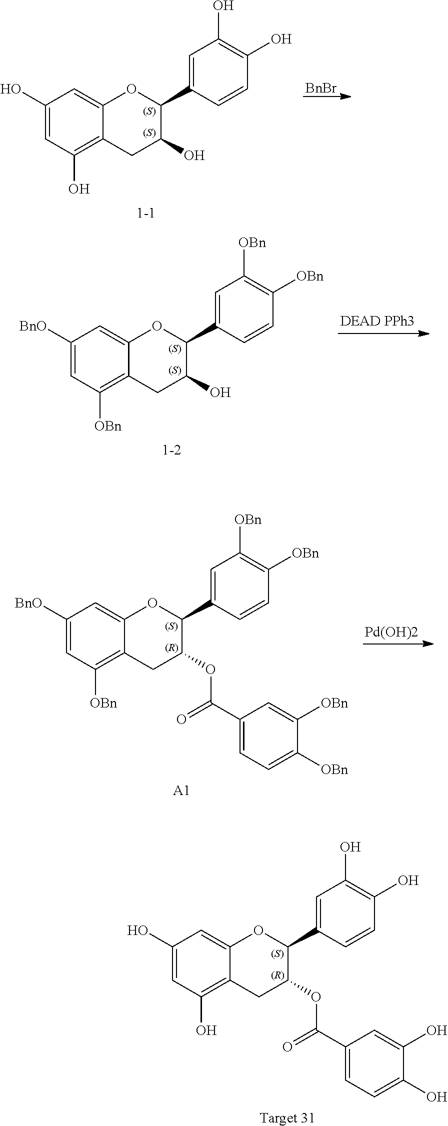

Step 1: Synthesis of (2S,3R)-5,7-bis(benzyloxy)-2-(3,4-bis(benzyloxy)phenyl)chroman-3-ol(1-2). To a solution of compound 1-1 (1 g, 3.45 mmol) in DMF (20 mL) was added NaH (579 mg, 14.47 mmol, 60% wt. in mineral oil) at 0° C. The mixture was stirred at 0° C. for 20 minutes. BnCl (1.83 g, 14.47 mmol) was added at 0° C. and the solution was stirred at room temperature overnight. The reaction mixture was diluted with H2O (50 mL) and extracted with EA (50 mL×2). The combined organic layers were washed with brine (30 mL×2), dried over Na2SO4, filtered and concentrated. The residue was purified by flash chromatography on silica gel (PE/EA=3/1) to give compound 1-2 (1.72 g, 77% yield) as yellow oil. MS Calcd.: 650.3. MS Found: 651.0 [M+H]+.

Step 2: Synthesis of (2R,3S)-5,7-bis(benzyloxy)-2-(3,4-bis(benzyloxy)phenyl)chroman-3-yl3,4-bis(benzyloxy)benzoate(1-3). To a solution of compound 1-2 (600 mg, 0.92 mmol) in THF (15 mL) was added PPh3 (362 mg, 1.38 mmol), compound A1-3 (370 mg, 1.11 mmol) and DEAD (240 mg, 1.38 mmol) at 0° C. The reaction mixture was stirred at room temperature overnight. The reaction mixture was diluted with water (30 mL) and extracted with EA (30 mL×3). The combined organic layers were dried over Na2SO4 and filtered. The filtrate was concentrated to give a crude product, which was purified by flash chromatography on reverse phase silica gel (ACN/H2O=5%-95%, 214 nm, 30 min) to give compound 1-3 (100 mg, 11% yield) as a yellow solid. MS Calcd.: 966.4. MS Found: 967 [M+H]+.

Step 3: Synthesis of Compound 31. To a mixture of compound 1-3 (100 mg, 0.1 mmol) in EA (10 mL) was added Pd(OH)2 (10% wt., 10 mg). The mixture was stirred at room temperature under H2 of balloon for 5 hours. The reaction mixture was filtered and concentrated. The residue was purified by flash chromatography on reverse phase silica gel (ACN/H2O=5%-95%, 214 nm, 30 min) to give (2R,3S)-2-(3,4-dihydroxyphenyl)-5,7-dihydroxychroman-3-yl 3,4-dihydroxybenzoate (21.6 mg, 49% yield) as a white solid. 1H NMR (400 MHZ, CD3OD) δ: 7.34-7.30 (m, 2H), 6.86-6.69 (m, 4H), 5.96-5.93 (m, 2H), 5.40-5.33 (m, 1H), 5.07-5.03 (m, 1H), 2.88-2.83 (m, 1H), 2.74-2.68 (m, 1H). MS Calcd.: 426.1. MS Found: 427.1 [M+H]+.

### Compound 33

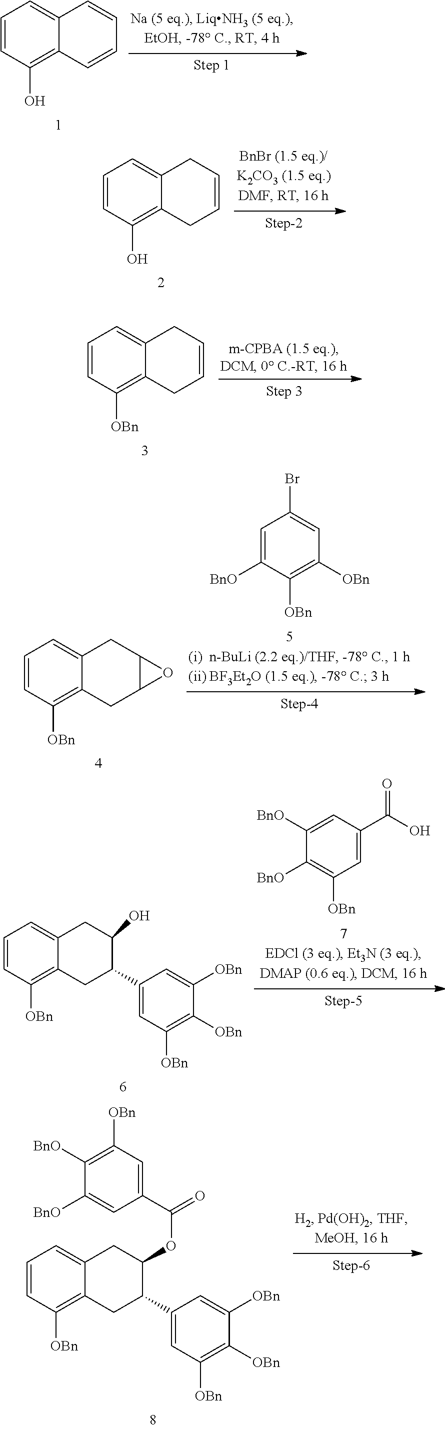

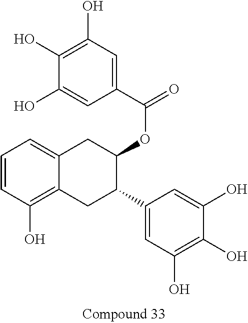 [SMILES: O=C(O[C@@H]1Cc2cccc(O)c2C[C@H]1c1cc(O)c(O)c(O)c1)c1cc(O)c(O)c(O)c1]

Step 1: Synthesis of 5,8-dihydronaphthalen-1-ol (2). To a solution of naphthalen-1-ol (1 g, 6.944 mmol, 1 eq.), in ethanol (40 mL) was added ammonia in THF (0.7 mL, 34.72 mmol, 5 eq.). Then reaction mixture was cool to −70° C. followed by added metallic sodium (0.798 g, 34.72 mmol, 5 eq.) in small pieces over a period of 10 min slowly the deep green naphthalene/sodium complex was observed. After quenching the entire complex with t-BuOH, the solution was stirred for 4 hours at room temperature. Solid portion was removed by filtration and washed with ethanol. The combined solution was concentrated, obtained crude was dissolved in diethyl ether (50 ml) and washed with water, brine, dried over Na2SO4, filtered and evaporated under reduced pressure. The crude compound was purified by flash column chromatography product was eluted with 10% EtOAc in hexane as an eluent affords to obtain desired 5,8-dihydronaphthalen-1-ol as a pale yellow color solid (0.8 g, 80% yield). ¹HNMR (400 MHz, DMSO-d₆): δ 7.05-6.96 (m, 1H), 6.70 (q, J=6.8 Hz, 1H), 6.60 (t, J=4.8 Hz, 1H), 5.90 (t, J=12.8 Hz, 1H), 4.65 (d, J=14.0 Hz, 1H), 3.39 (d, J=6.0 Hz, 1H), 3.26 (d, J=6.0 Hz, 1H), 2.63 (t, J=6.0 Hz, 2H), 1.86-1.73 (m, 1H).

Step 2: 5-(benzyloxy)-1,4-dihydronaphthalene (3). To a solution of 5,8-dihydronaphthalen-1-ol (0.8 g, 5.479 mmol, 1 eq.) in 10 mL DMF was added K₂CO₃ (1.1 g, 8.219 mmol, 1.5 eq.) and benzyl bromide (0.8 mL, 8.219 mmol, 1.5 eq.) at 0° C., reaction mixture stirred at RT for 16 h. Reaction progress was monitor by TLC. Reaction mixture was quenched with cold water, extracted with EtOAc (3×150 mL), washed with brine and dried over anhydrous Na2SO4. Organic layer was evaporated under reduced pressure to obtained crude compound. The crude compound was purified by flash column chromatography eluted with 20% EtOAc in hexane as an eluent affords to obtain desired 5-(benzyloxy)-1,4-dihydronaphthalene as a yellow solid (0.7 g, 58% yield). ¹HNMR (400 MHZ, DMSO-d₆): δ 7.54-7.24 (m, 5H), 7.10 (t, J=8.0 Hz, 1H), 6.75-6.69 (m, 2H), 5.89 (q, J=10.4 Hz, 1H), 5.07 (s, 2H), 3.38 (d, J=16.8 Hz, 1H), 2.75 (d, J=2.4 Hz, 1H), 1.78 (d, J=1.6 Hz, 1H).

Step 3: Synthesis of 5-(benzyloxy)-1,4-dihydronaphthalene (4). To a solution of 5-(benzyloxy)-1,4-dihydronaphthalene (0.7 g, 2.966 mmol, 1 eq.) in 15 mL DCM was added m-CPBA (0.76 g, 4.449 mmol, 1.5 eq.) at 0° C., reaction mixture stirred at RT for 16 h. Reaction progress was monitor by TLC. Reaction mixture was quenched with hypo solution, extracted with EtOAc (3×150 mL), washed with brine and dried over anhydrous Na₂SO₄. Organic layer was evaporated under reduced pressure to obtained crude compound. The crude compound was purified by flash column chromatography, eluted with 15% EtOAc in hexane as an eluent affords to obtain desired compound 3-(benzyloxy)-1a,2,7,7a-tetrahydronaphtho[2,3-b]oxirene as a yellow solid (0.1 g, 14% yield). ¹HNMR (400 MHZ, DMSO-d₆) δ 7.42-7.32 (m, 4H), 7.09 (t, J=7.6 Hz, 1H), 6.74 (d, J=8.8 Hz, 1H), 6.69 (d, J=8.8 Hz, 1H), 5.03 (s, 2H), 3.62 (d, J=18.6 Hz, 1H), 3.48 (d, J=8.0 Hz, 1H), 3.33 (d, J=18.4 Hz, 1H), 3.18 (d, J=18 Hz, 1H), 2.87 (d, J=18.6 Hz, 1H).

Step 4: Synthesis of (2R,3S)-5-(benzyloxy)-3-(3,4,5-tris(benzyloxy)phenyl)-1,2,3,4-tetrahydronaphthalen-2-ol (6). To a solution of (((5-bromobenzene-1,2,3-triyl) tris(oxy))tris(methylene)) tribenzene (0.37 g, 0.796 mmol, 2 eq.), in 6 mL THF was added n-BuLi (1.6M, 0.3 mL, 0.871 mmol, 2.2 eq.) at −70° C. and reaction mixture stirred at −70° C. for 2 h. After this time, Bromobenzene 3-(benzyloxy)-1a,2,7,7a-tetrahydronaphtho[2,3-b]oxirene (0.1 g, 0.396 mmol, 1 eq.), and BF₃-Et₂O (0.084 g, 0.594 mmol, 1.5 eq.) was added at −70° C. to the above reaction mass. Reaction mixture stirred at 0° C. for 3 h. Reaction progress was monitor by TLC. Reaction mixture was quenched with aq NH₄Cl solution, extracted with EtOAc (2×150 mL), washed with brine and dried over anhydrous Na2SO4. Organic layer was concentrated under reduced pressure to obtained crude compound. The crude compound was purified by flash column chromatography, eluted with 30% EtOAc in hexane as an eluent affords to obtain desired compound (2R,3S)-5-(benzyloxy)-3-(3,4,5-tris(benzyloxy)phenyl)-1,2,3,4-tetrahydronaphthalen-2-ol as a white solid (0.05 g, 20% yield). ¹HNMR (400 MHZ, DMSO-d₆) δ 7.42-7.26 (m, 20H), 7.07 (d, J=7.2 Hz, 1H), 6.84 (d, J=8.0 Hz, 1H), 6.73 (d, J=13.6 Hz, 3H), 5.07 (s, 6H), 4.88 (s, 2H), 4.62 (d, J=3.6 Hz, 1H), 4.07 (s, 1H), 2.99 (d, J=16.4 Hz, 2H), 2.72 (s, 1H), 2.65 (d, J=15.6 Hz, 2H).

Step 5: Synthesis of (2R,3S)-5-(benzyloxy)-3-(3,4,5-tris(benzyloxy)phenyl)-1,2,3,4-tetrahydronaphthalen-2-yl 3,4,5-tris(benzyloxy)benzoate (8). To a solution of 6(2R,3S)-5-(benzyloxy)-3-(3,4,5-tris(benzyloxy)phenyl)-1,2,3,4-tetrahydronaphthalen-2-ol (0.5 g, 0.661 mmol, 1 eq.), in 10 mL DCM was added 3,4,5-tris(benzyloxy)benzoic acid (0.43 g, 1.322 mmol, 2 eq.), EDCI (0.39 g, 3.305 mmol, 3 eq.), Et₃N (0.5 mL, 3.305 mmol, 3 eq.) and DMAP (0.05 g, 0.396 mmol, 0.6 eq.) at 0° C. and reaction mixture stirred at RT for 16 h. Reaction progress was monitor by TLC. Reaction mixture was quenched with hypo solution, extracted with DCM (3×50 mL), washed with brine and dried over anhydrous Na₂SO₄. Organic layer was concentrated under reduced pressure to obtained crude compound. The crude compound was purified by flash column chromatography, eluted with 20% EtOAc in hexane as an eluent affords to obtain desired compound (2R,3S)-5-(benzyloxy)-3-(3,4,5-tris(benzyloxy)phenyl)-1,2,3,4-tetrahydronaphthalen-2-yl 3,4,5-tris(benzyloxy)benzoate as a white solid (0.5 g, 71% yield). ¹HNMR (400 MHZ, DMSO-d₆) δ 7.45-7.28 (m, 35H), 7.22 (t, J=6.8 Hz, 2H), 7.11 (d, J=8.8 Hz, 1H), 6.87 (s, 2H), 6.42 (s, 2H), 6.28 (s, 1H) 5.40 (d, J=6.4 Hz, 1H), 5.21 (s, 2H), 5.16 (d, J=8.8 Hz, 1H), 5.07 (s, 6H), 5.00 (s, 2H), 4.89 (s, 2H), 4.85 (s, 2H), 2.91 (dd, J=5.2 Hz, 2H), 2.76 (dd, J=6.8 Hz, 2H).

Step 6: Synthesis of Compound 33. To a solution of (2S,3S)-5-(benzyloxy)-3-(3,4,5-tris(benzyloxy)phenyl)-1,2,3,4-tetrahydronaphthalen-2-yl 3,4,5-tris(benzyloxy)benzoate (0.2 g, 0.186 mmol, 1 eq.), in 12 mL of 1:1; THF:MeOH was added palladium hydroxide (20 wt. %., 0.42 g) at RT and the reaction mixture was stirred under hydrogen atmosphere for 16 h. Then the mixture was passed through a pad of celite to remove the catalyst. The filtrate was concentrated under reduced pressure. Obtained crude compound was purified by Prep-HPLC to obtain (2S,3S)-5-hydroxy-3-(3,4,5-trihydroxyphenyl)-1,2,3,4-tetrahydronaphthalen-2-yl 3,4,5-trihydroxybenzoate as a grey solid (0.025 g, 31% yield). ¹HNMR (400 MHZ, DMSO-d₆): δ 6.93 (t, J=8.0 Hz, 1H), 6.63 (d, J=8.0 Hz, 1H), 6.54 (t, J=7.2 Hz, 1H), 6.17 (s, 2H), 5.25 (d, J=6.0 Hz, 1H), 3.06 (t, J=6.8 Hz, 1H), 2.98 (t, J=8.4 Hz, 2H), 2.81 (dd, J=7.2 Hz, 1H), 2.69 (dd, J=7.6 Hz, 2H). LCMS: (M−H⁻): m/Z: 439.1.

### Compound 32

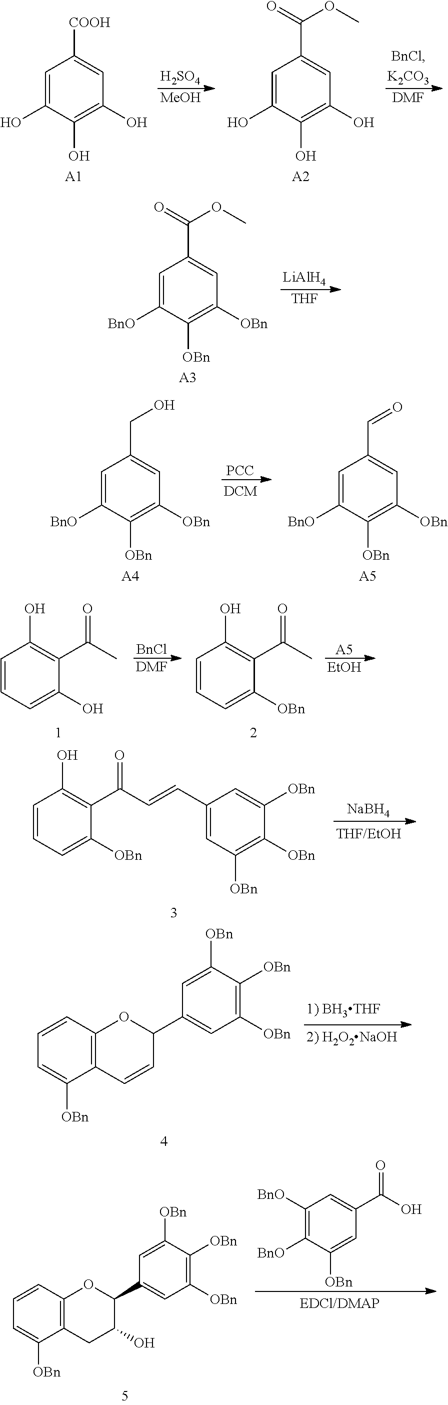

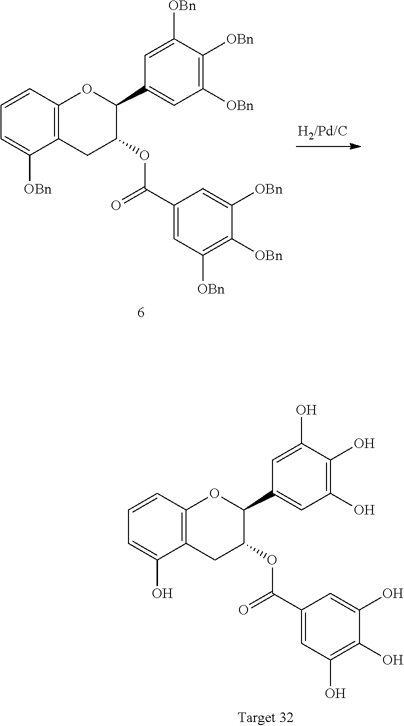 [SMILES: O=C(O[C@@H]1Cc2c(OCc3ccccc3)cccc2O[C@H]1c1cc(OCc2ccccc2)c(OCc2ccccc2)c(OCc2ccccc2)c1)c1cc(OCc2ccccc2)c(OCc2ccccc2)c(OCc2ccccc2)c1 ; O=C(O[C@@H]1Cc2c(O)cccc2O[C@H]1c1cc(O)c(O)c(O)c1)c1cc(O)c(O)c(O)c1]

Step 1: Synthesis of methyl 3,4,5-trihydroxybenzoate (A2). To a solution of compound A1 (20 g, 0.12 mol) in MeOH (200 mL) was added con.H2SO4 (6 mL) at 0° C. The mixture was stirred at 80° C. overnight. After cooling down to room temperature, the reaction mixture was neutralized with sat. Na2CO3 solution at 0° C. and extracted with EA (100 mL×3). The combined organic layers were washed with brine (100 mL×2), dried over Na2SO4, filtered and concentrated to give compound A2 (15 g, 70% yield) as a yellow solid. MS Calcd.: 184; MS Found: 185 [M+H]+.

Step 2: Synthesis of methyl 3,4,5-tris(benzyloxy)benzoate (A3). To a solution of compound A2 (1.8 g, 9.77 mmol) and K2CO3 (5.4 g, 39.13 mmol) in DMF (20 mL) was added BnCl (5.54 g, 43.97 mmol) at 0° C. The solution was stirred at 60° C. for 4 hours. The reaction mixture was diluted with H2O (50 mL) and extracted with EA (50 mL×2). The combined organic layers were washed with brine (30 mL×2), dried over Na2SO4, filtered and concentrated. The residue was purified by flash chromatography on silica gel (PE/EA=5/1) to give compound A3 (4.05 g, 91% yield) as a white solid. MS Calcd.: 454. MS Found: 455 [M+H]+.

Step 3: Synthesis of (3,4,5-tris(benzyloxy)phenyl)methanol (A4). A mixture of compound A3 (4.0 g, 8.81 mmol) in THF (50 mL) was added LiAlH4 (502 mg, 13.2 mol). The solution was stirred at room temperature for 3 hours. The reaction mixture was diluted with H2O (0.5 mL) and 15% NaOH solution (1 mL) at 0° C., extracted with DCM (30 mL×3). The combined organic phases were washed with brine (30 mL×2), dried over Na2SO4, filtered and concentrated to give compound A4 (3.6 g, 96% yield) as a yellow solid. MS Calcd.: 426; MS Found: 427 [M+H]+.

Step 4: Synthesis of 3,4,5-tris(benzyloxy)benzaldehyde (A5). A mixture of compound A4 (3.6 g, 8.45 mmol) in DCM (30 mL) was added PCC (2.73 g, 12.67 mmol). The reaction mixture was stirred at room temperature for 3 hours. The reaction mixture was concentrated and the residue was purified by flash chromatography on silica gel (PE/EA=5/1-3/1) to give compound A5 (2.98 g, 83% yield) as a white solid. MS Calcd.: 424. MS Found: 425 [M+H]+.

Step 5: Synthesis of 1-(2-(benzyloxy)-6-hydroxyphenyl)ethanone (2). To a solution of compound 1 (5.0 g, 32.9 mmol) in DMF (50 mL) was added K2CO3 (5.5 g, 39.47 mmol) and BnCl (5.0 g, 39.47 mmol) was added at 0° C. and the reaction mixture was stirred at 60° C. overnight. The reaction mixture was diluted with water (50 mL) and extracted with EA (30 mL×2). The combined organic layers were dried over Na2SO4 and filtered. The filtrate was concentrated to give the crude product which was purified by flash chromatography on silica gel (PE/EA=10/1-5/1) to give compound 2 (5.6 g, 70% yield) as a yellow solid. MS Calcd.: 242; MS Found: 243 [M+H]+.

Step 6: Synthesis of (E)-1-(2-(benzyloxy)-6-hydroxyphenyl)-3-(3,4,5-tris(benzyloxy)phenyl)prop-2-en-1-one (3). To a solution of compound 2 (1.4 g, 5.78 mmol) in EtOH (20 mL) was added compound A5 (2.7 g, 6.36 mmol) and KOH (1.62 g, 28.9 mmol). The mixture was stirred at room temperature overnight. The reaction mixture was concentrated. The residue was diluted with H2O (50 mL) and extracted with EA (50 mL×3). The combined organic layers were washed with brine (30 mL×2), dried over Na2SO4, filtered and concentrated. The solid was triturated with EtOH (20 mL), filtered, washed with EtOH (10 mL), then dried to give compound 3 (3.25 g, 87% yield) as a yellow solid. MS Calcd.: 648; MS Found: 649 [M+H]+.

Step 7: Synthesis of 5-(benzyloxy)-2-(3,4,5-tris(benzyloxy)phenyl)-2H-chromene (4). To a solution of compound 3 (1.0 g, 1.54 mmol) in THF/EtOH (20 mL/6 mL) was added CeCl3 (951 mg, 3.86 mmol) and NaBH4 (147 mg, 3.86 mmol) at 0° C. The reaction mixture was stirred at room temperature overnight. The reaction mixture was diluted with water (30 mL) and extracted with DCM (20 mL×2). The combined organic layers were dried over Na2SO4 and filtered. The filtrate was concentrated to give the crude product which was purified by flash chromatography on silica gel (PE/EA=10/1) to give compound 4 (692 mg, 71% yield) as a yellow solid. MS Calcd.: 632. MS Found: 633 [M+H]+.

Step 8: Synthesis of (2S,3R)-5-(benzyloxy)-2-(3,4,5-tris(benzyloxy)phenyl)chroman-3-ol (5). A solution of compound 4 (50 mg, 0.08 mmol) in 15 mL of THF was added BH3·THF (1M, 0.8 mL) at ice-water bath for 2 hours. The mixture was added 3 N of NaOH solution (0.22 mL, 0.68 mmol) and 30% aqueous solution of H2O2 (77 mg, 0.68 mmol) at 0° C. The reaction was stirred at 65° C. overnight. The reaction mixture was then diluted with H2O (10 mL) and extracted with EA (20 mL×2). The organic phase was concentrated and purified by flash chromatography on silica gel (PE/EA=3/1) to afforded compound 5 (40 mg, 80% yield) as a yellow solid. 1H NMR (400 MHZ, DMSO-d6) δ: 7.48-7.38 (m, 18H), 7.35-7.28 (m, 3H), 7.10 (t, J=4.2 Hz, 1H), 7.06 (s, 2H), 6.64 (d, J=8.0 Hz, 1H), 6.48 (d, J=8.0 Hz, 1H), 5.14-5.09 (m, 6H), 4.93 (s, 2H), 4.69 (d, J=7.6 Hz, 1H), 4.11-4.04 (m, 1H), 2.90-2.84 (m, 1H), 2.61-2.55 (m, 1H). MS Calcd.: 650. MS Found: 651 [M+H]+.

Step 9: Synthesis of (2S,3R)-5-(benzyloxy)-2-(3,4,5-tris(benzyloxy)phenyl)chroman-3-yl 3,4,5-tris(benzyloxy)benzoate (6). To a mixture of compound 5 (288 mg, 0.44 mmol) in DCM (30 mL) was added 3,4,5-tris(benzyloxy)benzoic acid (234 mg, 0.53 mmol), EDCI (226 mg, 1.32 mmol) and DMAP (54 mg, 0.44 mmol) at ice-water bath. The solution was stirred at room temperature overnight. The reaction mixture was diluted with H2O (50 mL) and extracted with DCM (30 mL×2). The combined organic layers were washed with brine (30 mL×2), dried over Na2SO4, filtered and concentrated. The residue was purified by flash chromatography on reverse phase silica gel (ACN/H2O=5%-95%, 254 nm, 40 min) to give compound 6 (295 mg, 62% yield) as yellow oil. MS Calcd.: 1072. MS Found: 1073 [M+H]+.

Step 10: Synthesis of Compound 32. To a mixture of compound 6 (295 mg, 0.28 mmol) in EA (20 mL) was added Pd(OH)2 (10% wt., 30 mg). The mixture was stirred at room temperature under H2 of balloon overnight. The reaction mixture was filtered and concentrated. The residue was purified by pre-HPLC to give (2R,3R)-2-(4-((ethylcarbamoyl)oxy)-3,5-dihydroxyphenyl)-5,7-dihydroxychroman-3-yl 3,4,5-trihydroxybenzoate (35 mg, 29% yield) as a white solid. 1H NMR (400 MHZ, CD3OD) δ: 6.99-6.95 (m, 3H), 6.46 (d, J=8.4 Hz, 1H), 6.41-6.36 (m, 3H), 5.45-5.41 (m, 1H), 5.10 (d, J=5.6 Hz, 1H), 2.85 (t, J=4.6 Hz, 2H). MS Calcd.: 442. MS Found: 441 [M+H]−.

### Scaffold 1

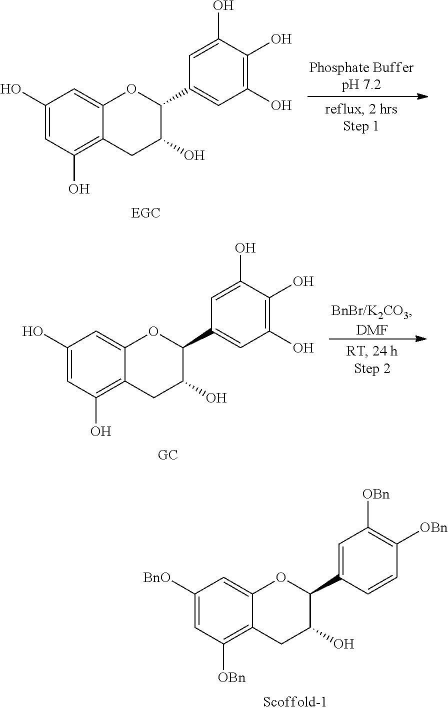 [SMILES: Oc1cc(O)c2c(c1)O[C@H](c1cc(O)c(O)c(O)c1)[C@H](O)C2 ; "Phosphate BufferpH 7.2" ; Oc1cc(O)c2c(c1)O[C@@H](c1cc(O)c(O)c(O)c1)[C@H](O)C2 ; "BnBr/K2CO3,DMF" ; O[C@@H]1Cc2c(OCc3ccccc3)cc(OCc3ccccc3)cc2O[C@H]1c1ccc(OCc2ccccc2)c(OCc2ccccc2)c1]

Step 1: Synthesis of GalloCatechin (GC). (2R,3R)-2-(3,4,5-trihydroxyphenyl)-3,4-dihydro-2H-chromene-3,5,7-triol (EGC) (50 g) was treated with Phosphate Buffer pH=7.2 (c=0.1M, 140 mL). The solution was refluxed for 2 hrs and after cooling a white precipitate of GalloCatechin was obtained. After filtration, the solid was crystallized with water (500 mL) which gave in good yield and good purity the desired GC.

Step 2: Synthesis of Scaffold 1. To a stirred solution of (2S,3R)-2-(3,4,5-trihydroxyphenyl)chromane-3,5,7-triol (GC) (5.0 g, 16.33 mmol, 1 eq.) in dry DMF (30 mL) was added K₂CO₃ (11.30 g, 81.63 mmol, 5.0 eq.) and stirred at RT for 0.5 h. To this was slowly added BnBr (9.2 mL, 81.63 mmol, 5.0 eq.) drop wise at −20° C. The suspension was slowly warmed to RT and allowed to stir at RT for 24 h. After complete consumption of the starting material, the reaction mixture was filtered through pad of celite to remove K₂CO₃. The celite pad was washed with EtOAc (100 mL). The combined organic phase was washed with cold H₂O (2×50 mL), dried over Na₂SO₄, filtered and concentrated. The obtained residue was purified by [symbol] flash column chromatography with (EtOAc:Hexane, (6:1), to afford (2S,3R)-5,7-bis(benzyloxy)-2-(3,4,5-tris(benzyloxy)phenyl)chroman-3-ol (4.5 g, 36% yield) as white color solid. ¹H NMR (400 MHZ, CDCl₃): δ 7.48-7.20 (m, 25H), 6.82 (s, 2H), 6.34 (s, 1H), 6.13 (s, 1H), 5.07 (s, 8H), 5.04 (s, 1H), 4.91 (s, 2H), 4.64 (d, J=7.2 Hz, 1H), 4.03 (bs, 1H), 2.78 (dd, J=16.0 Hz, 4.8 Hz, 1H), 2.46 (dd, J=16.4 Hz, 4.8 Hz, 1H).

### Scaffold 2

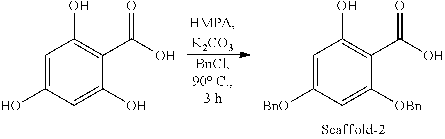

To a solution of 1-(2,4,6-trihydroxyphenyl)ethan-1-one (10 g, 59.52 mmol, 1.0 eq.) in HMPA (85 mL) was added K₂CO₃ (24.65 g, 178.56 mmol, 3.0 eq.) at RT. Then BnCl (15.0 mL, 130.95 mmol, 2.2 eq.) was added at 0° C. and stirred for 10 min at RT. Further the resulting mixture was stirred at 90° C. for 3 h. The reaction mixture was filtered and filtrate poured into ice-cold water (100 mL). Then acidified with 3N HCl (pH=4). The formed precipitate was filtered and obtained solid was dried to give 1-(2,4-bis(benzyloxy)-6-hydroxyphenyl)ethan-1-one (4.0 g, 19.3% yield) as a white color solid. ¹H NMR (400 MHZ, DMSO-d₆): δ 13.75 (s, 1H), 7.51-7.31 (m, 10H), 6.31 (d, J=2.4 Hz, 1H), 6.18 (d, J=2.4 Hz, 1H), 5.18 (s, 2H), 5.16 (s, 2H), 2.48 (s, 3H).

### Scaffold 3

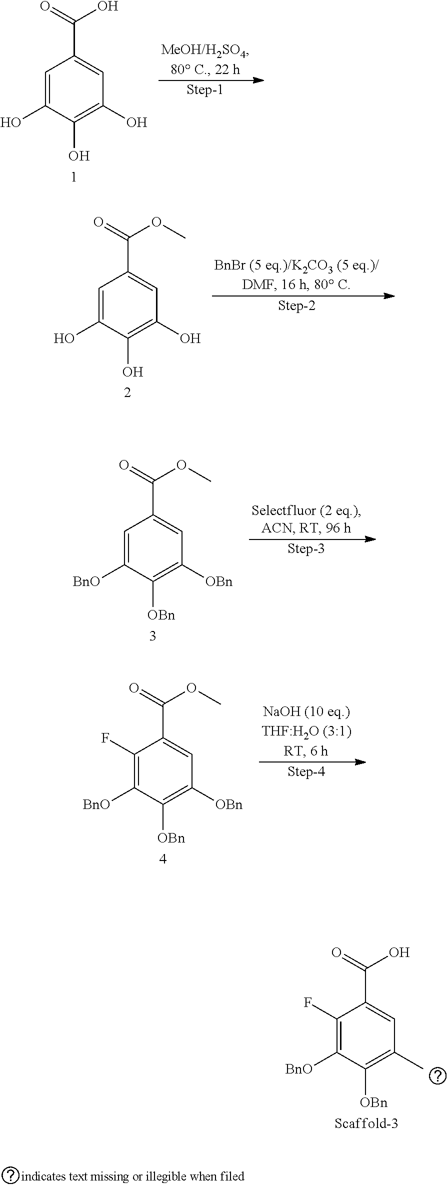

Step 1: Synthesis of methyl 3,4,5-trihydroxybenzoate (2). To a solution of methyl 3,4,5-trihydroxybenzoate (20 g, 117.564 mmol, 1 eq.), in 200 mL MeOH was added H2SO4 (11.5 mL, 117.564 mmol, 2 eq.) at 0° C. and the reaction mixture was stirred at 80° C. for 22 h. Reaction progress was monitor by TLC. After this time, reaction mixture was concentrated under reduced pressure and obtained crude diluted with cold-water to get desired product as a solid. Obtained solid was filtered and washed with water, the wet cake was dried to give the methyl 3,4,5-trihydroxybenzoate as a white solid (20 g, 92% yield). ¹HNMR (400 MHZ, DMSO-d₆) δ 9.29 (s, 3H), 6.92 (s, 2H), 3.72 (s, 3H).

Step 2: Synthesis of methyl 3,4,5-tris(benzyloxy)benzoate (3). To a suspension of compound methyl 3,4,5-trihydroxybenzoate (19 g, 103.182 mmol, 1 eq.) in DMF (200 mL) was added K₂CO₃ (71.304 g, 515.591 mmol, 5 eq.) followed by benzyl bromide (61 mL, 515.591 mmol, 5 eq.) at 0° C. The mixture was heated at 80° C. for 16 h. After this time, ice was added to reaction mass to get the desired product as solid. The obtained solid was filtered washed with water and dried to get methyl 3,4,5-tris(benzyloxy)benzoate as a white solid (30 g, 64% yield). ¹HNMR (400 MHZ, DMSO-d₆) δ 7.43-7.26 (m, 17H), 5.33 (s, 2H), 5.16 (s, 2H), 5.01 (s, 2H), 3.83 (s, 3H).

Step 3: Synthesis of methyl 3,4,5-tris(benzyloxy)-2-fluorobenzoate (4). To a solution methyl 3,4,5-tris(benzyloxy)benzoate (30 g, 66.006 mmol, 1 eq.) in 200 mL of ACN was added selectfluor (46.7 g, 132.013 mmol, 2 eq.) at 0° C. and stirred at RT for 96 h. Reaction progress was monitor by TLC. After this time, Reaction mixture was quenched with saturated solution of NaHCO₃ and product extracted with EtOAc (3×100 mL). Organic layer was washed with brine, dried over anhydrous Na₂SO₄, filtered and concentrated under reduced pressure to get crude compound. Obtained crude compound was purified by flash column chromatography, eluted with 10% EtOAc in hexane, as an eluent affords to obtain methyl 3,4,5-tris(benzyloxy)-2-fluorobenzoate as a pale brown solid (7 g, 22% yield). ¹H NMR (400 MHZ, DMSO-d₆) δ 7.42-7.28 (m, 16H), 5.33 (s, 2H), 5.16 (s, 2H), 5.14 (s, 2H), 3.81 (s, 3H), ¹⁹F NMR (400 MHZ, DMSO-d₆) δ−134.52.

Step 4: Synthesis of Scaffold 3. To a solution of methyl 3,4,5-tris(benzyloxy)-2-fluorobenzoate (7 g, 14.814 mmol, 1 eq.) in THF/H₂O (3:1) (50 mL) was added NaOH (5.9 g, 148.145 mmol, 10 eq.) and stirred at 80° C. for 6 h. The reaction mixture was concentrated under reduced pressure, obtained residue was diluted with H₂O (30 mL) and product was a extracted with EtOAc (2×80 mL). The aqueous phase pH was adjusted to <3 with 1N HCl. Then the mixture was filtered and the filter cake was dried. The crude compound was purified by flash column chromatography, eluted with 10% MeOH in DCM, as an eluent affords to obtain 3,4,5-tris(benzyloxy)-2-fluorobenzoic acid as a white solid (3.8 g, 60% yield). ¹H NMR (400 MHz, DMSO-d₆): δ 13.22 (s, 1H), 7.42 (d, J=1.2 Hz, 2H), 7.44-7.26 (m, 10H), 5.14 (s, 2H), 5.12 (s, 2H), 3.81 (s, 3H).

### Scaffold 4

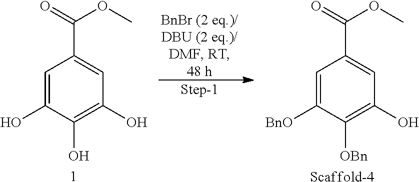

To a suspension of methyl 3,4,5-trihydroxybenzoate (50 g, 294.110 mmol, 1 eq.) in DMF (250 mL) was added DBU (70 mL, 588.200 mmol, 2 eq.) followed by benzyl bromide (178 mL, 588.200 mmol, 2 eq.) at 0° C. The reaction mass was allowed to stir at RT for 48 h. After this time, reaction mixture was diluted with ice cold water, precipitated solid was filtered washed and washed with water. Obtained crude compound was purified by column chromatography using EtOAc in hexane to get methyl 3,4-bis(benzyloxy)-5-hydroxybenzoate as a yellow solid (9.5 g, 9% yield). ¹HNMR (400 MHZ, DMSO-d₆) δ 9.77 (s, 1H), 7.45-7.33 (m, 7H), 7.29-7.27 (m, 3H), 17.5 (d, J=2.0 Hz, 2H), 5.12 (s, 2H), 5.02 (s, 2H), 3.79 (s, 3H).

### Scaffold 5

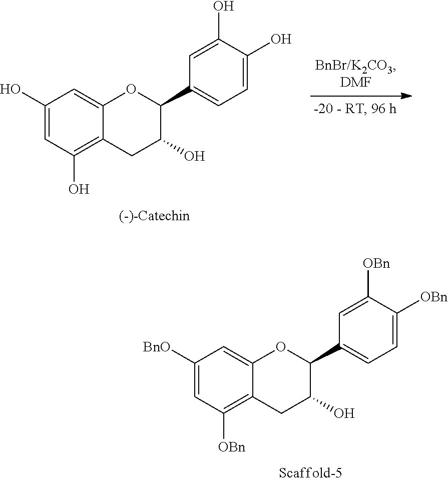 [SMILES: Oc1cc(O)c2c(c1)O[C@@H](c1ccc(O)c(O)c1)[C@H](O)C2 ; O[C@@H]1Cc2c(OCc3ccccc3)cc(OCc3ccccc3)cc2O[C@H]1c1ccc(OCc2ccccc2)c(OCc2ccccc2)c1]

To a solution of (2S,3R)-2-(3,4-dihydroxyphenyl)chromane-3,5,7-triol (1.26 g, 4.36 mmol, 1.0 eq.) in dry DMF (15 mL) was added K₂CO₃ (2.41 g, 17.44 mmol, 4.0 eq.) and stirred at RT for 0.5 h. To this was slowly added BnBr (2.1 mL, 17.44 mmol, 4.0 eq.) drop wise at −20° C. The suspension was slowly warmed to RT and allowed to stir at RT for 96 h. After complete consumption of the starting material monitored by TLC, the reaction mixture was filtered through pad of celite to remove K₂CO₃. The celite pad was washed with EtOAc (100 mL). The combined organic phase was washed with cold H₂O (2×50 mL) and brine (50 mL), dried over Na₂SO₄, filtered and concentrated. The obtained residue was purified by flash column chromatography with (EtOAc: Hexane, (5:1), to afford (2R,3R)-5,7-bis(benzyloxy)-2-(3,4,5-tris(benzyloxy)phenyl)chroman-3-ol (1.28 g, 45% yield) as an off white solid. ¹H NMR (400 MHz, DMSO-d₆): δ 7.50-7.26 (m, 20H), 7.16-6.98 (m, 2H), 6.87 (d, J=8.4 Hz, 1H), 6.32 (d, J=2.0 Hz, 1H), 6.12 (d, J=2.0 Hz, 1H), 5.16-4.98 (m, 9H), 4.63 (d, J=7.6 Hz, 1H), 4.02-3.90 (m, 1H), 2.76 (dd, J=16.8 Hz, 5.6 Hz, 1H), 2.56-2.41 (m, 1H).

### Compound 34

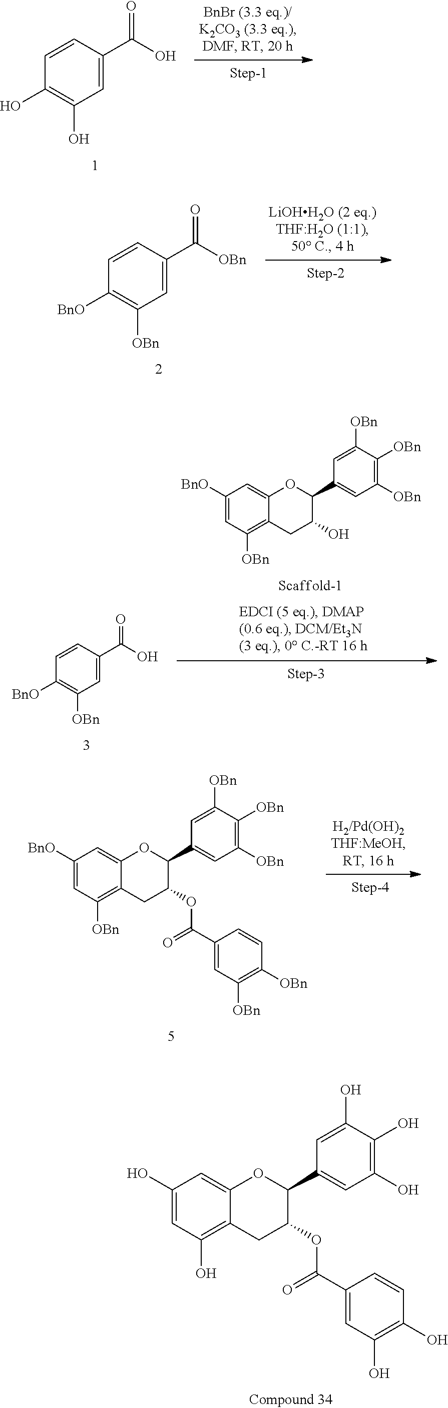 [SMILES: O=C(O)c1ccc(O)c(O)c1 ; O=C(OCc1ccccc1)c1ccc(OCc2ccccc2)c(OCc2ccccc2)c1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1ccc(OCc2ccccc2)c(OCc2ccccc2)c1 ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1ccc(OCc2ccccc2)c(OCc2ccccc2)c1 ; O=C(O[C@@H]1Cc2c(O)cc(O)cc2O[C@H]1c1cc(O)c(O)c(O)c1)c1ccc(O)c(O)c1]

Step 1: Synthesis of benzyl 3,4-bis(benzyloxy)benzoate (2). To a suspension of 3,4-dihydroxybenzoic acid (2 g, 12.976 mmol, 1 eq.) in DMF (40 mL) was added K₂CO₃ (5.9 g, 43.602 mmol, 3.3 eq.) followed by benzyl bromide (5.2 mL, 43.602 mmol, 3.3 eq.) at 0° C. The mixture was stirred at RT for 20 h until TLC showed the reaction had been completed. Reaction mixture was diluted with water and extracted with EtOAc. The solvent was evaporated, and the residue was purified by flash chromatography eluted with 10% of EtOAc in hexane as an eluent affords to obtain benzyl 3,4-bis(benzyloxy)benzoate as a white solid (3 g, 54% yield). ¹HNMR (400 MHZ, DMSO-d₆): δ 7.67 (s, 2H) 7.47-7.30 (m, 15), 6.93 (d, J=8.8 Hz, 1H), 5.32 (s, 2H), 5.23 (s, 2H), 5.19 (s, 2H).

Step 3: Synthesis of 3,4-bis(benzyloxy)benzoic acid (3). A mixture of benzyl 3,4-bis(benzyloxy)benzoate (0.5 g, 1.179 mmol, 1 eq.) in THF/H2O (1:1) (10 mL) was added LiOH·H₂O (0.098 g, 2.358 mmol, 2 eq.). The solution was stirred at RT 50° C. for 4 h. The reaction mixture was concentrated to remove THF. Then the mixture was diluted with H₂O (30 mL) and extracted with EA (20 mL×1). The aqueous phase pH was adjusted to <3 with 1N HCl. Then the mixture was filtered and the filter cake was dried to give the 3,4-bis(benzyloxy)benzoic acid as a white solid (0.35 g, 89% yield). ¹HNMR (400 MHZ, DMSO-d₆): δ 12.67 (s, 1H), 7.53 (d, J=7.2 Hz, 2H), 7.46-7.29 (m, 10), 7.14 (d, J=8.8 Hz, 1H), 5.21 (s, 2H), 5.16 (s, 2H).

Step 4: Synthesis of (2S,3R)-5,7-bis(benzyloxy)-2-(3,4,5-tris(benzyloxy)phenyl)chroman-3-yl 3,4-bis(benzyloxy)benzoate (4). To a solution of (2S,3R)-5,7-bis(benzyloxy)-2-(3,4,5-tris(benzyloxy)phenyl)chroman-3-ol (0.5 g, 0.661 mmol, 1 eq.), in 10 mL DCM was added 3,4-bis(benzyloxy)benzoic acid (0.43 g, 1.322 mmol, 2 eq.), EDCI (0.606 g, 3.305 mmol, 5 eq.), Et₃N (0.5 mL, 3.305 mmol, 5 eq.) and DMAP (0.05 g, 0.396 mmol, 0.6 eq.) at 0° C. and the reaction mixture was stirred at RT for 16 h. Reaction progress was monitor by TLC. After this time, reaction mixture was quenched with hypo solution, extracted with DCM (3×50 mL), washed with brine and dried over anhydrous Na₂SO₄. Organic layer was concentrated under reduced pressure to obtained crude compound. The crude compound was purified by flash column chromatography eluted with 20% EtOAc in hexane as an eluent affords to obtain desired (2S,3R)-5,7-bis(benzyloxy)-2-(3,4,5-tris(benzyloxy)phenyl)chroman-3-yl 3,4-bis(benzyloxy)benzoate as a white solid (0.5 g, 71% yield). ¹HNMR (400 MHZ, DMSO-d₆): 7.45-7.28 (s, 32H), 7.22 (t, J=6.8 Hz, 3H), 7.11 (d, J=8.8 Hz, 1H), 6.87 (s, 2H), 6.42 (s, 1H), 6.28 (s, 1H), 5.40 (d, J=6.8 Hz, 1H), 5.21 (s, 2H), 5.16 (d, J=8.8 Hz, 1H), 5.07 (s, 6H), 4.97 (s, 2H), 4.92 (s, 2H), 4.85 (s, 2H), 2.91 (dd, J=5.2 Hz, 1H), 2.74 (dd, J=6.8 Hz, 1H).

Step 5: Synthesis of Compound 34. To a solution of (2S,3R)-5,7-bis(benzyloxy)-2-(3,4,5-tris(benzyloxy)phenyl)chroman-3-yl 3,4-bis(benzyloxy)benzoate (0.45 g, 0.419 mmol, 1 eq.), in 4 mL of 1:1 THF:MeOH was added palladium hydroxide (20 wt. %, 0.94 g) at RT and the reaction mixture was stirred under hydrogen atmosphere for 16 h. Then the mixture was passed through a pad of celite to remove the catalyst. The filtrate was concentrated under reduced pressure. Obtained crude compound was purified by Prep-HPLC to obtain (2S,3R)-5,7-dihydroxy-2-(3,4,5-trihydroxyphenyl)chroman-3-yl 3,4-dihydroxybenzoate as a gray solid (0.025 g, 13% yield). ¹HNMR (400 MHZ, DMSO-d₆): 7.23 (s, 1H), 7.18 (d, J=8.4 Hz, 1H), 6.74 (d, J=8.8 Hz, 1H), 6.25 (s, 2H), 5.91 (s, 1H), 5.80 (s, 1H), 5.20 (q, J=4.8 Hz, 1H), 4.99 (d, J=5.2 Hz, 1H), 2.60 (dd, J=4.0 Hz, 2H). LCMS: (M−H+): m/Z: 441.0.

### Compound 35

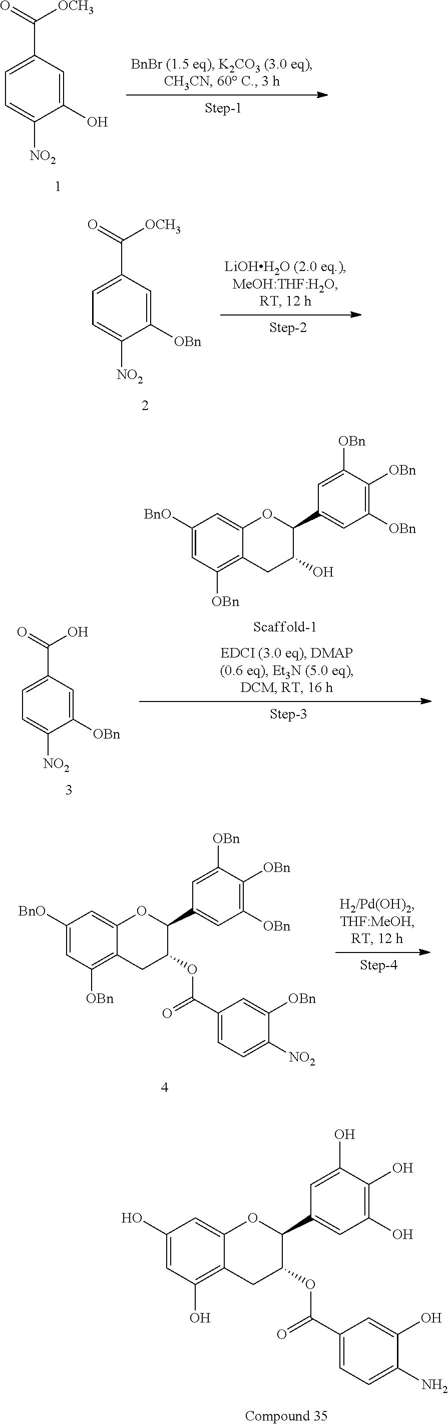 [SMILES: COC(=O)c1ccc([N+](=O)[O-])c(O)c1 ; COC(=O)c1ccc([N+](=O)[O-])c(OCc2ccccc2)c1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1ccc([N+](=O)[O-])c(OCc2ccccc2)c1 ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1ccc([N+](=O)[O-])c(OCc2ccccc2)c1 ; Nc1ccc(C(=O)O[C@@H]2Cc3c(O)cc(O)cc3O[C@H]2c2cc(O)c(O)c(O)c2)cc1O]

Step 1: Synthesis of methyl 3-(benzyloxy)-4-nitrobenzoate (2). To a stirred suspension of methyl 3-hydroxy-4-nitrobenzoate (2.5 g, 12.69 mmol, 1.0 eq.) and K₂CO₃ (5.25 g, 38.07 mmol, 3.0 eq.) in dry CH₃CN (25 mL) was added BnBr (2.26 ml, 19.03 mmol, 1.5 eq.) dropwise at 0° C. The mixture was stirred at 60° C. for 3 h and cooled to RT. The reaction mixture was filtered through pad of celite, washed with EtOAc (100 mL). The combined organic phase was washed with H₂O (50 mL), brine (50 mL), dried over Na₂SO₄, filtered and concentrated. The residue was purified by flash column chromatography on silica gel (PE/EA=10/1) to give methyl 3-(benzyloxy)-4-nitrobenzoate (2.0 g, 54.9% yield) as pale yellow solid. ¹HNMR (400 MHZ, CDCl₃): δ 7.90-7.80 (m, 2H), 7.70 (dd, J=8.4 Hz, 1.6 Hz, 1H), 7.50-7.30 (m, 5H), 5.28 (s, 2H), 3.96 (s, 3H).

Step 2: Synthesis of 3-(benzyloxy)-4-nitrobenzoic acid (3). To a solution of methyl 3-(benzyloxy)-4-nitrobenzoate (1.4 g, 4.87 mmol, 1.0 eq.) in MeOH:THF:H₂O (1:1:1) (15 mL) was added LiOH. H₂O (0.41 g, 9.75 mmol, 2.0 eq.) at RT and stirred at the same temperature for 12 h. The solvent was evaporated from the reaction mixture, diluted with H₂O (30 mL). The aqueous layer was acidified with 1N HCl (pH<3), obtained solid was filtered and washed with H₂O to give 3-(benzyloxy)-4-nitrobenzoic acid (1.10 g, 82.7% yield) as pale yellow solid. ¹H NMR (400 MHZ, DMSO-d₆); δ 13.65 (s, 1H), 7.98 (d, J=8.4 Hz, 1H), 7.86 (s, 1H), 7.65 (d, J=8.4 Hz, 1H), 7.46-7.30 (m, 5H), 5.38 (s, 2H).

Step 3: Synthesis of (2S,3R)-5,7-bis(benzyloxy)-2-(3,4,5-tris(benzyloxy)phenyl)chroman-3-yl 3-(benzyloxy)-4-nitrobenzoate (4). To a mixture of (2S,3R)-5,7-bis(benzyloxy)-2-(3,4,5-tris(benzyloxy)phenyl)chroman-3-ol (0.05 g, 0.06 mmol, 1.0 eq.) and 3-(benzyloxy)-4-nitrobenzoic acid (0.036 g, 0.13 mmol, 2.0 eq.) in CH₂Cl₂ (10 mL) was added EDCI (0.038 g, 0.19 mmol, 3.0 eq.), DMAP (0.005 g, 0.04 mmol, 0.6 eq.) and TEA (0.05 mL, 0.33 mmol, 5.0 eq.) under ice-water bath. The reaction mixture was stirred at room temperature for 16 h. The reaction mixture was diluted with H₂O (10 mL) and CH₂Cl₂ (30 mL). Organic layer was separated, washed with brine (10 mL), dried over Na₂SO₄, filtered and concentrated. The residue was purified by flash column chromatography on silica gel (PE/EA=5/1) to give (2S,3R)-5,7-bis(benzyloxy)-2-(3,4,5-tris(benzyloxy)phenyl)chroman-3-yl 3-(benzyloxy)-4-nitrobenzoate (0.025 g, 37.3% yield) as a yellow color solid. ¹H NMR (400 MHZ, CDCl₃) δ 7.76 (d, J=8.4 Hz, 1H), 7.62 (d, J=1.2 Hz, 1H), 7.51 (dd, J=8.4 Hz, 1.2 Hz, 1H), 7.44-7.19 (m, 30H), 6.70 (s, 2H), 6.31 (d, J=6.4 Hz, 1H), 6.51 (q, J=5.6, 1H), 5.15 (s, 2H), 5.12 (d, J=6.0 Hz, 1H), 5.08-4.93 (m, 10H), 2.91 (dq, J=19.2, 5.2 Hz, 2H).

Step 4: Synthesis of Compound 35. To a mixture of compound (2S,3R)-5,7-bis(benzyloxy)-2-(3,4,5-tris(benzyloxy)phenyl)chroman-3-yl 3-(benzyloxy)-4-nitrobenzoate (0.2 g, 0.19 mmol, 1.0 eq.) in THF (3 mL) and MeOH (3 mL) was added Pd(OH)2 (20 wt. %, 0.02 g). The mixture was stirred at room temperature under H2 atmosphere for 12 h. Then the mixture was passed through a pad of celite to remove the catalyst. The filtrate was concentrated under reduced pressure. Obtained crude compound was purified by Prep-HPLC to obtain (2S,3R)-5,7-dihydroxy-2-(3,4,5-trihydroxyphenyl)chroman-3-yl 4-amino-3-hydroxybenzoate (0.030 g, 33% yield) as grey color solid. ¹H NMR (400 MHZ, DMSO-d₆) δ 9.39 (bs, 1H), 9.29 (s, 1H), 9.06 (s, 1H), 8.87 (bs, 1H), 7.13 (s, 2H), 6.53 (d, J=8.4 Hz, 1H), 6.25 (s, 2H), 5.91 (s, 1H), 5.80 (s, 1H), 5.38 (s, 2H), 5.20 (d, J=5.2 Hz, 1H), 5.00 (d, J=5.2 Hz, 1H), 2.59 (m, 2H).

### Compound 36

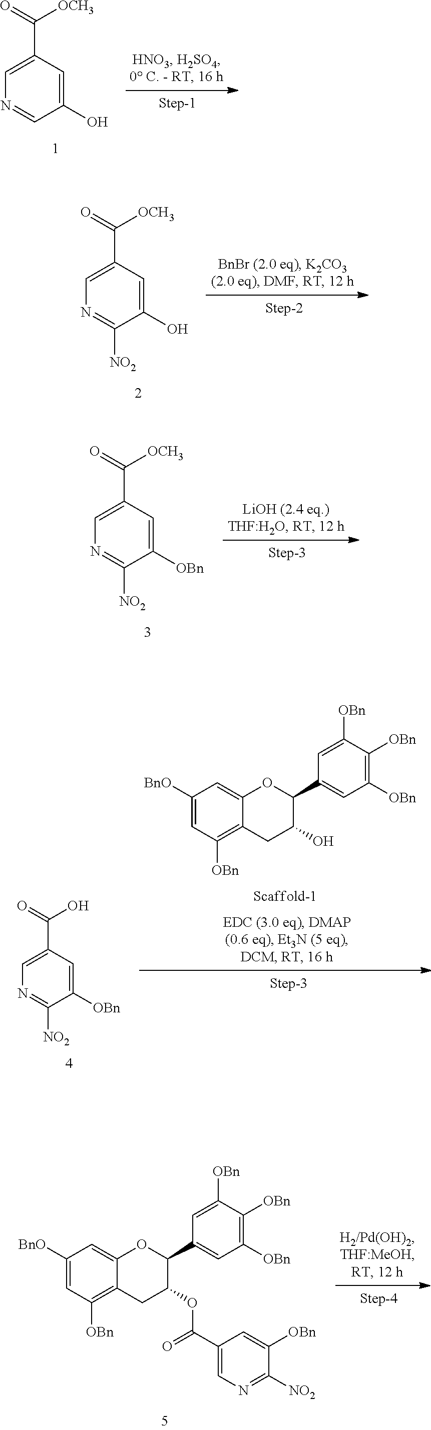 [SMILES: COC(=O)c1cncc(O)c1 ; COC(=O)c1cnc([N+](=O)[O-])c(O)c1 ; COC(=O)c1cnc([N+](=O)[O-])c(OCc2ccccc2)c1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1cnc([N+](=O)[O-])c(OCc2ccccc2)c1 ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1cnc([N+](=O)[O-])c(OCc2ccccc2)c1]

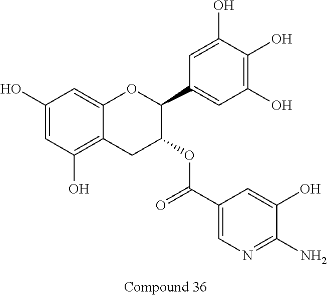 [SMILES: Nc1ncc(C(=O)O[C@@H]2Cc3c(O)cc(O)cc3O[C@H]2c2cc(O)c(O)c(O)c2)cc1O]

Step 1: Synthesis of methyl 5-hydroxy-6-nitronicotinate (2). To a solution of methyl 5-hydroxynicotinate (1.0 g, 6.53 mmol, 1.0 eq) in H2SO4 (10 mL) was added HNO3 (0.8 g, 13.06 mmol) at 0° C. The reaction mixture was stirred at room temperature overnight. The reaction mixture was poured into 100 mL of ice-water and stirred for 20 minutes. The mixture was filtered to give methyl 5-hydroxy-6-nitronicotinate (0.390 g, 30.2% yield) as a yellow color solid. ¹H NMR (400 MHZ, DMSO-d₆): δ 12.21 (bs, 1H), 8.44 (s, 1H), 8.03 (d, J=1.2 Hz, 1H), 3.90 (s, 3H).

Step 2: Synthesis of methyl 5-(benzyloxy)-6-nitronicotinate (3). To a solution of methyl 5-hydroxy-6-nitronicotinate (0.3 g, 1.51 mmol, 1.0 eq) and K₂CO₃ (0.418 g, 3.03 mmol, 2.0 eq) in DMF (5 mL) was added BnBr (0.518 g, 3.03 mmol, 2.0 eq) at 0° C. The reaction mixture was stirred at room temperature for 12 h. The reaction mixture was diluted with H₂O (20 mL) and extracted with EtOAc (50 mL×2). The combined organic layers was washed with brine (30 mL), dried over Na₂SO₄, filtered and concentrated. The residue was purified by flash column chromatography on silica gel (PE/EA=9/1) to give methyl 5-(benzyloxy)-6-nitronicotinate (0.33 g, 76.7% yield) as a yellow solid. ¹H NMR (400 MHZ, DMSO-d₆): 8.60 (s, 1H), 8.39 (s, 1H), 7.44-7.28 (m, 5H), 5.46 (s, 2H), 3.93 (s, 3H).

Step 3: Synthesis of 5-(benzyloxy)-6-nitronicotinic acid (4). To a mixture of methyl 5-(benzyloxy)-6-nitronicotinate (0.4 g, 1.38 mmol, 1.0 eq) in THF (15 mL) and H₂O (2 mL) was added LiOH H₂O (0.140 g, 3.34 mmol, 2.4 eq). The solution was stirred at room temperature for overnight. The reaction mixture was concentrated in vacuum to remove the THF. The mixture was diluted with H₂O (30 mL) and extracted with EtOAc (2×30 mL). The aqueous phase was acidified with (pH<3) 1 N HCl. The obtained solid was filtered and washed with cold H2O to give 5-(benzyloxy)-6-nitronicotinic acid (0.32 g, 84% yield) as a white solid. 1H NMR (400 MHZ, DMSO-d₆): 14.14 (bs, 1H), 8.57 (s, 1H), 8.35 (s, 1H), 7.44-7.28 (m, 5H), 5.46 (s, 2H).

Step 4: Synthesis of 2S, 3R)-5,7-bis(benzyloxy)-2-(3,4,5-tris(benzyloxy)phenyl)chroman-3-yl 5-(benzyloxy)-6-nitronicotinate (5). To a mixture of (2S,3R)-5,7-bis(benzyloxy)-2-(3,4,5-tris(benzyloxy)phenyl)chroman-3-ol (0.5 g, 0.66 mmol, 1.0 eq.) and 5-(benzyloxy)-6-nitronicotinic acid (0.215 g, 0.79 mmol, 1.2 eq.) in CH₂Cl₂ (10 mL) was added EDCI·HCl (0.378 g, 1.98 mmol, 3.0 eq.), DMAP (0.048 g, 0.39 mmol, 0.6 eq.) and TEA (0.5 mL, 3.30 mmol, 5.0 eq.) at 0° C. The reaction mixture was stirred at room temperature for 16 h. The reaction mixture was diluted with H₂O (20 mL) and the phases were separated. The organic phase was washed with brine (20 mL), dried over Na₂SO₄, filtered and concentrated. The residue was purified by flash chromatography on silica gel (PE/EA=4/1) to give 2S,3R)-5,7-bis(benzyloxy)-2-(3,4,5-tris(benzyloxy)phenyl)chroman-3-yl 5-(benzyloxy)-6-nitronicotinate (0.2 g, 29.8% yield) as a pale yellow color solid. ¹HNMR (400 MHZ, DMSO-d₆) δ: 8.46 (s, 1H), 8.18 (s, 1H), 7.45-7.17 (m, 31H), 6.94 (s, 2H), 6.44 (s, 1H), 6.28 (s, 1H), 5.52 (q, J=6.0 Hz, 1H), 5.34 (q, J=12.0 Hz, 2H), 5.19 (q, J=7.6 Hz, 1H), 5.13-4.93 (m, 8H), 4.87 (s, 2H), 3.08-2.78 (m, 2H).

Step 5: Synthesis of Compound 36. To a mixture of 2S,3R)-5,7-bis(benzyloxy)-2-(3,4,5-tris(benzyloxy)phenyl)chroman-3-yl 5-(benzyloxy)-6-nitronicotinate (0.280 g, 0.27 mmol, 1.0 eq.) in THF (5 mL) and MeOH (5 mL) was added Pd (OH)₂ (20 wt. %, 0.100 g). The mixture was stirred at room temperature under H₂ atmosphere for overnight. The reaction mixture was filtered and the filtrate was concentrated. The residue was purified by prep-HPLC to give (2S,3R)-5,7-dihydroxy-2-(3,4,5-trihydroxyphenyl)chroman-3-yl 6-amino-5-hydroxynicotinate (0.040 g, 31.2% yield) as grey color solid. ¹HNMR (400 MHZ, DMSO-d₆) δ: 9.99 (bs, 1H), 9.31 (s, 1H), 9.07 (s, 1H), 8.85 (d, J=4.4 Hz, 2H), 8.09 (s, 1H), 7.95 (d, J=1.6 Hz, 1H), 7.13 (s, 1H), 6.51 (bs, 2H), 6.25 (s, 2H), 5.91 (d, J=2.4 Hz, 1H), 5.79 (d, J=2.4 Hz, 1H), 5.21 (q, J=5.2 Hz, 1H), 5.00 (d, J=5.2 Hz, 1H), 2.65-2.57 (m, 2H).

### Compound 37

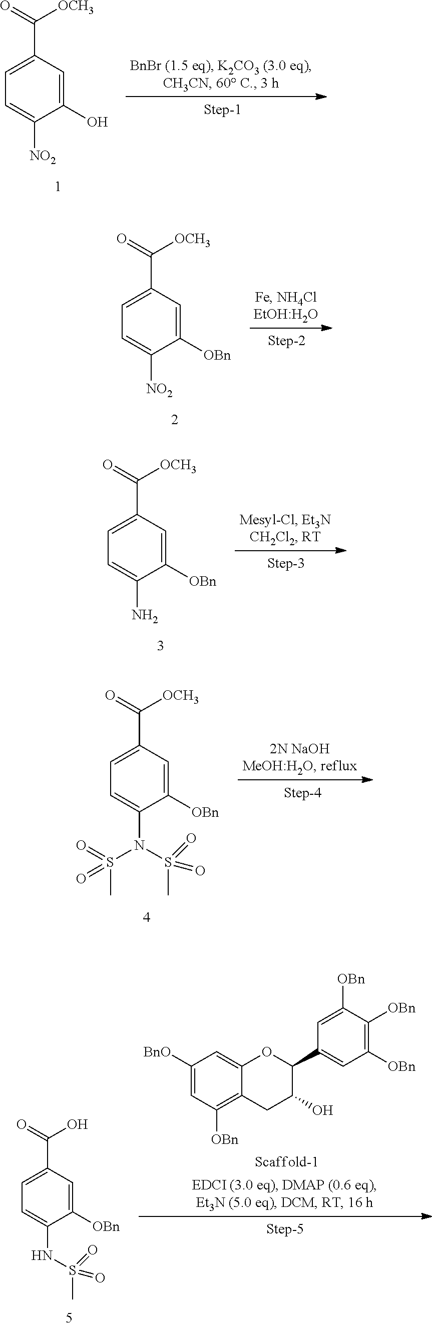 [SMILES: COC(=O)c1ccc([N+](=O)[O-])c(O)c1 ; COC(=O)c1ccc([N+](=O)[O-])c(OCc2ccccc2)c1 ; COC(=O)c1ccc(N)c(OCc2ccccc2)c1 ; COC(=O)c1ccc(N(S(C)(=O)=O)S(C)(=O)=O)c(OCc2ccccc2)c1 ; CS(=O)(=O)Nc1ccc(C(=O)O)cc1OCc1ccccc1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1]

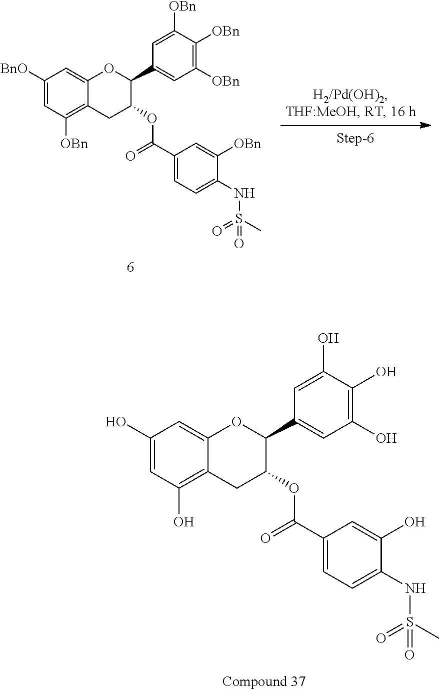 [SMILES: CS(=O)(=O)Nc1ccc(C(=O)O[C@@H]2Cc3c(OCc4ccccc4)cc(OCc4ccccc4)cc3O[C@H]2c2cc(OCc3ccccc3)c(OCc3ccccc3)c(OCc3ccccc3)c2)cc1OCc1ccccc1 ; CS(=O)(=O)Nc1ccc(C(=O)O[C@@H]2Cc3c(O)cc(O)cc3O[C@H]2c2cc(O)c(O)c(O)c2)cc1O]

Step 1: Synthesis of methyl 3-(benzyloxy)-4-nitrobenzoate (2). To a stirred suspension of methyl 3-hydroxy-4-nitrobenzoate (2.5 g, 12.69 mmol, 1 eq.) and K₂CO₃ (5.25 g, 38.07 mmol, 3.0 eq.) in dry CH₃CN (25 mL) was added BnBr (2.26 ml, 19.03 mmol, 1.5 eq.) dropwise at 0° C. The mixture was stirred at 60° C. for 3 h and cooled to RT. The reaction mixture was filtered through pad of celite, washed with (100 mL). The combined organic phase was washed with H₂O (50 mL), brine (50 mL), dried over Na₂SO₄, filtered and concentrated. The residue was purified by flash column chromatography on silica gel (PE/EA=10/1) to give methyl 3-(benzyloxy)-4-nitrobenzoate (2.0 g, 54.9% yield) as pale yellow solid. ¹H NMR (400 MHz, CDCl₃): δ 7.90-7.80 (m, 2H), 7.70 (dd, J=8.4 Hz, 1.6 Hz, 1H), 7.50-7.30 (m, 5H), 5.28 (s, 2H), 3.96 (s, 3H).

Step 2: Synthesis of methyl 4-amino-3-(benzyloxy) benzoate (3). A mixture of NH₄Cl (0.093 g, 1.74 mmol, 1.0 eq.) and Fe (0.486 g, 8.71 mmol, 5.0 eq.) in 20 mL of EtOH: H₂O (5:1) was stirred at 90° C. for 1 h. Then methyl 3-(benzyloxy)-4-nitrobenzoate (0.5 g, 1.74 mmol, 1.0 eq.) in CH₃CN (10 mL) was slowly added at RT and the resulting mixture was stirred at 90° C. for 3 h. The reaction mixture was cooled to RT and filtered. The filtrate was concentrated to give crude product, which was diluted with H₂O (30 mL) and extracted with EtOAc (50 mL×2). The combined organic layers were washed with brine (50 mL), dried over Na₂SO₄, filtered and concentrated to give methyl 4-amino-3-(benzyloxy) benzoate (0.400 g, 84.2% yield) as white solid. ¹H NMR (400 MHZ, DMSO-d6); δ 7.50 (d, J=7.2 Hz, 2H), 7.41-7.36 (m, 4H), 7.32 (q, J=8.8 Hz, 1H), 6.66 (d, J=8.8 Hz, 1H), 5.65 (s, 2H), 5.13 (s, 2H), 3.72 (s, 3H).

Step 3: Synthesis of methyl 3-(benzyloxy)-4-(N-(methylsulfonyl)methylsulfonamido)benzoate (4). To a solution of methyl 4-amino-3-(benzyloxy) benzoate (0.4 g, 1.55 mmol, 1.0 eq.) in CH₂Cl₂ (4 mL) was added Et₃N (1.3 mL, 9.32 mmol, 6.0 eq.) and mesyl chloride (0.534 g, 4.66 mmol, 3.0 eq.) at 0° C. The mixture was stirred at RT for 12 h. The reaction mixture was neutralized with sat. aq. NaHCO₃ (10 mL) solution and extracted with CH₂Cl₂ (2×50 mL). The combined organic layers were washed with H₂O (20 mL), brine (20 mL), dried over Na₂SO₄, filtered and concentrated. The residue was purified by flash column chromatography on silica gel (PE/EA=9/1) to give methyl 3-(benzyloxy)-4-(N-(methylsulfonyl)methylsulfonamido)benzoate (0.6 g, 93% yield) as a white solid. ¹H NMR (400 MHZ, DMSO-d₆): δ 7.71 (d, J=1.6 Hz, 1H), 7.68 (d, J=8.0 Hz, 1H), 7.60 (dd, J=8.0 Hz, J=1.6 Hz, 1H), 7.51 (d, J=7.2 Hz, 2H), 7.41-7.30 (m, 3H), 5.31 (s, 2H), 3.87 (s, 3H), 3.42 (s, 6H).

Step 4: Synthesis of 3-(benzyloxy)-4-(methylsulfonamido)benzoic acid (5). To a solution of methyl 3-(benzyloxy)-4-(N-(methylsulfonyl)methylsulfonamido)benzoate (0.6 g, 1.45 mmol, 1.0 eq.) in MeOH: H₂O (1:1) (20 mL) was added 2N NaOH (20 mL) at RT, then stirred at reflux for 2 h. After completion of the reaction, solvent was evaporated from the reaction mixture. The reaction mixture was diluted with H₂O (30 mL) and extracted with ethyl acetate (50 mL). The Aqueous layer was acidified with 2N HCl and extracted with EtOAc (2×50 mL). The combined organic phase was dried over Na₂SO₄, filtered and concentrated, obtained 3-(benzyloxy)-4-(methylsulfonamido)benzoic acid (0.42 g, 90% yield) as a pale yellow solid. 1H NMR (400 MHZ, DMSO-d₆): δ 12.88 (s, 1H), 9.22 (s, 1H), 7.59-7.52 (m, 4H), 7.42-7.29 (m, 4H), 5.23 (s, 2H), 3.01 (s, 3H).

Step 5: Synthesis of (2S,3R)-5,7-bis(benzyloxy)-2-(3,4,5-tris(benzyloxy)phenyl)chroman-3-yl 3-(benzyloxy)-4-(methylsulfonamido)benzoate (6). To a mixture of (2S,3R)-5,7-bis(benzyloxy)-2-(3,4,5-tris(benzyloxy)phenyl)chroman-3-ol (0.6 g, 0.79 mmol, 1.0 eq.) and 3-(benzyloxy)-4-(methylsulfonamido)benzoic acid (0.306 g, 0.95 mmol, 1.2 eq.) in DCM (8 mL) was added EDCI (0.454 g, 2.37 mmol, 3.0 eq.), DMAP (0.058 g, 0.47 mmol, 0.6 eq.) and TEA (0.6 mL, 3.96 mmol, 5.0 eq.) at 0° C. The reaction mixture was stirred at room temperature for 24 h. The reaction progress was monitored by TLC. The reaction mixture was diluted with H₂O (20 mL) and CH₂Cl₂ (50 mL). The phases were separated. The organic layer was washed with brine (20 mL), dried over Na₂SO₄, filtered and concentrated. The residue was purified by flash column chromatography on silica gel (PE/EA=5/1) to give (2S,3R)-5,7-bis(benzyloxy)-2-(3,4,5-tris(benzyloxy)phenyl)chroman-3-yl 3-(benzyloxy)-4-(methylsulfonamido)benzoate (0.22 g, 26% yield) as white solid. ¹H NMR (400 MHZ, CDCl₃) δ 7.60-7.50 (m, 3H), 7.44-7.27 (m, 26H), 7.26-7.19 (m, 4H), 7.02 (s, 1H), 6.72 (s, 2H), 6.30 (dd, J=7.6, 2.4 Hz, 2H), 5.50 (d, J=5.6, 1H), 5.10 (d, J=6.8, 1H), 5.08-5.02 (m, 6H), 4.99-4.93 (m, 6H), 3.08-2.81 (m, 2H), 2.88 (d, J=3.6, 1H).

Step 6: Synthesis of Compound 37. To a mixture of (2S,3R)-5,7-bis(benzyloxy)-2-(3,4,5-tris(benzyloxy)phenyl)chroman-3-yl 3-(benzyloxy)-4-(methylsulfonamido)benzoate (0.2 g, 0.18 mmol, 1.0 eq.) in THF (4 mL) and MeOH (4 mL) was added Pd(OH)₂ (20 wt. %., 0.038 g). The mixture was stirred at room temperature under H₂ atmosphere for overnight. The reaction mixture was filtered and the filtrate was concentrated. The residue was purified by prep-HPLC to give (2S,3R)-5,7-dihydroxy-2-(3,4,5-trihydroxyphenyl)chroman-3-yl 3-hydroxy-4-(methylsulfonamido)benzoate (0.058 g, 59.5% yield) as a grey color solid. ¹H NMR (400 MHZ, DMSO-d₆) δ 9.08 (bs, 7H), 7.34 (s, 1H), 7.30-7.26 (m, 2H), 6.27 (s, 2H), 5.92 (d, J=2.4 Hz, 1H), 5.80 (d, J=2.0 Hz, 1H), 5.27 (q, J=5.2 Hz, 1H), 5.04 (d, J=5.2 Hz, 1H), 2.99 (s, 3H), 2.63 (m, 2H).

### Compound 38

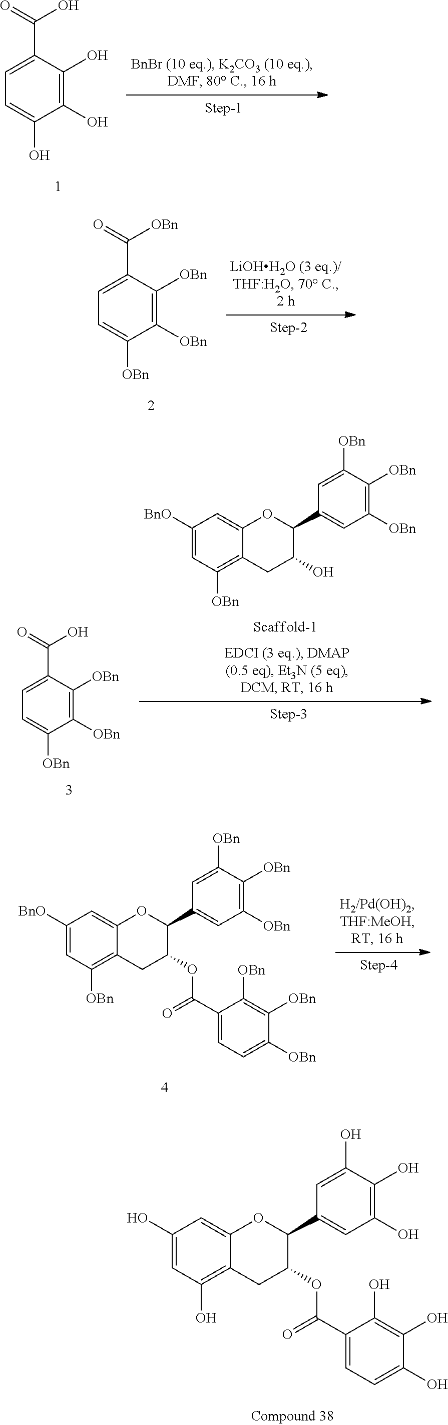 [SMILES: O=C(O)c1ccc(O)c(O)c1O ; O=C(OCc1ccccc1)c1ccc(OCc2ccccc2)c(OCc2ccccc2)c1OCc1ccccc1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1ccc(OCc2ccccc2)c(OCc2ccccc2)c1OCc1ccccc1 ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1ccc(OCc2ccccc2)c(OCc2ccccc2)c1OCc1ccccc1 ; O=C(O[C@@H]1Cc2c(O)cc(O)cc2O[C@H]1c1cc(O)c(O)c(O)c1)c1ccc(O)c(O)c1O]

Step 1: Synthesis of Benzyl 2,3,4-tris(benzyloxy)benzoate (2). To a solution of compound 2,3,4-trihydroxybenzoic acid (10 g, 58.780 mmol, 1 eq.) and K₂CO₃ (3.24 g, 558.235 mmol, 10 eq.) in DMF (100 mL) was added BnBr (69.4 mL, 558.235 mmol, 10 eq.) at 0° C. The mixture was stirred at 80° C. for 16 h. The reaction mixture was diluted with cold H₂O (500 mL) get the free solid, filtered off & dried under vacuumed affords to obtain desired compound Benzyl 2,3,4-tris(benzyloxy)benzoate as a brown solid (27.1 g, 87% yield). ¹HNMR (400 MHZ, DMSO-d₆): δ 7.57 (d, J=8.8 Hz, 1H), 7.48 (d, J=7.2 Hz, 2H), 7.41-7.28 (m, 18), 7.26 (d, J=5.6 Hz, 1H), 5.26 (s, 2H), 5.22 (s, 2H), 4.96 (s, 2H). LC-MS m/z (M+H): 355.10.

Step 2: Preparation of 2,3,4-tris(benzyloxy)benzoic acid (3). A mixture of compound Benzyl 2,3,4-tris(benzyloxy)benzoate (1 g, 1.88 mmol, 1.0 eq.) in THF/H₂O (1:1) (20 mL) was added LiOH H₂O (0.237 g, 5.65 mmol, 3.0 eq.). The solution was stirred at 70° C. for 2 h. The reaction mixture was concentrated to remove THF. Then the mixture was diluted with H₂O (30 mL) and extracted with EA (2×80 mL). The aqueous phase pH was adjusted to <3 with 1 N HCl. Then the mixture was filtered and the filter cake was dried to give the 2,3,4-tris(benzyloxy)benzoic acid (0.8 g, 96% yield) as a white solid. ¹HNMR (400 MHZ, DMSO-d₆); δ 12.62 (s, 1H), 7.53 (d, J=8.8 Hz, 1H), 7.51 (d, J=8.0 Hz, 2H), 7.47-7.32 (m, 10H), 7.31-7.28 (m, 3H), 7.04 (d, J=8.8 Hz, 1H), 5.21 (s, 2H), 4.99 (s, 2H), 4.96 (s, 2H). LC-MS m/z (M+H): 441.20.

Step 3: Synthesis of (2S,3R)-5,7-bis(benzyloxy)-2-(3,4,5-tris(benzyloxy)phenyl)chroman-3-yl 2,3,4-tris(benzyloxy)benzoate (4). To a mixture of (2S,3R)-5,7-bis(benzyloxy)-2-(3,4,5-tris(benzyloxy)phenyl)chroman-3-ol (0.1 g, 0.13 mmol, 1 eq.) and 2,3,4-tris(benzyloxy)benzoic acid (0.116 g, 0.264 mmol, 2 eq.) in DCM (3 mL) was added EDCI (0.075 g, 0.393 mmol, 3.0 eq.), DMAP (0.08 g, 0.066 mmol, 0.5 eq.) and TEA (0.09 mL, 0.665 mmol, 5 eq.) under ice-water bath. The reaction mixture was stirred at room temperature for overnight. The reaction mixture was diluted with H₂O (20 mL) and the phases were separated. The organic layer was washed with brine (20 mL×2), dried over Na₂SO₄, filtered and concentrated. The crude compound was purified by flash column chromatography eluted with 15% EtOAc in hexane as an eluent affords to obtain desired compound (2S,3R)-5,7-bis(benzyloxy)-2-(3,4,5-tris(benzyloxy)phenyl)chroman-3-yl 2,3,4-tris(benzyloxy)benzoate (0.050 g, 32% yield) as a white solid. ¹HNMR (400 MHZ, CDCl₃) δ 7.44-7.19 (m, 42H), 6.70 (s, 2H), 6.65-6.64 (m, 1H), 6.25 (dd, J=8.0, 2.0 Hz, 2H), 5.52 (d, J=5.6 Hz, 1H), 5.08-4.93 (m, 16H), 2.99 (dd, J=17.2, 5.6 Hz, 1H), 2.82 (dd, J=16.8, 6.8 Hz, 1H). LC-MS m/z (M+H): 1179.53.

Step 4: Synthesis of Compound 38. To a mixture of (2S,3R)-5,7-bis(benzyloxy)-2-(3,4,5-tris(benzyloxy)phenyl)chroman-3-yl 2,3,4-tris(benzyloxy)benzoate (0.25 g, 0.217 mmol) in THF (15 mL) and MeOH (15 mL) was added Pd(OH)₂ (20 wt. %, 0.25 g). The mixture was stirred at room temperature under H₂ atmosphere overnight. The reaction mixture was filtered and the filtrate was concentrated. The residue was purified by prep-HPLC to give (2S,3R)-5,7-dihydroxy-2-(3,4,5-trihydroxyphenyl)chroman-3-yl 2,3,4-trihydroxybenzoate (0.027 g, 27% yield) as a gray solid. ¹HNMR (400 MHZ, DMSO-d₆) δ 9.08 (bs, 8H), 6.98 (d, J=8.8 Hz, 1H), 6.33 (d, J=8.8 Hz, 1H), 6.27 (s, 2H), 5.92 (d, J=2.0 Hz, 1H), 5.80 (d, J=2.0 Hz, 1H), 5.28 (q, J=5.6 Hz, 1H), 5.06 (d, J=5.6 Hz, 1H), 2.65 (m, 2H). LC-MS m/z (M+H): 459.1.

### Compound 39

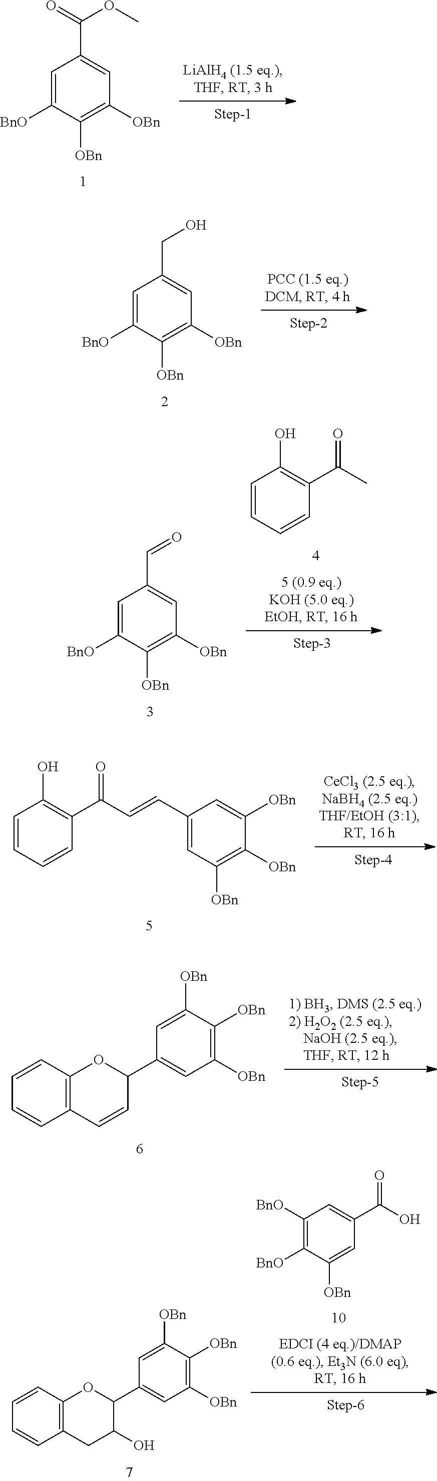

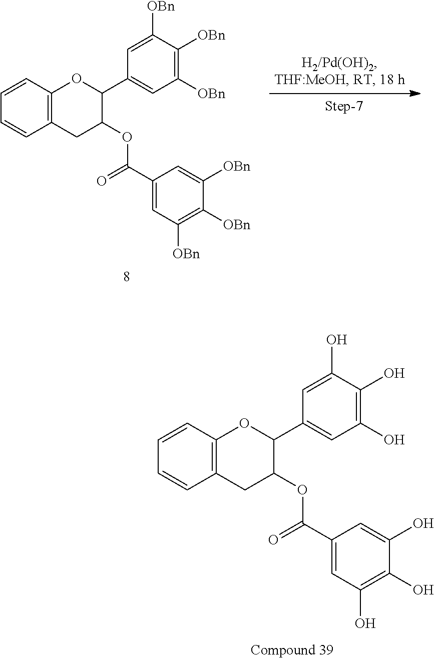 [SMILES: O=C(OC1Cc2ccccc2OC1c1cc(OCc2ccccc2)c(OCc2ccccc2)c(OCc2ccccc2)c1)c1cc(OCc2ccccc2)c(OCc2ccccc2)c(OCc2ccccc2)c1 ; O=C(OC1Cc2ccccc2OC1c1cc(O)c(O)c(O)c1)c1cc(O)c(O)c(O)c1]

Step 1: Synthesis of (3,4,5-tris(benzyloxy)phenyl)methanol (2). To a solution of methyl 3,4,5-tris(benzyloxy)benzoate (4.0 g, 8.81 mmol, 1.0 eq.) in THF (40 mL) was added LiAlH₄ (0.5 g, 13.2 mmol, 1.5 eq.) at 0° C. The mixture was stirred at room temperature for 3 h. The reaction mixture was quenched with H₂O (0.5 mL) and 15% NaOH (1 mL) at 0° C. The resulting mixture was filtered through pad of celite, filtrate was concentrated and residue was purified by flash column chromatography on silica gel (PE/EA=4/1) to give (3,4,5-tris(benzyloxy)phenyl)methanol (3.6 g, 94% yield) as a white solid ¹HNMR (400 MHZ, DMSO-d₆): δ 7.48-7.20 (m, 15H), 6.75 (s, 2H), 5.17 (t, J=17.2 Hz, 1H), 5.09 (s, 4H), 4.91 (s, 2H), 4.41 (d, J=5.6 Hz, 2H).

Step 2: Synthesis of 3,4,5-tris(benzyloxy)benzaldehyde (3). A mixture of (3,4,5-tris(benzyloxy)phenyl)methanol (3.6 g, 8.29 mmol, 1.0 eq.) in CH₂Cl₂ (20 mL) was added PCC (2.68 g, 12.44 mmol, 1.5 eq.) at 0° C. The mixture was stirred at room temperature for 4 h, solvent was evaporated from the reaction mixture under reduced pressure. The obtained residue was purified by flash column chromatography to give the 3,4,5-tris(benzyloxy)benzaldehyde (2.8 g, 78% yield) as a white solid. ¹HNMR (400 MHZ, DMSO-d₆): δ 9.85 (s, 1H), 7.48-7.20 (m, 17H), 5.21 (s, 4H), 5.06 (s, 2H).

Step 3: Synthesis of (E)-1-(2-hydroxyphenyl)-3-(3,4,5-tris(benzyloxy)phenyl)prop-2-en-1-one (5). To a mixture of 3,4,5-tris(benzyloxy)benzaldehyde (1.8 g, 4.16 mmol, 1.0 eq.) and compound 1-(2-hydroxyphenyl)ethan-1-one (0.51 g, 3.75 mmol, 0.9 eq.) in EtOH (40 mL) was added KOH (1.16 g, 20.83 mmol, 5.0 eq.). The mixture was stirred at RT for 16 h. The reaction mixture was concentrated. The residue was diluted with H₂O (50 mL) and extracted with EtOAc (50 mL×3). The combined organic layers were washed with brine (50 mL), dried over Na₂SO₄, filtered and concentrated. The obtained residue was purified by flash column chromatography to give (E)-1-(2-hydroxyphenyl)-3-(3,4,5-tris(benzyloxy)phenyl)prop-2-en-1-one (1.2 g, 53% yield) as yellow solid. ¹HNMR (400 MHZ, CDCl₃): δ 12.81 (s, 1H), 7.86 (d, J=7.6, 1H), 7.77 (d, J=15.2, 1H), 7.50-7.20 (m, 17H), 7.10-6.90 (m, 2H), 6.93 (s, 2H), 5.16 (s, 4H), 5.13 (s, 2H).

Step 4: Synthesis of 2-(3,4,5-tris(benzyloxy)phenyl)-2H-chromene (6). To a solution of (E)-1-(2-hydroxyphenyl)-3-(3,4,5-tris(benzyloxy)phenyl)prop-2-en-1-one (1.2 g, 2.21 mmol, 1.0 eq.) in THF (20 mL) and EtOH (6 mL) was added anhydrous CeCl3 (1.36 g, 5.53 mmol, 2.5 eq.) and NaBH₄ (0.21 g, 5.53 mmol, 2.5 eq.) at 0° C. The mixture was stirred at RT for 16 h. The reaction progress was monitored by TLC. The reaction mixture was diluted with H₂O (50 mL) and extracted with CH₂Cl₂ (50 mL×3). The combined organic layers were washed with brine (50 mL), dried over Na₂SO₄, filtered and concentrated. The residue was purified by flash column chromatography (PE/EA=10/1) to give 2-(3,4,5-tris(benzyloxy)phenyl)-2H-chromene (0.81 g, 69% yield) as a colorless liquid. ¹HNMR (400 MHZ, CDCl₃): 7.48-7.24 (m, 14H), 7.12 (m, 1H), 7.02 (m, 1H), 6.89 (m, 1H), 6.78 (m, 1H), 6.76 (s, 2H), 6.51 (d, J=9.2, 1H), 5.79 (s, 1H), 5.72 (m, 1H), 5.07 (s, 4H), 5.13 (s, 2H).

Step 5: Synthesis of 2-(3,4,5-tris(benzyloxy)phenyl)chroman-3-ol (7). To a solution of 2-(3,4,5-tris(benzyloxy)phenyl)-2H-chromene (0.8 g, 1.52 mmol, 1.0 eq.) in dry THF (10 mL) was added BH3. DMS (2M, 1.9 mL, 3.80 mmol, 2.5 eq.) at 0° C. for 10 min. The mixture was stirred at room temperature until the starting material was disappeared. Then 3N NaOH aq. solution (1.26 mL, 3.80 mmol, 2.5 eq.) and 30% aq. H₂O2 (0.05 mL, 3.80 mmol, 2.5 eq.) was added drop wise at 0° C. The mixture was stirred at RT for 12 h. The reaction mixture was diluted with H₂O (50 mL) and extracted with EtOAc (50 mL×2). The combined organic layers were washed with brine (50 mL), dried over Na₂SO₄, filtered and concentrated. The residue was purified by flash column chromatography (PE/EA=10/1) to give 2-(3,4,5-tris(benzyloxy)phenyl)chroman-3-ol (0.22 g, 26.6% yield) as a white solid. ¹HNMR (400 MHZ, CDCl₃): 7.48-7.24 (m, 14H), 7.20-7.09 (m, 3H), 6.93-6.89 (m, 2H), 6.76 (s, 2H), 5.20-5.01 (m, 7H), 4.65 (d, J=8.0, 1H), 3.99 (m, 1H), 3.10-2.81 (m, 2H).

Step 6: Synthesis of 2-(3,4,5-tris(benzyloxy)phenyl)chroman-3-yl 3,4,5-tris(benzyloxy)benzoate (8). To a mixture of 2-(3,4,5-tris(benzyloxy)phenyl)chroman-3-ol (0.26 g, 0.47 mmol, 1.0 eq.) and 3,4,5-tris(benzyloxy)benzoic acid (0.52 g, 1.19 mmol, 2.5 eq.) in CH₂Cl₂ (10 mL) was added EDCI (0.36 g, 1.91 mmol, 4.0 eq.), DMAP (0.03 g, 0.29 mmol, 0.6 eq.) and TEA (0.4 mL, 2.86 mmol, 6.0 eq.) under ice-water bath. The reaction mixture was stirred at room temperature overnight. The reaction mixture was diluted with H₂O (30 mL) and extracted with CH₂Cl₂ (2×50 mL). The combined organic layer was washed with brine (30 mL), dried over Na₂SO₄, filtered and concentrated. The residue was purified by flash chromatography on silica gel (PE/EA=5/1) to give 2-(3,4,5-tris(benzyloxy)phenyl)chroman-3-yl 3,4,5-tris(benzyloxy)benzoate (0.185 g, 40% yield) as a white solid. ¹HNMR (400 MHZ, CDCl₃): δ 7.44-7.19 (m, 33H), 7.13-6.93 (m, 3H), 6.68 (s, 2H), 5.45 (d, J=4.8, 1H), 5.21 (d, J=6.0, 1H), 5.10-4.91 (m, 12H), 2.97 (dq, J=16.4, 4.0, 2H).

Step 7: Synthesis of Compound 39. To a mixture of 2-(3,4,5-tris(benzyloxy)phenyl)chroman-3-yl 3,4,5-tris(benzyloxy)benzoate (0.180 g, 0.18 mmol, 1.0 eq.) in THF (2.5 mL) and MeOH (2.5 mL) was added Pd(OH)₂/C (20 wt. %, 26 mg). The mixture was stirred at room temperature under H₂ atmosphere for 18 h. The reaction mixture was filtered and the filtrate was concentrated. The residue was purified by prep-HPLC to give 2-(3,4,5-trihydroxyphenyl)chroman-3-yl 3,4,5-trihydroxybenzoate (0.035 mg, 44.3% yield) as a grey color solid. ¹HNMR (400 MHZ, DMSO-d₆) δ 8.83 (bs, 6H), 7.22-7.04 (m, 2H), 6.96-6.83 (m, 2H), 6.82 (s, 2H), 6.26 (s, 2H), 5.31 (d, J=4.8 Hz, 1H), 5.18 (d, J=4.4 Hz, 1H), 2.88 (m, 2H).

### Compound 40

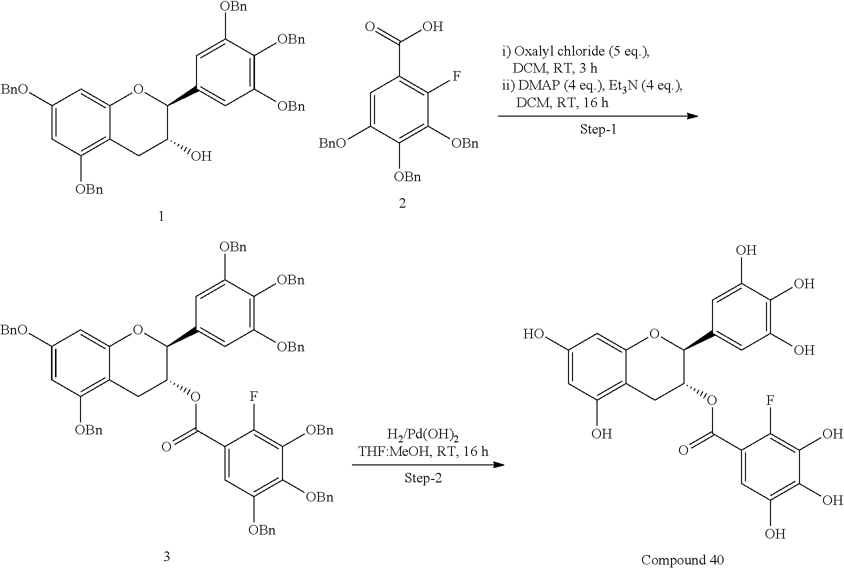 [SMILES: O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1cc(OCc2ccccc2)c(OCc2ccccc2)c(OCc2ccccc2)c1F ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1cc(OCc2ccccc2)c(OCc2ccccc2)c(OCc2ccccc2)c1F ; O=C(O[C@@H]1Cc2c(O)cc(O)cc2O[C@H]1c1cc(O)c(O)c(O)c1)c1cc(O)c(O)c(O)c1F]

Step 1: Synthesis of (2S,3R)-5,7-bis(benzyloxy)-2-(3,4,5-tris(benzyloxy)phenyl)chroman-3-yl 3,4,5-tris(benzyloxy)-2-fluorobenzoate (1). Under an N₂ atmosphere, to stirred solution of 3,4,5-tris(benzyloxy)-2-fluorobenzoic acid (2.2 g, 4.761 mmol, 1.2 eq.) in DCM (10 mL) was added oxalyl chloride (2.1 mL, 19.840 mmol, 5 eq.) and two drop of DMF at 0° C. The reaction mixture was stirred at RT for 3 h. After this time, the reaction mixture was concentrated under reduced pressure to get acid chloride. Obtained acid chloride was added to a solution of (2S,3R)-5,7-bis(benzyloxy)-2-(3,4,5-tris(benzyloxy)phenyl)chroman-3-ol (3.0 g, 3.968 mmol, 1 eq.), DMAP (1.93 g, 15.870 mmol, 4 eq.) and Et₃N (2.2 mL, 15.870 mmol, 4 eq.) in CH₂Cl₂ (10 mL) at 0° C. Then the reaction mixture was stirred at RT 16 h. Finally, the reaction was quenched with saturated aqueous NaHCO₃ solution (5 mL). The organic layer was separated and the aqueous layer was extracted with CH₂Cl₂ (30 mL). Combined organic phase was dried over MgSO₄, filtered and concentrated under reduced pressure. Obtained crude compound was purified by flash column chromatography (EtOAc in hexane) to get (2S,3R)-5,7-bis(benzyloxy)-2-(3,4,5-tris(benzyloxy)phenyl)chroman-3-yl 3,4,5-tris(benzyloxy)-2-fluorobenzoate as a white solid (1.2 g, 70% yield). ¹HNMR (400 MHZ, DMSO-d₆) δ 7.44-7.22 (m, 40H), 7.04 (d, J=5.6 Hz, 1H), 6.90 (s, 2H), 6.34 (s, 1H), 6.28 (s, 1H), 5.48 (d, J=5.2 Hz, 1H), 5.18 (d, J=7.2 Hz, 1H), 5.12 (s, 2H), 5.06 (s, 2H), 5.01 (s, 4H), 4.94 (s, 6H), 4.87 (s, 2H), 2.98 (dd, J=5.2 Hz, 1H), 2.81 (dd, J=7.6 Hz, 1H), ¹⁹F NMR (400 MHZ, DMSO-d₆) 0-133.68. LCMS: (M+H⁺): m/Z: 1197.5.

Step 2: Synthesis of Compound 40. To a solution of (2S,3R)-5,7-bis(benzyloxy)-2-(3,4,5-tris(benzyloxy)phenyl)chroman-3-yl 3,4,5-tris(benzyloxy)-2-fluorobenzoate (2.0 g, 1.670 mmol, 1 eq.), in 20 mL of (1:1; THF:MeOH, palladium hydroxide on carbon powder, Pd(OH)₂ (20 wt. %, 2.0 g) was added at RT and the reaction mixture was stirred under hydrogen atmosphere for 16 h. Then the mixture was passed through a pad of celite to remove the catalyst. The filtrate was concentrated under reduced pressure. Obtained crude compound was purified by Prep-HPLC to obtain (2S,3R)-5,7-dihydroxy-2-(3,4,5-trihydroxyphenyl)chroman-3-yl 2-fluoro-3,4,5-trihydroxybenzoate as an off-white color solid (0.46 g, 60% yield). ¹HNMR (400 MHz, DMSO-d₆): 6.67 (d, J=6.4 Hz, 1H), 6.24 (s, 2H), 5.90 (d, J=2.4 Hz, 1H), 5.79 (d, J=2.4 Hz, 1H), 5.29 (q, J=5.2 Hz, 1H), 5.03 (d, J=4.8 Hz, 1H), 2.58 (m, 2H), ¹⁹F NMR (400 MHZ, DMSO-d₆) δ−140.76. LCMS: (M−H+): m/Z: 475.20.

### Compound 41

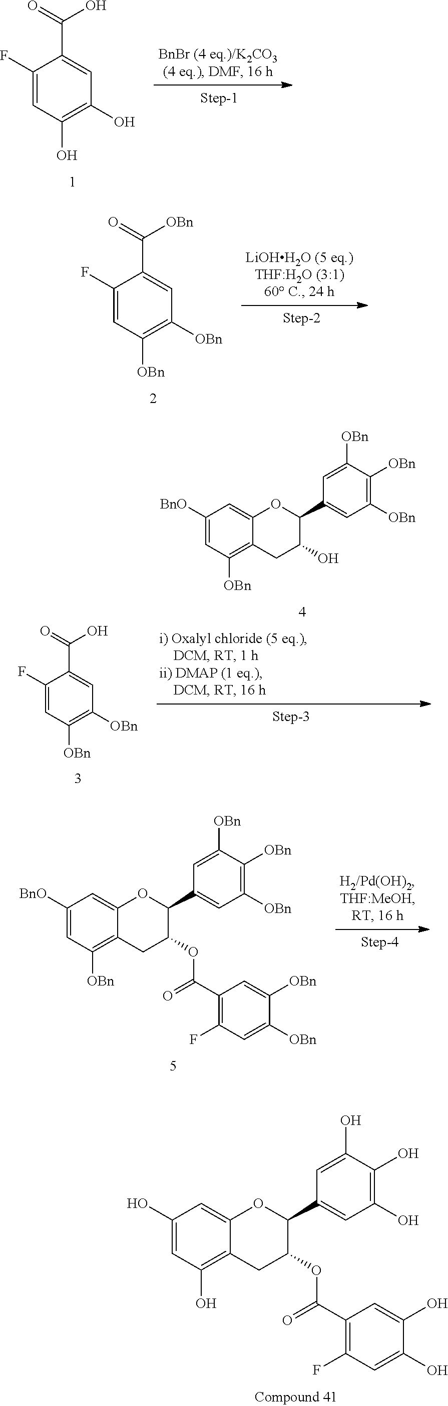 [SMILES: O=C(O)c1cc(O)c(O)cc1F ; O=C(OCc1ccccc1)c1cc(OCc2ccccc2)c(OCc2ccccc2)cc1F ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1cc(OCc2ccccc2)c(OCc2ccccc2)cc1F ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1cc(OCc2ccccc2)c(OCc2ccccc2)cc1F ; O=C(O[C@@H]1Cc2c(O)cc(O)cc2O[C@H]1c1cc(O)c(O)c(O)c1)c1cc(O)c(O)cc1F]

Step 1: Synthesis of benzyl 4,5-bis(benzyloxy)-2-fluorobenzoate (2). To a suspension of 2-fluoro-4,5-dihydroxybenzoic acid (0.5 g, 2.906 mmol, 1 eq.) in DMF (10 mL) was added K₂CO₃ (1.6 g, 11.626 mmol, 4 eq.) stirred 30 min at RT, followed by benzyl bromide (1.4 mL, 11.626 mmol, 4 eq.) at 0° C. The mixture was stirred for 16 h until TLC showed the reaction had been completed. Reaction mixture was diluted with water and extracted with EtOAc. The solvent was evaporated, and the residue was purified by flash chromatography eluted with 15% EtOAc in hexane as an eluent affords to obtain desired compound benzyl 4,5-bis(benzyloxy)-2-fluorobenzoate as a white solid (0.8 g, 62% yield). ¹HNMR (400 MHZ, DMSO-d₆): δ 6.51 (d, J=7.2 Hz, 1H), 7.42-7.25 (m, 15), 6.67 (d, J=12 Hz, 1H), 5.23 (s, 2H), 5.17 (s, 2H), 5.12 (s, 2H).

Step 2: Synthesis of 4,5-bis(benzyloxy)-2-fluorobenzoic acid (3). A mixture of benzyl 4,5-bis(benzyloxy)-2-fluorobenzoate (0.94 g, 2.126 mmol, 1.0 eq.) in THF/H₂O (3:1) (20 mL) was added LiOH H₂O (0.446 g, 10.629 mmol, 5.0 eq.). The solution was stirred at 60° C. for 24 h. The reaction mixture was concentrated to remove THF. Then the mixture was diluted with H₂O (20 mL) and extracted with EA (10 mL×1). The aqueous phase pH was adjusted to <3 with 1N HCl. Then the mixture was filtered and the filter cake was dried to give the 4,5-bis(benzyloxy)-2-fluorobenzoic acid as a white solid (0.7 g, 97% yield). ¹HNMR (400 MHZ, DMSO-d₆): δ 12.92 (s, 1H), 7.46-7.28 (m, 11), 7.09 (d, J=12.4 Hz, 1H), 5.22 (s, 2H), 5.12 (s, 2H).

Step 3: Synthesis of (2S,3R)-5,7-bis(benzyloxy)-2-(3,4,5-tris(benzyloxy)phenyl)chroman-3-yl 4,5-bis(benzyloxy)-2-fluorobenzoate (5). Under an N₂ atmosphere, to a stirred solution of 4,5-bis(benzyloxy)-2-fluorobenzoic acid (0.4 g, 1.136 mmol, 1 eq.) in DCM (6 mL) was added oxalyl chloride (0.4 mL, 5.681 mmol, 5 eq.) and two drop of DMF at 0° C. The reaction mixture was stirred at RT for 1 h. After this time, the reaction mixture was concentrated under reduced pressure to get acid chloride. Obtained acid chloride was added to a solution of (2S,3R)-5,7-bis(benzyloxy)-2-(3,4,5-tris(benzyloxy)phenyl)chroman-3-ol (0.68 g, 0.900 mmol, 0.8 eq.), DMAP (0.03 g, 1.36 mmol, 1 eq.) in DCM (6 mL) at 0° C. Then, the reaction mixture was stirred at RT 16 h. Finally, the reaction was quenched with saturated aqueous NaHCO₃ solution (5 mL). The organic layer was separated and the aqueous layer was extracted with CH₂Cl₂ (30 mL). Combined organic phase was dried over MgSO₄, filtered and concentrated under reduced pressure. Obtained crude compound was purified by flash column chromatography (EtOAc in hexane) to get (2S,3R)-5,7-bis(benzyloxy)-2-(3,4,5-tris(benzyloxy)phenyl)chroman-3-yl 4,5-bis(benzyloxy)-2-fluorobenzoate as a white solid (0.25 g, 20% yield). ¹HNMR (400 MHZ, DMSO-d₆): 7.41-7.03 (m, 34H), 7.09 (d, J=12.0 Hz, 1H), 6.86 (s, 2H), 6.42 (s, 1H), 6.27 (s, 1H), 5.74 (s, 2H), 5.42 (d, J=6.0 Hz, 1H), 5.15 (d, J=10 Hz, 1H), 5.10 (s, 2H), 5.06 (s, 4H), 4.97 (s, 6H), 4.84 (s, 2H), 2.91 (dd, J=4.4 Hz, 1H), 2.77 (dd, J=8.0 Hz, 1H), ¹⁹F NMR (375 MHZ, DMSO-d₆) δ−139.80, −138.95.

Step 4: Synthesis of Compound 41. To a solution of (2S,3R)-5,7-bis(benzyloxy)-2-(3,4,5-tris(benzyloxy)phenyl)chroman-3-yl 4,5-bis(benzyloxy)-2-fluorobenzoate (0.35 g, 0.321 mmol, 1 eq.), in 10 mL of (1:1; THF:MeOH) was added palladium hydroxide (20 wt. %, 0.35 g) at RT, reaction mixture stirred under a hydrogen atmosphere for 16 h. After this time, the mixture was filtered to remove the catalyst. The filtrate was evaporated in vacuum. The crude compound was purified by Prep-HPLC to obtain (2S,3R)-5,7-dihydroxy-2-(3,4,5-trihydroxyphenyl)chroman-3-yl 2-fluoro-4,5-dihydroxybenzoate as an off-white solid (0.078 g, 52% yield). ¹HNMR (400 MHZ, DMSO-d₆): 9.12 (bs, 7H), 7.18 (d, J=7.2 Hz, 1H), 7.18 (d, J=12.0 Hz, 1H), 6.25 (s, 2H), 5.90 (d, J=2.0 Hz, 1H), 5.79 (d, J=2.0 Hz, 1H), 5.27 (d, J=5.2 Hz, 1H), 5.01 (d, J=5.2 Hz, 1H), 2.60 (dd, J=5.6 Hz, 2H). LCMS: (M+H⁺): m/Z: 461.1.

### Compound 42

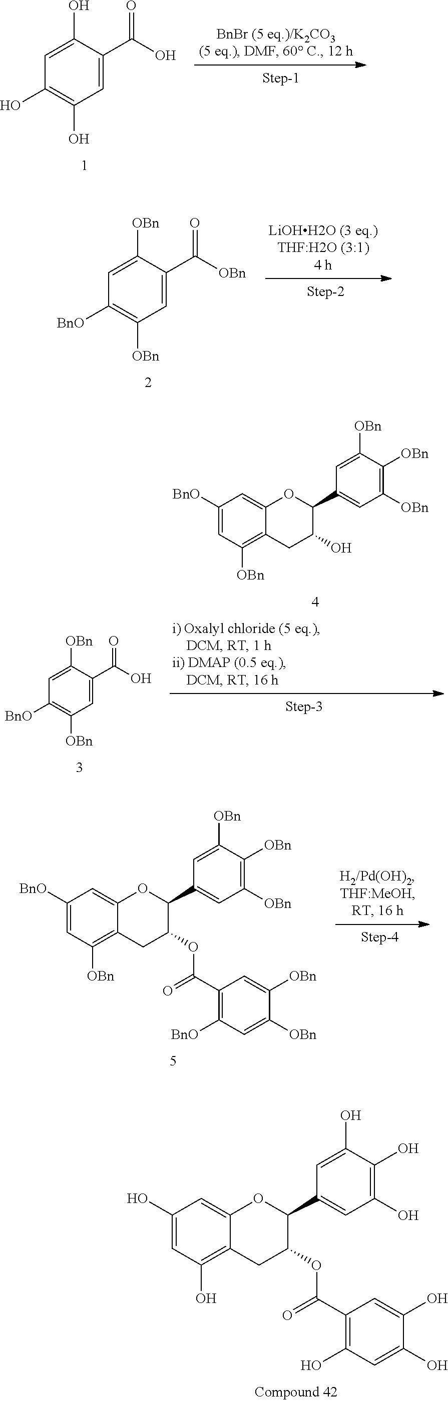 [SMILES: O=C(O)c1cc(O)c(O)cc1O ; O=C(OCc1ccccc1)c1cc(OCc2ccccc2)c(OCc2ccccc2)cc1OCc1ccccc1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1cc(OCc2ccccc2)c(OCc2ccccc2)cc1OCc1ccccc1 ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1cc(OCc2ccccc2)c(OCc2ccccc2)cc1OCc1ccccc1 ; O=C(O[C@@H]1Cc2c(O)cc(O)cc2O[C@H]1c1cc(O)c(O)c(O)c1)c1cc(O)c(O)cc1O]

Step 1: Synthesis of benzyl 2,4,5-tris(benzyloxy)benzoate (2). To a suspension of 2,4,5-trihydroxybenzoic acid (0.5 g, 2.939 mmol, 1 eq.) in DMF (10 mL) was added K₂CO₃ (2 g, 14.695 mmol, 5 eq.) followed by benzyl bromide (1.7 mL, 14.695 mmol, 5 eq.) at 0° C. The mixture was heated to 60° C. for 12 h until TLC showed the reaction had been completed. Reaction mixture diluted with water and extracted with EtOAc. The solvent was evaporated, and the residue was purified by flash chromatography eluted with 15% EtOAc in hexane as an eluent affords to obtain benzyl 2,4,5-tris(benzyloxy)benzoate as a white solid (1.3 g, 84% yield). ¹H NMR (400 MHZ, DMSO-d₆): δ 7.54 (s, 1H), 7.41 (d, J=6.8 Hz, 2H), 7.37-7.30 (m, 18), 6.56 (s, 1H), 5.30 (s, 2H), 5.12 (s, 2H), 5.09 (s, 2H), 5.00 (s, 2H).

Step 2: Synthesis of 2,4,5-tris(benzyloxy)benzoic acid (3). A mixture of benzyl 2,4,5-tris(benzyloxy)benzoate (1 g, 1.886 mmol, 1 eq.) in THF/H₂O (1:1) (20 mL) was added LiOH·H₂O (0.237 g, 5.660 mmol, 3 eq.). The solution was stirred at RT for 4 h. The reaction mixture was concentrated to remove THF. Then the mixture was diluted with H₂O (40 mL) and extracted with EA (15 mL). The aqueous phase pH was adjusted to <3 with 1N HCl. Then the mixture was filtered and the filter cake was dried to obtained 2,4,5-tris(benzyloxy)benzoic acid as a white solid (0.68 g, 82% yield). ¹H NMR (400 MHZ, DMSO-d₆): δ 7.48-7.45 (m, 4H), 7.41-7.29 (m, 12), 6.95 (s, 1H), 5.20 (s, 2H), 5.13 (s, 2H), 5.05 (s, 2H).

Step 3: Synthesis of (2S,3R)-5,7-bis(benzyloxy)-2-(3,4,5-tris(benzyloxy)phenyl)chroman-3-yl 2,4,5-tris(benzyloxy)benzoate (4). Under an N₂ atmosphere, to a stirred solution of 2,4,5-tris(benzyloxy)benzoic acid (0.510 g, 1.157 mmol, 1 eq.) in DCM (8 mL) was added oxalyl chloride (0.49 mL, 5.795 mmol, 5 eq.) and two drop of DMF at 0° C. The reaction mixture was stirred at RT for 1 h. After this time, the reaction mixture was concentrated under reduced pressure to get acid chloride. Obtained acid chloride was added to a solution of (2S,3R)-5,7-bis(benzyloxy)-2-(3,4,5-tris(benzyloxy)phenyl)chroman-3-ol (0.7 g, 0.925 mmol, 0.8 eq.), DMAP (0.564 g, 4.628 mmol, 0.5 eq.), in CH₂Cl₂ (12 mL) at 0° C. Then the reaction mixture was stirred at RT 16 h. Finally, the reaction was quenched with saturated aqueous NaHCO₃ solution (5 mL). The organic layer was separated and the aqueous layer was extracted with CH₂Cl₂ (30 mL). Combined organic phase was dried over MgSO₄, filtered and concentrated under reduced pressure. Obtained crude compound was purified by flash column chromatography (EtOAc in hexane) to get (2S,3R)-5,7-bis(benzyloxy)-2-(3,4,5-tris(benzyloxy)phenyl)chroman-3-yl 2,4,5-tris(benzyloxy)benzoate as a light brown solid (0.3 g, 22% yield). ¹H NMR (400 MHZ, DMSO-d₆): 7.40-7.11 (s, 41H), 7.11 (s, 1H), 6.91 (d, J=3.2 Hz, 1H), 6.84 (d, J=6.0 Hz, 1H), 6.41 (s, 1H), 6.26 (s, 1H), 5.44 (d, J=6.8 Hz, 1H), 5.16 (d, J=8.8 Hz, 1H), 5.15 (s, 8H), 4.91 (s, 2H), 4.88 (s, 2H), 4.83 (s, 4H), 2.64 (dd, J=6.8 Hz, 2H).

Step 4: Synthesis of Compound 42. To a solution of (2S,3R)-5,7-bis(benzyloxy)-2-(3,4,5-tris(benzyloxy)phenyl)chroman-3-yl 2,4,5-tris(benzyloxy)benzoate (0.3 g, 0.254 mmol, 1 eq.), in 10 mL of (1:1; THF:MeOH) was added palladium hydroxide (20 wt. %, 0.3 g) at RT and the reaction mixture was stirred under hydrogen atmosphere for 16 h. Then the mixture was passed through a pad of celite to remove the catalyst. The filtrate was concentrated under reduced pressure. Obtained crude compound was purified by Prep-HPLC to obtain (2S,3R)-5,7-dihydroxy-2-(3,4,5-trihydroxyphenyl)chroman-3-yl 2,4,5-trihydroxybenzoate as a light brown solid (0.03 g, 26% yield). ¹H NMR (400 MHZ, DMSO-d₆): 9.02 (s, 8H), 6.93 (bs, 1H), 6.25 (d, J=5.6 Hz, 3H), 5.91 (d, J=2.0 Hz, 1H), 5.81 (d, J=2.4 Hz, 1H), 5.34 (q, J=4.8 Hz, 1H), 5.11 (d, J=4.8 Hz, 1H), 2.59 (dd, J=4.0 Hz, 2H). LCMS: (M−H+): m/Z: 457.1.

### Compounds 43 and 44

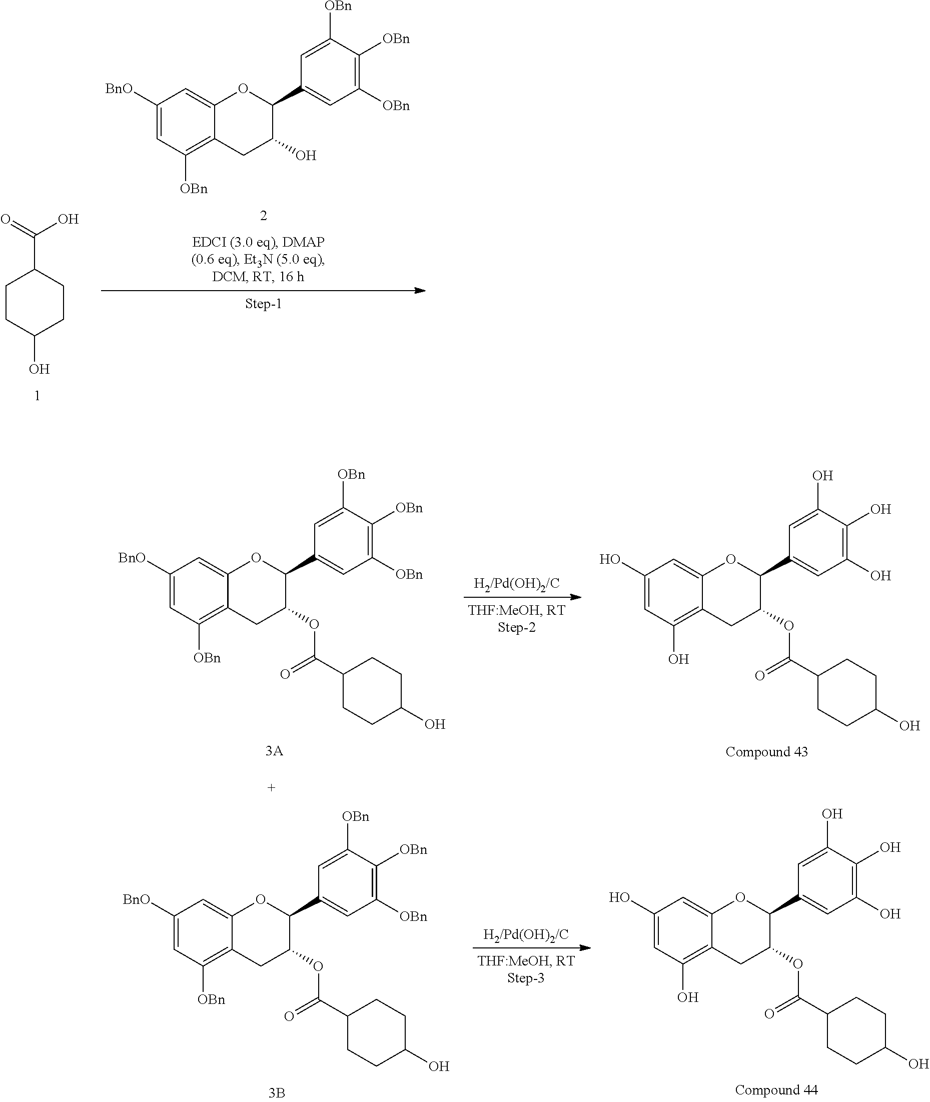 [SMILES: O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)C1CCC(O)CC1 ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)C1CCC(O)CC1 ; O=C(O[C@@H]1Cc2c(O)cc(O)cc2O[C@H]1c1cc(O)c(O)c(O)c1)C1CCC(O)CC1 ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)C1CCC(O)CC1 ; O=C(O[C@@H]1Cc2c(O)cc(O)cc2O[C@H]1c1cc(O)c(O)c(O)c1)C1CCC(O)CC1]

Step 1: Synthesis of (2S,3R)-5,7-bis(benzyloxy)-2-(3,4,5-tris(benzyloxy)phenyl)chroman-3-yl (1r,4R)-4-hydroxycyclohexane-1-carboxylate (3A & 3B). To a mixture of (2S,3R)-5,7-bis(benzyloxy)-2-(3,4,5-tris(benzyloxy)phenyl)chroman-3-ol (0.1 g, 0.13 mmol, 1.0 eq.) and 4-hydroxycyclohexane-1-carboxylic acid (0.02 g, 0.10 mmol, 0.8 eq.) in CH₂Cl₂ (5 mL) was added EDCI (0.07 g, 0.39 mmol, 3.0 eq.), DMAP (0.01 g, 0.08 mmol, 0.6 eq.) and TEA (0.1 mL, 0.66 mmol, 5.0 eq.) at 0° C. The reaction mixture was stirred at room temperature for overnight. The reaction mixture was diluted with H₂O (20 mL) and extracted with CH₂Cl₂ (2×50 mL). The combined organic layer was washed with brine (30 mL), dried over Na₂SO₄, filtered and concentrated. The residue was purified by flash chromatography on silica gel (PE/EA=5/1) to give major isomer (30 mg) & minor isomer (15 mg) overall 38.7% yield as white solids.

Major-analytical Data: ¹H NMR (400 MHZ, CDCl₃): δ 7.48-7.20 (m, 25H), 6.67 (s, 2H), 6.27 (d, J=2.4 Hz, 1H), 6.24 (d, J=2.0 Hz, 1H), 5.31 (q, J=6.8 Hz, 1H), 5.10-4.90 (m, 11H), 3.51 (m, 1H), 2.80 (dq, J=16.8, 5.2 Hz, 2H), 2.11 (m, 1H), 2.00-1.80 (m, 3H), 1.80-1.70 (m, 1H), 1.50-1.11 (m, 4H). Minor-analytical Data: ¹H NMR (400 MHZ, CDCl₃): δ 7.48-7.30 (m, 25H), 6.69 (s, 2H), 6.27 (d, J=2.4 Hz, 1H), 6.24 (d, J=2.0 Hz, 1H), 5.31 (q, J=6.8 Hz, 1H), 5.15-4.90 (m, 12H), 3.76 (m, 1H), 2.70 (dq, J=16.8, 5.6 Hz, 2H), 2.27 (m, 1H), 2.10-1.95 (m, 3H), 1.90-1.70 (m, 2H), 1.60-1.16 (m, 3H).

Step 2: Synthesis of Compound 43. To a mixture of compound (2S,3R)-5,7-bis(benzyloxy)-2-(3,4,5-tris(benzyloxy)phenyl)chroman-3-yl 4-hydroxycyclohexane-1-carboxylate (120 mg, 0.13 mmol, 1.0 eq.) in THF (3 mL) and MeOH (3 mL) was added Pd(OH)₂ (20 wt. %, 0.024 g). The mixture was stirred at room temperature under H₂ atmosphere for overnight. The reaction mixture was passed through celite bed and the filtrate was concentrated. The residue was purified by prep-HPLC to give (2S,3R)-5,7-dihydroxy-2-(3,4,5-trihydroxyphenyl)chroman-3-yl 4-hydroxycyclohexane-1-carboxylate (0.022 g, 37.9% yield) as off-white solid. ¹H NMR (400 MHZ, DMSO-d₆): δ 9.36 (bs, 1H), 9.10 (bs, 1H), 8.88 (bs, 2H), 8.15 (bs, 1H), 6.21 (s, 2H), 5.89 (d, J=2.0 Hz, 1H), 5.75 (d, J=2.4 Hz, 1H), 5.03 (q, J=6.0 Hz, 1H), 4.80 (d, J=6.0 Hz, 1H), 4.36 (s, 1H), 3.59 (bs, 1H), 2.70-2.40 (m, 2H), 2.22 (m, 1H), 1.80-1.55 (m, 2H), 1.55-1.30 (m, 6H).

Step 2: Synthesis of Compound 44. To a mixture of (2S,3R)-5,7-bis(benzyloxy)-2-(3,4,5-tris(benzyloxy)phenyl)chroman-3-yl 4-hydroxycyclohexane-1-carboxylate (0.190 g, 0.21 mmol, 1.0 eq.) in THF (5 mL) and MeOH (5 mL) was added Pd(OH)₂ (20 wt. %, 0.030 g) at RT and the reaction mixture was stirred under hydrogen atmosphere for 16 h. Then the mixture was passed through a pad of celite to remove the catalyst. The filtrate was concentrated under reduced pressure. Obtained crude compound was purified by Prep-HPLC to obtain (2S,3R)-5,7-dihydroxy-2-(3,4,5-trihydroxyphenyl)chroman-3-yl 4-hydroxycyclohexane-1-carboxylate (0.050 g, 53.7% yield) as off-white solid. ¹H NMR (400 MHZ, DMSO-de): δ 9.32 (s, 1H), 9.05 (s, 1H), 8.85 (bs, 2H), 8.09 (bs, 1H), 6.21 (s, 2H), 5.90 (d, J=2.4 Hz, 1H), 5.74 (d, J=2.0 Hz, 1H), 5.01 (q, J=6.0 Hz, 1H), 4.78 (d, J=6.4 Hz, 1H), 4.52 (d, J=4.0 Hz, 1H), 3.27 (m, 1H), 2.70-2.40 (m, 2H), 2.11-2.01 (m, 1H), 1.80-1.60 (m, 4H), 1.40-1.01 (m, 4H).

### Compound 45

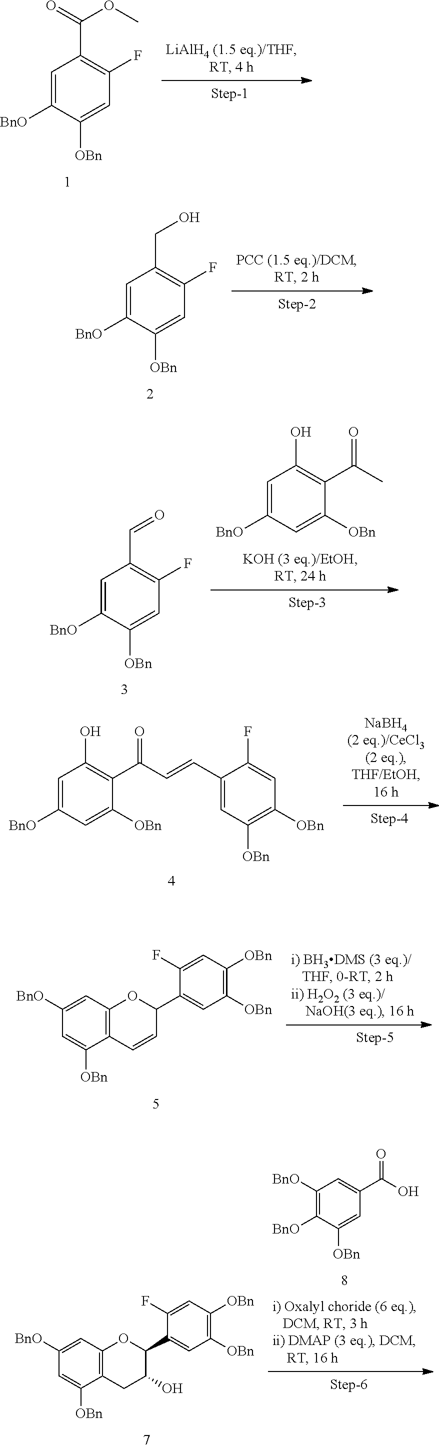

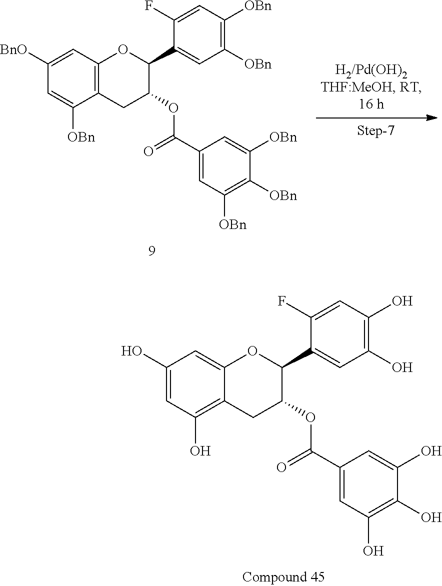 [SMILES: O=C(O[C@@H]1Cc2c(OCc3ccccc3)cc(OCc3ccccc3)cc2O[C@H]1c1cc(OCc2ccccc2)c(OCc2ccccc2)cc1F)c1cc(OCc2ccccc2)c(OCc2ccccc2)c(OCc2ccccc2)c1 ; O=C(O[C@@H]1Cc2c(O)cc(O)cc2O[C@H]1c1cc(O)c(O)cc1F)c1cc(O)c(O)c(O)c1]

Step 1: Synthesis of (4,5-bis(benzyloxy)-2-fluorophenyl)methanol (2). A mixture of methyl 4,5-bis(benzyloxy)-2-fluorobenzoate (0.3 g, 0.678 mmol, 1 eq.) in THF (10 mL) was added LiAlH₄ (2M, 0.5 mL, 1.017 mmol) at 0° C. The reaction mixture was stirred at room temperature for 4 h. After this time, reaction mixture was diluted with H₂O (0.5 mL) and 15% NaOH solution (1 mL) at 0° C., extracted with DCM (3×30 mL). The combined organic phases were washed with brine (2×30 mL), dried over Na₂SO₄, filtered and concentrated to give (4,5-bis(benzyloxy)-2-fluorophenyl)methanol as a white solid (0.19 g, 83% yield). ¹HNMR (400 MHZ, DMSO-d₆): δ 7.43-7.31 (m, 10H), 7.11 (d, J=7.6 Hz, 1H), 6.94 (d, J=11.6 Hz, 1H), 5.12 (s, 2H), 5.05 (s, 2H), 4.41 (d, J=5.6 Hz, 2H).

Step 2: Synthesis of 4,5-bis(benzyloxy)-2-fluorobenzaldehyde (3). A mixture of compound (4,5-bis(benzyloxy)-2-fluorophenyl)methanol (0.1 g, 0.295 mmol, 1 eq.) in DCM (3 mL) was added PCC (0.096 g, 0.443 mmol, 1.5 eq.). The reaction mixture was stirred at room temperature for 2 hours. The reaction mixture was concentrated and the residue was purified by flash chromatography eluted with 50% EtOAc in hexane as an eluent affords to obtain 4,5-bis(benzyloxy)-2-fluorobenzaldehyde as a white solid (0.088 g, 89% yield). ¹HNMR (400 MHZ, DMSO-d₆) δ 10.18 (s, 1H), 7.47-7.25 (m, 11H), 6.69 (d, J=11.6 Hz, 1H), 5.21 (s, 2H), 5.14 (s, 2H), 5.09 (s, 2H).

Step 3: Synthesis of (E)-3-(4,5-bis(benzyloxy)-2-fluorophenyl)-1-(2,4-bis(benzyloxy)-6-hydroxyphenyl)prop-2-en-1-one (4). To a solution of 1-(2,4-bis(benzyloxy)-6-hydroxyphenyl)ethan-1-one (0.072 g, 0.208 mmol, 1 eq.) in EtOH (2 mL) was added KOH (0.316 g, 0.624 mmol, 3 eq.) at RT. The mixture was stirred at room temperature for 30 min. Then added compound 4,5-bis(benzyloxy)-2-fluorobenzaldehyde (0.07 g, 0.208 mmol, 1 eq.) to the above reaction mixture. The reaction mixture was stirred at RT for 24 h. After this time, the reaction mixture was concentrated, obtained crude was diluted with H₂O (15 mL) and extracted with EA (20 mL×3). The combined organic layers were washed with brine (20 mL×2), dried over Na₂SO₄, filtered and concentrated. The solid was triturated with EtOH (20 mL), filtered, washed with EtOH (10 mL), then dried obtained (E)-3-(4,5-bis(benzyloxy)-2-fluorophenyl)-1-(2,4-bis(benzyloxy)-6-hydroxyphenyl)prop-2-en-1-one as a yellow solid (0.029 g, 21% yield). ¹HNMR (400 MHZ, DMSO-d₆) δ 12.97 (s, 1H), 7.62 (d, J=14.4 Hz, 1H), 7.46-7.29 (s, 17H), (s, 1H), 7.24 (d, J=6.4 Hz, 1H), 7.19 (t, J=4.0 Hz, 3H), 6.07 (d, J=22.0 Hz, 1H), 6.37 (d, J=2.0 Hz, 1H), 6.23 (d, J=2.0 Hz, 1H), 5.21 (s, 2H), 5.19 (s, 2H), 5.16 (s, 2H), 4.92 (s, 2H).

Step 4: Synthesis of Synthesis of 5,7-bis(benzyloxy)-2-(3,4,5-tris(benzyloxy)-2-fluorophenyl)-2H-chromene (5). To a solution of (E)-3-(4,5-bis(benzyloxy)-2-fluorophenyl)-1-(2,4-bis(benzyloxy)-6-hydroxyphenyl)prop-2-en-1-one (0.5 g, 0.750 mmol, 1 eq.) in THF/EtOH (4 mL/2 mL) was added CeCl₃ (0.46 g, 1.876 mmol, 2 eq.) and NaBH₄ (0.71 g, 1.876 mmol, 2 eq.) at 0° C. The reaction mixture was stirred at room temperature 16 h. The reaction mixture was diluted with water (30 mL) and extracted with DCM (2×20 mL). The combined organic layers were dried over Na₂SO₄ and filtered. The filtrate was concentrated to give the crude product the residue was purified by flash chromatography eluted with 30% EtOAc in hexane as an eluent affords to obtain 5,7-bis(benzyloxy)-2-(3,4,5-tris(benzyloxy)-2-fluorophenyl)-2H-chromene as a light yellow solid (0.25 g, 5% yield). ¹HNMR (400 MHZ, DMSO-d₆) δ 7.46-7.24 (m, 24H), 7.04-6.99 (m, 3H), 6.94-6.85 (m, 2H) 6.82 (dd, J=13.6 Hz, 1H), 6.52 (s, 1H) 5.62 (dd, J=4.0 Hz, 1H), 5.14 (s, 4H), 5.08 (s, 2H), 4.87 (s, 2H), 3.73 (q, J=14.0 Hz, 1H).

Step 5: Synthesis of (2S,3R)-5,7-bis(benzyloxy)-2-(4,5-bis(benzyloxy)-2-fluorophenyl)chroman-3-ol (6). A solution of 5,7-bis(benzyloxy)-2-(4,5-bis(benzyloxy)-2-fluorophenyl)-2H-chromene (1.2 g, 1.846 mmol, 1 eq.) in 12 mL of THF was added BH3: DMS (2M, 2.8 mL, 5.538 mmol, 3 eq.) at ice-water bath for 2 hours. The mixture was added 3 N of NaOH solution (0.228 g, 5.538 mmol, 3 eq.) and 30% aqueous solution of H₂O₂ (0.62 mL, 5.538 mmol, 3 eq.) at 0° C. The reaction was stirred at RT for 16 h. After this time, the reaction mixture was diluted with H₂O (20 mL) and extracted with EA (2×30 mL). The organic phase was concentrated and crude product the residue was purified by flash chromatography eluted with 30% EtOAc in hexane, as an eluent affords to obtain (2S,3R)-5,7-bis(benzyloxy)-2-(4,5-bis(benzyloxy)-2-fluorophenyl)chroman-3-ol as a white solid (1.0 g g, 83% yield). ¹HNMR (400 MHz, DMSO-d₆) δ 7.45-7.27 (m, 18H), 7.07 (d, J=6.8 Hz, 1H), 6.99 (d, J=6.4 Hz, 1H), 6.34 (d, J=2.0 Hz, 1H), 6.10 (d, J=2.0 Hz, 1H), 5.14 (d, J=5.2 Hz, 1H), 5.10 (s, 2H), 5.07 (s, 2H), 4.99 (s, 4H), 3.79 (d, J=8.4 Hz, 1H), 2.86 (dd, J=4.8 Hz, 2H).

Step 6: Synthesis of (2S,3R)-5,7-bis(benzyloxy)-2-(4,5-bis(benzyloxy)-2-fluorophenyl)chroman-3-yl 3,4,5-tris(benzyloxy)benzoate (8). Under an N₂ atmosphere, to a stirred solution of 3,4,5-tris(benzyloxy)benzoic acid (0.8 g, 1.807 mmol, 3 eq.) in DCM (8 mL) was added oxalyl chloride (0.4 mL, 3.612 mmol, 6 eq.) and two drop of DMF at 0° C. The reaction mixture was stirred at RT for 1 h. After this time, the reaction mixture was concentrated under reduced pressure to get acid chloride. Obtained acid chloride was added to a solution of (2S,3R)-5,7-bis(benzyloxy)-2-(4,5-bis(benzyloxy)-2-fluorophenyl)chroman-3-ol (0.4 g, 0.602 mmol, 1 eq.), DMAP (0.1 g, 3.611 mmol, 3 eq.) in CH₂Cl₂ (10 mL) at 0° C. Then the reaction mixture was stirred at RT 16 h. Finally, the reaction was quenched with saturated aqueous NaHCO₃ solution (5 mL). The organic layer was separated and the aqueous layer was extracted with CH₂Cl₂ (30 mL). Combined organic phase was dried over MgSO₄, filtered and concentrated under reduced pressure. Obtained crude compound was purified by flash column chromatography (EtOAc in hexane) to get (2S,3R)-5,7-bis(benzyloxy)-2-(4,5-bis(benzyloxy)-2-fluorophenyl)chroman-3-yl 3,4,5-tris(benzyloxy)benzoate as a light yellow solid (0.25 g, 35% yield). ¹HNMR (400 MHZ, DMSO-d₆): δ 7.49-7.16 (m, 43H), 7.06-6.99 (m, 2H), 6.41 (dd, J=2.0 Hz, 1H), 6.19 (dd, J=2.0 Hz, 1H), 5.36 (q, J=6.0 Hz, 1H), 5.24 (d, J=7.2 Hz, 1H), 5.14 (s, 2H), 5.08 (s, 2H), 5.05 (s, 2H), 4.98 (s, 2H), 2.89 (dd, J=7.6 Hz, 1H), 2.66 (dd, J=7.6 Hz, 1H).

Step 7: Synthesis of Compound 45. To a solution of (2S,3R)-5,7-bis(benzyloxy)-2-(4,5-bis(benzyloxy)-2-fluorophenyl)chroman-3-yl 3,4,5-tris(benzyloxy)benzoate (0.2 g, 0.183 mmol, 1 eq.), in 8 mL of 1:1; THF:MeOH was added palladium hydroxide (20 wt. %, 0.2 g) at RT and the reaction mixture was stirred under hydrogen atmosphere for 16 h. Then the mixture was passed through a pad of celite to remove the catalyst. The filtrate was concentrated under reduced pressure. Obtained crude compound was purified by Prep-HPLC to obtain (2S,3R)-2-(2-fluoro-4,5-dihydroxyphenyl)-5,7-dihydroxychroman-3-yl 3,4,5-trihydroxybenzoate as an grey color solid (0.020 g, 23% yield). ¹HNMR (400 MHZ, DMSO-d₆): δ 9.24 (s, 7H), 6.81 (s, 2H), 6.67 (d, J=7.2 Hz, 1H), 6.52 (d, J=11.6 Hz, 1H), 5.95 (d, J=2.0 Hz, 1H), 6.41 (d, J=2.0 Hz, 1H), 5.25 (q, J=6.8 Hz, 1H), 5.21 (d, J=6.0 Hz, 1H), 2.77 (dd, J=7.6 Hz, 1H), 2.57 (dd, J=7.6 Hz, 1H). LCMS: (M+H⁺): m/Z: 461.0.

### Compound 46

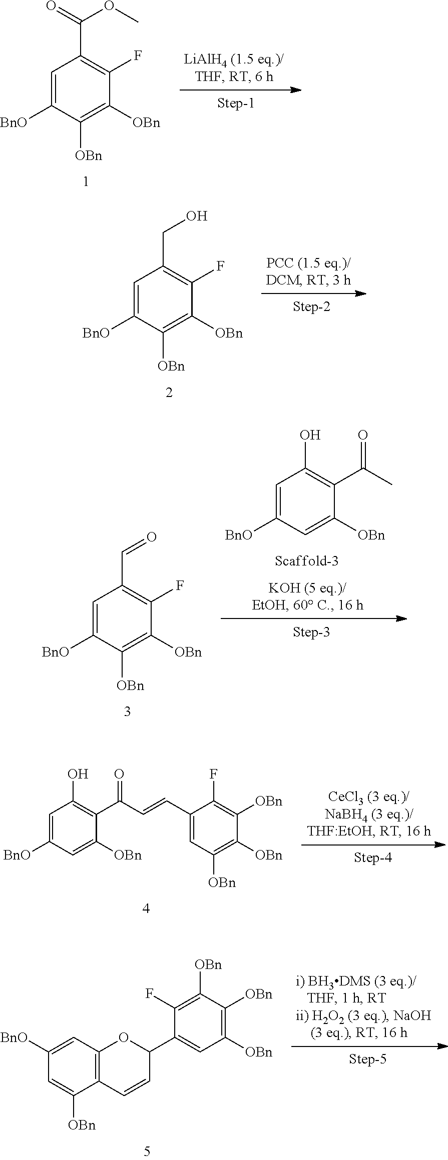 [SMILES: COC(=O)c1cc(OCc2ccccc2)c(OCc2ccccc2)c(OCc2ccccc2)c1F ; OCc1cc(OCc2ccccc2)c(OCc2ccccc2)c(OCc2ccccc2)c1F ; CC(=O)c1c(O)cc(OCc2ccccc2)cc1OCc1ccccc1 ; O=Cc1cc(OCc2ccccc2)c(OCc2ccccc2)c(OCc2ccccc2)c1F ; O=C(/C=C/c1cc(OCc2ccccc2)c(OCc2ccccc2)c(OCc2ccccc2)c1F)c1c(O)cc(OCc2ccccc2)cc1OCc1ccccc1 ; Fc1c(C2C=Cc3c(OCc4ccccc4)cc(OCc4ccccc4)cc3O2)cc(OCc2ccccc2)c(OCc2ccccc2)c1OCc1ccccc1]

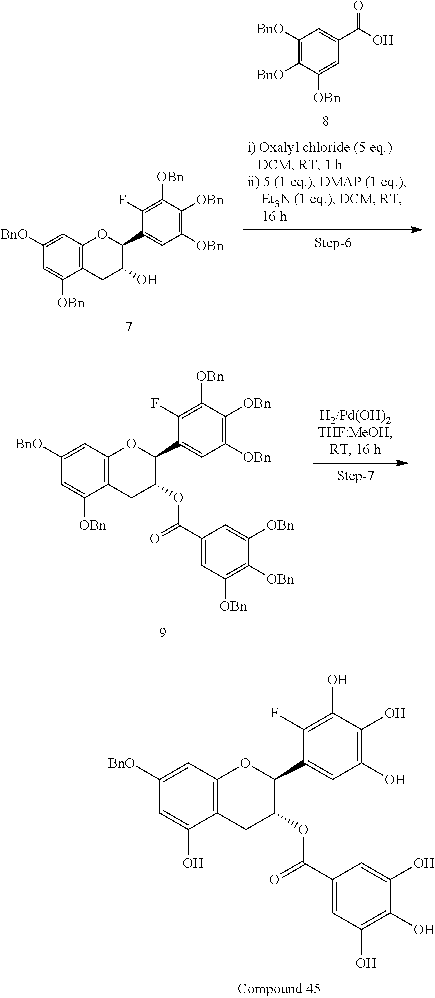 [SMILES: O=C(O)c1cc(OCc2ccccc2)c(OCc2ccccc2)c(OCc2ccccc2)c1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1F ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1F)c1cc(OCc2ccccc2)c(OCc2ccccc2)c(OCc2ccccc2)c1 ; O=C(O[C@@H]1Cc2c(O)cc(OCc3ccccc3)cc2O[C@H]1c1cc(O)c(O)c(O)c1F)c1cc(O)c(O)c(O)c1]

Step 1: Synthesis of (3,4,5-tris(benzyloxy)-2-fluorophenyl)methanol (2). A mixture of compound methyl 3,4,5-tris(benzyloxy)-2-fluorobenzoate (5 g, 10.550 mmol, 1 eq.) in THF (30 mL) was added LiAlH₄ (2M, 7.9 mL, 15.820 mmol, 1.5 eq.) at 0° C. The solution was stirred at room temperature for 6 h. After this time, the reaction mixture was diluted with H₂O (8 mL) and 15% NaOH solution (2 mL) at 0° C., extracted with DCM (30 mL×3). The combined organic phases were washed with brine (30 mL×2), dried over Na₂SO₄, filtered and concentrated to give compound (3,4,5-tris(benzyloxy)-2-fluorophenyl)methanol as a white solid (3.0 g, 65% yield). ¹HNMR (400 MHZ, DMSO-d₆) δ 7.46 (d, J=7.2 Hz, 2H), 7.40-7.21 (m, 13H), 6.97 (d, J=1.2 Hz, 1H), 5.10 (s, 2H), 5.03 (s, 2H), 4.91 (s, 2H), 4.35 (s, 1H), 3.37 (d, J=4.8 Hz, 2H).

Step 2: Synthesis of 3,4,5-tris(benzyloxy)benzaldehyde (3). A mixture of (3,4,5-tris(benzyloxy)-2-fluorophenyl)methanol (3 g, 6.756 mmol, 1 eq.) in DCM (30 mL) was added PCC (2.17 g, 10.135 mmol 1.5 eq.). The reaction mixture was stirred at room temperature for 3 h. The reaction mixture was concentrated and the residue was purified by flash chromatography eluted with 30% EtOAc in hexane as an eluent affords to obtain desired compound 3,4,5-tris(benzyloxy)benzaldehyde as a white solid (2 g, 68% yield). ¹HNMR (400 MHZ, DMSO-d₆) δ 10.11 (s, 1H), 7.47-7.25 (m, 16H), 5.19 (s, 2H), 5.15 (s, 2H), 5.09 (s, 2H).

Step 3: Synthesis of (E)-1-(2,4-bis(benzyloxy)-6-hydroxyphenyl)-3-(3,4,5-tris(benzyloxy)-2-fluorophenyl)prop-2-en-1-one (4). To a solution of 1-(2,4-bis(benzyloxy)-6-hydroxyphenyl)ethan-1-one (2 g, 5.429 mmol, 1.2 eq.) in EtOH (10 mL) was added KOH (1.2 g, 21.25 mmol, 5 eq.). The mixture was stirred at room temperature for 30 min. Then added 3,4,5-tris(benzyloxy)-2-fluorobenzaldehyde (2 g, 4.524 mmol, 1 eq.). The reaction mixture was stirred at 60° C. for 16 h. The reaction mixture was concentrated. The residue was diluted with H₂O (50 mL) and extracted with EA (50 mL×3). The combined organic layers were washed with brine (30 mL×2), dried over Na₂SO₄, filtered and concentrated. The solid was triturated with EtOH (20 mL), filtered, washed with EtOH (10 mL), then dried to obtained (E)-1-(2,4-bis(benzyloxy)-6-hydroxyphenyl)-3-(3,4,5-tris(benzyloxy)-2-fluorophenyl)prop-2-en-1-one as a yellow solid (3.2 g, 96% yield). ¹HNMR (400 MHZ, DMSO-d₆) δ 12.83 (s, 1H), 7.73 (d, J=9.2 Hz, 1H), 7.46-7.31 (14) 7.19-7.14 (m, 10H), 6.61 (s, 1H), 6.38 (s, 1H), 6.25 (s, 1H), 5.28 (d, J=8.0 Hz, 1H), 5.19 (s, 2H), 5.11 (s, 2H), 5.10 (s, 2H), 5.03 (s, 2H), 4.93 (s, 2H).

Step 4: Synthesis of 5,7-bis(benzyloxy)-2-(3,4,5-tris(benzyloxy)-2-fluorophenyl)-2H-chromene (5). To a solution of (E)-1-(2,4-bis(benzyloxy)-6-hydroxyphenyl)-3-(3,4,5-tris(benzyloxy)-2-fluorophenyl)prop-2-en-1-one (3 g, 4.464 mmol, 1 eq.) in THF/EtOH (15 mL/3 mL) was added CeCl₃ (3.3 g, 13.390 mmol, 3 eq.) and NaBH₄ (0.5 g, 13.390 mmol, 3 eq.) at 0° C. The reaction mixture was stirred at room temperature overnight. The reaction mixture was diluted with water (30 mL) and extracted with DCM (20 mL×2). The combined organic layers were dried over Na₂SO₄ and filtered. The filtrate was concentrated to give the crude product the residue was purified by flash chromatography eluted with 30% EtOAc in hexane as an eluent affords to obtain 5,7-bis(benzyloxy)-2-(3,4,5-tris(benzyloxy)-2-fluorophenyl)-2H-chromene as a white solid (2.0 g, 68% yield). ¹HNMR (400 MHZ, DMSO-d₆) δ 7.47-7.26 (m, 24H), 7.06-6.98 (m, 3H), 6.88 (d, J=6.8 Hz, 1H), 6.78 (d, J=11.2 Hz, 1H), 6.38 (d, J=2.0 Hz, 1H), 6.13 (d, J=2.0 Hz, 1H), 5.62 (dd, J=13.6 Hz, 1H), 5.13 (s, 2H), 4.50 (s, 2H), 4.99 (s, 4H), 4.98 (s, 2H), 3.73 (d, J=14.8 Hz, 1H).

Step 5: Synthesis of (2S,3R)-5,7-bis(benzyloxy)-2-(3,4,5-tris(benzyloxy)-2-fluorophenyl)chroman-3-ol (6). A solution of 5,7-bis(benzyloxy)-2-(3,4,5-tris(benzyloxy)-2-fluorophenyl)-2H-chromene (2 g, 4.385 mmol, 1 eq.) in 20 mL of THF was added BH3 DMS (1M, 13.157 mL, 13.157 mmol, 3 eq.) at 0° C. for 1 h. After this time, added 3 N of NaOH solution (0.52 g, 13.157 mmol, 3 eq.) and 30% aqueous solution of H₂O₂ (1.8 mL, 13.157 mmol, 3 eq.) at 0° C. The reaction was stirred at RT for 16 h. The reaction mixture was then diluted with H₂O (20 mL) and extracted with EA (20 mL×2). The organic phase was concentrated and crude product the residue was purified by flash chromatography eluted with 30% EtOAc in hexane as an eluent affords to obtain (2S,3R)-5,7-bis(benzyloxy)-2-(3,4,5-tris(benzyloxy)-2-fluorophenyl)chroman-3-ol as a white solid (1.1 g, 33% yield). ¹HNMR (400 MHZ, DMSO-d₆) δ 7.46-7.28 (m, 24H), 7.13 (d, J=6.8 Hz, 2H), 6.96 (d, J=6.4 Hz, 1H), 6.37 (d, J=2.0 Hz, 1H), 6.13 (d, J=2.0 Hz, 1H), 5.22 (dd, J=5.2 Hz, 1H), 5.11 (s, 2H), 5.05 (s, 4H), 5.00 (s, 2H), 4.97 (s, 2H), 3.79 (q, J=7.2 Hz, 1H), 2.90 (dd, J=4.8 Hz, 2H).

Step 6: Synthesis of (2S,3R)-5-(benzyloxy)-2-(3,4,5-tris(benzyloxy)phenyl)chroman-3-yl 3,4,5-tris(benzyloxy)benzoate (7). Under an N₂ atmosphere, to a stirred solution of 3,4,5-tris(benzyloxy)benzoic acid (0.17 g, 0.387 mmol, 3 eq.) in DCM (4 mL) was added oxalyl chloride (0.08 mL, 0.645 mmol, 5 eq.) and two drop of DMF stirred at RT at 0° C. The reaction mixture was stirred at RT for 1 h. After this time, the reaction mixture was concentrated under reduced pressure to get acid chloride. Obtained acid chloride was added to a solution of (2S,3R)-5,7-bis(benzyloxy)-2-(3,4,5-tris(benzyloxy)-2-fluorophenyl)chroman-3-ol (0.1 g, 0.129 mmol, 1 eq.), DMAP (0.015 g, 0.129 mmol, 1 eq.) in CH₂Cl₂ (10 mL) at 0° C. Then the reaction mixture was stirred at RT 16 h. Finally, the reaction was quenched with saturated aqueous NaHCO₃ solution (5 mL). The organic layer was separated and the aqueous layer was extracted with CH₂Cl₂ (30 mL). Combined organic phase was dried over MgSO₄, filtered and concentrated under reduced pressure. Obtained crude compound was purified by flash column chromatography (EtOAc in hexane) to get (2S,3R)-5-(benzyloxy)-2-(3,4,5-tris(benzyloxy)phenyl)chroman-3-yl 3,4,5-tris(benzyloxy)benzoate as a light yellow solid (0.12 g, 26% yield). ¹HNMR (400 MHZ, DMSO-d₆): δ 7.46-7.23 (m, 39H), 7.15 (d, J=6.8 Hz, 2H), 6.88 (d, J=6.8 Hz, 1H), 6.63 (d, J=6.4 Hz, 1H), 6.42 (d, J=2.0 Hz, 1H), 6.21 (d, J=2.0 Hz, 1H), 5.51 (q, J=6.0 Hz, 1H), 5.29 (d, J=7.2 Hz, 1H), 5.18 (s, 2H), 5.15 (s, 2H), 5.11 (s, 4H), 4.00 (s, 4H), 4.96 (s, 4H), 2.84 (dd, J=7.6 Hz, 1H), 2.72 (dd, J=7.6 Hz, 1H).

Step 7: Synthesis of Compound 46. To a solution of (2S,3R)-5,7-bis(benzyloxy)-2-(3,4,5-tris(benzyloxy)-2-fluorophenyl)chroman-3-yl 3,4,5-tris(benzyloxy)benzoate (0.4 g, 0.334 mmol, 1 eq.) in 8 mL of 1:1 THF:MeOH was added palladium hydroxide (20 wt. %, 0.040 g) was added at RT and the reaction mixture was stirred under hydrogen atmosphere for 16 h. Then the mixture was passed through a pad of celite to remove the catalyst. The filtrate was concentrated under reduced pressure. Obtained crude compound was purified by Prep-HPLC to obtain (2S,3R)-2-(2-fluoro-3,4,5-trihydroxyphenyl)-5,7-dihydroxychroman-3-yl 3,4,5-trihydroxybenzoate as a grey color solid (0.05 g, 3% yield). LCMS: (M+H⁺): m/Z: 477.0.

### Compound 47

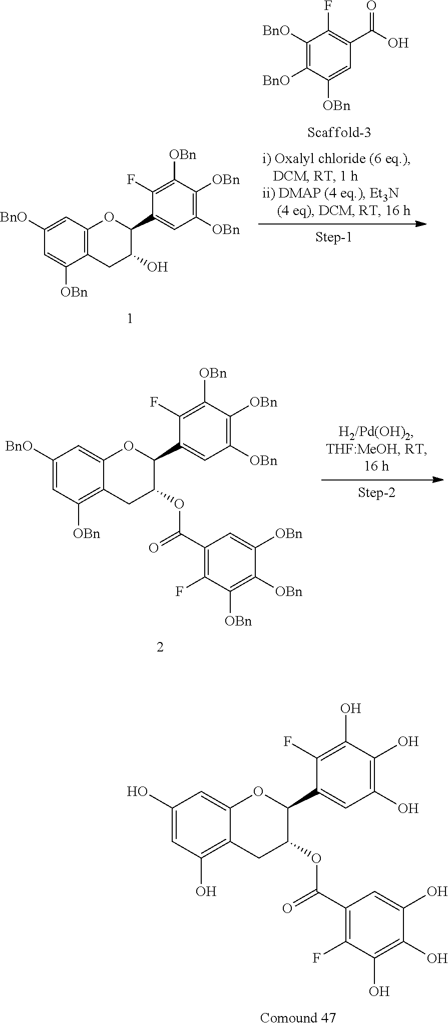 [SMILES: O=C(O)c1cc(OCc2ccccc2)c(OCc2ccccc2)c(OCc2ccccc2)c1F ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1F ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1F)c1cc(OCc2ccccc2)c(OCc2ccccc2)c(OCc2ccccc2)c1F ; O=C(O[C@@H]1Cc2c(O)cc(O)cc2O[C@H]1c1cc(O)c(O)c(O)c1F)c1cc(O)c(O)c(O)c1F]

Step 1: Synthesis of (2S,3R)-5,7-bis(benzyloxy)-2-(3,4,5-tris(benzyloxy)-2-fluorophenyl)chroman-3-yl 3,4,5-tris(benzyloxy)-2-fluorobenzoate (2). Under an N₂ atmosphere, to a stirred solution of 3,4,5-tris(benzyloxy)-2-fluorobenzoic acid (0.88 g, 1.937 mmol, 3 eq.) in DCM (5 mL) was added oxalyl chloride (2.5 mL, 3.874 mmol, 6 eq.) and two drop of DMF at 0° C. The reaction mixture was stirred at RT for 1 h. After this time, the reaction mixture was concentrated under reduced pressure to get acid chloride. Obtained acid chloride was added to a solution of (2S,3R)-5,7-bis(benzyloxy)-2-(3,4,5-tris(benzyloxy)-2-fluorophenyl)chroman-3-ol (0.5 g, 0.645 mmol, 1 eq.), DMAP (0.314 g, 2.580 mmol, 4 eq.) and Et₃N (0.36 mL, 2.580 mmol, 4 eq.) in CH₂Cl₂ (5 mL) at 0° C. Then the reaction mixture was stirred at RT 16 h. Finally, the reaction was quenched with saturated aqueous NaHCO₃ solution (5 mL). The organic layer was separated and the aqueous layer was extracted with CH₂Cl₂ (30 mL). Combined organic phase was dried over MgSO₄, filtered and concentrated under reduced pressure. Obtained crude compound was purified by flash column chromatography (EtOAc in hexane) to get (2S,3R)-5,7-bis(benzyloxy)-2-(3,4,5-tris(benzyloxy)-2-fluorophenyl)chroman-3-yl 3,4,5-tris(benzyloxy)-2-fluorobenzoate as a white solid (0.22 g, 28% yield). ¹H NMR (400 MHZ, DMSO-d₆) δ 7.55-7.16 (m, 40H), 7.06 (d, J=6.4 Hz, 1H), 6.45 (d, J=2 Hz, 1H), 6.26 (d, J=2 Hz, 1H), 5.48 (d, J=6 Hz, 1H), 5.37 (d, J=8.4 Hz, 1H), 5.16 (s, 2H), 5.10 (s, 4H), 4.98 (s, 6H), 4.94 (s, 2H), 2.49 (dd, J=1.6 Hz, 2H). ¹⁹F NMR (400 MHZ, DMSO-d₆) δ−123.50, −81.87. LCMS: (M+H⁺): m/Z: 1215.

Step 2: Synthesis of Compound 47. To a solution of (2S,3R)-5,7-bis(benzyloxy)-2-(3,4,5-tris(benzyloxy)-2-fluorophenyl)chroman-3-yl 3,4,5-tris(benzyloxy)-2-fluorobenzoate (0.210 g, 0.175 mmol, 1 eq.), in 12 mL of 1:1 THF:MeOH was added palladium hydroxide (20 wt. %, 0.21 g) at RT and stirred under a hydrogen atmosphere for 16 h. Then the mixture was passed through celite pad to remove the catalyst. The filtrate was concentrated under vacuum. Obtained crude compound was purified by Prep-HPLC to obtained (2S,3R)-2-(2-fluoro-3,4,5-trihydroxyphenyl)-5,7-dihydroxychroman-3-yl 2-fluoro-3,4,5-trihydroxybenzoate as an off-white color solid (0.010 g, 11% yield). ¹H NMR (400 MHZ, DMSO-d₆): 6.62 (d, J=6.8 Hz, 1H), 6.17 (d, J=6.4 Hz, 1H), 5.93 (d, J=2.0 Hz, 1H), 5.78 (d, J=2.4 Hz, 1H), 5.32 (q, J=6.0 Hz, 1H), 5.23 (d, J=6.4 Hz, 1H), 2.70 (dd, J=5.5 Hz, 1H), 2.61 (dd, J=6.4 Hz, 1H), ¹⁹F NMR (400 MHz, DMSO-d₆) δ−141.04, −149.92. LCMS: (M−H⁻): m/Z: 494.

### Compound 48

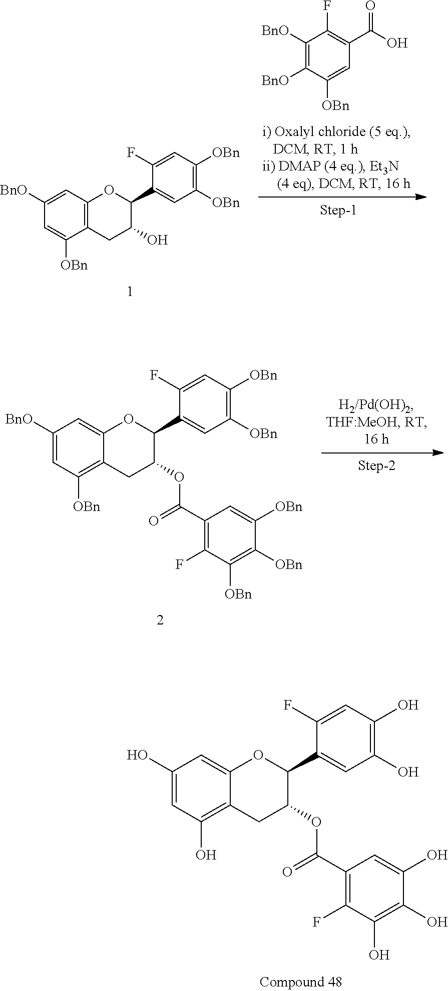 [SMILES: O=C(O)c1cc(OCc2ccccc2)c(OCc2ccccc2)c(OCc2ccccc2)c1F ; O[C@@H]1Cc2c(OCc3ccccc3)cc(OCc3ccccc3)cc2O[C@H]1c1cc(OCc2ccccc2)c(OCc2ccccc2)cc1F ; O=C(O[C@@H]1Cc2c(OCc3ccccc3)cc(OCc3ccccc3)cc2O[C@H]1c1cc(OCc2ccccc2)c(OCc2ccccc2)cc1F)c1cc(OCc2ccccc2)c(OCc2ccccc2)c(OCc2ccccc2)c1F ; O=C(O[C@@H]1Cc2c(O)cc(O)cc2O[C@H]1c1cc(O)c(O)cc1F)c1cc(O)c(O)c(O)c1F]

Step 1: Synthesis of (2S,3R)-5,7-bis(benzyloxy)-2-(4,5-bis(benzyloxy)-2-fluorophenyl)chroman-3-yl 3,4,5-tris(benzyloxy)-2-fluorobenzoate (2). Under an N₂ atmosphere, to a stirred solution of 3,4,5-tris(benzyloxy)-2-fluorobenzoic acid (1 g, 2.245 mmol, 3 eq.) in DCM (12 mL) was added oxalyl chloride (4.3 mL, 4.488 mmol, 5 eq.) and two drop of DMF at 0° C. The reaction mixture was stirred at RT for 1 h. The excess oxalyl chloride was removed by distillation and the residue was dried under in the presence of organ gas to give acid chloride. This solution was added dropwise to a solution of (2S,3R)-5,7-bis(benzyloxy)-2-(4,5-bis(benzyloxy)-2-fluorophenyl)chroman-3-ol (0.5 g, 0.748 mmol, 1 eq.), DMAP (0.36 g, 2.992 mmol, 4 eq.) and Et₃N (0.4 mL, 2.992 mmol, 4 eq.) in CH₂Cl₂ (15 mL) at 0° C. The mixture was stirred at RT 16 h, and then saturated NaHO₃ aqueous solution was added. The organic layer was separated, and the aqueous layer was extracted with CH₂Cl₂. The organic phases were combined, dried (MgSO₄) and evaporated. The crude compound was purified by flash column chromatography eluted with 20% EtOAc in hexane as an eluent affords to obtain (2S,3R)-5,7-bis(benzyloxy)-2-(4,5-bis(benzyloxy)-2-fluorophenyl)chroman-3-yl 3,4,5-tris(benzyloxy)-2-fluorobenzoate as a white solid (0.22 g, 24% yield). ¹H NMR (400 MHZ, DMSO-de): δ 7.47-7.15 (m, 36H), 7.01 (s, 2H), 6.61 (d, J=2.4 Hz, 1H), 6.22 (d, J=1.6 Hz, 1H), 5.32 (d, J=8 Hz, 1H), 5.19 (d, J=4.8 Hz, 1H), 5.12 (s, 2H), 5.08 (s, 4H), 4.98 (s, 6H), 4.94 (s, 2H), 2.49 (dd, J=1.6 Hz, 2H). ¹⁹F NMR (400 MHZ, DMSO-d₆) δ−123.50, −81.87. LCMS: (M+H⁺): m/Z: 1197.46.

Step 2: Synthesis of Compound 48. To a solution of (2S,3R)-5,7-bis(benzyloxy)-2-(4,5-bis(benzyloxy)-2-fluorophenyl)chroman-3-yl 3,4,5-tris(benzyloxy)-2-fluorobenzoate (0.22 g, 0.183 mmol, 1 eq.), in 8 mL of (1:1; THF:MeOH, was added palladium hydroxide (20 wt. %, 0.22 g) at RT and stirred under a hydrogen atmosphere for 16 h. Then the mixture was passed through a pad of celite to remove the catalyst. The filtrate was concentrated under vacuum. The crude compound was purified by Prep-HPLC to obtain (2S,3R)-2-(2-fluoro-4,5-dihydroxyphenyl)-5,7-dihydroxychroman-3-yl 2-fluoro-3,4,5-trihydroxybenzoate as an off-white color solid (0.05 g, 5% yield). LCMS: (M−H⁺): m/Z: 479.0.

### Compound 49

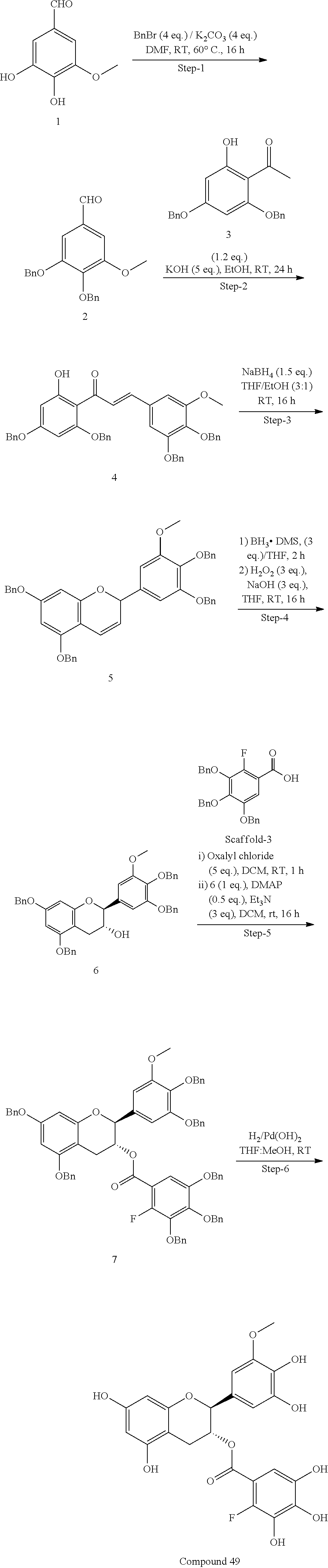

Step 1: Synthesis of 3,4-bis(benzyloxy)-5-methoxybenzaldehyde (2). To a suspension of 3,4-dihydroxy-5-methoxybenzaldehyde (1 g, 5.952 mmol, 1 eq.) in DMF (10 mL) was added K₂CO₃ (3.3 g, 23.808 mmol, 4 eq.) followed by benzyl bromide (2.6 mL, 23.808 mmol, 4 eq.) at 0° C. The reaction mixture was stirred at 60° C. for 16 h until TLC showed the reaction was completed. Reaction mass was diluted with water (15 mL) and EtOAc (45 mL). Organic layer was separated, washed with brine solution (10 mL), dried over Na₂SO₄, filtered and concentrated under vacuum. Obtained residue was purified by flash chromatography eluted with 15% EtOAc in hexane as an eluent affords to obtain desired compound 3,4-bis(benzyloxy)-5-methoxybenzaldehyde as a white solid (1.2 g, 57% yield). ¹H NMR (400 MHZ, DMSO-d₆) δ 9.86 (s, 1H), 7.46-7.27 (m, 12H), 5.19 (s, 2H), 5.04 (s, 2H), 3.86 (s, 3H).

Step 2: Synthesis of (E)-3-(3,4-bis(benzyloxy)-5-methoxyphenyl)-1-(2,4-bis(benzyloxy)-6-hydroxyphenyl)prop-2-en-1-one (3). To a solution of 1-(2,4-bis(benzyloxy)-6-hydroxyphenyl)ethan-1-one (1.2 g, 7.183 mmol, 1 eq.) in MeOH (20 mL) was added KOH (1.2 g, 35.915 mmol, 5 eq.). The mixture was stirred at room temperature for 30 min. Then added compound 3,4-bis(benzyloxy)-5-methoxybenzaldehyde (2.5 g, 7.183 mmol, 1 eq.). The reaction mixture was stirred at 60° C. for 16 h. After this time, the reaction mixture was concentrated. Obtained residue was diluted with H₂O (50 mL) and extracted with EtOAcA (50 mL×3). The combined organic layers was washed with brine (30 mL×2), dried over Na₂SO₄, filtered and concentrated. The solid was triturated with EtOH (20 mL), filtered, washed with EtOH (10 mL), then dried obtained (E)-3-(3,4-bis(benzyloxy)-5-methoxyphenyl)-1-(2,4-bis(benzyloxy)-6-hydroxyphenyl)prop-2-en-1-one as a yellow solid (2.1 g, 38% yield). ¹H NMR (400 MHZ, DMSO-d₆): δ 7.65 (d, J=15.6 Hz, 1H), 7.53 (d, J=13.6 Hz, 1H), 7.46-7.28 (m, 14H), 7.22-7.16 (m, 4H), 7.02 (d, J=4.0 Hz, 3H), 6.92 (d, J=2.0 Hz, 1H), 6.39 (d, J=2.0 Hz, 1H), 6.24 (d, J=2.0 Hz, 1H), 5.20 (s, 2H), 5.16 (s, 2H), 4.98 (s, 2H), 4.96 (s, 2H), 3.69 (s, 3H).

Step 3: Synthesis of 5,7-bis(benzyloxy)-2-(3,4-bis(benzyloxy)-5-methoxyphenyl)-2H-chromene (4). To a solution of (E)-3-(3,4-bis(benzyloxy)-5-methoxyphenyl)-1-(2,4-bis(benzyloxy)-6-hydroxyphenyl)prop-2-en-1-one (0.25 g, 0.368 mmol, 1 eq.) in THF/EtOH (15 mL/5 mL) was added CeCl₃ (0.27 g, 1.106 mmol, 3 eq.) and NaBH₄ (0.43 g, 1.106 mmol, 3 eq.) at 0° C. The reaction mixture was stirred at RT for 16 h. After this time, the reaction mixture was diluted with water (30 mL) and extracted with DCM (30 mL×2). The combined organic layers was dried over Na₂SO₄, filtered and concentrated to give the crude product. Obtained crude product was purified by flash chromatography; eluted with 30% EtOAc in hexane, as an eluent affords to obtain 5,7-bis(benzyloxy)-2-(3,4-bis(benzyloxy)-5-methoxyphenyl)-2H-chromene as a yellow solid (0.18 g, 74% yield). ¹H NMR (400 MHZ, DMSO-d₆): δ 7.45-7.28 (m, 17H), 7.72 (d, J=14.0 Hz, 1H), 6.83 (d, J=2.0 Hz, 1H), 6.73 (dd, J=2.0 Hz, 2H), 6.35 (d, J=2.0 Hz, 1H), 6.82 (d, J=2.0 Hz, 1H), 5.79 (d, J=1.6 Hz, 1H), 5.72 (dd, J=3.6 Hz, 1H), 5.13 (d, J=13.6 Hz, 1H), 5.09 (s, 2H) 5.05 (s, 4H), 4.88 (s, 2H), 4.47 (d, J=5.6 Hz, 1H), 3.74 (s, 3H).

Step 4: Synthesis of (2S,3R)-5,7-bis(benzyloxy)-2-(3,4-bis(benzyloxy)-5-methoxyphenyl)chroman-3-ol (5). To a solution of 5,7-bis(benzyloxy)-2-(3,4-bis(benzyloxy)-5-methoxyphenyl)-2H-chromene (1 g, 1.515 mmol, 1 eq.) in 10 mL of THF was added BH3.DMS (2M, 2.2 mL, 4.540 mmol, 3 eq.) at 0° C. and stirred for 2 hours. To the above reaction mixture was added 3 N of NaOH solution (0.18 g, 4.545 mmol, 3 eq.) and 30% aqueous solution of H₂O₂ (0.49 mL, 4.545 mmol, 3 eq.) at 0° C. The reaction was stirred at RT for 16 h. After this time, the reaction mixture was diluted with H₂O (20 mL) and extracted with EA (30 mL×2). The organic phase was concentrated and crude product was purified by flash chromatography eluted with 20% EtOAc in hexane as an eluent afford (2S,3R)-5,7-bis(benzyloxy)-2-(3,4-bis(benzyloxy)-5-methoxyphenyl)chroman-3-ol as a light yellow solid (0.36 g, 36% yield). ¹H NMR (400 MHZ, DMSO-d₆): δ 7.45-7.28 (m, 21H), 6.81 (s, 1H), 6.71 (s, 1H), 6.34 (d, J=2.0 Hz, 1H), 6.14 (d, J=2 Hz, 1H), 5.06 (d, J=3.6 Hz, 1H), 5.05 (s, 2H) 5.03 (s, 4H), 4.89 (s, 2H), 4.02 (t, J=6.8 Hz, 1H), 3.75 (s, 3H), 2.78 (dd, J=5.6 Hz, 2H).

Step 5: Synthesis of (2S,3R)-5,7-bis(benzyloxy)-2-(3,4-bis(benzyloxy)-5-methoxyphenyl)chroman-3-yl 3,4,5-tris(benzyloxy)-2-fluorobenzoate (5). Under an N₂ atmosphere, to a stirred solution of 3,4,5-tris(benzyloxy)-2-fluorobenzoic acid (0.29 g, 0.635 mmol, 1.2 eq.) in DCM (8 mL) was added oxalyl chloride (0.23 mL, 2.640 mmol, 5 eq.) and two drops of DMF at 0° C. The reaction mixture was stirred at RT for 1 h. The excess oxalyl chloride were removed by distillation and the residue was dried to give acid chloride. This solution was added dropwise to a solution of (2S,3R)-5,7-bis(benzyloxy)-2-(3,4-bis(benzyloxy)-5-methoxyphenyl)chroman-3-ol (0.36 g, 0.529 mmol, 1 eq.), DMAP (0.26 g, 2.116 mmol, 4 eq.) and Et₃N (0.3 mL, 2.116 mmol, 4 eq.) in CH₂Cl₂ (10 mL) at 0° C. The mixture was stirred at RT overnight, then saturated NaHO₃ aqueous solution was added. The organic layer was separated, and the aqueous layer was extracted with CH₂Cl₂. Combined organic phase was dried over MgSO₄ and concentrated under vacuum. The crude compound was purified by flash column chromatography, desired product was eluted with 12% EtOAc in hexane as an eluent to obtain (2S,3R)-5,7-bis(benzyloxy)-2-(3,4-bis(benzyloxy)-5-methoxyphenyl)chroman-3-yl 3,4,5-tris(benzyloxy)-2-fluorobenzoate as a white solid (0.38 g, 53% yield). ¹H NMR (400 MHZ, DMSO-d₆): δ 7.43-7.23 (m, 37H), 7.04 (d, J=6.4 Hz, 1H), 6.87 (s, 1H), 6.7 (s, 1H), 6.42 (d, J=2.0 Hz, 1H), 6.28 (d, J=2.0 Hz, 1H), 5.48 (q, J=6.0 Hz, 1H), 5.17 (d, J=7.2 Hz, 1H), 5.03 (s, 4H) 4.98 (s, 4H), 4.92 (s, 2H), 4.85 (s, 2H), 3.67 (s, 3H), 2.99 (dd, J=7.6 Hz, 1H), 2.28 (dd, J=7.6 Hz, 1H).

Step 6: Synthesis of Compound 49. To a solution of (2S,3R)-5,7-bis(benzyloxy)-2-(3,4-bis(benzyloxy)-5-methoxyphenyl)chroman-3-yl 3,4,5-tris(benzyloxy)-2-fluorobenzoate (1 g, 0.836 mmol, 1 eq.), in 10 mL of 1:1 THF:MeOH was added palladium hydroxide (20 wt. %, 1.0 g) at RT. The reaction mixture was stirred under hydrogen atmosphere for 16 h. After this time reaction mixture was passed through a pad of celite to remove the catalyst. The filtrate was concentrated under vacuum. The crude compound was purified by Prep-HPLC to obtain (2S,3R)-2-(3,4-dihydroxy-5-methoxyphenyl)-5,7-dihydroxychroman-3-yl 2-fluoro-3,4,5-trihydroxybenzoate as an off-white color solid (0.48 g, 16% yield). ¹H NMR (400 MHZ, DMSO-d₆): δ 9.17 (bs, 7H) 6.68 (d, J=6.4 Hz, 1H), 6.44 (d, J=1.6 Hz, 1H), 6.41 (s, 1H), 5.92 (d, J=2.0 Hz, 1H), 5.80 (d, J=2.0 Hz, 1H), 5.30 (q, J=6.0 Hz, 1H), 5.02 (d, J=6.0 Hz, 1H), 3.66 (s, 3H), 2.67 (dd, J=6.0 Hz, 1H), 2.58 (dd, J=6.0 Hz, 1H). LCMS: (M+H⁺): m/Z: 491.0.

### Compound 50

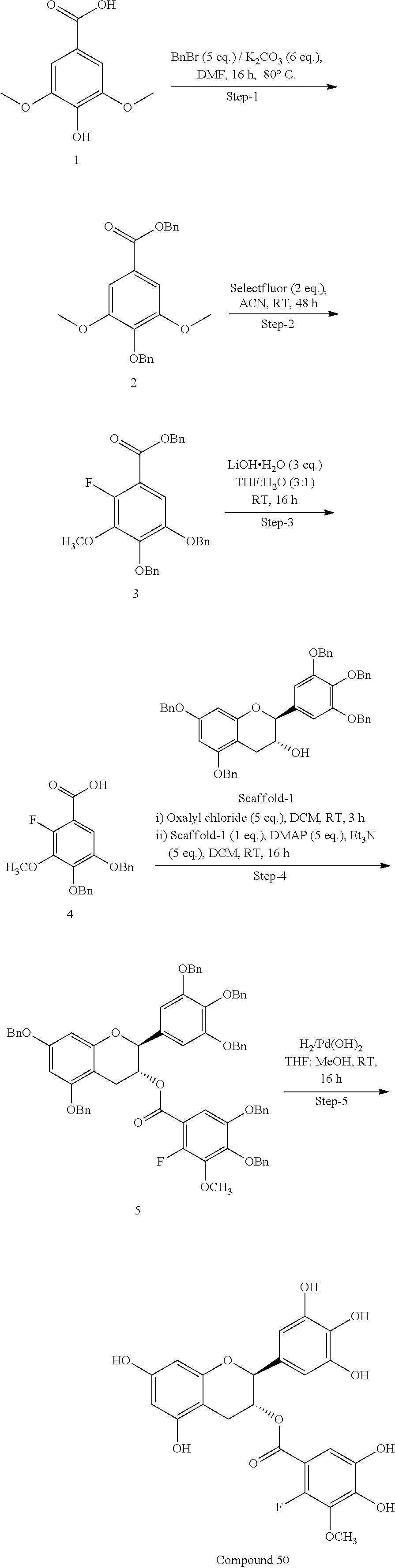

Step 1: Synthesis of benzyl 3,4-bis(benzyloxy)-5-methoxybenzoate (2). To a solution of 3,4-dihydroxy-5-methoxybenzoic acid (5 g, 27.159 mmol) in DMF (50 mL) was added K₂CO₃ (22.4 g, 162.950 mmol, 6 eq.) followed by benzyl bromide (16 mL, 1135.790 mmol, 5 eq.) at 0° C. The mixture was heated at 80° C. for 16 h until TLC showed the reaction was completed. Reaction mixture diluted with water and extracted with EtOAc. Organic layer was concentrated under vacuum to get crude product, purified by flash chromatography, desired product was eluted at 15% EtOAc in hexane and obtained benzyl 3,4-bis(benzyloxy)-5-methoxybenzoate as a yellow liquid (10.1 g, 82% yield). ¹H NMR (400 MHZ, DMSO-d₆) δ 7.43-7.26 (m, 17H), 5.33 (s, 2H), 5.14 (s, 2H), 5.01 (s, 2H), 3.83 (s, 3H).

Step 2: Synthesis of benzyl 4,5-bis(benzyloxy)-2-fluoro-3-methoxybenzoate (3). To a solution of benzyl 3,4-bis(benzyloxy)-5-methoxybenzoate (11 g, 24.240 mmol, 1 eq.) in ACN (100 mL) was added selectfluor (17.1 g, 4.400 mmol, 2 eq.) at 0° C. and the reaction mixture stirred at RT for 48 h. Reaction progress was monitor by TLC. After this time, reaction mixture was quenched with cold water, extracted with EtOAc (3×100 mL). Organic layer was washed with brine solution and dried over anhydrous Na₂SO₄, filtered and concentrated under reduced pressure to get crude compound. The crude compound was purified by flash column chromatography. Desired product was eluted with 10% EtOAc in hexane. Fractions were concentrated to get benzyl 4,5-bis(benzyloxy)-2-fluoro-3-methoxybenzoate as a yellow solid (1.1 g, 9% yield). ¹HNMR (400 MHZ, DMSO-d₆): δ 7.42-7.28 (m, 16H), 5.33 (s, 2H), 5.16 (s, 2H), 5.14 (s, 2H), 3.81 (s, 3H), ¹⁹F NMR (400 MHZ, DMSO-d₆) δ−134.52.

Step 3: Synthesis of 4,5-bis(benzyloxy)-2-fluoro-3-methoxybenzoic acid (4). To a solution of benzyl 4,5-bis(benzyloxy)-2-fluoro-3-methoxybenzoate (1 g, 2.118 mmol, 1.0 eq.) in THF/H₂O (3:1) (20 mL) was added LiOH H₂O (0.88 g, 21.186 mmol, 10.0 eq.). The solution was stirred at RT for 16 h. The reaction mixture was concentrated and the obtained crude was diluted with H₂O (30 mL) and extracted with EtOAc (80 mL×2). The aqueous phase pH was adjusted to <3 with 1N HCl. Obtained solid was filtered and the cake was dried. The crude compound was purified by flash column chromatography, eluted with 10% EtOAc in hexane as an eluent affords to obtain 4,5-bis(benzyloxy)-2-fluoro-3-methoxybenzoic acid as a white solid (0.502 g, 62% yield). ¹H NMR (400 MHZ, DMSO-d₆): δ 13.22 (s, 1H), 7.42 (d, J=1.2 Hz, 2H), 7.44-7.26 (m, 10H), 5.14 (s, 2H), 5.12 (s, 2H), 3.81 (s, 3H).

Step 4: Synthesis of (2S,3R)-5,7-bis(benzyloxy)-2-(3,4,5-tris(benzyloxy)phenyl)chroman-3-yl 4,5-bis(benzyloxy)-2-fluoro-3-methoxybenzoate (5). Under an N₂ atmosphere, to a stirred solution of 4,5-bis(benzyloxy)-2-fluoro-3-methoxybenzoic acid (0.265 g, 0.693 mmol, 1 eq.) in DCM (5 mL) was added oxalyl chloride (0.22 mL, 2.665 mmol, 5 eq.) and two drops of DMF at 0° C. The reaction mixture was stirred at RT for 3 h. The excess oxalyl chloride were removed by distillation and the residue was dried to give acid chloride. Generated acid chloride was added dropwise to a solution of (2S,3R)-5,7-bis(benzyloxy)-2-(3,4,5-tris(benzyloxy)phenyl)chroman-3-ol (0.203 g, 0.533 mmol, 1 eq.), DMAP (0.325 g, 2.665 mmol, 5 eq.) and Et₃N (0.36 mL, 2.665 mmol, 5 eq.) in CH₂Cl₂ (6 mL) at 0° C. The mixture was stirred at RT for overnight. After this time, saturated NaHO₃ aqueous solution was added to the reaction mass. Then organic layer was separated, and the aqueous layer was extracted with CH₂Cl₂. Combined organic phases was dried over MgSO₄, filtered and concentrated under radiused pressure. Obtained crude compound was purified by flash column chromatography, eluted with 15% EtOAc in hexane, as an eluent afford desired compound (2S,3R)-5,7-bis(benzyloxy)-2-(3,4,5-tris(benzyloxy)phenyl)chroman-3-yl4,5-bis(benzyloxy)-2-fluoro-3-methoxybenzoate as a light yellow solid (0.506 g, 64% yield). ¹H NMR (400 MHZ, DMSO-d₆) δ 7.43-7.21 (m, 35H), 7.01 (d, J=9.6 Hz, 1H), 6.89 (s, 2H), 6.42 (s, 1H), 6.27 (s, 1H), 5.51 (d, J=5.2 Hz, 1H), 5.18 (d, J=7.2 Hz, 1H), 5.11 (s, 2H), 5.06 (s, 2H), 5.04 (s, 4H), 4.97 (s, 4H), 4.87 (s, 2H), 3.75 (s, 3H), 2.62 (dd, J=7.6 Hz, 2H). LCMS: (M+H⁺): m/Z: 1121.1.

Step 5: Synthesis of Compound 50. To a solution of (2S,3R)-5,7-bis(benzyloxy)-2-(3,4,5-tris(benzyloxy)phenyl)chroman-3-yl 4,5-bis(benzyloxy)-2-fluoro-3-methoxybenzoate (0.5 g, 0.351 mmol, 1 eq.) in 8 mL of 1:1 THF:MeOH was added palladium hydroxide (20 wt. %, 0.5 g) at RT and was stirred under hydrogen atmosphere for 16 h. Then the mixture was passed through a pad of celite to remove the catalyst. The filtrate was concentrated under vacuum. The crude compound was purified by Prep-HPLC to obtain desired compound (2S,3R)-5,7-dihydroxy-2-(3,4,5-trihydroxyphenyl)chroman-3-yl 2-fluoro-4,5-dihydroxy-3-methoxybenzoate as an off-white color solid (0.058 g, 27% yield). ¹H NMR (400 MHZ, DMSO-d₆): 9.02 (s, 5H), 5.88 (d, J=6.8 Hz, 1H), 6.25 (s, 2H), 5.91 (d, J=2.0 Hz, 1H), 5.79 (d, J=2.4 Hz, 1H), 5.28 (q, J=5.2 Hz, 1H), 5.01 (d, J=5.2 Hz, 1H), 3.73 (s, 3H), 2.58 (dd, J=4 Hz, 2H). LCMS: (M−H+): m/Z: 490.1.

### Compound 51

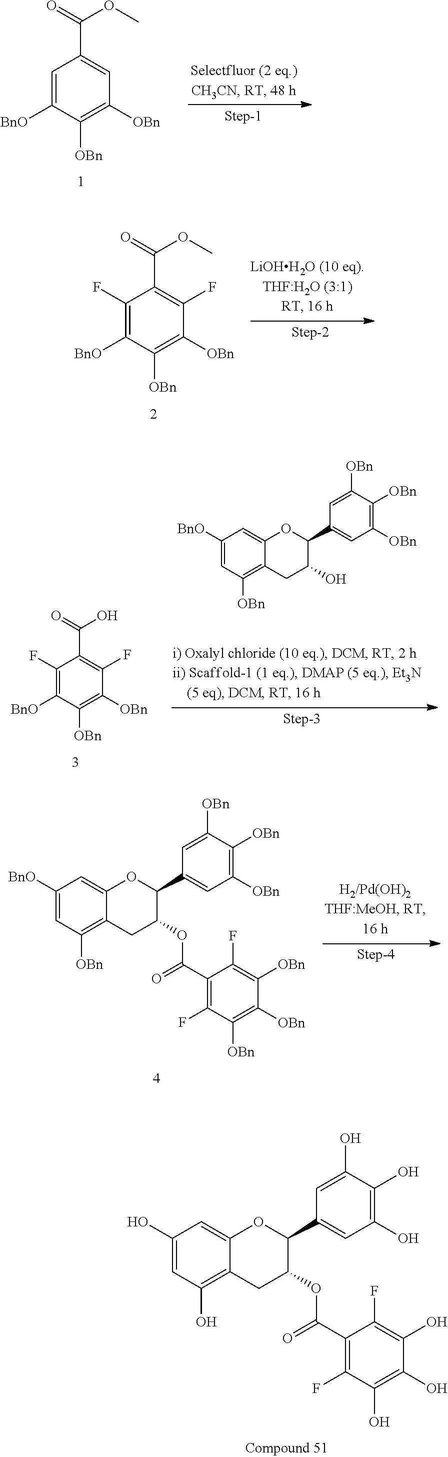 [SMILES: COC(=O)c1cc(OCc2ccccc2)c(OCc2ccccc2)c(OCc2ccccc2)c1 ; COC(=O)c1c(F)c(OCc2ccccc2)c(OCc2ccccc2)c(OCc2ccccc2)c1F ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1c(F)c(OCc2ccccc2)c(OCc2ccccc2)c(OCc2ccccc2)c1F ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1c(F)c(OCc2ccccc2)c(OCc2ccccc2)c(OCc2ccccc2)c1F ; O=C(O[C@@H]1Cc2c(O)cc(O)cc2O[C@H]1c1cc(O)c(O)c(O)c1)c1c(F)c(O)c(O)c(O)c1F]

Step 1: Synthesis of methyl 3,4,5-tris(benzyloxy)-2,6-difluorobenzoate (2). To a solution of methyl 3,4,5-tris(benzyloxy)benzoate (50 g, 110.132 mmol, 1 eq.) in ACN (60 mL) was added selectfluor (77 g, 220.264 mmol, 2 eq.), at 0° C. and the reaction mixture was stirred at RT for 48 h. Reaction progress was monitor by TLC. After this time, reaction mixture was quenched with cold water, extracted with EtOAc (3×100 mL), washed with brine and dried over anhydrous Na₂SO₄, filtered and concentrated under reduced pressure to get crude compound. Obtained crude compound was purified by flash column chromatography, eluted with 5% EtOAc in hexane as an eluent affords to obtain methyl 3,4,5-tris(benzyloxy)-2,6-difluorobenzoate as a yellow solid (0.6 g, 1% yield). ¹H NMR (400 MHZ, DMSO-d₆) δ 7.38-7.34 (m, 15H), 5.27 (s, 2H), 5.02 (s, 4H), 3.85 (s, 3H), ¹⁹F NMR (400 MHZ, DMSO-d₆) δ−133.38.

Step 2: Synthesis of 3,4,5-tris(benzyloxy)-2,6-difluorobenzoic acid (3). To a solution of methyl 3,4,5-tris(benzyloxy)-2,6-difluorobenzoate (0.6 g, 1.224 mmol, 1 eq.) in THF/H₂O (3:1) (12 mL) was added LiOH H₂O (0.513 g, 12.240 mmol, 10 eq.) and stirred at RT for 16 h. The reaction mixture was concentrated, obtained crude was diluted with H₂O (30 mL) and extracted with EA (10 mL×1). The aqueous phase pH was adjusted to <3 with 1N HCl. Obtained solid was filtered and dried to obtain 3,4,5-tris(benzyloxy)-2,6-difluorobenzoic acid as a yellow solid (0.352 g, 60% yield). ¹H NMR (400 MHZ, DMSO-d₆) δ 13.85 (s, 1H), 7.35-7.33 (m, 15H), 5.15 (s, 2H), 5.02 (s, 4H), ¹⁹F NMR (400 MHZ, DMSO-d₆) δ−134.14.

Step 3: Synthesis of (2S,3R)-5,7-bis(benzyloxy)-2-(3,4,5-tris(benzyloxy)phenyl)chroman-3-yl 3,4,5-tris(benzyloxy)-2,6-difluorobenzoate (4). Under an N₂ atmosphere, to a stirred solution of 3,4,5-tris(benzyloxy)-2,6-difluorobenzoic acid (0.35 g, 0.735 mmol, 1 eq.) in DCM (8 mL) was added oxalyl chloride (0.31 mL, 7.350 mmol, 10 eq.) in dry CH₂Cl₂ (8 mL) and two drops of DMF at 0° C. The reaction mixture was stirred at RT for 2 h. Then excess oxalyl chloride was concentrated and the residue was dried to get acid chloride. This solution was added dropwise to a solution of (2S,3R)-5,7-bis(benzyloxy)-2-(3,4,5-tris(benzyloxy)phenyl)chroman-3-ol (0.44 g, 0.588 mmol, 0.8 eq.), DMAP (0.448 g, 3.675 mmol, 5 eq.) and Et₃N (0.53 mL, 3.675 mmol, 5 eq.) in CH₂Cl₂ (12 mL) at 0° C. The mixture was stirred at RT for overnight. After this time, saturated NaHO₃ aqueous solution was added. The organic layer was separated and the aqueous layer was extracted with CH₂Cl₂. Combined organic phase was dried over MgSO₄ filtered and evaporated. Obtained crude compound was purified by flash column chromatography, eluted with 12% EtOAc in hexane as an eluent affords to obtain (2S,3R)-5,7-bis(benzyloxy)-2-(3,4,5-tris(benzyloxy)phenyl)chroman-3-yl3,4,5-tris(benzyloxy)-2,6 difluorobenzoate as a red solid (0.109 g, 12% yield). ¹H NMR (400 MHZ, DMSO-d₆) δ 7.41-7.21 (m, 40H), 6.86 (s, 2H), 6.40 (d, J=2 Hz, 1H), 6.26 (d, J=1.6 Hz, 1H), 5.63 (d, J=5.2 Hz, 1H), 5.17 (s, 4H), 5.11 (s, 2H), 5.06 (s, 4H), 5.02 (d, J=5.2 Hz, 1H), 4.95 (s, 4H), 4.98 (s, 2H), 2.80 (dd, J=4.0 Hz, 2H), ¹⁹F NMR (400 MHZ, DMSO-de) δ−133.17. LCMS: (M+H⁺): m/Z: 1215.47.

Step 4: Synthesis of Compound 51. To a solution of (2S,3R)-5,7-bis(benzyloxy)-2-(3,4,5-tris(benzyloxy)phenyl)chroman-3-yl 3,4,5-tris(benzyloxy)-2,6-difluorobenzoate (0.1 g, 0.082 mmol, 1 eq.) in 10 mL of 1:1 THF:MeOH was added palladium hydroxide (20 wt. %, 0.1 g) at RT and the reaction mixture was stirred under hydrogen atmosphere for 16 h. Then the mixture was passed through a pad of celite to remove the catalyst. The filtrate was concentrated under vacuum. Obtained crude compound was purified by Prep-HPLC to obtain (2S,3R)-5,7-dihydroxy-2-(3,4,5-trihydroxyphenyl)chroman-3-yl 2,6-difluoro-3,4,5-trihydroxybenzoate as an off-white color solid (0.026 g, 65% yield). ¹H NMR (400 MHZ, DMSO-d₆): δ 6.23 (s, 2H), 5.89 (d, J=2.0 Hz, 1H), 5.77 (d, J=2.0 Hz, 1H), 5.33 (q, J=4.8 Hz, 1H), 5.01 (d, J=4.8 Hz, 1H), 2.58 (dd, J=4.0 Hz, 2H), ¹⁹F NMR (400 MHZ, DMSO-d₆) δ−145.35. LCMS: (M−H+): m/Z: 494.95.

### Compound 52

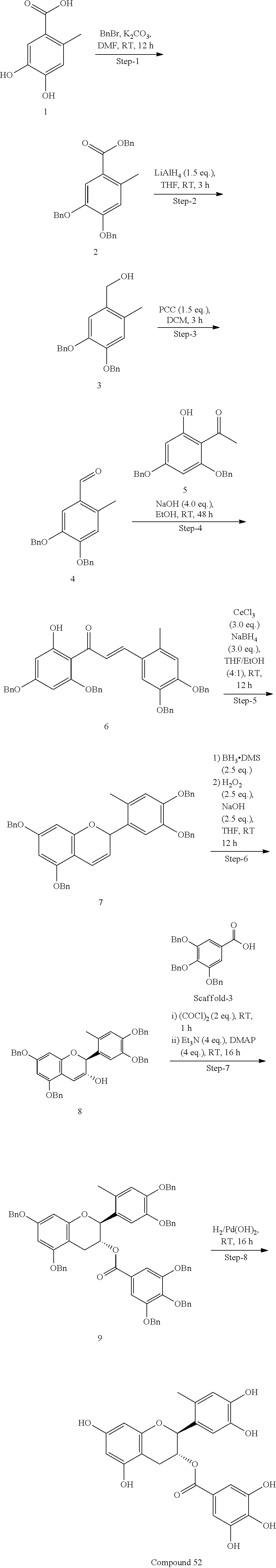

Step 1: Synthesis of benzyl 4,5-bis(benzyloxy)-2-methylbenzoate (2). To a solution of 4,5-dihydroxy-2-methylbenzoic acid (0.2 g, 1.17 mmol, 1, 0 eq.) in dry DMF (5 mL) was added K₂CO₃ (0.81 g, 5.88 mmol, 5.0 eq.) and BnBr (0.7 ml, 5.88 mmol, 5.0 eq.) dropwise at 0° C. The mixture was stirred at RT for 12 h. After completion of the reaction, reaction mixture was diluted with H₂O (20 mL) and extracted with EtOAc (2×50 mL). The combined organic phase was washed with brine (30 mL), dried over Na₂SO₄, filtered and concentrated. The residue was purified by flash column chromatography on silica gel (PE/EA=9/1) to give benzyl 4,5-bis(benzyloxy)-2-methylbenzoate (0.47 g, 90% yield) as a white solid. ¹H NMR (400 MHZ, CDCl₃): δ 7.61 (s, 1H), 7.46-7.25 (m, 15H), 6.76 (s, 1H), 5.28 (s, 2H), 5.19 (s, 2H), 5.14 (s, 2H), 2.52 (s, 3H).

Step 2: Synthesis of (4,5-bis(benzyloxy)-2-methylphenyl)methanol (3). To a solution of benzyl 4,5-bis(benzyloxy)-2-methylbenzoate (6.0 g, 13.69 mmol, 1.0 eq.) in THF (60 mL) was added LiAlH₄ (0.78 g, 20.53 mmol, 1.5 eq.) at 0° C. The mixture was stirred at room temperature for 3 h. The reaction mixture was quenched with sat. aq. Na₂SO₄ (5 mL) and filtered through pad of celite. The filtrate was concentrated under reduced pressure. The obtained residue was purified by flash column chromatography on silica gel (PE/EA=5/1) to give (4,5-bis(benzyloxy)-2-methylphenyl)methanol (3.3 g, 72% yield) as a white solid. ¹H NMR (400 MHZ, CDCl₃): δ 7.46-7.40 (m, 4H), 7.39-7.27 (m, 6H), 6.98 (s, 1H), 6.78 (s, 1H), 5.13 (s, 4H), 4.57 (d, J=5.6 Hz, 2H), 2.25 (s, 3H), 1.43 (t, J=5.6 Hz, 1H).

Step 3: Synthesis of 4,5-bis(benzyloxy)-2-methylbenzaldehyde (4). A mixture of (4,5-bis(benzyloxy)-2-methylphenyl)methanol (0.2 g, 0.59 mmol, 1.0 eq.) in CH₂Cl₂ (4 mL) was added PCC (0.19 g, 0.89 mmol, 1.5 eq.). The solution was stirred at room temperature for 3 h. The reaction mixture was concentrated and the residue was purified by flash column chromatography give the 4,5-bis(benzyloxy)-2-methylbenzaldehyde (0.165 g, 83% yield) as a white solid. ¹H NMR (400 MHZ, CDCl₃): δ 10.14 (s, 1H), 7.48-7.28 (m, 11H), 6.75 (s, 1H), 5.23 (s, 2H), 5.17 (s, 2H), 2.57 (s, 3H).

Step 4: Synthesis of (E)-3-(4,5-bis(benzyloxy)-2-methylphenyl)-1-(2,4-bis(benzyloxy)-6-hydroxyphenyl)prop-2-en-1-one (6). A mixture of compound 4,5-bis(benzyloxy)-2-methylbenzaldehyde (0.05 g, 0.15 mmol, 1.0 eq.) and 1-(2,4-bis(benzyloxy)-6-hydroxyphenyl)ethan-1-one (0.05 g, 0.15 mmol, 1.0 eq.) in EtOH (2 mL) was added 50% aq. NaOH (0.5 mL, 0.63 mmol, 4.0 eq.). The mixture was stirred at 50° C. for 5 h then at RT for 48 h. The reaction mixture was diluted with H₂O (20 mL) and acidified with 1N HCl (5 mL). The formed precipitate was filtered. The yellow precipitate was dissolved in EtOAc (30 mL), washed with H₂O (10 mL) and brine (10 mL), dried over Na₂SO₄, filtered and concentrated. The obtained solid was purified by column chromatography to give (E)-3-(4,5-bis(benzyloxy)-2-methylphenyl)-1-(2,4-bis(benzyloxy)-6-hydroxyphenyl)prop-2-en-1-one (0.086 g, 80% yield) as a yellow solid. ¹H NMR (400 MHZ, CDCl₃): 14.28 (s, 1H), 8.01 (d, J=15.2 Hz, 1H), 7.69 (d, J=15.2 Hz, 1H), 7.48-7.24 (m, 17H), 7.23-7.16 (m, 3H), 6.91 (s, 1H), 6.75 (s, 1H), 6.22 (d, J=2.4 Hz, 1H), 6.15 (d, J=2.4 Hz, 1H), 5.16 (s, 2H), 5.10 (s, 2H), 5.08 (s, 2H), 4.67 (s, 2H), 2.36 (s, 3H).

Step 5: Synthesis of 5,7-bis(benzyloxy)-2-(4,5-bis(benzyloxy)-2-methylphenyl)-2H-chromene (7). A solution of (E)-3-(4,5-bis(benzyloxy)-2-methylphenyl)-1-(2,4-bis(benzyloxy)-6-hydroxyphenyl)prop-2-en-1-one (0.2 g, 0.30 mmol, 1.0 eq.) in THF:EtOH (4:1) (5 mL) was sequentially added anhydrous CeCl₃ (0.22 g, 0.90 mmol, 3.0 eq.) and NaBH₄ (0.035 g, 0.90 mmol, 3.0 eq.) at 0° C. The mixture was stirred at RT for overnight. The reaction mixture was diluted with H₂O (30 mL) and extracted with EtOAc (50 mL×2). The combined organic phase was washed with brine (30 mL), dried over Na₂SO₄, filtered and concentrated. The residue was purified by flash column chromatography (PE/EA=10/1) to give 5,7-bis(benzyloxy)-2-(4,5-bis(benzyloxy)-2-methylphenyl)-2H-chromene (0.11 g, 56% yield) as a white solid. ¹H NMR (400 MHZ, CDCl₃): 7.48-7.14 (m, 19H), 7.12 (s, 1H), 6.87 (dd, J=10.0, 2.0 Hz, 1H), 6.78 (s, 1H), 6.19 (d, J=2.4 Hz, 1H), 6.10 (d, J=2.5 Hz, 1H), 5.95 (t, J=2.4 Hz, 1H), 5.46 (dd, J=10.0, 3.2 Hz, 1H), 5.13 (s, 2H), 5.07 (q, J=12.8 Hz, 2H), 5.04 (s, 2H), 4.97 (s, 2H), 2.33 (s, 3H).

Step 6: Synthesis of (2S,3R)-5,7-bis(benzyloxy)-2-(4,5-bis(benzyloxy)-2-methylphenyl)chroman-3-ol (8). To a solution of 5,7-bis(benzyloxy)-2-(4,5-bis(benzyloxy)-2-methylphenyl)-2H-chromene (1.0 g, 1.54 mmol, 1.0 eq.) in dry THF (10 mL) was added BH3: DMS (2 mL, 3.86 mmol, 2.5 eq.) at 0° C. for 10 min. The mixture was stirred at room temperature until the starting material was disappeared. Then 3N aq. NaOH (1.3 mL, 3.86 mmol, 2.5 eq.) and 30% aq. H₂O₂ (0.4 mL, 3.86 mmol, 2.5 eq.) was added drop wise at 0° C. The mixture was stirred at RT for 12 h. The reaction mixture was diluted with H₂O (50 mL) and extracted with EtOAc (50 mL×3). The combined organic layers were washed with brine (50 mL), dried over Na₂SO₄, filtered and concentrated. The residue was purified by flash column chromatography (PE/EA=10/1) to give (2S,3R)-5,7-bis(benzyloxy)-2-(4,5-bis(benzyloxy)-2-methylphenyl)chroman-3-ol (0.31 g, 30% yield) as a white solid. ¹H NMR (400 MHZ, CDCl₃): 7.48-7.24 (m, 20H), 7.01 (s, 1H), 6.79 (s, 1H), 6.28 (d, J=2.4 Hz, 1H), 6.18 (d, J=2.0 Hz, 1H), 5.16 (s, 2H), 5.12 (q, J=7.2 Hz, 2H), 5.04 (s, 2H), 4.99 (s, 2H), 4.89 (d, J=8.4 Hz, 1H), 4.02 (m, 1H), 3.17 (dd, J=16.4, 5.6 Hz, 1H), 3.17 (dd, J=16.4, 9.2 Hz, 1H), 2.31 (s, 3H).

Step 7: Synthesis of (2S,3R)-5,7-bis(benzyloxy)-2-(4,5-bis(benzyloxy)-2-methylphenyl)chroman-3-yl 3,4,5-tris(benzyloxy)benzoate (9). To a solution of 3,4,5-tris(benzyloxy)-2-fluorobenzoic acid (0.26 g, 0.56 mmol, 1.5 eq) in CH₂Cl₂ (5 mL) was added (COCl)₂ (0.1 mL, 0.75 mmol, 2.0 eq.) and 2 drops of dry DMF at 0° C. The reaction mixture was stirred at RT for 1 h. After this time, the reaction mixture was concentrated under reduced pressure to get acid chloride. Obtained acid chloride was added to a solution of (2S,3R)-5,7-bis(benzyloxy)-2-(4,5-bis(benzyloxy)-2-methylphenyl)chroman-3-ol (0.25 g, 0.37 mmol, 1.0 eq.), DMAP (0.18 g, 1.50 mmol, 4.0 eq.) and TEA (0.22 mL, 1.50 mmol, 4.0 eq.) in CH₂Cl₂ (10 mL) at 0° C. The resulting mixture was stirred at RT for 16 h. The reaction mixture was diluted with H₂O (30 mL) and extracted with CH₂Cl₂ (2×50 mL). The combined organic layer was washed with brine (30 mL), dried over Na₂SO₄, filtered and concentrated. The residue was purified by flash chromatography on silica gel (PE/EA=6/1) to give (2S,3R)-5,7-bis(benzyloxy)-2-(4,5-bis(benzyloxy)-2-methylphenyl)chroman-3-yl 3,4,5-tris(benzyloxy)benzoate (0.33 g, 79.5% yield) as a white solid. ¹H NMR (400 MHZ, CDCl₃): 7.48-7.14 (m, 35H), 6.99 (s, 1H), 6.97 (d, J=6.4 Hz, 1H), 6.71 (s, 1H), 6.29 (d, J=2.0 Hz, 1H), 6.23 (d, J=2.0 Hz, 1H), 5.50 (q, J=5.6 Hz, 1H), 5.31 (d, J=7.6 Hz, 1H), 5.08-4.94 (m, 12H), 4.89 (s, 2H), 3.15 (dd, J=16.8, 5.6 Hz, 1H), 3.17 (dd, J=16.4, 7.2 Hz, 1H), 2.35 (s, 3H).

Step 8: Synthesis of Compound 52. To a solution of (2S,3R)-5,7-bis(benzyloxy)-2-(4,5-bis(benzyloxy)-2-methylphenyl)chroman-3-yl 3,4,5-tris(benzyloxy)benzoate (0.3 g, 0.27 mmol, 1.0 eq.) in THF (3 mL) and MeOH (3 mL) was added Pd(OH)₂ (20 wt. %, 0.039 g). The mixture was stirred at room temperature under H₂ atmosphere for overnight. Then the mixture was passed through a pad of celite to remove the catalyst. The filtrate was concentrated under reduced pressure. Obtained crude compound was purified by Prep-HPLC to obtain (2S,3R)-2-(4,5-dihydroxy-2-methylphenyl)-5,7-dihydroxychroman-3-yl 3,4,5-trihydroxybenzoate (0.040 g, 31% yield) as off white solid. ¹H NMR (400 MHZ, DMSO-d₆): 9.88-8.80 (bs, 7H), 6.62 (s, 1H), 6.61 (s, 1H), 6.50 (s, 1H), 5.93 (d, J=2.4 Hz, 1H), 5.75 (d, J=2.0 Hz, 1H), 5.22 (q, J=5.6 Hz, 1H), 5.17 (d, J=6.4 Hz, 1H), 2.80 (dd, J=16.0, 4.8 Hz, 1H), 2.63 (dd, J=16.4, 6.8 Hz, 1H), 2.20 (s, 3H).

### Compound 54

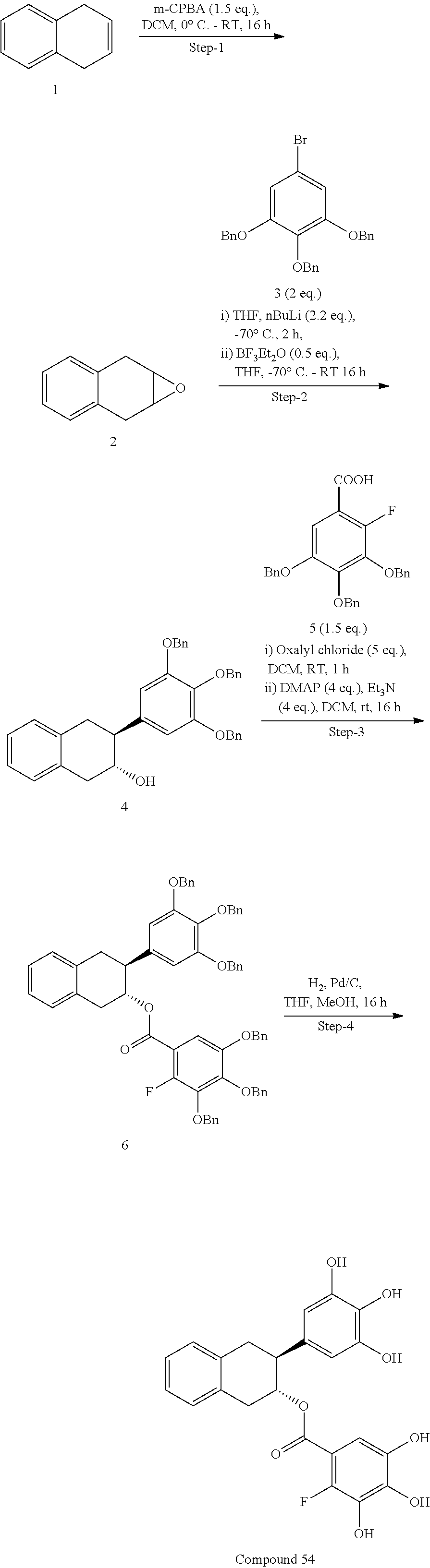

Step 1: Synthesis of 1a,2,7,7a-tetrahydronaphtho[2,3-b]oxirene (2). To a solution of 1,4-dihydronaphthalene (1.8 g, 13.800 mmol, 1 eq.) in DCM (25 mL) was added m-CPBA (3.57 g, 20.700 mmol, 1.5 eq.) in small portions over a period of 20 min at 0° C. and the reaction mass was stirred for 16 hours at room temperature. The excess of mCPBA was removed by washed with aq NaHCO₃. The combined organic layer was washed with water, brine, dried over Na₂SO₄, filtered and concentrated under reduced pressure. Obtained crude compound was purified by flash column chromatography (EtOAc in hexane) to get 1a,2,7,7a-tetrahydronaphtho[2,3-b]oxirene as a white solid (1.53 g, 75% yield). ¹HNMR (400 MHZ, DMSO-d₆): δ 7.10-7.07 (m, 2H), 7.04-7.00 (m, 2H), 3.41 (t, J=1.2 Hz, 2H), 3.15 (t, J=18.8 Hz, 4H).

Step 2: Synthesis of (2R,3S)-3-(3,4,5-tris(benzyloxy)phenyl)-1,2,3,4-tetrahydronaphthalen-2-ol (4). To a solution of (5-bromobenzene-1,2,3-triyl) tris(oxy)) tris(methylene))tribenzene (1.23 g, 2.599 mmol, 1 eq.), in 10 mL THF was added n-BuLi (2M in hexanes 3.2 mL, 5.198 mmol, 2 eq.) at −70° C. and stirred at −70° C. for 2 h. Then 1a,2,7,7a-tetrahydronaphtho[2,3-b]oxirene (380 mg, 2.599 mmol, 1 eq.) and BF₃-Et₂O (0.1 mL, 0.779 mmol, 0.3 eq.) were added at −70° C. to the above reaction mass and allowed to stirred for RT 16 h. Reaction mixture was quenched with aqueous NH₄Cl solution product was extracted with EtOAc (2×50 mL). Organic layer was washed with brine and dried over anhydrous Na₂SO₄, filtered and concentrated under reduced pressure. Obtained crude compound was purified by flash column chromatography (EtOAc in hexane) to get desired compound (2R,3S)-3-(3,4,5-tris(benzyloxy)phenyl)-1,2,3,4-tetrahydronaphthalen-2-ol as a red solid (0.310 g, 22% yield). ¹H NMR (400 MHZ, DMSO-d₆) δ 7.40-7.30 (m, 11H), 7.25 (d, J=1.2 Hz, 2H), 7.24 (d, J=2.0 Hz, 2H), 7.06 (t, J=8.0 Hz, 4H), 6.69 (s, 2H), 5.03 (s, 4H), 4.84 (s, 2H), 4.02 (q, J=5.6 Hz, 1H), 2.99-2.87 (m, 2H), 2.81 (t, J=8.8 Hz, 1H), 2.71 (t, J=7.6 Hz, 1H), 2.49 (d, J=1.6 Hz, 1H). LCMS: (M+H⁺): m/Z: 625.05.

Step 3: Synthesis of (2R,3S)-3-(3,4,5-tris(benzyloxy)phenyl)-1,2,3,4-tetrahydronaphthalen-2-yl 3,4,5-tris(benzyloxy)-2-fluorobenzoate (5). To a stirred solution of 3,4,5-tris(benzyloxy)-2-fluorobenzoic acid (0.428 g, 0.935 mmol, 1.3 eq.) in DCM (8 mL) was added oxalyl chloride (0.3 mL, 3.597 mmol, 5 eq.) and two drops of DMF at 0° C. The reaction mixture was stirred at RT for 1 h. After this time, the reaction mixture was concentrated under reduced pressure to get acid chloride. Obtained acid chloride was added to a solution of (2R,3S)-3-(3,4,5-tris(benzyloxy)phenyl)-1,2,3,4-tetrahydronaphthalen-2-ol (0.390 g, 0.719 mmol, 1 eq.), DMAP (0.439 g, 3.597 mmol, 5 eq.) and Et₃N (0.49 mL, 3.597 mmol, 5 eq.) in CH₂Cl₂ (12 mL) at 0° C. Then the reaction mixture was stirred at RT 16 h. Finally the reaction was quenched with saturated NaHO₃ aqueous solution. The organic layer was separated and the aqueous layer was extracted with CH₂Cl₂. Combined organic phase was dried over MgSO₄ filtered and concentrated under reduced pressure. Obtained crude compound was purified by flash column chromatography (EtOAc in hexane) to get (2R,3S)-3-(3,4,5-tris(benzyloxy)phenyl)-1,2,3,4-tetrahydronaphthalen-2-yl 3,4,5-tris(benzyloxy)-2-fluorobenzoate as a brownish solid (0.302 g, 43% yield). ¹H NMR (400 MHZ, DMSO-d₆) δ 7.43-7.15 (m, 30H), 6.91 (s, 2H), 6.79 (d, J=6.0 Hz, 2H), 5.32 (q, J=8 Hz, 1H), 5.06 (d, J=4.8 Hz, 1H), 5.03 (s, 4H), 4.98 (s, 4H), 4.81 (s, 4H), 3.26 (dd, J=1.6 Hz, 2H), 3.10 (dd, J=1.6 Hz, 2H). ¹⁹F NMR (400 MHZ, DMSO-d₆) δ−134.71.

Step 4: Synthesis of Compound 54. To a solution of (2R,3S)-3-(3,4,5-tris(benzyloxy)phenyl)-1,2,3,4-tetrahydronaphthalen-2-yl 3,4,5-tris(benzyloxy)-2-fluorobenzoate (0.3 g, 0.305 mmol, 1 eq.) in 8 mL of 1:1 THF:MeOH was added palladium hydroxide (20 wt. %, 0.3 g) at RT and stirred under hydrogen atmosphere for 16 h. Then the mixture was passed through a pad of celite to remove the catalyst. The filtrate was concentrated under vacuum. Obtained crude compound was purified by Prep-HPLC to obtain (2R,3S)-3-(3,4,5-trihydroxyphenyl)-1,2,3,4-tetrahydronaphthalen-2-yl 2-fluoro-3,4,5-trihydroxybenzoate as an off-white color solid (0.072 g, 53% yield). ¹H NMR (400 MHZ, DMSO-d₆): 8.84 (s, 6H), 7.15-7.09 (m, 4H), 6.81 (s, 2H), 6.17 (s, 2H), 5.32 (q, J=5.6 Hz, 1H), 3.10 (d, J=4.4 Hz, 1H), 3.05 (d, J=4.8 Hz, 2H), 2.93 (t, J=8.4 Hz, 1H), 2.85 (dd, J=6.8 Hz, 1H). LCMS: (M−H+): m/Z: 441.1.

### Compound 53

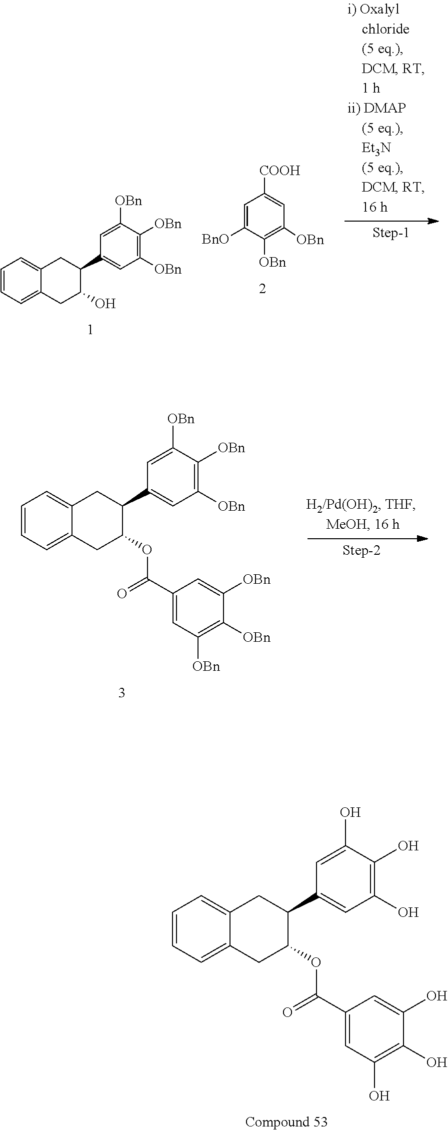 [SMILES: O[C@@H]1Cc2ccccc2C[C@H]1c1cc(OCc2ccccc2)c(OCc2ccccc2)c(OCc2ccccc2)c1 ; O=C(O)c1cc(OCc2ccccc2)c(OCc2ccccc2)c(OCc2ccccc2)c1 ; O=C(O[C@@H]1Cc2ccccc2C[C@H]1c1cc(OCc2ccccc2)c(OCc2ccccc2)c(OCc2ccccc2)c1)c1cc(OCc2ccccc2)c(OCc2ccccc2)c(OCc2ccccc2)c1 ; O=C(O[C@@H]1Cc2ccccc2C[C@H]1c1cc(O)c(O)c(O)c1)c1cc(O)c(O)c(O)c1]

Step 1. Synthesis of (2R,3S)-3-(3,4,5-tris(benzyloxy)phenyl)-1,2,3,4-tetrahydronaphthalen-2-yl 3,4,5-tris(benzyloxy)benzoate (3). To a solution of 3,4,5-tris(benzyloxy)benzoic acid (0.411 g, 0.719 mmol, 1 eq.) in DCM (8 mL) was added oxalyl chloride (0.3 mL, 3.597 mmol, 5 eq.) and two drops of DMF at 0° C. The reaction mixture was stirred at RT for 1 h. The reaction mixture was stirred at RT for 1 h. After this time, the reaction mixture was concentrated under reduced pressure to get acid chloride. Obtained acid chloride was added to a solution of (2R,3S)-3-(3,4,5-tris(benzyloxy)phenyl)-1,2,3,4-tetrahydronaphthalen-2-ol (0.411 g, 0.935 mmol, 1 eq.), DMAP (0.439 g, 3.597 mmol, 5 eq.) and Et₃N (0.49 mL, 3.597 mmol, 5 eq.) in CH₂Cl₂ (12 mL) at 0° C. Then the reaction mixture was stirred at RT 16 h. Finally, the reaction was quenched with saturated NaHO₃ aqueous solution (5 mL). The organic layer was separated and the aqueous layer was extracted with CH₂Cl₂ (30 mL). Combined organic phase was dried over MgSO₄ filtered and concentrated under reduced pressure. Obtained crude compound was purified by flash column chromatography (EtOAc in hexane) to get (2R,3S)-3-(3,4,5-tris(benzyloxy)phenyl)-1,2,3,4-tetrahydronaphthalen-2-yl 3,4,5-tris(benzyloxy)benzoate as an off-white solid (0.310 g, 45% yield). ¹H NMR (400 MHZ, DMSO-de) δ 7.46-7.18 (m, 34H), 7.09 (s, 2H), 6.94 (s, 2H), 5.48 (q, J=8 Hz, 1H), 5.09 (s, 4H), 5.03 (s, 4H), 4.91 (s, 2H), 4.78 (s, 2H), 3.44 (d, J=1.6 Hz, 1H), 3.24 (d, J=1.6 Hz, 2H), 3.06 (dd, J=1.6 Hz, 2H).

Step 2: Synthesis of Compound 53. To a solution of (2R,3S)-3-(3,4,5-tris(benzyloxy)phenyl)-1,2,3,4-tetrahydronaphthalen-2-yl 3,4,5-tris(benzyloxy)benzoate (0.3 g, 0.3110 mmol, 1 eq.) in 8 mL of 1:1; THF:MeOH was added palladium hydroxide (20 wt. %, 0.3 g) at RT and the reaction mixture was stirred under hydrogen atmosphere for 16 h. Then the mixture was passed through a pad of celite to remove the catalyst. The filtrate was concentrated under vacuum. Obtained crude compound was purified by Prep-HPLC to obtain 2R,3S)-3-(3,4,5-trihydroxyphenyl)-1,2,3,4-tetrahydronaphthalen-2-yl 3,4,5-trihydroxybenzoate as an off-white color solid (0.101 g, 77% yield). ¹H NMR (400 MHZ, DMSO-d₆): 8.84 (s, 6H), 7.15-7.09 (s, 4H), 6.81 (s, 1H), 6.17 (s, 2H), 5.32 (q, J=5.6 Hz, 1H), 3.10 (d, J=4.4 Hz, 1H), 3.05 (d, J=4.8 Hz, 2H), 2.93 (t, J=8.4 Hz, 1H), 2.85 (dd, J=6.8 Hz, 1H). LCMS: (M−H+): m/Z: 423.1.

### Compound 55

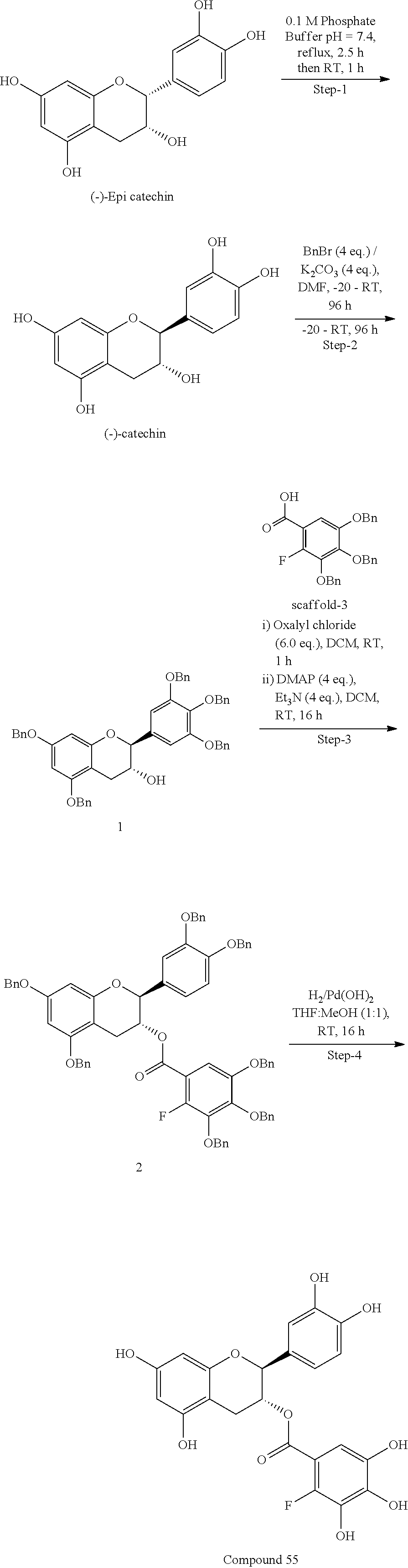 [SMILES: Oc1cc(O)c2c(c1)O[C@H](c1ccc(O)c(O)c1)[C@H](O)C2 ; Oc1cc(O)c2c(c1)O[C@@H](c1ccc(O)c(O)c1)[C@H](O)C2 ; O=C(O)c1cc(OCc2ccccc2)c(OCc2ccccc2)c(OCc2ccccc2)c1F ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O[C@@H]1Cc2c(OCc3ccccc3)cc(OCc3ccccc3)cc2O[C@H]1c1ccc(OCc2ccccc2)c(OCc2ccccc2)c1)c1cc(OCc2ccccc2)c(OCc2ccccc2)c(OCc2ccccc2)c1F ; O=C(O[C@@H]1Cc2c(O)cc(O)cc2O[C@H]1c1ccc(O)c(O)c1)c1cc(O)c(O)c(O)c1F]

Step 1: Synthesis of (−)-catechin. A mixture of (2R,3R)-2-(3,4-dihydroxyphenyl)chromane-3,5,7-triol (4.7 g, 16.19 mmol, 1.0 eq.) and 0.1 M phosphate buffer (40 mL) was heated at 110° C. for 2.5 h, under dark conditions. Then reaction mixture was allowed to RT and stirred for another 1 h. Further reaction mixture was kept stand for 10 h at RT and filtered. The obtained solid was purified by prep-HPLC to give (2R,3R)-5,7-bis(benzyloxy)-2-(3,4,5-tris(benzyloxy)phenyl)chroman-3-ol (1.28 g, 27% yield) as white solid. ¹H NMR (400 MHz, DMSO-d₆): δ 9.16 (s, 1H), 8.92 (s, 1H), 8.84 (s, 1H), 8.80 (s, 1H), 6.75-6.52 (m, 3H), 5.87 (d, J=2.0 Hz, 1H), 5.66 (d, J=2.4 Hz, 1H), 4.84 (d, J=5.2 Hz, 1H), 4.45 (d, J=7.6 Hz, 1H), 3.79 (quintet, J=5.2 Hz, 1H), 2.63 (dd, J=16.0 Hz, 5.2 Hz, 1H), 2.33 (q, J=16.4, 1H).

Step 2: Synthesis of (2R,3R)-5,7-bis(benzyloxy)-2-(3,4,5-tris(benzyloxy)phenyl)chroman-3-ol (1). To a solution of (2R,3R)-5,7-bis(benzyloxy)-2-(3,4,5-tris(benzyloxy)phenyl)chroman-3-ol (1.26 g, 4.36 mmol, 1.0 eq.) in dry DMF (15 mL) was added K₂CO₃ (2.41 g, 17.44 mmol, 4.0 eq.) and stirred at RT for 0.5 h. To this was slowly added BnBr (2.1 mL, 17.44 mmol, 4.0 eq.) drop wise at −20° C. The suspension was slowly warmed to RT and allowed to stir at RT for 96 h. After complete consumption of the starting material monitored by TLC, the reaction mixture was filtered through pad of celite to remove K₂CO₃. The celite pad was washed with EtOAc (100 mL). The combined organic phase was washed with cold H₂O (2×50 mL) and brine (50 mL), dried over Na₂SO₄, filtered and concentrated. The obtained residue was purified by flash column chromatography with (EtOAc:Hexane, (5:1), to afford (2R,3R)-5,7-bis(benzyloxy)-2-(3,4,5-tris(benzyloxy)phenyl)chroman-3-ol (1.28 g, 45% yield) as an off white solid. ¹H NMR (400 MHZ, DMSO-de): δ 7.50-7.26 (m, 20H), 7.16-6.98 (m, 2H), 6.87 (d, J=8.4 Hz, 1H), 6.32 (d, J=2.0 Hz, 1H), 6.12 (d, J=2.0 Hz, 1H), 5.16-4.98 (m, 9H), 4.63 (d, J=7.6 Hz, 1H), 4.02-3.90 (m, 1H), 2.76 (dd, J=16.8 Hz, 5.6 Hz, 1H), 2.56-2.41 (m, 1H).

Step 3: Synthesis of (2R,3R)-5,7-bis(benzyloxy)-2-(3,4,5-tris(benzyloxy)phenyl)chroman-3-yl 3,4,5-tris(benzyloxy)-2-fluorobenzoate (2). To a solution of 3,4,5-tris(benzyloxy)-2-fluorobenzoic acid (2.32 g, 5.07 mmol, 1.5 eq.) in CH₂Cl₂ (30 mL) was added (COCl)₂ (1.8 mL, 20.29 mmol, 6.0 eq.) and 2 drops of dry DMF at 0° C. The reaction mixture was stirred at RT for 1 h. After this time, the reaction mixture was concentrated under reduced pressure to get acid chloride. Obtained acid chloride was added to a solution of (2R,3R)-5,7-bis(benzyloxy)-2-(3,4,5-tris(benzyloxy)phenyl)chroman-3-ol (2.2 g, 3.38 mmol, 1.0 eq.), DMAP (1.65 g, 13.52 mmol, 4.0 eq.) and TEA (1.9 mL, 13.52 mmol, 4.0 eq.) dissolved in CH₂Cl₂ (40 mL) at 0° C. The resulting mixture was stirred at RT for 16 h. The reaction mixture was diluted with H₂O (50 mL) and extracted with CH₂Cl₂ (2×100 mL). The combined organic phase was washed with brine (50 mL), dried over Na₂SO₄, filtered and concentrated. The residue was purified by flash column chromatography on silica gel (PE/EA=6/1) to give (2R,3R)-5,7-bis(benzyloxy)-2-(3,4,5-tris(benzyloxy)phenyl)chroman-3-yl 3,4,5-tris(benzyloxy)-2-fluorobenzoate (3.04 g, 82% yield) as a white solid. ¹H NMR (400 MHZ, CDCl₃): δ 7.43-7.21 (m, 35H), 7.06-7.01 (m, 2H), 6.94 (d, J=1.6 Hz, 1H), 6.88 (d, J=8.4 Hz, 1H), 6.28 (dd, J=6.0, 2.0 Hz, 2H), 5.48 (q, J=7.2 Hz, 1H), 5.12-4.90 (m, 15H), 3.14-2.80 (dq, J=16.4 Hz, 5.2 Hz, 2H).

Step 4: Synthesis of Compound 55. To a mixture of (2R,3R)-5,7-bis(benzyloxy)-2-(3,4,5-tris(benzyloxy)phenyl)chroman-3-yl 3,4,5-tris(benzyloxy)-2-fluorobenzoate (3.0 g, 2.75 mmol, 1.0 eq.) in THF (15 mL) and MeOH (15 mL) was added Pd(OH)₂/C (20 wt. %, 0.39 g). The mixture was stirred at room temperature under H₂ atmosphere for overnight. The reaction mixture was filtered through pad of celite and the filtrate was concentrated. The residue was purified by prep-HPLC to give (2R,3R)-5,7-dihydroxy-2-(3,4,5-trihydroxyphenyl)chroman-3-yl 2-fluoro-3,4,5-trihydroxybenzoate (0.65 g, 51.3% yield) as an off-white solid. ¹H NMR (400 MHZ, DMSO-d₆) δ 9.12 (bs, 7H), 6.72 (d, J=1.6 Hz, 1H), 6.66 (d, J=2.8 Hz, 1H), 6.65 (s, 1H), 0.59 (dd, J=8.4, 2.0 Hz, 1H), 5.91 (d, J=2.0 Hz, 1H), 5.78 (d, J=2.0 Hz, 1H), 5.28 (q, J=5.6 Hz, 1H), 5.04 (d, J=6.0 Hz, 1H), 2.70-2.54 (dq, J=9.2, 5.6 Hz, 2H).

### Compounds 55 and 56

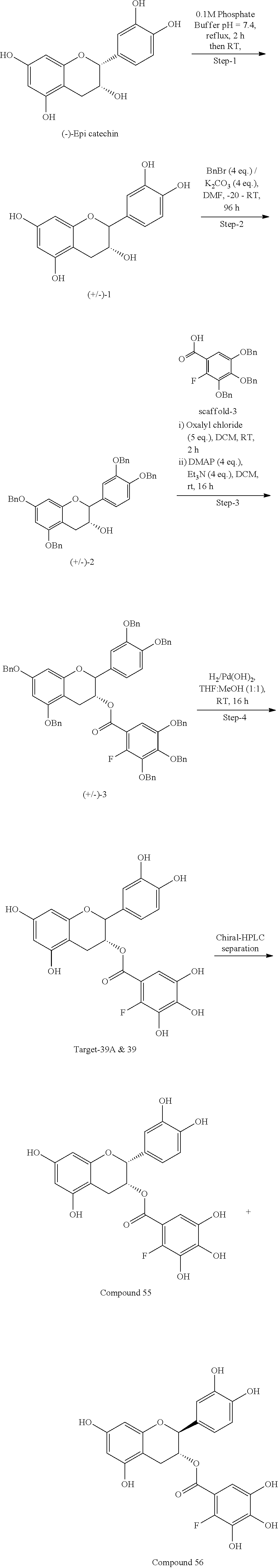

Step 1: Synthesis of (3R)-2-(3,4-dihydroxyphenyl)chromane-3,5,7-triol (1). A stirred mixture of (2R,3R)-2-(3,4-dihydroxyphenyl)chromane-3,5,7-triol (5.0 g, 17.22 mmol, 1.0 eq.) and 0.1 M phosphate buffer (50 mL) was heated at 110° C. for 2 h. The reaction progress was monitored by LCMS. Then reaction mixture was allowed to RT and kept for lyophilization to obtain (3R)-2-(3,4-dihydroxyphenyl)chromane-3,5,7-triol (5.0 g) as brown color solid.

Step 2: Synthesis of (3R)-5,7-bis(benzyloxy)-2-(3,4-bis(benzyloxy)phenyl)chroman-3-ol (2). To a solution of (3R)-2-(3,4-dihydroxyphenyl)chromane-3,5,7-triol (3.0 g, 10.33 mmol, 1.0 eq.) in dry DMF (100 mL) was added K₂CO₃ (5.71 g, 41.34 mmol, 4.0 eq.) and stirred at RT for 0.5 h. To this was slowly added BnBr (4.9 mL, 41.34 mmol, 4.0 eq.) dropwise at −20° C. The suspension was allowed to RT and stirred for 96 h. The consumption of the starting material was monitored by TLC. After complete consumption of the starting material, the reaction mixture was filtered through pad of celite to remove K₂CO₃. The celite pad was washed with EtOAc (100 mL). The combined organic phase was washed with cold H₂O (2×50 mL) and brine (50 mL), dried over Na₂SO₄, filtered and concentrated. The obtained residue was purified by flash column chromatography with (EtOAc:Hexane, (6:1), to afford (3R)-5,7-bis(benzyloxy)-2-(3,4-bis(benzyloxy)phenyl)chroman-3-ol (0.8 g, 11% yield) as pale yellow solid.

Step 3: Synthesis of ((3R)-5,7-bis(benzyloxy)-2-(3,4-bis(benzyloxy)phenyl)chroman-3-yl 3,4,5-tris(benzyloxy)-2-fluorobenzoate (3). To a solution of (3R)-5,7-bis(benzyloxy)-2-(3,4-bis(benzyloxy)phenyl)chroman-3-ol (0.64 g, 1.39 mmol, 1.3 eq.) in CH₂Cl₂ (10 mL) was added (COCl)₂ (0.5 mL, 5.28 mmol, 5.0 eq.) and 2 drops of dry DMF at 0° C. The reaction mixture was stirred at RT for 2 h. After this time, the reaction mixture was concentrated under reduced pressure to get acid chloride. Obtained acid chloride was added to a solution of (3R)-5,7-bis(benzyloxy)-2-(3,4-bis(benzyloxy)phenyl)chroman-3-ol (0.7 g, 1.07 mmol, 1.0 eq.), DMAP (0.53 g, 4.30 mmol, 4.0 eq.) and TEA (0.9 mL, 4.30 mmol, 4.0 eq.) in CH₂Cl₂ (20 mL) at 0° C. The resulting mixture was stirred at RT for 16 h. The reaction mixture was diluted with H₂O (30 mL) and extracted with CH₂Cl₂ (2×50 mL). The combined organic phase was washed with brine (30 mL), dried over Na₂SO₄, filtered and concentrated. The obtained residue was purified by flash column chromatography on silica gel (PE/EA=9/1) to give ((3R)-5,7-bis(benzyloxy)-2-(3,4-bis(benzyloxy)phenyl)chroman-3-yl 3,4,5-tris(benzyloxy)-2-fluorobenzoate (0.88 g, 75% yield) as off white solid.

Step 4: Synthesis of Compounds 55 and 56. To a solution of ((3R)-5,7-bis(benzyloxy)-2-(3,4-bis(benzyloxy)phenyl)chroman-3-yl 3,4,5-tris(benzyloxy)-2-fluorobenzoate (0.88 g, 0.80 mmol, 1.0 eq.) in THF (15 mL) and MeOH (15 mL) was added Pd(OH)₂/C (20 wt. %, 0.88 g) and HCOOH (0.8 mL). The mixture was stirred at room temperature under H₂ atmosphere for 16 h. The reaction mixture was filtered through pad of celite and the filtrate was concentrated. The residue was purified by prep-HPLC and separated by chiral-HPLC to give 2-(3,4-dihydroxyphenyl)-5,7-dihydroxychroman-3-yl 2-fluoro-3,4,5-trihydroxybenzoate (0.045 g, 12% yield as black solid) and (2S,3R)-2-(3,4-dihydroxyphenyl)-5,7-dihydroxychroman-3-yl 2-fluoro-3,4,5-trihydroxybenzoate (0.120 g, 32% yield as grey solid).

Compound 55: 1H NMR (400 MHZ, DMSO-d₆) δ 9.12 (bs, 7H), 6.72 (d, J=1.6 Hz, 1H), 6.66 (d, J=2.8 Hz, 1H), 6.65 (s, 1H), 6.59 (dd, J=8.4, 2.0 Hz, 1H), 5.91 (d, J=2.0 Hz, 1H), 5.78 (d, J=2.0 Hz, 1H), 5.28 (q, J=5.6 Hz, 1H), 5.04 (d, J=6.0 Hz, 1H), 2.70-2.54 (dq, J=9.2, 5.6 Hz, 2H).

Compound 56: ¹H NMR (400 MHZ, DMSO-d₆) δ 9.48 (bs, 1H), 9.29 (bs, 3H), 9.03 (bs, 1H), 8.86 (bs, 1H), 8.76 (bs, 1H), 6.87 (s, 1H), 6.73 (d, J=8.0 Hz, 1H), 6.69-6.62 (m, 2H), 5.91 (d, J=2.0 Hz, 1H), 5.79 (d, J=2.0 Hz, 1H), 5.38 (s, 1H), 5.01 (s, 1H), 2.92 (dd, J=17.6, 4.4 Hz, 1H), 2.92 (d, J=16.0 Hz, 1H).

### Compound 57

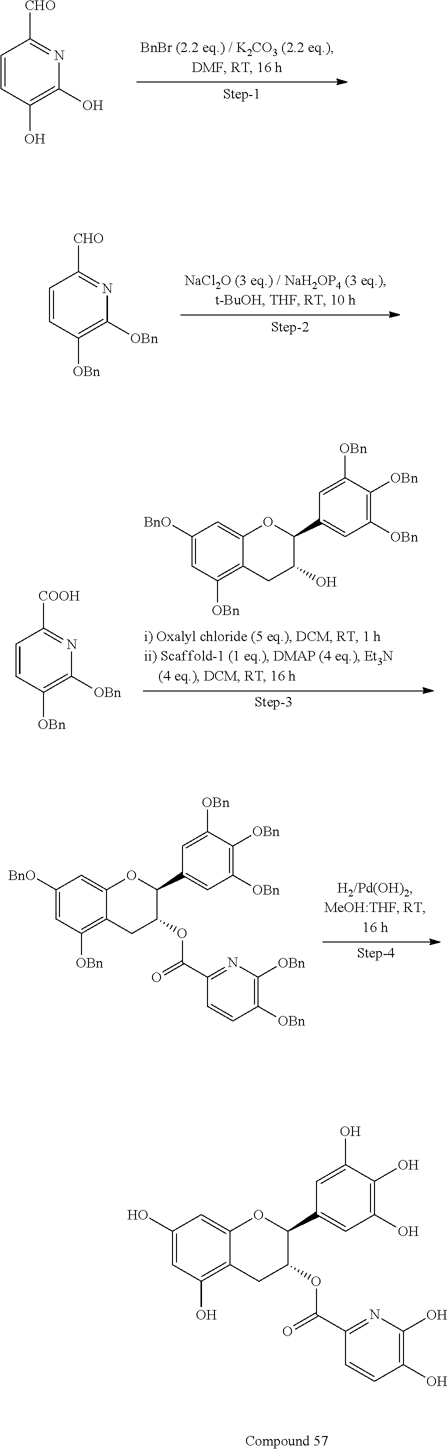 [SMILES: O=Cc1ccc(O)c(O)n1 ; O=Cc1ccc(OCc2ccccc2)c(OCc2ccccc2)n1 ; O=C(O)c1ccc(OCc2ccccc2)c(OCc2ccccc2)n1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1ccc(OCc2ccccc2)c(OCc2ccccc2)n1 ; O=C(O[C@@H]1Cc2c(O)cc(O)cc2O[C@H]1c1cc(O)c(O)c(O)c1)c1ccc(O)c(O)n1]

Step 1: Synthesis of 5,6-bis(benzyloxy)picolinaldehyde (2). To a stirred solution of 5,6-dihydroxypicolinaldehyde (0.64 g, 4.672 mmol) and potassium carbonate (1.7 g, 10.279 mmol, 2.2 eq.) in dry DMF (15 mL) was drop wise added benzyl bromide (1.2 mL, 10.279 mmol, 2.2 eq.) mixture was stirred for overnight at rt. The solution was diluted with EtOAc, washed with brain, and dried over Na₂SO₄. After removal of the solvent under reduced pressure. The crude compound was purified by flash column chromatography, eluted with 15% EtOAc in hexane, as an eluent affords to obtain 5,6-bis(benzyloxy)picolinaldehyde as a pale brown solid (1 g, 80%). ¹H NMR (400 MHZ, DMSO-d₆): δ 9.41 (s, 1H), 7.47-7.34 (m, 5H), 7.27 (t, J=4.0 Hz, 2H), 7.20 (t, J=4.0 Hz, 3H), 7.07 (d, J=7.8 Hz, 2H), 5.67 (s, 2H), 5.18 (s, 2H).

Step 2: Synthesis of 5,6-bis(benzyloxy)picolinic acid (3). To a stirred solution of 5,6-bis(benzyloxy)picolinaldehyde (7 g, 3.134 mmol, 1 eq.) in t-BuOH: THF (1:1, 20 mL) at 20° C. were sequentially added 2-methyl-2-butene (0.65 g, 9.404 mmol, 3 eq.), aq. NaH₂PO₄ solution (1.1 g, 9.404 mmol, 3 eq.), and aq. NaCl₂O solution (0.84 g, 9.404 mmol, 3 eq.). The reaction mixture was stirred at that temperature for 10 h before it was quenched with aq. NaHSO₃ solution (3 mL, 1.0 M). The resulting mixture was extracted with DCM (3×100 mL) and the combined organic phase was dried over Na₂SO₄ and filtered. After removal of the solvent under vacuum, the residue was purified by flash column chromatography, eluted with 5% methanol in DCM, as an eluent affords to obtained 5,6-bis(benzyloxy)picolinic acid as a white solid (0.61 g, 55%). ¹H NMR (400 MHZ, DMSO-d₆) δ 7.45-7.34 (m, 3H), 7.27 (t, J=6.8 Hz, 2H), 7.20 (t, J=7.6 Hz, 1H), 7.04 (d, J=7.2 Hz, 2H), 6.97 (s, 2H), 5.63 (s, 2H), 5.09 (s, 2H).

Step 3: Synthesis of (2S,3R)-5,7-bis(benzyloxy)-2-(3,4,5-tris(benzyloxy)phenyl)chroman-3-yl 5,6-bis(benzyloxy)picolinate (4). Under an N₂ atmosphere, to a stirred solution of 5,6-bis(benzyloxy)picolinic acid (0.6 g, 1.791 mmol, 1 eq.) in DCM (10 mL) was added oxalyl chloride (0.76 mL, 8.955 mmol, 5 eq.) and two drops DMF at 0° C. The reaction mixture was stirred at RT for 1 h. The excess oxalyl chloride were removed by distillation and the residue was dried under in the presence of organ gas to give acid chloride. This solution was added dropwise to a solution of (2S,3R)-5,7-bis(benzyloxy)-2-(3,4,5-tris(benzyloxy)phenyl)chroman-3-ol (1.3 g, 1.791 mmol, 1 eq.), DMAP (0.87 g, 7.164 mmol, 4 eq.) and Et₃N (1 mL, 7.164 mmol, 4 eq.) in CH₂Cl₂ (12 mL) at 0° C. Then the reaction mixture was stirred at RT 16 h. Finally, the reaction was quenched with saturated aqueous NaHCO₃ solution (5 mL). The organic layer was separated and the aqueous layer was extracted with CH₂Cl₂ (30 mL). Combined organic phase was dried over MgSO₄, filtered and concentrated under reduced pressure. Obtained crude compound was purified by flash column chromatography (EtOAc in hexane) to get (2S,3R)-5,7-bis(benzyloxy)-2-(3,4,5-tris(benzyloxy)phenyl)chroman-3-yl 5,6-bis(benzyloxy)picolinate as a white solid (0.28 g, 15% yield). ¹H NMR (400 MHZ, DMSO-d₆): δ 8.14 (s, 1H), 7.43-7.24 (m, 36H), 6.80 (s, 2H) 6.39 (s, 1H), 6.23 (s, 1H), 5.42 (d, J=5.6 Hz, 1H), 5.13 (d, J=6.4 Hz, 1H), 5.12 (s, 6H), 5.04 (s, 6H), 4.90 (s, 2H), 2.77 (dd, J=7.6 Hz, 2H). LCMS: (M+H⁺): m/Z: 1074.3.

Step 4: Synthesis of Compound 57. To a solution of (2S,3R)-5,7-bis(benzyloxy)-2-(3,4,5-tris(benzyloxy)phenyl)chroman-3-yl 5,6-bis(benzyloxy)picolinate (0.26 g, 0.242 mmol, 1 eq.), in 12 mL of 1:1 THF:MeOH was added palladium hydroxide (20 wt. %, 0.26 g) at RT and the reaction mixture was stirred under hydrogen atmosphere for 16 h. Then the mixture was passed through a pad of celite to remove the catalyst. The filtrate was concentrated under vacuum. Obtained crude compound was purified by Prep-HPLC to obtain to obtain (2S,3R)-5,7-dihydroxy-2-(3,4,5-trihydroxyphenyl)chroman-3-yl 5,6-dihydroxypicolinate as an off-white color solid (0.040 g, 84% yield). ¹H NMR (400 MHZ, DMSO-d₆): 9.35 (s. 2H), 9.07 (s, 5H), 6.77 (d, J=7.2 Hz, 1H), 6.67 (d, J=7.2 Hz, 1H), 6.26 (s, 2H), 5.92 (d, J=2.0 Hz, 1H), 5.78 (d, J=2.0 Hz, 1H), 5.20 (q, J=5.6 Hz, 1H), 4.97 (d, J=6.0 Hz, 1H), 2.66 (dd, J=4.4 Hz, 2H). LCMS: (M−H+): m/Z: 442.1.

### Compound 58

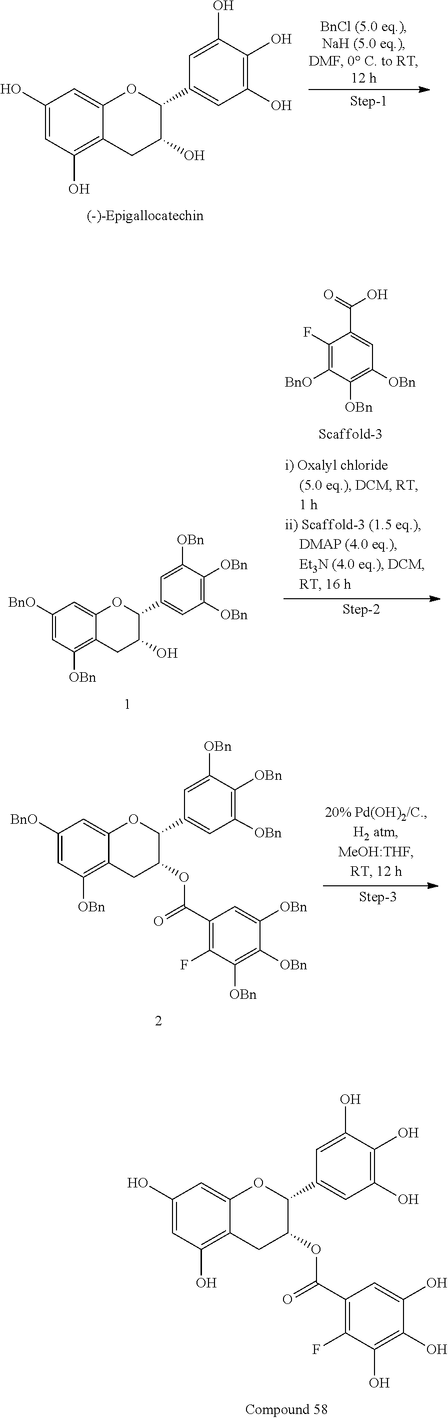 [SMILES: Oc1cc(O)c2c(c1)O[C@H](c1cc(O)c(O)c(O)c1)[C@H](O)C2 ; O=C(O)c1cc(OCc2ccccc2)c(OCc2ccccc2)c(OCc2ccccc2)c1F ; O[C@@H]1Cc2c(OCc3ccccc3)cc(OCc3ccccc3)cc2O[C@@H]1c1cc(OCc2ccccc2)c(OCc2ccccc2)c(OCc2ccccc2)c1 ; O=C(O[C@@H]1Cc2c(OCc3ccccc3)cc(OCc3ccccc3)cc2O[C@@H]1c1cc(OCc2ccccc2)c(OCc2ccccc2)c(OCc2ccccc2)c1)c1cc(OCc2ccccc2)c(OCc2ccccc2)c(OCc2ccccc2)c1F ; O=C(O[C@@H]1Cc2c(O)cc(O)cc2O[C@@H]1c1cc(O)c(O)c(O)c1)c1cc(O)c(O)c(O)c1F]

Step 1: Synthesis of (2R,3R)-5,7-bis(benzyloxy)-2-(3,4,5-tris(benzyloxy)phenyl)chroman-3-ol (1). To a stirred solution of (2R,3R)-2-(3,4,5-trihydroxyphenyl)chromane-3,5,7-triol (1.0 g, 3.26 mmol, 1.0 eq.) in dry DMF (10 mL) was added NaH (60%. wt in mineral oil) (0.65 g, 16.33 mmol, 5.0 eq.) at 0° C. and stirred for 0.5 h. To this was added BnCl (1.9 ml, 16.33 mmol, 5.0 eq.) drop wise at 0° C. The suspension was allowed to stir at RT for 12 h. After complete consumption of the starting material, the reaction mixture was quenched with sat. aq. Na₂SO₄ (2 mL) and filtered through pad of celite. The celite pad was washed with EtOAc (100 mL). The combined solvents were sequentially washed with H₂O (50 mL) and brine (30 mL). The organic layer was dried over Na₂SO₄, filtered and concentrated. The obtained residue was purified by flash chromatography on silica gel (PE/EA=6/1) to give (2R,3R)-5,7-bis(benzyloxy)-2-(3,4,5-tris(benzyloxy)phenyl)chroman-3-ol (1.02 g, 41% yield) as a white solid. ¹H NMR (400 MHZ, DMSO-d₆): δ 7.50-7.19 (m, 25H), 6.94 (s, 2H), 6.34 (d, J=2.4 Hz, 1H), 6.17 (d, J=2.0 Hz, 1H), 5.20-5.01 (m, 8H), 5.00-4.88 (m, 9H), 4.84 (d, J=4.4 Hz, 1H), 4.15 (d, J=3.6 Hz, 1H), 2.88-2.62 (m, 2H).

Step 2: Synthesis of (2R,3R)-5,7-bis(benzyloxy)-2-(3,4,5-tris(benzyloxy)phenyl)chroman-3-yl 3,4,5-tris(benzyloxy)-2-fluorobenzoate (2). To a solution of 3,4,5-tris(benzyloxy)-2-fluorobenzoic acid (0.27 g, 0.59 mmol, 1.5 eq.) in CH₂Cl₂ (5 mL) was added (COCl)₂ (0.3 mL, 1.98 mmol, 5.0 eq.) and 2 drops of dry DMF at 0° C. The reaction mixture was stirred at RT for 1 h. After this time, the reaction mixture was concentrated under reduced pressure to get acid chloride. Obtained acid chloride was added to a solution of (2R,3R)-5,7-bis(benzyloxy)-2-(3,4,5-tris(benzyloxy)phenyl)chroman-3-ol (0.3 g, 0.39 mmol, 1.0 eq.), DMAP (0.19 g, 1.58 mmol, 4.0 eq.) and TEA (0.3 mL, 1.58 mmol, 4.0 eq.) dissolved in CH₂Cl₂ (5 mL), at 0° C. The resulting mixture was stirred at RT for 16 h. The reaction mixture was diluted with H₂O (20 mL) and extracted with CH₂Cl₂ (2×50 mL). The combined organic layers were washed with brine (30 mL), dried over Na₂SO₄, filtered and concentrated. The residue was purified by flash chromatography on silica gel (PE/EA=7/1) to give (2R,3R)-5,7-bis(benzyloxy)-2-(3,4,5-tris(benzyloxy)phenyl)chroman-3-yl 3,4,5-tris(benzyloxy)-2-fluorobenzoate (0.37 g, 78% yield) as a white solid. ¹H NMR (400 MHZ, DMSO-d₆): δ 7.50-7.17 (m, 25H), 7.07 (d, J=6.4 Hz, 1H), 6.97 (s, 2H), 6.43 (s, 1H), 6.33 (d, J=2.0 Hz, 1H), 5.65 (s, 1H), 5.27 (s, 1H), 5.12 (s, 2H), 5.07 (s, 2H), 5.02-4.93 (m, 4H), 4.90 (s, 4H), 4.88-4.78 (m, 4H), 3.22-2.88 (m, 2H).

Step 3: Synthesis of Compound 58. To a mixture of (2R,3R)-5,7-bis(benzyloxy)-2-(3,4,5-tris(benzyloxy)phenyl)chroman-3-yl 3,4,5-tris(benzyloxy)-2-fluorobenzoate (0.5 g, 0.41 mmol, 1.0 eq.) in THF (5 mL) and MeOH (5 mL) was added Pd(OH)₂/C (20 wt. %, 0.059 g). The mixture was stirred at room temperature under H₂ atmosphere for overnight. The reaction mixture was filtered and the filtrate was concentrated. The residue was purified by prep-HPLC to give (2R,3R)-5,7-dihydroxy-2-(3,4,5-trihydroxyphenyl)chroman-3-yl 2-fluoro-3,4,5-trihydroxybenzoate (0.068 g, 34% yield) as an off-white solid. ¹H NMR (400 MHZ, DMSO-d₆) δ 9.45 (bs, 1H), 9.25 (s, 1H), 9.19 (s, 2H), 9.00 (s, 1H), 8.68 (bs, 2H), 8.00 (bs, 1H), 6.64 (d, J=6.4 Hz, 1H), 6.39 (s, 2H), 5.92 (d, J=2.4 Hz, 1H), 5.80 (d, J=2.4 Hz, 1H), 5.41 (s, 1H), 4.94 (s, 1H), 2.98-2.57 (m, 2H).

### Compound 59

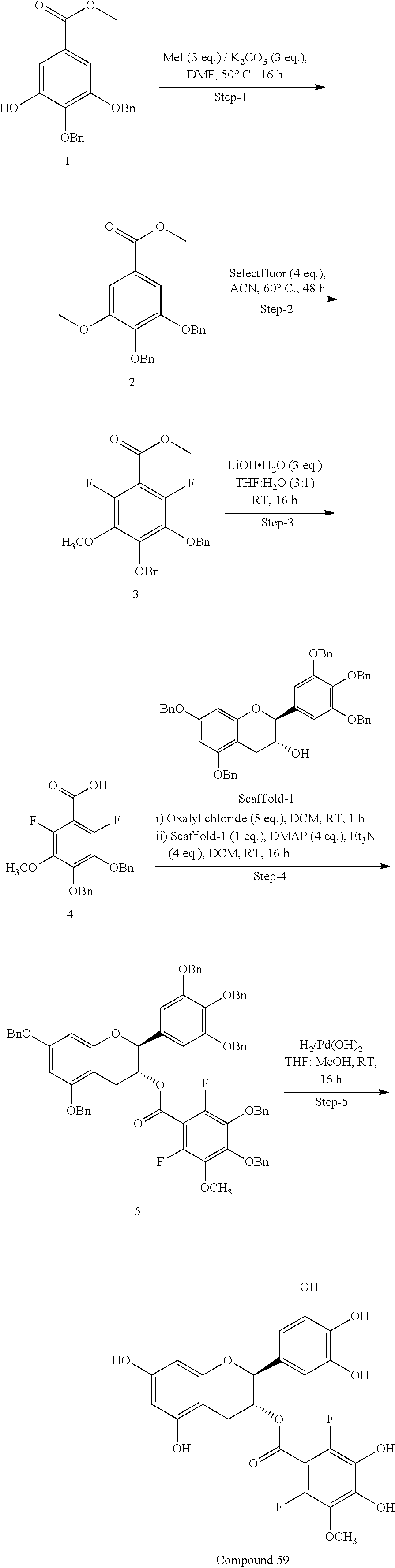

Step 1: Synthesis of methyl 3,4-bis(benzyloxy)-5-methoxybenzoate (2). To a suspension of methyl 3,4-bis(benzyloxy)-5-hydroxybenzoate (10.0 g, 27.472 mmol) in DMF (50 mL) was added K₂CO₃ (8.5 g, 82.413 mmol, 3 eq.) followed by iodomethane (3.8 mL, 82.413 mmol, 3 eq.) at 0° C. The mixture was heated to 50° C. for 16 h until TLC showed the reaction had been completed. Reaction mixture diluted with water and extracted with EtOAc. The solvent was evaporated, and the residue was purified by flash chromatography eluted with 15% EtOAc in hexane, as an eluent affords to obtain methyl 3,4-bis(benzyloxy)-5-methoxybenzoate as a white solid (10 g, 96% yield). ¹H NMR (400 MHZ, DMSO-d₆) δ 7.46 (t, J=1.6 Hz, 2H), 7.42-7.38 (m, 4H), 7.36 (d, J=2 Hz, 2H), 7.33-7.25 (m, 4H), 5.17 (s, 2H), 5.02 (s, 2H), 3.84 (s, 3H).

Step 2: Synthesis of methyl 3,4-bis(benzyloxy)-2,6-difluoro-5-methoxybenzoate (3). To a solution of methyl 3,4-bis(benzyloxy)-5-methoxybenzoate (14.5 g, 38.317 mmol, 1 eq.) in ACN (150 mL) was added selectfluor (54.2 g, 153.200 mmol, 4 eq.) at 0° C. and the reaction mixture stirred at 60° C. for 48 h. Reaction progress was monitor by TLC. Reaction mixture was quenched with cold water, extracted with EtOAc (3×150 mL), washed with brine and dried over anhydrous Na₂SO₄. Organic layer was evaporated under reduced pressure to obtained crude compound. The crude compound was purified by flash column chromatography, eluted with 5% EtOAc in hexane, as an eluent affords to obtain methyl 3,4-bis(benzyloxy)-2,6-difluoro-5-methoxybenzoate as a yellow solid (1.2 g, 8% yield). ¹H NMR (400 MHZ, DMSO-d₆): δ 7.43 (d, J=6.4 Hz, 2H), 7.39-7.27 (m, 8H), 5.20 (s, 2H), 5.01 (s, 2H), 3.86 (s, 3H), 3.79 (s, 3H), ¹⁹F NMR (400 MHZ, DMSO-d₆) δ−133.84, −133.85, −134.78, −134.79.

Step 3: Synthesis of 3,4-bis(benzyloxy)-2,6-difluoro-5-methoxybenzoic acid (4). A mixture of methyl 3,4-bis(benzyloxy)-2,6-difluoro-5-methoxybenzoate (1 g, 2.50 mmol, 1.0 eq.) in THF/H₂O (1:1) (20 mL) was added LiOH H₂O (0.31 g, 7.50 mmol, 3.0 eq.). The solution was stirred at RT for 16 h. The reaction mixture was concentrated to remove THF. Then the mixture was diluted with H₂O (30 mL) and extracted with EA (80 mL×2). The aqueous phase pH was adjusted to <3 with 1 N HCl. Obtained solid was filtered and the filter cake was dried to give the compound 3,4-bis(benzyloxy)-2,6-difluoro-5-methoxybenzoic acid as a white solid (0.85 g, 85% yield). ¹H NMR (400 MHZ, DMSO-d₆) δ 13.82 (s, 1H), 7.42 (d, J=1.2 Hz, 2H), 7.37-7.29 (m, 8H), 5.17 (s, 2H), 5.01 (s, 2H), 3.81 (s, 3H), ¹⁹F NMR (400 MHZ, DMSO-d₆) δ−134.65, −135.57.

Step 4: Synthesis of (2S,3R)-5,7-bis(benzyloxy)-2-(3,4,5-tris(benzyloxy)phenyl)chroman-3-yl 3,4-bis(benzyloxy)-2,6-difluoro-5-methoxybenzoate (5). Under an N₂ atmosphere, to a stirred solution of 3,4-bis(benzyloxy)-2,6-difluoro-5-methoxybenzoic acid (0.6 g, 1.637 mmol, 1 eq.) in DCM (5 mL) was added oxalyl chloride (1.0 mL, 8.168 mmol, 5 eq.) and two drops of DMF at 0° C. The reaction mixture was stirred at RT for 1 h. After this time, the reaction mixture was concentrated under reduced pressure to get acid chloride. Obtained acid chloride was added to a solution of (2S,3R)-5,7-bis(benzyloxy)-2-(3,4,5-tris(benzyloxy)phenyl)chroman-3-ol (1.0 g, 1.637 mmol, 1 eq.), DMAP (0.79 g, 6.548 mmol, 4 eq.) and Et₃N (0.9 mL, 6.548 mmol, 4 eq.) in CH₂Cl₂ (12 mL) at 0° C. Then the reaction mixture was stirred at RT 16 h. Finally, the reaction was quenched with saturated aqueous NaHCO₃ solution (5 mL). The organic layer was separated and the aqueous layer was extracted with CH₂Cl₂ (30 mL). Combined organic phase was dried over MgSO₄, filtered and concentrated under reduced pressure. Obtained crude compound was purified by flash column chromatography (EtOAc in hexane) to get (2S,3R)-5,7-bis(benzyloxy)-2-(3,4,5-tris(benzyloxy)phenyl)chroman-3-yl 3,4-bis(benzyloxy)-2,6-difluoro-5-methoxybenzoate as a yellow solid (0.7 g, 23% yield). ¹H NMR (400 MHZ, DMSO-d₆) δ 7.44-7.25 (m, 35H), 6.87 (s, 2H), 6.42 (d, J=2 Hz, 1H), 6.28 (d, J=1.6 Hz, 1H), 5.48 (d, J=5.2 Hz, 1H), 5.17 (d, J=7.2 Hz, 1H), 5.14 (s, 2H), 5.12 (s, 2H), 5.07 (s, 2H), 5.03 (s, 4H), 4.95 (s, 2H), 4.91 (s, 2H), 3.77 (s, 3H), 2.84 (dd, J=7.6 Hz, 2H), ¹⁹F NMR (400 MHZ, DMSO-d₆) δ−134.46, 134.45, 133.59, 133.58. LCMS: (M+H⁺): m/Z: 1139.

Step 5: Synthesis of Compound 59. To a solution of (2S,3R)-5,7-bis(benzyloxy)-2-(3,4,5-tris(benzyloxy)phenyl)chroman-3-yl 3,4-bis(benzyloxy)-2,6-difluoro-5-methoxybenzoate (0.4 g, 0.351 mmol, 1 eq.) in 8 mL of 1:1; THF:MeOH was added palladium hydroxide (20 wt. %, 0.40 g) at RT and the reaction mixture was stirred under hydrogen atmosphere for 16 h. Then the mixture was passed through a pad of celite to remove the catalyst. The filtrate was concentrated under vacuum. Obtained crude compound was purified by Prep-HPLC to obtain (2S,3R)-5,7-dihydroxy-2-(3,4,5-trihydroxyphenyl)chroman-3-yl 2,6-difluoro-3,4-dihydroxy-5-isopropoxybenzoate as an off-white color solid (0.075 g, 29% yield). ¹H NMR (400 MHZ, DMSO-d₆): 9.05 (bs, 7H), 6.26 (s, 2H), 5.92 (d, J=2.0 Hz, 1H), 5.80 (d, J=2.0 Hz, 1H), 5.38 (q, J=5.2 Hz, 1H), 5.03 (d, J=5.2 Hz, 1H), 3.73 (s, 3H), 2.56 (t, J=4 Hz, 2H), ¹⁹F NMR (400 MHZ, DMSO-d₆) δ−141.56, −141.54, −138.98, −138.91. LCMS: (M−H+): m/Z: 508.91.

### Compound 60

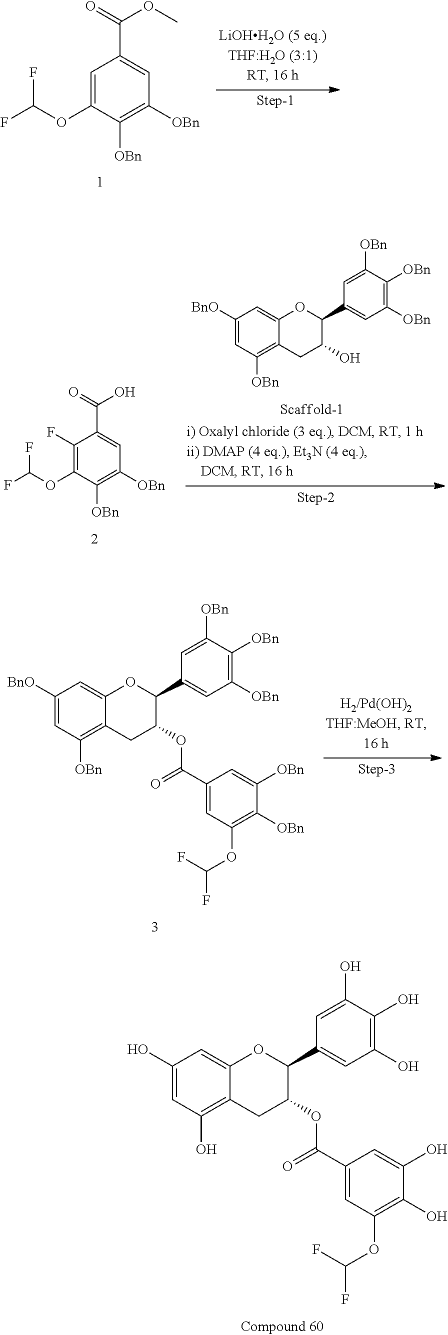 [SMILES: COC(=O)c1cc(OCc2ccccc2)c(OCc2ccccc2)c(OC(F)F)c1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1cc(OCc2ccccc2)c(OCc2ccccc2)c(OC(F)F)c1F ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1cc(OCc2ccccc2)c(OCc2ccccc2)c(OC(F)F)c1 ; O=C(O[C@@H]1Cc2c(O)cc(O)cc2O[C@H]1c1cc(O)c(O)c(O)c1)c1cc(O)c(O)c(OC(F)F)c1]

Step 1: Synthesis of 3,4-bis(benzyloxy)-5-(difluoromethoxy)benzoic acid (2). A mixture of methyl 3,4-bis(benzyloxy)-5-(difluoromethoxy)benzoate (1 g, 2.415 mmol, 1.0 eq.) in THF/H₂O (1:1) (20 mL) was added LiOH H₂O (0.25 g, 12.070 mmol, 5.0 eq.). The solution was stirred at RT for 16 h. The reaction mixture was concentrated to remove THF. Then the mixture was diluted with H₂O (25 mL) and extracted with EA (30 mL×2). The aqueous phase pH was adjusted to <3 with 1N HCl. Obtained solid was filtered and the filtered cake was dried to give the compound 3,4-bis(benzyloxy)-5-(difluoromethoxy)benzoic acid as a white solid (0.7 g, 72% yield). ¹H NMR (400 MHZ, DMSO-d₆) δ 7.64 (d, J=1.2 Hz, 1H), 7.46 (d, J=6 Hz, 3H), 7.39-7.34 (m, 5H), 7.30 (t, J=2.4 Hz, 3H), 7.11 (s, 1H), 5.15 (s, 2H), 5.01 (s, 2H), ¹⁹F NMR (400 MHz, DMSO-d₆) δ−80.92, −80.72.

Step 2: Synthesis of (2S,3R)-5,7-bis(benzyloxy)-2-(3,4,5-tris(benzyloxy)phenyl)chroman-3-yl 3,4-bis(benzyloxy)-5-(difluoromethoxy)benzoate (3). Under an N₂ atmosphere, to a stirred solution of 3,4-bis(benzyloxy)-5-(difluoromethoxy)benzoic acid (0.71 g, 1.785 mmol, 1.5 eq.) in DCM (6 mL) was added oxalyl chloride (0.5 mL, 5.950 mmol, 5 eq.) and two drops of DMF at 0° C. The reaction mixture was stirred at RT for 1 h. After this time, the reaction mixture was concentrated under reduced pressure to get acid chloride. Obtained acid chloride was added to a solution of to a solution of (2S,3R)-5,7-bis(benzyloxy)-2-(3,4,5-tris(benzyloxy)phenyl)chroman-3-ol (1.1 g, 1.190 mmol, 1 eq.), DMAP (0.58 g, 4.760 mmol, 4 eq.) and Et₃N (0.7 mL, 4.760 mmol, 4 eq.) in CH₂Cl₂ (5 mL) at 0° C. Then the reaction mixture was stirred at RT for 16 h. Finally, the reaction was quenched with saturated NaHO₃ aqueous solution (5 mL). The organic layer was separated and the aqueous layer was extracted with CH₂Cl₂ (30 mL). Combined organic phase was dried over MgSO₄ filtered and concentrated under reduced pressure. Obtained crude compound was purified by flash column chromatography (EtOAc in hexane) to get (2S,3R)-5,7-bis(benzyloxy)-2-(3,4,5-tris(benzyloxy)phenyl)chroman-3-yl 3,4-bis(benzyloxy)-5-(difluoromethoxy)benzoate as a light yellow solid (1 g, 75% yield). ¹H NMR (400 MHZ, DMSO-d₆): δ 7.45-7.21 (m, 36H), 7.14 (s, 1H), 6.94 (s, 2H), 6.45 (d, J=2 Hz, 1H), 6.29 (d, J=1.6 Hz, 1H), 5.44 (d, J=5.2 Hz, 1H), 5.22 (d, J=7.2 Hz, 1H), 5.20 (s, 2H), 5.18 (s, 2H), 5.14 (s, 2H), 5.12 (s, 2H), 5.08 (s, 2H), 4.96 (s, 2H), 4.87 (s, 2H), 3.04 (dd, J=7.2 Hz, 2H), 2.82 (dd, J=7.2 Hz, 2H), ¹⁹F NMR (400 MHZ, DMSO-d₆) δ−81.7, −81.38. LCMS: (M+H⁺): m/z: 1139.53.

Step 3: Synthesis of Compound 60. To a solution of (2S,3R)-5,7-bis(benzyloxy)-2-(3,4,5-tris(benzyloxy)phenyl)chroman-3-yl 3,4-bis(benzyloxy)-5-(difluoromethoxy)benzoate (1 g, 0.878 mmol, 1 eq.) in 8 mL of 1:1; THF:MeOH was added palladium hydroxide (20 wt. %, 1.0 g) at RT and the reaction mixture was stirred under hydrogen atmosphere for 16 h. Then the mixture was passed through a pad of celite to remove the catalyst. The filtrate was concentrated under vacuum. Obtained crude compound was purified by Prep-HPLC to obtain (2S,3R)-5,7-dihydroxy-2-(3,4,5-trihydroxyphenyl)chroman-3-yl 3-(difluoromethoxy)-4,5-dihydroxybenzoate as an off-white color solid (0.104 g, 31% yield). ¹H NMR (400 MHZ, DMSO-d₆): 9.09 (bs, 7H), 6.26 (s, 2H), 5.92 (d, J=2.0 Hz, 1H), 5.80 (d, J=2.0 Hz, 1H), 5.38 (q, J=5.2 Hz, 1H), 5.03 (d, J=5.2 Hz, 1H), 3.73 (s, 3H), 2.62 (dd, J=4 Hz, 1H), 2.55 (dd, J=4 Hz, 1H), ¹⁹F NMR (400 MHZ, DMSO-d₆) δ−81.11. LCMS: (M−H+): m/Z: 508.88.

### Compound 61

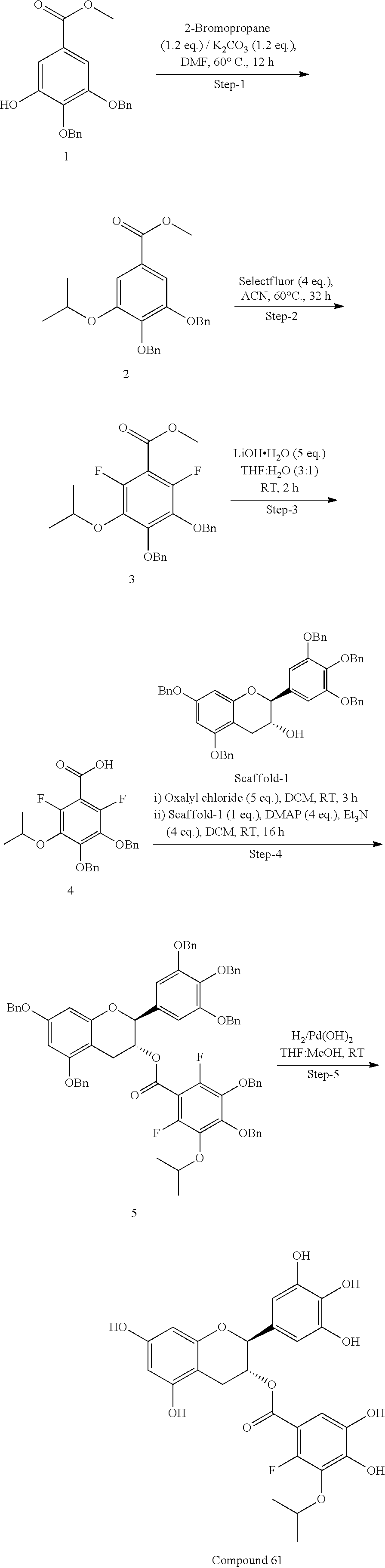

Step 1: Synthesis of methyl 3,4-bis(benzyloxy)-5-isopropoxybenzoate (2). To a suspension of methyl 3,4-bis(benzyloxy)-5-hydroxybenzoate (10.0 g, 27.470 mmol) in DMF (100 mL) was added K₂CO₃ (5.73 g, 41.20 mmol, 1.2 eq.) followed by 2-bromopropane (5.08 g, 41.20 mmol, 1.2 eq.) at 0° C. The reaction mixture was heated to 60° C. for 12 h. After this time, reaction mass was diluted with water and extracted with EtOAc. Organic layer was evaporated, and the residue was purified by flash chromatography eluted with 25% EtOAc in hexane, as an eluent to get methyl 3,4-bis(benzyloxy)-5-isopropoxybenzoate as a white solid (8.2 g, 73% yield). ¹H NMR (400 MHZ, DMSO-d₆) δ 7.47 (d, J=1.2 Hz, 1H), 7.45-7.35 (m, 4H), 7.34-7.30 (m, 5H), 7.23 (d, J=2.0 Hz, 2H), 5.16 (s, 2H), 5.02 (s, 2H), 4.66-4.60 (m, 1H), 3.82 (s, 3H), 1.27 (s, 3H), 1.28 (s, 3H).

Step 2. Synthesis of methyl 3,4-bis(benzyloxy)-2,6-difluoro-5-isopropoxybenzoate (3). To a solution of methyl 3,4-bis(benzyloxy)-5-isopropoxybenzoate (12.2 g, 30.185 mmol, 1 eq.) in 60 mL ACN was added selectfluor (42.7 g, 120.743 mmol, 4 eq.) at 0° C. and reaction mixture was stirred at 60° C. for 32 h. Reaction progress was monitor by TLC. After this time, reaction mixture was quenched with cold water, extracted with EtOAc (3×100 mL). Combined organic layer was washed with brine and dried over anhydrous Na₂SO₄, filtered and concentrated under reduced pressure to obtained crude compound. The crude compound was purified by flash column chromatography to get methyl 3,4-bis(benzyloxy)-2,6-difluoro-5-isopropoxybenzoate as a green solid (1.1 g, 8% yield). ¹H NMR (400 MHZ, DMSO-d₆) δ 7.43-7.30 (m, 10H), 5.18 (s, 2H), 5.04 (s, 2H), 4.43-4.28 (m, 1H), 3.56 (s, 3H), 1.24 (s, 3H), 1.16 (s, 3H).

Step 3: Synthesis of 3,4-bis(benzyloxy)-2,6-difluoro-5-isopropoxybenzoic acid (4). A mixture of methyl 3,4-bis(benzyloxy)-2,6-difluoro-5-isopropoxybenzoate (1 g, 2.260 mmol, 1.0 eq.) in THF/H₂O (1:1) (20 mL) was added LiOH H₂O (0.284 g, 11.300 mmol, 5.0 eq.). The solution was stirred at RT for 2 h. The reaction mixture was concentrated to remove THF. Then the mixture was diluted with H₂O (30 mL) and extracted with EA (20 mL×1). The aqueous phase pH was adjusted to <3 with 1N HCl. Obtained solid was filtered and the solid was dried to give 3,4-bis(benzyloxy)-2,6-difluoro-5-isopropoxybenzoic acid as a white solid (0.91 g, 94% yield). ¹H NMR (400 MHZ, DMSO-d₆) δ 13.82 (s, 1H), 7.43-7.30 (m, 10H), 5.15 (s, 2H), 5.04 (s, 2H), 4.39-4.36 (m, 1H), 1.21 (s, 3H), 1.20 (s, 3H). ¹⁹F NMR (400 MHZ, DMSO-d₆) δ 134.17, 134.18, 134.48, 134.48.

Step 4: Synthesis of (2S,3R)-5,7-bis(benzyloxy)-2-(3,4,5-tris(benzyloxy)phenyl)chroman-3-yl 3,4-bis(benzyloxy)-2,6-difluoro-5-isopropoxybenzoate (5). Under an N₂ atmosphere, to a stirred solution of 3,4-bis(benzyloxy)-2,6-difluoro-5-isopropoxybenzoic acid (0.9 g, 2.102 mmol, 1 eq.) in DCM (8 mL) was added oxalyl chloride (0.53 mL, 6.308 mmol, 3 eq.) and two drops of DMF at 0° C. The reaction mixture was stirred at RT for 1 h. After this time, the reaction mixture was concentrated under reduced pressure to get acid chloride. Obtained acid chloride was added to a solution of (2S,3R)-5,7-bis(benzyloxy)-2-(3,4,5-tris(benzyloxy)phenyl)chroman-3-ol (0.079 g, 1.051 mmol, 0.5 eq.), DMAP (1 g, 8.411 mmol, 4 eq.) and Et₃N (1.1 mL, 8.411 mmol, 4 eq.) in CH₂Cl₂ (15 mL) at 0° C. Then the reaction mixture was stirred at RT 16 h. Finally, the reaction was quenched with saturated aqueous NaHCO₃ solution (5 mL). The organic layer was separated and the aqueous layer was extracted with CH₂Cl₂ (30 mL). Combined organic phase was dried over MgSO₄, filtered and concentrated under reduced pressure. Obtained crude compound was purified by flash column chromatography (EtOAc in hexane) to get (2S,3R)-5,7-bis(benzyloxy)-2-(3,4,5-tris(benzyloxy)phenyl)chroman-3-yl 3,4-bis(benzyloxy)-2,6-difluoro-5-isopropoxybenzoate as a pale green solid (0.4 g, 32% yield). LCMS: (M+H⁺): m/Z: 1167.43.

Step 5: Synthesis of Compound 61. To a solution of (2S,3R)-5,7-bis(benzyloxy)-2-(3,4,5-tris(benzyloxy)phenyl)chroman-3-yl 3,4-bis(benzyloxy)-2,6-difluoro-5-isopropoxybenzoate (0.39 g, 0.334 mmol, 1 eq.), in 10 mL of 1:1 THF:MeOH was added palladium hydroxide (20 wt. %, 0.039 g) at RT and the reaction mixture was stirred under hydrogen atmosphere for 16 h. Then the mixture was passed through a pad of celite to remove the catalyst. The filtrate was concentrated under vacuum. Obtained crude compound was purified by Prep-HPLC to get (2S,3R)-5,7-dihydroxy-2-(3,4,5-trihydroxyphenyl)chroman-3-yl 2,6-difluoro-3,4-dihydroxy-5-isopropoxybenzoate as an off-white color solid (0.094 g, 52% yield). ¹H NMR (400 MHZ, DMSO-d₆): 9.06 (bs, 7H), 6.24 (s, 2H), 5.90 (d, J=2.0 Hz, 1H), 5.78 (d, J=2.4 Hz, 1H), 5.36 (q, J=4.8 Hz, 1H), 5.02 (d, J=4.8 Hz, 1H), 4.28-4.22 (s, 1H), 2.60 (d, J=4.0 Hz, 2H), 1.19 (s, 3H), 1.17 (s, 3H), ¹⁹F NMR (400 MHZ, DMSO-d₆)-139.80, −138.95. LCMS: (M−H⁺): m/Z: 536.96.

### Compound 62

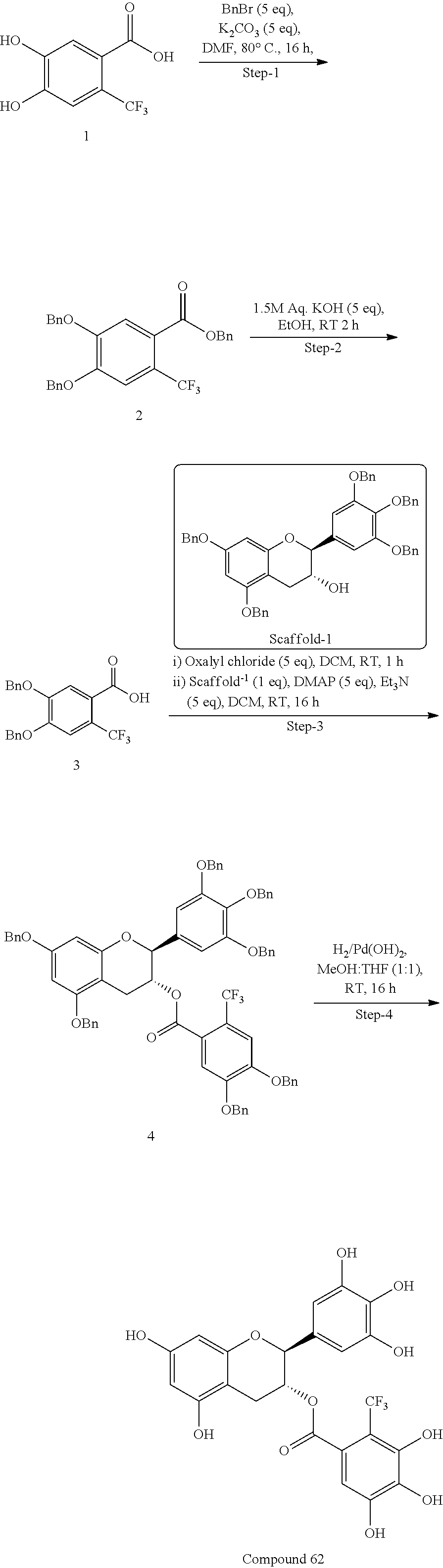 [SMILES: O=C(O)c1cc(O)c(O)cc1C(F)(F)F ; O=C(OCc1ccccc1)c1cc(OCc2ccccc2)c(OCc2ccccc2)cc1C(F)(F)F ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1cc(OCc2ccccc2)c(OCc2ccccc2)cc1C(F)(F)F ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1cc(OCc2ccccc2)c(OCc2ccccc2)cc1C(F)(F)F ; O=C(O[C@@H]1Cc2c(O)cc(O)cc2O[C@H]1c1cc(O)c(O)c(O)c1)c1cc(O)c(O)c(O)c1C(F)(F)F]

Step 1: Synthesis of benzyl 4,5-bis(benzyloxy)-2-(trifluoromethyl)benzoate (2). To a solution of 4,5-dihydroxy-2-(trifluoromethyl)benzoic acid (0.5 g, 2.251 mmol, 1 eq.) and K₂CO₃ (1.56 g, 11.26 mmol, 4 eq.) in DMF (20 mL) was added BnBr (1.34 ml, 11.26 mmol, 4 eq.) at 0° C., reaction mixture was stirred at 60° C. for 10 h. Reaction progress was monitored by TLC. After this time, reaction mixture was diluted with ice-cold water (50 mL) and extracted with ethyl acetate (50 mL×3), washed with brine and dried over Na₂SO₄. Organic layer was evaporated under reduced pressure to obtain crude compound. The crude was purified by flash chromatography, eluted with 10% EtOAc in hexane as an eluent affords to obtain benzyl 4,5-bis(benzyloxy)-2-(trifluoromethyl)benzoate (0.710 g, 64% yield) as an off-white color solid. 1H NMR (400 MHZ, DMSO-d₆): 7.45-7.32 (m, 15H), 7.27 (s, 1H), 7.26 (s, 1H), 5.32 (s, 2H), 5.21 (s, 2H), 5.20 (s, 1H).

Step 2: Synthesis of 4,5-bis(benzyloxy)-2-(trifluoromethyl)benzoic acid (3). To a solution of benzyl 4,5-bis(benzyloxy)-2-(trifluoromethyl)benzoate (0.7 g, 1.42 mmol, 1.0 eq.) in ethanol (24 mL) was added 1.5M Aq. KOH (0.39 g, 7.106 mmol, 5 eq.) at 25° C., and was stirred at RT 2 h. Reaction progress was monitored by TLC. After this time, reaction mixture was concentrated under reduced pressure to obtained crude compound. Obtained crude compound was diluted with water and the aqueous layer pH was adjusted with 1N HCl up to 2, precipitate was formed. The solid compound was collected through filtration and dried at high vacuum to obtain 4,5-bis(benzyloxy)-2-(trifluoromethyl)benzoic acid (0.69 mg, 98% yield) as an off-white color solid. ¹H NMR (400 MHZ, DMSO-d₆): 13.33 (Br s, 1H), 7.46-7.32 (m, 10H), 5.29 (s, 2H), 5.27 (s, 2H).

Step 3: Synthesis of (2S,3R)-5,7-bis(benzyloxy)-2-(3,4,5-tris(benzyloxy)phenyl)chroman-3-yl 4,5-bis(benzyloxy)-2-(trifluoromethyl)benzoate (4). To a solution of 4,5-bis(benzyloxy)-2-(trifluoromethyl)benzoic acid (3 g, 0.560 g, 1.4 mmol, 1 eq.) in DCM (10 mL) was added oxalyl chloride (0.6 mL, 0.70 mmol, 5 eq) and two drops of DMF at 0° C. and was stirred at RT for 1 h. After this time, the reaction mixture was concentrated under reduced pressure to get acid chloride. Obtained acid chloride was added to a solution of (2S,3R)-5,7-bis(benzyloxy)-2-(3,4,5-tris(benzyloxy)phenyl)chroman-3-ol (0.6 g, 0.79 mmol, 0.8 eq.), Et₃N (0.97 ml, 0.7 mmol, 5 eq.) and DMAP (0.85 g, 0.7 mmol, 5 eq) in DCM (10 mL) at 0° C. Then the reaction mixture was stirred at RT 16 h. Finally, the reaction was quenched with saturated aqueous NaHCO₃ solution (5 mL). The organic layer was separated and the aqueous layer was extracted with CH₂Cl₂ (30 mL). Combined organic phase was dried over MgSO₄. filtered and concentrated under reduced pressure. Obtained crude compound was purified by flash column chromatography (EtOAc in hexane) to get (2S,3R)-5,7-bis(benzyloxy)-2-(3,4,5-tris(benzyloxy)phenyl)chroman-3-yl 4,5-bis(benzyloxy)-2-(trifluoromethyl)benzoate (0.315 g, 20% yield) as pale green color solid. LCMS: 74.60%, (M+H=1141.48).

Step 4: Synthesis of Compound 62. To a solution of (2S,3R)-5,7-bis(benzyloxy)-2-(3,4,5-tris(benzyloxy)phenyl)chroman-3-yl 4,5-bis(benzyloxy)-2-(trifluoromethyl)benzoate (0.31 g, 0.271 mmol) in THF (15 mL) and MeOH (15 mL) was added Pd(OH)₂ (20 wt. %, 0.31 g) at 25° C. Reaction mixture was stirred at 25° C., under H₂ atmosphere for 16 h. Reaction progress was monitored by LCMS. Reaction mixture was passed through celite-pad and filtrate was evaporated under reduced pressure to obtain crude compound. The crude compound was purified by prep-HPLC to obtain (2S,3R)-5,7-dihydroxy-2-(3,4,5-trihydroxyphenyl)chroman-3-yl 4,5-dihydroxy-2-(trifluoromethyl)benzoate (0.074 g, 53% yield) as a pale pink color solid. ¹H NMR (400 MHZ, DMSO-d₆): δ 9.32 (bs, 7H), 7.01 (s, 2H), 6.25 (s, 2H), 5.91-5.90 (d, J=2 Hz, 1H), 5.79-5.78 (d, J=2.0 Hz, 1H), 5.33-5.30 (q, J=4.8 Hz, 1H), 5.04-5.03 (d, J=4.8 Hz, 1H), 2.66-2.54 (m, 2H). LCMS: 99.73%, (M+H=510.83).

### Compound 63

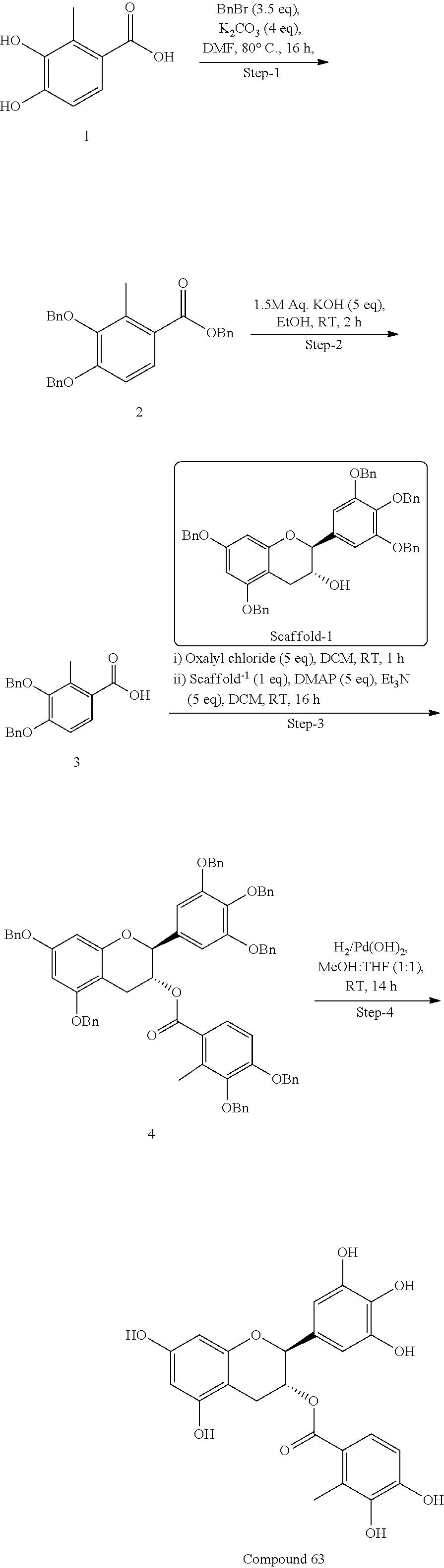 [SMILES: Cc1c(C(=O)O)ccc(O)c1O ; Cc1c(C(=O)OCc2ccccc2)ccc(OCc2ccccc2)c1OCc1ccccc1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; Cc1c(C(=O)O)ccc(OCc2ccccc2)c1OCc1ccccc1 ; Cc1c(C(=O)O[C@@H]2Cc3c(OCc4ccccc4)cc(OCc4ccccc4)cc3O[C@H]2c2cc(OCc3ccccc3)c(OCc3ccccc3)c(OCc3ccccc3)c2)ccc(OCc2ccccc2)c1OCc1ccccc1 ; Cc1c(C(=O)O[C@@H]2Cc3c(O)cc(O)cc3O[C@H]2c2cc(O)c(O)c(O)c2)ccc(O)c1O]

Step 1: Synthesis of benzyl 3,4-bis(benzyloxy)-2-methylbenzoate (2). To a solution of 3,4-dihydroxy-2-methylbenzoic acid (0.5 g, 2.97 mmol, 1 eq.) and K₂CO₃ (1.64 g, 10.416 mmol, 4 eq.) in DMF (20 mL) was added BnBr (1.42 ml, 11.88 mmol, 4 eq.) at 0° C. and was stirred at 80° C. for 16 h. Reaction progress was monitored by TLC. Reaction mixture was diluted with ice-cold water (50 mL) and product was extracted with ethyl acetate (3×50 mL), washed with brine and dried over Na₂SO₄. Organic layer was concentrated under reduced pressure to obtain crude compound. The crude was purified by flash chromatography, eluted with 10% EtOAc in hexane, as an eluent affords to obtain benzyl 3,4-bis(benzyloxy)-2-methylbenzoate (1.1 g, 85% yield) as an off-white color solid. ¹H NMR (400 MHZ, DMSO-d₆): 7.76-7.74 (d, J=8.4 Hz, 1H), 7.45-7.31 (m, 15H), 6.86-6.84 (d, J=8.8 Hz, 1H), 5.31 (s, 2H), 5.17 (s, 2H), 4.94 (s, 2H), 2.53 (s, 3H).

Step 2: Synthesis of 3,4-bis(benzyloxy)-2-methylbenzoic acid (3). To a solution of benzyl 3,4-bis(benzyloxy)-2-methylbenzoate (1.2 g, 2.736 mmol, 1.0 eq.) in ethanol (20 mL) was added 1.5M Aq. KOH (0.768 g, 13.682 mmol, 5 eq.) at 25° C. and was stirred at rt for 2 h. Reaction progress was monitored by TLC. After this time, reaction mixture was concentrated under reduced pressure to obtained crude compound. Obtained crude compound was diluted with water, the aqueous layer pH was adjusted with 1N HCl up to 2, precipitate was formed. The solid compound was collected through filtration and dried at high vacuum to obtain 3,4-bis(benzyloxy)-2-methylbenzoic acid (0.94 g, 98% yield) as an off-white color solid. ¹H NMR (400 MHZ, DMSO-d₆): δ 12.53 (bs, 1H), 7.66-7.64 (d, J=8.4 Hz, 1H), 7.52-7.50 (m, 3H), 7.43-7.32 (m, 8H), 7.11-7.09 (d, J=8.8 Hz, 1H), 5.24 (s, 2H), 4.90 (s, 2H), 2.40 (s, 3H).

Step 3: Synthesis of (2S,3R)-5,7-bis(benzyloxy)-2-(3,4,5-tris(benzyloxy)phenyl)chroman-3-yl 3,4-bis(benzyloxy)-2-methylbenzoate (4). To a stirred solution of 3,4-bis(benzyloxy)-2-methylbenzoic acid (0.86 g, 2.468 mmol, 1 eq.) in DCM (10 mL) was added oxalyl chloride (1.0 mL, 12.342 mmol, 5 eq.) and two drops DMF at 0° C. The reaction mixture was stirred at RT for 1 h. After this time, the reaction mixture was concentrated under reduced pressure to get acid chloride. Obtained acid chloride was added to a solution of (2S,3R)-5,7-bis(benzyloxy)-2-(3,4,5-tris(benzyloxy)phenyl)chroman-3-ol (1.49 g, 1.974 mmol, 0.8 eq.), Et₃N (0.32 ml, 12.342 mmol, 5 eq.) and DMAP (1.5 g, 12.342 mmol, 5 eq) in DCM (10 mL) at 0° C., Then the reaction mixture was stirred at RT 16 h. Finally, the reaction was quenched with saturated aqueous NaHCO₃ solution (5 mL). The organic layer was separated and the aqueous layer was extracted with CH₂Cl₂ (30 mL). Combined organic phase was dried over MgSO₄, filtered and concentrated under reduced pressure. Obtained crude compound was purified by flash column chromatography (EtOAc in hexane) to get (2S,3R)-5,7-bis(benzyloxy)-2-(3,4,5-tris(benzyloxy)phenyl)chroman-3-yl 3,4-bis(benzyloxy)-2-methylbenzoate (0.24 g, 19% yield) as an off-white color solid. LCMS: 71.08%, (M−H=1087.35).

Step 4: Synthesis of Compound 63. To a solution of (2S,3R)-5,7-bis(benzyloxy)-2-(3,4,5-tris(benzyloxy)phenyl)chroman-3-yl 3,4-bis(benzyloxy)-2-methylbenzoate (0.58 g, 0.533 mmol) in THF (15 mL) and MeOH (15 mL) was added Pd(OH)₂ (10 wt. %, 0.58 g) at RT. Reaction mixture was stirred at 25° C., under H₂ atmosphere for 16 h. Reaction progress was monitored by LCMS. Reaction mixture was filtered through celite-pad and filtrate was evaporated under reduced pressure to obtain crude compound. The crude compound was purified by prep-HPLC to obtain (2S,3R)-5,7-dihydroxy-2-(3,4,5-trihydroxyphenyl)chroman-3-yl 3,4-dihydroxy-2-methylbenzoate (0.062 g, 25% yield) as a pale pink color solid. ¹H NMR (400 MHz, DMSO-d₆): δ 9.05 (bs, 7H), 7.04-7.02 (d, J=8.8 Hz, 1H), 6.61-6.59 (d, J=8.8 Hz, 1H), 5.92-5.91 (d, J=1.6 Hz, 1H), 5.79-5.78 (d, J=2.0 Hz, 1H), 5.24-5.20 (q, J=5.2 Hz, 1H), 5.00-4.99 (d, J=5.6 Hz, 1H), 2.68-2.53 (m, 2H), 2.21 (s, 3H). LCMS: 99.72%, (M+H=456.85).

### Compound 64

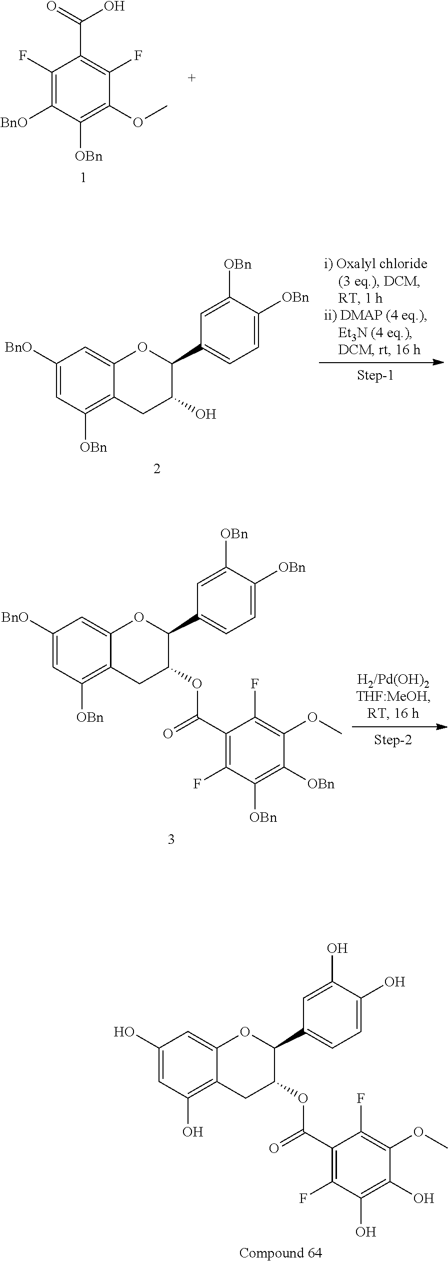 [SMILES: COc1c(F)c(C(=O)O)c(F)c(OCc2ccccc2)c1OCc1ccccc1 ; O[C@@H]1Cc2c(OCc3ccccc3)cc(OCc3ccccc3)cc2O[C@H]1c1ccc(OCc2ccccc2)c(OCc2ccccc2)c1 ; COc1c(F)c(C(=O)O[C@@H]2Cc3c(OCc4ccccc4)cc(OCc4ccccc4)cc3O[C@H]2c2ccc(OCc3ccccc3)c(OCc3ccccc3)c2)c(F)c(OCc2ccccc2)c1OCc1ccccc1 ; COc1c(O)c(O)c(F)c(C(=O)O[C@@H]2Cc3c(O)cc(O)cc3O[C@H]2c2ccc(O)c(O)c2)c1F]

Step 2: Synthesis of (2S,3R)-5,7-bis(benzyloxy)-2-(3,4-bis(benzyloxy)phenyl)chroman-3-yl 3,4-bis(benzyloxy)-2,6-difluoro-5-methoxybenzoate (3). Under an N₂ atmosphere, to a stirred solution of 3,4-bis(benzyloxy)-2,6-difluoro-5-methoxybenzoic acid (1.2 g, 1.936 mmol, 1 eq.) in DCM (8 mL) was added oxalyl chloride (0.49 mL, 5.808 mmol, 3 eq.) and two drops of DMF at 0° C. The reaction mixture was stirred at RT for 1 h. After this time, the reaction mixture was concentrated under reduced pressure to get acid chloride. Obtained acid chloride was added to a solution of (2S,3R)-5,7-bis(benzyloxy)-2-(3,4-bis(benzyloxy)phenyl)chroman-3-ol (1 g, 2.710 mmol, 1.4 eq.), DMAP (0.9 g, 7.744 mmol, 4 eq.) and Et₃N (0.94 mL, 7.744 mmol, 4 eq.) in CH₂Cl₂ (15 mL) at 0° C. The mixture was stirred at RT overnight, and then saturated NaHO₃ aqueous solution was added. The organic layer was separated, and the aqueous layer was extracted with CH₂Cl₂. The organic phases were combined, dried (MgSO₄) and evaporated. The crude compound was purified by flash column chromatography, eluted with 20% EtOAc in hexane, as an eluent affords to obtain (2S,3R)-5,7-bis(benzyloxy)-2-(3,4-bis(benzyloxy)phenyl)chroman-3-yl3,4-bis(benzyloxy)-2,6-difluoro-5-methoxybenzoate as a white solid (0.41 g, 21% yield). ¹H NMR (400 MHZ, DMSO-d₆): 7.41-7.26 (m, 30H), 7.12 (s, 1H), 7.03 (d, J=8.4 Hz, 1H), 6.90 (d, J=8.4 Hz, 1H), 6.37 (d, J=2 Hz, 1H), 6.24 (d, J=1.6 Hz, 1H), 5.74 (s, 1H), 5.60 (d, J=5.6 Hz, 1H), 5.21 (d, J=6 Hz, 1H), 5.15 (s, 2H), 5.09 (s, 4H), 5.04 (s, 4H), 4.95 (s, 2H), 3.79 (s, 3H), 2.78 (dd, J=7.6 Hz, 2H), ¹⁹F NMR (400 MHZ, DMSO-d₆) δ−133.54, 134.49. LCMS: (M+H⁺): m/Z: 1033.36.

Step 2: Synthesis of Compound 64. To a solution of (2S,3R)-5,7-bis(benzyloxy)-2-(3,4-bis(benzyloxy)phenyl)chroman-3-yl 3,4-bis(benzyloxy)-2,6-difluoro-5-methoxybenzoate (0.39 g, 0.377 mmol, 1 eq.) in 10 mL of 1:1 THF:MeOH was added palladium hydroxide (20 wt. %, 0.39 g) at RT and the reaction mixture was stirred under hydrogen atmosphere for 16 h. Then the mixture was passed through a pad of celite to remove the catalyst. The filtrate was concentrated under reduced pressure. Obtained crude compound was purified by Prep-HPLC to obtain (2S,3R)-5,7-dihydroxy-2-(3,4,5-trihydroxyphenyl)chroman-3-yl 3-fluoro-4,5-dihydroxybenzoate as an pale pink color solid (0.09 g, 49% yield). ¹H NMR (400 MHZ, DMSO-d₆): 9.18 (bs, 6H), 6.71 (d, J=4 Hz, 1H), 6.67 (d, J=8.4 Hz, 1H), 6.58 (d, J=8 Hz, 1H), 5.89 (d, J=2.4 Hz, 1H), 5.79 (d, J=2.4 Hz, 1H), 5.36 (q, J=5.2 Hz, 1H), 5.01 (d, J=4.8 Hz, 1H), 3.67 (s, 3H), 2.58 (dd, J=5.6 Hz, 2H), ¹⁹F NMR (400 MHZ, DMSO-d₆) δ−141.56, 139.02. LCMS: (M−H+): m/Z: 493.02.

### Compound 65

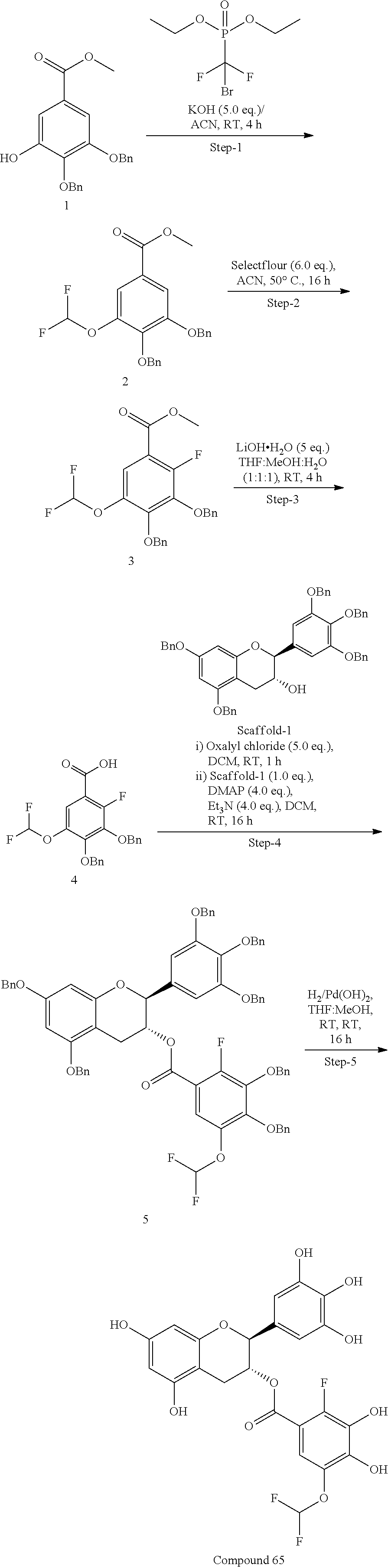

Step 1: Synthesis of methyl 3, 4-bis(benzyloxy)-5-(difluoromethoxy)benzoate (2). To a solution of methyl 3,4-bis(benzyloxy)-5-hydroxybenzoate (1.2 g, 3.29 mmol, 1.0 eq.) in CH₃CN:H₂O (6:4) (10 mL) was added KOH (0.92 g, 16.48 mmol, 5.0 eq.) at room temperature and stirred for 20 min. Then the mixture was cooled to −78° C. and added diethyl (bromodifluoromethyl)phosphonate (2.64 g, 9.89 mmol, 3.0 eq). The mixture was allowed to RT and stirred for 4 h. Finally, the reaction mixture was diluted with H₂O (50 mL), neutralized with 1N HCl and extracted with ethyl acetate (100 mL×2). The combined organic layers were washed with brine (50 mL), dried over Na₂SO₄, filtered and concentrated. The residue was purified by flash column chromatography on silica gel (PE/EA=9/1) to give methyl 3, 4-bis(benzyloxy)-5-(difluoromethoxy)benzoate (0.48 g, 35% yield) as a yellow solid. ¹H NMR (400 MHz, DMSO-d₆): δ 7.60 (d, J=2.0 Hz, 1H), 7.52-7.47 (m, 2H), 7.45-7.30 (m, 9H), 7.20 (t, J=73.6 Hz, 1H), 5.26 (s, 2H), 5.09 (s, 2H), 3.85 (s, 3H).

Step 2: Synthesis of methyl 3,4-bis(benzyloxy)-5-(difluoromethoxy)-2-fluorobenzoate (3). A mixture of methyl 3, 4-bis(benzyloxy)-5-(difluoromethoxy)benzoate (1.2 g, 2.89 mmol, 1.0 eq.) in CH₃CN (12 mL) was added selectfluor (6.15 g, 17.39 mmol, 6.0 eq.) at 0° C. and stirred at RT for 1 h. Then reaction mixture was warmed to 50° C. and stirred for another 16 h. After completion of the reaction, reaction mass was cooled to RT, diluted with H₂O (50 mL) and extracted with EtOAc (2×100 mL). The combined organic layers was washed with brine (50 mL), dried over Na₂SO₄, filtered and concentrated. The residue was purified by flash column chromatography on silica gel (PE/EA=9/1) to give methyl 3,4-bis(benzyloxy)-5-(difluoromethoxy)-2-fluorobenzoate (0.051 g, 4% yield) as a pale yellow solid. ¹H NMR (400 MHz, CDCl₃): δ 7.50 (d, J=6.4 Hz, 1H), 7.45-7.32 (m, 9H), 6.38 (t, J=74.0 Hz, 1H), 5.15 (s, 2H), 5.11 (s, 2H), 3.92 (s, 3H).

Step 3: Synthesis of 3,4-bis(benzyloxy)-5-(difluoromethoxy)-2-fluorobenzoic acid (4). To a solution of methyl 3,4-bis(benzyloxy)-5-(difluoromethoxy)-2-fluorobenzoate (0.25 g, 0.57 mmol, 1.0 eq.) in MeOH:THF:H₂O (1:1:1) (6 mL) was added LiOH (0.07 g, 2.89 mmol, 5.0 eq.) at 0° C. and stirred the mixture at RT for 4 h. After completion of the reaction solvent was evaporated under reduced pressure. The obtained solid was diluted with H₂O (20 mL), acidified with 1N HCl (pH=2-3) and extracted with ethyl acetate (3×50 mL). The combined organic layers were dried over anhydrous Na₂SO₄, evaporated under reduced pressure to give 3,4-bis(benzyloxy)-5-(difluoromethoxy)-2-fluorobenzoic acid (0.215 g, 89% yield) as a white solid. ¹H NMR (400 MHZ, DMSO-d₆): 7.45-7.32 (m, 9H), 7.16 (t, J=73.2 Hz, 1H), 5.15 (s, 2H), 5.10 (s, 2H).

Step 4: Synthesis of (2S,3R)-5,7-bis(benzyloxy)-2-(3,4,5-tris(benzyloxy)phenyl)chroman-3-yl 3,4-bis(benzyloxy)-5-(difluoromethoxy)-2-fluorobenzoate (4). To a solution of 3,4-bis(benzyloxy)-5-(difluoromethoxy)-2-fluorobenzoic acid (0.215 g, 0.51 mmol, 1.0 eq.) in CH₂Cl₂ (5 mL) was added (COCl)₂ (0.25 mL, 2.57 mmol, 5.0 eq.) and 2 drops of dry DMF at 0° C. The mixture was stirred at RT for 1 h. After completion of acid-chloride formation, solvent was evaporated from the reaction mixture and dried under reduced pressure. To this was added a mixture of (2S,3R)-5,7-bis(benzyloxy)-2-(3,4,5-tris(benzyloxy)phenyl)chroman-3-ol (0.39 g, 0.51 mmol, 1.0 eq.), DMAP (0.250 g, 2.05 mmol, 4.0 eq.) and TEA (0.3 mL, 2.05 mmol, 4.0 eq.) in CH₂Cl₂ (5 mL) at 0° C. The resulting mixture was stirred at RT for 16 h. The reaction mixture was diluted with H₂O (30 mL) and extracted with CH₂Cl₂ (2×50 mL). The combined organic layers were washed with brine (30 mL), dried over Na₂SO₄, filtered and concentrated. The residue was purified by flash column chromatography on silica gel (PE/EA=6/1) to give 5 (0.21 g, 64% yield) as a white solid. ¹H NMR (400 MHZ, CDCl₃) δ 7.51-7.30 (m, 33H), δ 7.27-7.21 (m, 3H), 6.77 (s, 2H), 6.34 (t, J=74.0 Hz, 1H), 6.32 (d, J=2.0 Hz, 1H), 6.29 (d, J=2.0 Hz, 1H), 5.51 (q, J=7.2 Hz, 1H), 5.12-4.98 (m, 15H), 3.14 (dd, J=10.8, 5.6 Hz, 1H), 3.14 (dd, J=8.8, 8.0 Hz, 1H).

Step 5: Synthesis of Compound 65. To a mixture of (2S,3R)-5,7-bis(benzyloxy)-2-(3,4,5-tris(benzyloxy)phenyl)chroman-3-yl 3,4-bis(benzyloxy)-5-(difluoromethoxy)-2-fluorobenzoate (0.2 g, 0.17 mmol, 1.0 eq.) in THF (2.5 mL) and MeOH (2.5 mL) was added Pd(OH)₂/C (20 wt. %, 120 mg). The mixture was stirred at room temperature under H₂ atmosphere for overnight. The reaction mixture was passed through a pad of celite and the filtrate was concentrated. The residue was purified by prep-HPLC to give (2S,3R)-5,7-dihydroxy-2-(3,4,5-trihydroxyphenyl)chroman-3-yl 5-(difluoromethoxy)-2-fluoro-3,4-dihydroxybenzoate (40 mg, 44% yield) as a pale pink solid. ¹H NMR (400 MHZ, DMSO-d₆) δ 8.98 (bs, 7H), 6.97 (t, J=74.4 Hz, 1H), 6.97 (d, J=6.4 Hz, 1H), 6.25 (s, 2H), 5.91 (d, J=2.4 Hz, 1H), 5.78 (d, J=2.4 Hz, 1H) 5.29 (q, J=5.2 Hz, 1H), 5.02 (d, J=5.6 Hz, 1H), 2.62 (d, J=5.2 Hz, 2H).

### Compound 66

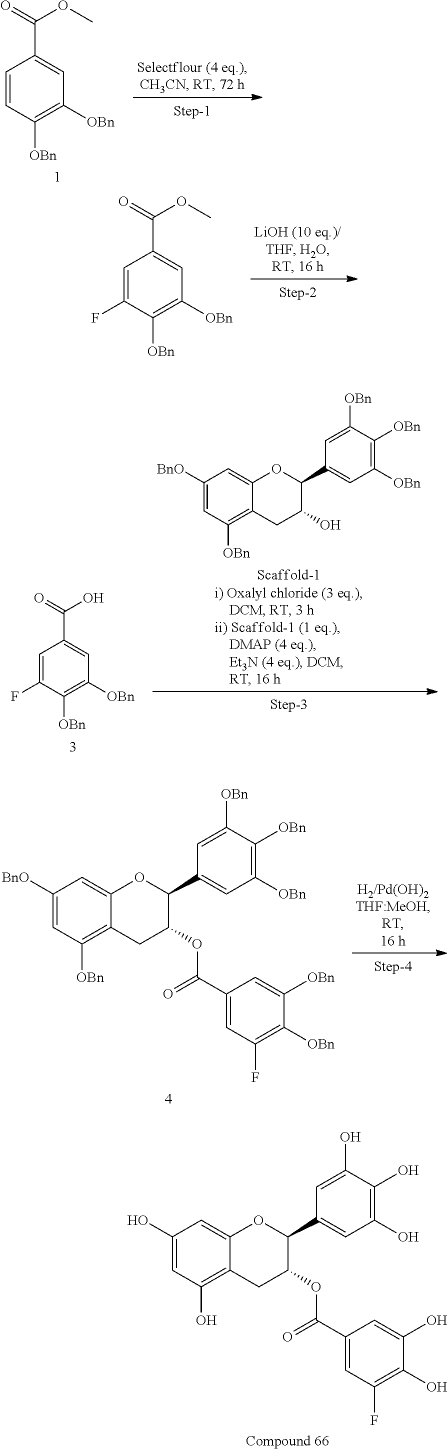 [SMILES: COC(=O)c1ccc(OCc2ccccc2)c(OCc2ccccc2)c1 ; COC(=O)c1cc(F)c(OCc2ccccc2)c(OCc2ccccc2)c1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; O=C(O)c1cc(F)c(OCc2ccccc2)c(OCc2ccccc2)c1 ; O=C(O[C@@H]1Cc2c(OCc3ccccc3)cc(OCc3ccccc3)cc2O[C@H]1c1cc(OCc2ccccc2)c(OCc2ccccc2)c(OCc2ccccc2)c1)c1cc(F)c(OCc2ccccc2)c(OCc2ccccc2)c1 ; O=C(O[C@@H]1Cc2c(O)cc(O)cc2O[C@H]1c1cc(O)c(O)c(O)c1)c1cc(O)c(O)c(F)c1]

Step 1: Synthesis of methyl 3,4-bis(benzyloxy)-5-fluorobenzoate (2). To a solution of methyl 3,4-bis(benzyloxy)benzoate (11.7 g, 33.620 mmol, 1 eq.), in ACN (50 mL) was added selectfluor (47.7 g, 134.48 mmol, 4 eq.) at 0° C. and reaction mixture was stirred at RT for 72 h. Reaction progress was monitor by TLC. After this time, reaction mixture was quenched with cold water, extracted with EtOAc (3×100 mL), washed with brine and dried over anhydrous Na₂SO₄. Organic layer was concentrated under reduced pressure to obtained crude compound. The crude compound was purified by flash column chromatography, eluted with 15% EtOAc in hexane as an eluent affords to obtain methyl 3,4-bis(benzyloxy)-5-fluorobenzoate as a yellow solid (1.8 g, 15% yield). ¹H NMR (400 MHZ, DMSO-d₆) δ 7.58 (d, J=2.0 Hz, 1H), 7.56 (d, J=1.6 Hz, 1H), 7.46-7.43 (m, 4H), 7.41-7.29 (m, 5H), 7.18 (d, J=9.2 Hz, 1H), 5.22 (s, 2H), 5.17 (s, 2H), 3.79 (s, 3H), ¹⁹F NMR (375 MHZ, DMSO-de) δ−114.10.

Step 2: Synthesis of 3,4-bis(benzyloxy)-5-fluorobenzoic acid (3). A mixture of methyl 3,4-bis(benzyloxy)-5-fluorobenzoate (1.8 g, 5.021 mmol, 1 eq.) in THF/H₂O (2:1) (30 mL) was added LiOH·H₂O (2.1 g, 50.210 mmol, 10 eq.). The solution was stirred at RT for 16 h. The reaction mixture was concentrated to remove THF. Obtained crude was diluted with H₂O (20 mL) and extracted with EA (1×20 mL). The aqueous phase pH was adjusted to <3 with 1N HCl. Then the mixture was filtered and the filter cake was dried to obtained 3,4-bis(benzyloxy)-5-fluorobenzoic acid as a white solid (0.8 g, 46% yield). ¹H NMR (400 MHZ, DMSO-d₆) δ 12.73 (s, 1H), 7.64 (s, 1H), 7.58 (d, J=2.0 Hz, 1H), 7.41-7.28 (m, 9H), 7.14 (d, J=8.8 Hz, 1H), 5.21 (s, 2H), 5.12 (s, 2H).

Step 3: Synthesis of (2S,3R)-5,7-bis(benzyloxy)-2-(3,4,5-tris(benzyloxy)phenyl)chroman-3-yl 3,4-bis(benzyloxy)-5-fluorobenzoate (4). Under an N₂ atmosphere, to a stirred solution of 3,4-bis(benzyloxy)-5-fluorobenzoic acid (0.77 g, 1.085 mmol, 1 eq.) in DCM (8 mL) was added oxalyl chloride (0.26 mL, 3.055 mmol, 3 eq.) and two drops of DMF at 0° C. The reaction mixture was stirred at RT for 1 h. The excess oxalyl chloride were removed by distillation and the residue was dried to give acid chloride. Obtained acid chloride was added dropwise to a solution of (2S,3R)-5,7-bis(benzyloxy)-2-(3,4,5-tris(benzyloxy)phenyl)chroman-3-ol (0.53 g, 1.527 mmol, 1.5 eq.), DMAP (0.497 g, 4.074 mmol, 4 eq.) and Et₃N (0.54 mL, 4.074 mmol, 4 eq.) in CH₂Cl₂ (10 mL) at 0° C. Then the reaction mixture was stirred at RT 16 h. Finally, the reaction was quenched with saturated aqueous NaHCO₃ solution (5 mL). The organic layer was separated and the aqueous layer was extracted with CH₂Cl₂ (30 mL). Combined organic phase was dried over MgSO₄, filtered and concentrated under reduced pressure. Obtained crude compound was purified by flash column chromatography (EtOAc in hexane) to get (2S,3R)-5,7-bis(benzyloxy)-2-(3,4,5-tris(benzyloxy)phenyl)chroman-3-yl 3,4-bis(benzyloxy)-5-fluorobenzoate as a yellow solid (0.36 g, 31% yield). ¹H NMR (400 MHZ, DMSO-d₆) δ 7.45-7.10 (m, 36H), 7.11 (d, J=2.4 Hz, 1H), 6.87 (d, J=2.0 Hz, 2H), 6.43 (d, J=2.4 Hz, 1H), 6.28 (d, J=2.4 Hz, 1H), 5.46 (q, J=5.6 Hz, 1H), 5.16 (d, J=8.8 Hz, 1H), 5.12 (s, 2H), 5.04 (s, 6H), 4.98 (s, 2H), 4.89 (s, 2H), 3.85 (s, 2H), 2.95 (dd, J=7.4 Hz, 1H), 2.77 (dd, J=8.8 Hz, 1H), ¹⁹F NMR (400 MHz, DMSO-d₆) δ−114.00. LCMS: (M+H⁺): m/Z: 1091.4.

Step 4: Synthesis of Compound 66. To a solution of (2S,3R)-5,7-bis(benzyloxy)-2-(3,4,5-tris(benzyloxy)phenyl)chroman-3-yl 3,4-bis(benzyloxy)-5-fluorobenzoate (0.35 g, 0.321 mmol, 1 eq.), in 10 mL of 1:1 THF:MeOH, palladium hydroxide (20 wt. %, 0.35 g) was added at RT, reaction mixture stirred under a hydrogen atmosphere for 16 h. Then the mixture was passed through a pad of celite to remove the catalyst. The filtrate was concentrated under reduced pressure. Obtained crude compound was purified by Prep-HPLC to get (2S,3R)-5,7-dihydroxy-2-(3,4,5-trihydroxyphenyl)chroman-3-yl 3-fluoro-4,5-dihydroxybenzoate as an off-white solid (0.040 g, 52% yield). ¹H NMR (400 MHZ, DMSO-d₆): δ 9.02 (bs, 7H), 7.07 (d, J=7.6 Hz, 1H), 6.52 (d, J=7.6 Hz, 1H), 6.25 (s, 2H), 5.91 (d, J=2.4 Hz, 1H), 5.79 (d, J=2.4 Hz, 1H), 5.26 (q, J=5.2 Hz, 1H), 5.01 (d, J=5.2 Hz, 1H), 2.60 (d, J=4.8 Hz, 2H), ¹⁹F NMR (400 MHZ, DMSO-d₆) δ−118.04. LCMS: (M−H⁺): m/Z: 459.0.

### Compound 67

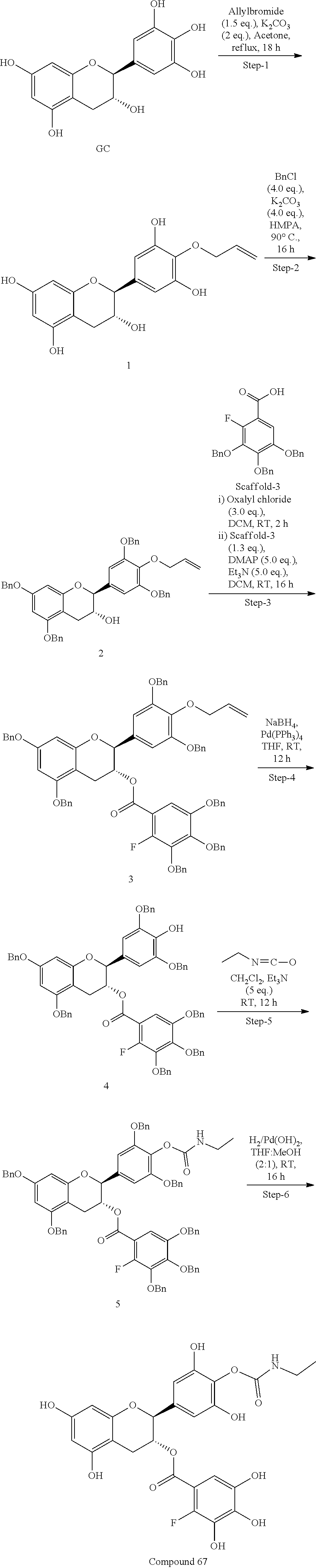

Step 1: Synthesis of (2S,3R)-2-(4-(allyloxy)-3,5-dihydroxyphenyl)chromane-3,5,7-triol (1). To a stirred solution of (2S,3R)-2-(3,4,5-trihydroxyphenyl)chromane-3,5,7-triol (12.0 g, 39.44 mmol, 1.0 eq.) in dry acetone (360 mL) was added K₂CO₃ (10.88 g, 78.89 mmol, 2.0 eq.) at 0° C. and stirred for 0.5 h at the same temperature. To this was added allyl bromide (4.09 mL, 47.33 mmol, 1.2 eq.) at 0° C. The resulting suspension was stirred at 55° C. for 18 h. Reaction progress was monitored by TLC. After complete consumption of the starting material, the solvent was evaporated under reduced pressure. Obtained residue was purified by reverse phase column chromatography (H₂O:CH₃CN, 1:9), to afford (2S,3R)-2-(4-(allyloxy)-3,5-dihydroxyphenyl)chromane-3,5,7-triol (2.34 g, 17.2% yield) as a brownish solid. ¹H NMR (400 MHz, MeOH-d₄): δ 6.46-6.37 (m, 2H), 6.20-6.06 (m, 1H), 5.92 (d, J=2.4 Hz, 1H), 5.86 (d, J=2.0 Hz, 1H), 5.29 (dd, J=17.2, 2.0 Hz, 1H), 5.15 (dd, J=9.6, 0.8 Hz, 1H), 4.65-4.48 (m, 3H), 3.97 (m, 1H), 3.34 (s, 2H), 2.80 (dd, J=16.4, 5.2 Hz, 1H), 2.50 (q, J=7.6 Hz, 1H).

Step 2: Synthesis of (2S,3R)-2-(4-(allyloxy)-3,5-bis(benzyloxy)phenyl)-5,7-bis(benzyloxy)chroman-3-ol (2). To a stirred solution of (2S,3R)-2-(4-(allyloxy)-3,5-dihydroxyphenyl) chromane-3,5,7-triol (7.0 g, 20.23 mmol, 1.0 eq.) in HMPA (70 mL) was added K₂CO₃ (11.16 g, 80.92 mmol, 4.0 eq.) at RT stirred for 15 min. Then cooled to 0° C. and added BnCl (9.26 mL, 80.92 mmol, 4.0 eq.) dropwise, at RT. The mixture was stirred at 90° C. for 16 h. After this time, the reaction mixture was diluted with H₂O (50 mL) and extracted with EtOAc (2×100 mL). The combined organic phase was washed with brine (50 mL), dried over anhydrous Na₂SO₄, filtered and evaporated. The crude compound was purified by column chromatography on silica gel (PE/EA=5/1) to give (2S,3R)-2-(4-(allyloxy)-3,5-bis(benzyloxy)phenyl)-5,7-bis(benzyloxy)chroman-3-ol (3.68 g, 25.7% yield) as a yellow solid. ¹H NMR (400 MHZ, MeOH-d₄): δ 7.46-7.25 (m, 20H), 6.79 (s, 2H), 6.34 (d, J=2.0 Hz, 1H), 6.12 (d, J=2.0 Hz, 1H), δ 6.04-5.92 (m, 1H), 5.26 (d, J=7.2 Hz, 1H), 5.12 (d, J=6.4 Hz, 1H), 5.15-5.00 (m, 9H), 4.62 (d, J=7.2 Hz, 1H), 4.43 (d, J=5.6 Hz, 2H), 4.01 (m, 1H), 2.76 (dd, J=16.4, 5.2 Hz, 1H), 2.55-2.41 (m, 1H).

Step 3: Synthesis of (2S,3R)-2-(4-(allyloxy)-3,5-bis(benzyloxy)phenyl)-5,7-bis(benzyloxy)chroman-3-yl 3,4,5-tris(benzyloxy)-2-fluorobenzoate (3). To a solution of 3,4,5-tris(benzyloxy)-2-fluorobenzoic acid (0.63 g, 1.38 mmol, 1.3 eq.) in CH₂Cl₂ (5 mL) was added (COCl)₂ (0.27 mL, 3.18 mmol, 3.0 eq.) and 2 drops of DMF at 0° C. The mixture was stirred at RT for 2 h. After completion of the acid chloride formation, solvent was evaporated from the reaction mixture and dried under reduced pressure. To this was added a mixture of (2S,3R)-2-(4-(allyloxy)-3,5-bis(benzyloxy)phenyl)-5,7-bis(benzyloxy)chroman-3-ol (0.75 g, 1.06 mmol, 1.0 eq.), DMAP (0.65 g, 5.31 mmol, 5.0 eq.) and TEA (0.73 mL, 5.31 mmol, 5.0 eq.) in CH₂Cl₂ (10 mL) at 0° C. The resulting mixture was stirred at room temperature for 16 h. The reaction mixture was diluted with H₂O (30 mL) and extracted with CH₂Cl₂ (2×80 mL). The combined organic phase was washed with brine (30 mL), dried over Na₂SO₄, filtered and concentrated. The residue was purified by flash chromatography on silica gel (PE/EA=7/1) to give (2S,3R)-2-(4-(allyloxy)-3,5-bis(benzyloxy)phenyl)-5,7-bis(benzyloxy)chroman-3-yl 3,4,5-tris(benzyloxy)-2-fluorobenzoate (0.992 g, 88% yield) as a pale yellow solid. ¹H NMR (400 MHZ, MeOH-d₄): δ 7.52-7.20 (m, 35H), 7.03 (d, J=6.0 Hz, 1H), 6.87 (s, 2H), 6.42 (s, 1H), 6.26 (s, 1H), 6.00-5.86 (m, 1H), 5.47 (q, J=6.0 Hz, 1H), 5.25-4.87 (m, 17H), 4.39 (d, J=5.6 Hz, 2H), 3.00-2.90 (m, 1H), 2.85-2.70 (m, 1H).

Step 4: Synthesis of (2S,3R)-5,7-bis(benzyloxy)-2-(3,5-bis(benzyloxy)-4-hydroxyphenyl) chroman-3-yl 3,4,5-tris(benzyloxy)-2-fluorobenzoate (4). To a solution of (2S,3R)-2-(4-(allyloxy)-3,5-bis(benzyloxy)phenyl)-5,7-bis(benzyloxy)chroman-3-yl 3,4,5-tris(benzyloxy)-2-fluorobenzoate (8.2 g, 7.14 mmol, 1.0 eq.) in THF (160 mL) was added NaBH₄ (0.405 g, 10.72 mmol, 1.5 eq.) at 0° C. Then Pd(PPh3)₄ (0.825 g, 0.71 mmol, 0.1 eq.) was added at RT. The resulting mixture was stirred at RT for 16 h. After completion of the reaction, reaction mixture was quenched H₂O (50 mL) and extracted with EtOAc (2×100 mL). The combined organic phase was washed with brine (50 mL), dried over Na₂SO₄, filtered and concentrated. The residue was purified by flash column chromatography on silica gel (PE/EA=4/1) to give (2S,3R)-5,7-bis(benzyloxy)-2-(3,5-bis(benzyloxy)-4-hydroxyphenyl)chroman-3-yl 3,4,5-tris(benzyloxy)-2-fluorobenzoate (1.6 g, 20% yield) as a pale green solid. ¹H NMR (400 MHz, MeOH-d₄): δ 8.51 (s, 1H), 7.47-7.14 (m, 35H), 6.98 (d, J=6.0 Hz, 1H), 6.79 (s, 2H), 6.41 (d, J=2.0 Hz, 1H), 6.24 (d, J=2.0 Hz, 1H), 5.42 (q, J=5.6 Hz, 1H), 5.20-4.85 (m, 15H), 2.91 (d, J=16.8, 5.6 Hz, 1H), 2.75 (d, J=16.4, 7.2 Hz, 1H).

Step 5: Synthesis of (2S,3R)-5,7-bis(benzyloxy)-2-(3,5-bis(benzyloxy)-4-((ethylcarbamoyl)oxy)phenyl)chroman-3-yl 3,4,5-tris(benzyloxy)-2-fluorobenzoate (5). To a solution of (2S,3R)-5,7-bis(benzyloxy)-2-(3,5-bis(benzyloxy)-4-hydroxyphenyl)chroman-3-yl 3,4,5-tris(benzyloxy)-2-fluorobenzoate (0.1 g, 0.09 mmol, 1.0 eq.) in CH₂Cl₂ (2 mL) was added Et₃N (0.07 mL, 0.45 mmol, 5.0 eq.) and ethyl isocyanate (0.02 g, 0.27 mmol, 3.0 eq.) at 0° C. The mixture was stirred at the 0° C. for 2 h, then allowed to RT and stirred for 12 h. After completion of the reaction, solvent was evaporated from the reaction mixture. The obtained residue was dissolved in THF (5 mL) added MeOH (10 mL), then formed precipitate was filtered and dried to obtain (2S,3R)-5,7-bis(benzyloxy)-2-(3,5-bis(benzyloxy)-4-((ethylcarbamoyl)oxy)phenyl)chroman-3-yl 3,4,5-tris(benzyloxy)-2-fluorobenzoate (0.085 g, 80% yield) as a white solid. ¹HNMR (400 MHZ, MeOH-d₄): 7.47-7.14 (m, 35H), 7.09 (d, J=6.0 Hz, 1H), 6.76 (s, 2H), 6.31 (d, J=6.8, 2.4 Hz, 1H), 5.50 (d, J=5.6 Hz, 1H), 5.16 (d, J=6.4 Hz, 1H), 5.10-4.93 (m, 14H), 3.26 (quintet, J=6.8 Hz, 2H), 3.04 (d, J=5.6 Hz, 1H), 2.88 (dd, J=17.2, 6.8 Hz, 1H), 1.45-1.25 (m, 1H).

Step 6: Synthesis of Compound 67. To a mixture of (2S,3R)-5,7-bis(benzyloxy)-2-(3,5-bis(benzyloxy)-4-((ethylcarbamoyl)oxy)phenyl)chroman-3-yl 3,4,5-tris(benzyloxy)-2-fluorobenzoate (0.8 g, 0.68 mmol, 1.0 eq.) in THF (10 mL) and MeOH (5 mL) was added Pd(OH)₂ (20 wt. %, 0.96 g). The mixture was stirred at room temperature under H₂ atmosphere for overnight. The reaction mixture was filtered and the filtrate was concentrated. The residue was purified by prep-HPLC to give (2S,3R)-2-(4-((ethylcarbamoyl)oxy)-3,5-dihydroxyphenyl)-5,7-dihydroxychroman-3-yl 2-fluoro-3,4,5-trihydroxybenzoate (150 mg, 40.4% yield) as off-white solid. ¹H NMR (400 MHZ, MeOH-d₄): 9.26 (bs, 7H), 7.41 (t, J=6.0 Hz, 1H), 6.66 (d, J=6.4 Hz, 1H), 6.31 (s, 2H), 5.91 (d, J=2.4 Hz, 1H), 5.82 (d, J=2.0 Hz, 1H), 5.33 (q, J=4.4 Hz, 1H), 5.14 (d, J=4.4 Hz, 1H), 3.02 (quintet, J=7.2 Hz, 2H), 2.63 (d, J=18.4, 1.2 Hz, 1H), 2.50 (dd, J=20.0, 4.0 Hz, 1H), 1.03 (t, J=7.2 Hz, 3H).

### Compound 68

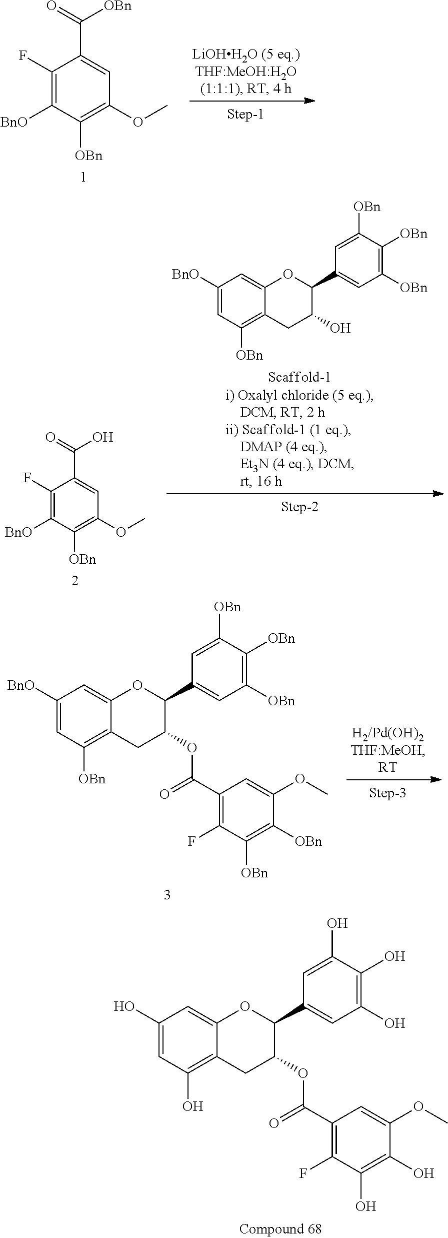 [SMILES: COc1cc(C(=O)OCc2ccccc2)c(F)c(OCc2ccccc2)c1OCc1ccccc1 ; O[C@@H]1Cc2c(OCc3ccccc3)cc(OCc3ccccc3)cc2O[C@H]1c1cc(OCc2ccccc2)c(OCc2ccccc2)c(OCc2ccccc2)c1 ; COc1cc(C(=O)O)c(F)c(OCc2ccccc2)c1OCc1ccccc1 ; COc1cc(C(=O)O[C@@H]2Cc3c(OCc4ccccc4)cc(OCc4ccccc4)cc3O[C@H]2c2cc(OCc3ccccc3)c(OCc3ccccc3)c(OCc3ccccc3)c2)c(F)c(OCc2ccccc2)c1OCc1ccccc1 ; COc1cc(C(=O)O[C@@H]2Cc3c(O)cc(O)cc3O[C@H]2c2cc(O)c(O)c(O)c2)c(F)c(O)c1O]

Step 1: Synthesis of 3, 4-bis(benzyloxy)-2-fluoro-5-methoxybenzoic acid (2). To a solution of benzyl 3,4-bis(benzyloxy)-2-fluoro-5-methoxybenzoate (4.70 g, 9.95 mmol, 1.0 eq.) in THF:MeOH: H₂O (1:1:1) (50 mL) was added anhydrous LiOH (1.2 g, 49.77 mmol, 5.0 eq.) at 0° C. and stirred at RT for 4 h. After completion of the starting material on TLC, solvent was evaporated from the reaction mixture. The obtained solid was diluted with H₂O (50 mL), washed with diethyl ether (50 mL). The aqueous layer was acidified with 1N HCl (pH=3-4) and product extracted with EtOAc (2×100 mL). The combined organic phase was dried over anhydrous Na₂SO₄, evaporated under reduced pressure to obtain 3, 4-bis(benzyloxy)-2-fluoro-5-methoxybenzoic acid (2.28 g, 60% yield) as a pale brown gummy solid. ¹H NMR (400 MHZ, DMSO-d₆): 7.50-7.21 (m, 10H), 7.17 (d, J=6.4 Hz, 1H), 5.09 (s, 2H), 5.02 (s, 2H), 3.83 (s, 3H).

Step 2: Synthesis of (2S,3R)-5,7-bis(benzyloxy)-2-(3,4,5-tris(benzyloxy)phenyl)chroman-3-yl 3,4-bis(benzyloxy)-2-fluoro-5-methoxybenzoate (3). To a solution of 3, 4-bis(benzyloxy)-2-fluoro-5-methoxybenzoic acid (1.13 g, 2.97 mmol, 1.5 eq.) in CH₂Cl₂ (12 mL) was added oxalyl chloride (0.9 mL, 9.92 mmol, 5.0 eq.) and 2 drops of dry DMF at 0° C. The mixture was stirred at RT for 2 h. After completion of acid-chloride formation, volatile portion was concentrated from the reaction mixture. Obtained acid chloride was added to a mixture of (2S,3R)-5,7-bis(benzyloxy)-2-(3,4,5-tris(benzyloxy)phenyl)chroman-3-ol (1.5 g, 1.98 mmol, 1.0 eq.), DMAP (0.96 g, 7.93 mmol, 4.0 eq.) and TEA (1.2 mL, 7.93 mmol, 4.0 eq.) in CH₂Cl₂ (20 mL) at 0° C. The resulting mixture was stirred at RT for 16 h. The reaction mixture was diluted with H₂O (50 mL) and extracted with CH₂Cl₂ (2×100 mL). The combined organic layer was washed with brine (50 mL), dried over Na₂SO₄, filtered and concentrated. The residue was purified by flash column chromatography on silica gel (PE/EA=6/1) to give (2S,3R)-5,7-bis(benzyloxy)-2-(3,4,5-tris(benzyloxy)phenyl)chroman-3-yl 3,4-bis(benzyloxy)-2-fluoro-5-methoxybenzoate (1.51 g, 68% yield) as a white solid. ¹H NMR (400 MHZ, CDCl₃) δ 7.50-7.20 (m, 35H), 7.00 (d, J=6.0 Hz, 1H), 6.79 (s, 2H), 6.32 (d, J=2.0 Hz, 1H), 6.30 (d, J=2.0 Hz, 1H), 5.50 (q, J=7.6 Hz, 1H), 5.09 (d, J=6.0 Hz, 1H), 5.08-4.98 (m, 14H), 3.76 (s, 3H), 3.17 (dd, J=16.8, 11.2 Hz, 1H), 2.88 (q, J=8.0, 1H).

Step 3: Synthesis of Compound 68. To a mixture of (2S,3R)-5,7-bis(benzyloxy)-2-(3,4,5-tris(benzyloxy)phenyl)chroman-3-yl 3,4-bis(benzyloxy)-2-fluoro-5-methoxybenzoate (1.50 g, 1.33 mmol, 1.0 eq.) in THF (10 mL) and MeOH (10 mL) was added Pd(OH)₂/C (20 wt. %, 190 mg). The mixture was stirred at room temperature under H₂ atmosphere for overnight. The reaction mixture was filtered and the filtrate was concentrated. The residue was purified by prep-HPLC to give (2S,3R)-5,7-dihydroxy-2-(3,4,5-trihydroxyphenyl)chroman-3-yl 2-fluoro-3,4-dihydroxy-5-methoxybenzoate (0.335 g, 51% yield) as an off-white solid. ¹H NMR (400 MHZ, DMSO-d₆) δ 9.11 (bs, 7H), 6.60 (d, J=6.0 Hz, 1H), 6.31 (s, 2H), 5.92 (d, J=2.4 Hz, 1H), 5.78 (d, J=2.0 Hz, 1H), 5.18 (q, J=6.0 Hz, 1H), 4.95 (d, J=6.4 Hz, 1H), 3.68 (s, 3H), 2.78 (dd, J=16.4, 5.2 Hz, 1H), 2.88 (dd, J=16.0, 6.8 Hz, 1H).

### Compound 72

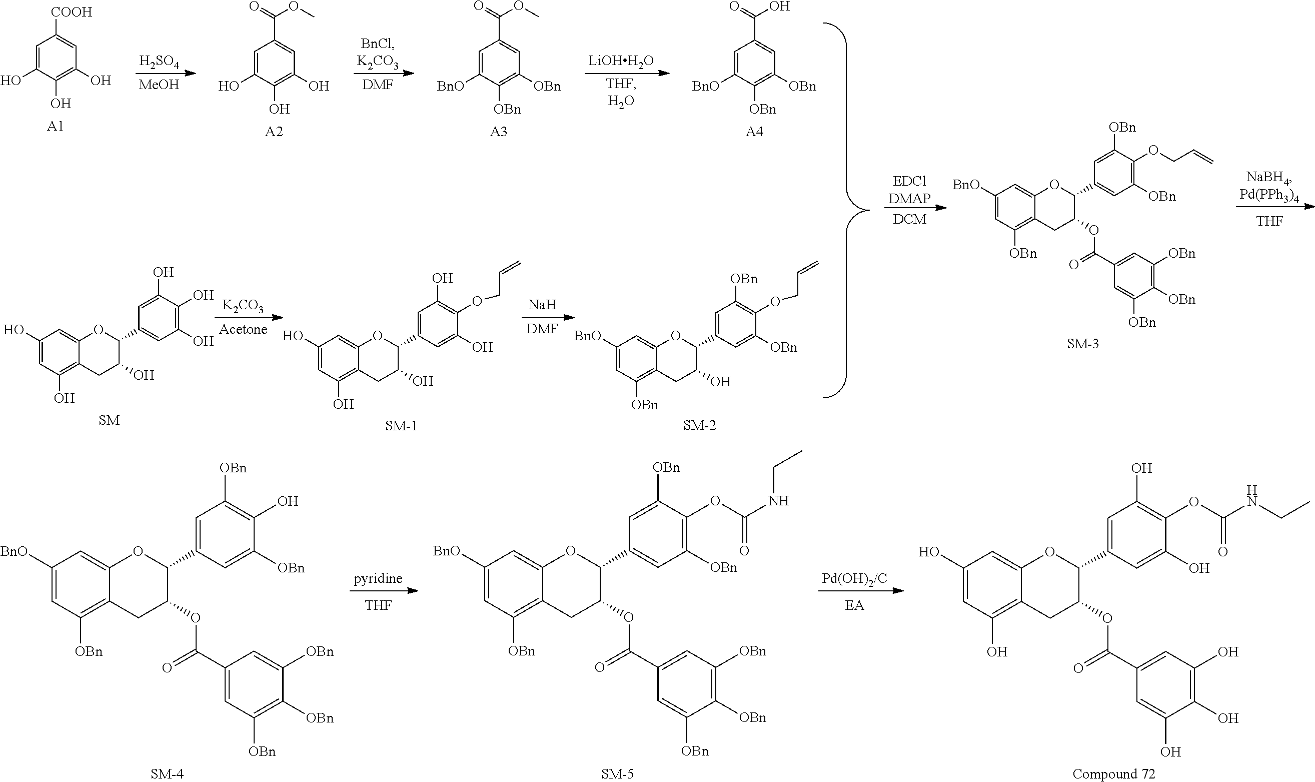

Step 1: Synthesis of methyl 3,4,5-trihydroxybenzoate (A2). To a solution of compound A1 (20 g, 0.12 mol) in MeOH (200 mL) was added con·H2SO4 (6 mL) at 0° C. The reaction mixture was stirred at 80° C. overnight. The reaction mixture was cooled, neutralized with Na2CO3 solution at 0° C. and extracted with EA (100 mL×3). The combined organic layers were washed with brine (100 mL×2), dried over Na2SO4, filtered and concentrated to give the crude compound A2 (15 g, 70% yield) as a yellow solid. MS Calcd.: 184. MS Found: 185 [M+H]+.

Step 2: Synthesis of methyl 3,4,5-tris(benzyloxy)benzoate (A3). To a solution of compound A2 (1.8 g, 9.77 mmol) and K2CO3 (5.4 g, 39.13 mmol) in DMF (20 mL) was added BnCl (5.54 g, 43.97 mmol) at 0° C. The solution was stirred at 60° C. for 4 hours. The reaction mixture was cooled, diluted with H2O (50 mL) and extracted with EA (50 mL×2). The combined organic layers were washed with brine (30 mL×2), dried over Na2SO4, filtered and concentrated. The residue was purified by flash chromatography on silica gel (PE/EA=5/1) to give compound A3 (4.05 g, 91% yield) as a white solid. MS Calcd.: 454. MS Found: 455 [M+H]+.

Step 3: Synthesis of 3,4,5-tris(benzyloxy)benzoic acid (A4). To a solution of compound A3 (4.0 g, 8.8 mmol) in THF (30 mL) and H2O (10 mL) was added LiOH·H2O (554 mg, 13.2 mmol). The solution was stirred at 50° C. overnight. The reaction mixture was concentrated. The residue was adjusted pH=3 with 2N HCl solution and filtered. The filtered cake was dried to give the crude compound A4 (3.29 g, 85% yield) as a yellow solid. MS Calcd.: 440. MS Found: 441 [M+H]+.

Step 4: Synthesis of (2R,3R)-2-(4-(allyloxy)-3,5-dihydroxyphenyl)chroman-3,5,7-triol (SM-1). To a solution of compound SM (10 g, 32.65 mmol) in acetone (100 mL) was added K2CO3 (8.79 g, 63.67 mmol) at 0° C. The mixture was stirred at 0° C. for 30 minutes. Then 3-bromoprop-1-ene (4.74 g, 39.18 mmol) was added at 0° C. and the solution was stirred at 55° C. overnight. The reaction mixture was concentrated. The residue was purified by flash chromatography on reverse phase silica gel (ACN/H2O=5%-95%, 254 nm, 30 minutes) to give compound SM-1 (3.5 g, 31% yield) as a yellow solid. 1H NMR (400 MHZ, CD3OD) δ: 6.55 (s, 2H), 6.21-6.12 (m, 1H), 5.96-5.94 (m, 2H), 5.34-5.29 (m, 1H), 5.19 (d, J=10.4 Hz, 1H), 4.79 (s, 1H), 4.56-4.54 (m, 2H), 4.22-4.20 (m, 1H), 2.90-2.85 (m, 1H), 2.77-2.67 (m, 1H). MS Calcd.: 346. MS Found: 347 [M+H]+.

Step 5: Synthesis of (2R,3R)-2-(4-(allyloxy)-3,5-bis(benzyloxy)phenyl)-5,7-bis(benzyloxy)chroman-3-ol (SM-2). To a solution of compound SM-1 (6.0 g, 19.35 mmol) in DMF (80 mL) was added NaH (2.91 g, 72.76 mmol, 60% wt. in mineral oil) at 0° C. The mixture was stirred at 0° C. for 20 minutes. BnCl (9.21 g, 72.76 mmol) was added at 0° C. and the solution was stirred at room temperature overnight. The reaction mixture was diluted with H2O (50 mL) and extracted with EA (50 mL×2). The combined organic layers were washed with brine (30 mL×2), dried over Na2SO4, filtered and concentrated. The residue was purified by flash chromatography on silica gel (PE/EA=2/1) to give the crude product which was further purify by flash chromatography on reverse phase silica gel (ACN/H2O=5%-95%, 254 nm, 45 minutes) to give compound SM-2 (2.55 g, 21% yield) as yellow oil. MS Calcd.: 706. MS Found: 707 [M+H]+.

Step 6: Synthesis of (2R,3R)-2-(4-(allyloxy)-3,5-bis(benzyloxy)phenyl)-5,7-bis(benzyloxy)chroman-3-yl 3,4,5-tris(benzyloxy)benzoate (SM-3). To a solution of compound SM-2 (5.2 g, 7.22 mmol) in DCM (50 mL) was added compound A4 (4.13 g, 9.38 mmol), EDCI (4.15 g, 21.66 mmol) and DMAP (882 mg, 7.22 mmol) at 0° C. The reaction mixture was stirred at room temperature overnight. The reaction mixture was diluted with water (30 mL) and extracted with DCM (20 mL×2). The combined organic layers were dried over Na2SO4 and filtered. The filtrate was concentrated to give the crude product which was purified by flash chromatography on reverse phase silica gel (ACN/H2O=5%-95%, 254 nm, 40 minutes) to give compound SM-3 (7.2 g, 88% yield) as yellow oil. MS Calcd.: 1128. MS Found: 1145 [M+NH4]+.

Step 7: Synthesis of (2R,3R)-5,7-bis(benzyloxy)-2-(3,5-bis(benzyloxy)-4-hydroxyphenyl)chroman-3-yl 3,4,5-tris(benzyloxy)benzoate (SM-4). To a solution of compound SM-3 (7.2 g, 6.38 mmol) in THF (50 mL) was added NaBH4 (363 mg, 9.56 mmol) at ice-water bath. After stirring for 5 minutes, Pd(PPh3)4 (737 mg, 0.638 mmol) was added. The mixture was stirred at room temperature overnight. The reaction mixture was diluted with water (30 mL) and extracted with DCM (30 mL×2). The combined organic layers were washed with brine (30 ml×2), dried over Na2SO4, filtered and concentrated to give compound SM-4 (5.31 g, 95% yield) as yellow oil. MS Calcd.: 1088. MS Found: 1105 [M+NH4]+.

Step 8: Synthesis of (2R,3R)-5,7-bis(benzyloxy)-2-(3,5-bis(benzyloxy)-4-((ethylcarbamoyl)oxy)phenyl)chroman-3-yl 3,4,5-tris(benzyloxy)benzoate (SM-5). To a mixture of compound SM-4 (1.4 g, 1.29 mmol) in THF (30 mL) was added pyridine (408 mg, 5.16 mmol) and Bis(trichloromethyl)Carbonate (153 mg, 0.52 mmol) at ice-water bath. After stirring for 10 minutes, ethanamine (87 mg, 1.93 mmol) was added at 0° C. The reaction mixture was stirred at room temperature for 1 hour. The reaction mixture was diluted with water (30 mL) and extracted with DCM (20 mL×2). The combined organic layers were dried over Na2SO4 and filtered. The filtrate was concentrated to give the crude product which was purified by flash chromatography on reverse phase silica gel (ACN/H2O=5%-95%, 254 nm, 40 minutes) to give compound SM-5 (0.92 g, 62% yield) as yellow oil. MS Calcd.: 1159. MS Found: 1177 [M+NH4]+.

Step 9: Synthesis of Compound 72. To a mixture of compound SM-5 (920 mg, 0.79 mmol) in EA (20 mL) was added Pd(OH)2/C (10% wt., 100 mg). The mixture was stirred at room temperature under H2 of balloon for 2 days. The reaction mixture was filtered and concentrated. The residue was purified by pre-HPLC to give (2R,3R)-2-(4-((ethylcarbamoyl)oxy)-3,5-dihydroxyphenyl)-5,7-dihydroxychroman-3-yl 3,4,5-trihydroxybenzoate (210 mg, 50% yield) as a white solid. 1H NMR (400 MHZ, CD3OD) δ: 6.84 (s, 2H), 6.47 (s, 2H), 5.86 (s, 2H), 5.45 (s, 1H), 4.92 (s, 1H), 3.12-3.07 (m, 2H), 2.92-2.87 (m, 1H), 2.78-2.73 (m, 1H), 1.06 (t, J=7.0 Hz, 3H). MS Calcd.: 529. MS Found: 530 [M+H]+.

### Compound 73

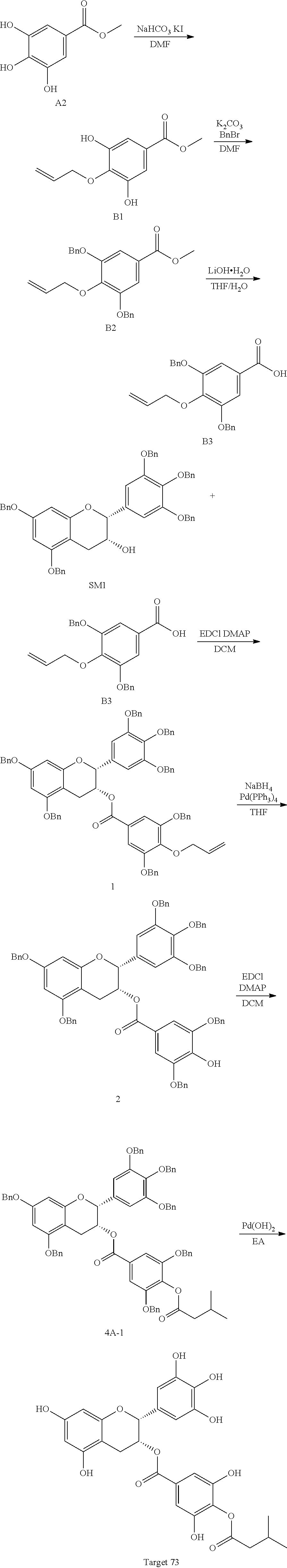

Step 1: Synthesis of methyl 4-(allyloxy)-3,5-dihydroxybenzoate (B1). To a solution of compound A2 (2.92 g, 15.87 mmol) in DMF (30 mL) was added 3-bromoprop-1-ene (1.92 g, 15.87 mmol) and NaHCO3(5.33 g, 63.46 mmol) and KI (2.63 g, 15.87 mmol). The reaction mixture was stirred at room temperature overnight. The reaction mixture was diluted with H2O (50 mL) and extracted with EA (50 mL×2). The combined organic layers were washed with brine (30 mL×2), dried over Na2SO4, filtered and concentrated. The residue was purified by flash chromatography on silica gel (PE/EA=5/1-1/1) to give compound B1 (2.4 g, 67% yield) as yellow oil. MS Calcd.: 224. MS Found: 225 [M+H]+.

Step 2: Synthesis of methyl 4-(allyloxy)-3,5-bis(benzyloxy)benzoate(B2). To a solution of compound B1 (1.8 g, 8.03 mmol) in DMF (30 mL) was added K2CO3 (2.22 g, 16.06 mmol) and BnBr (4.12 g, 24.1 mmol) at ice-water bath. The reaction mixture was stirred at 60° C. overnight. The reaction mixture was diluted with H2O (30 mL) and extracted with EA (50 mL×2). The combined organic layers were washed with brine (30 mL×2), dried over Na2SO4, filtered and concentrated. The residue was purified by flash chromatography on silica gel (PE/EA=5/1-3/1) to give compound B2 (2.98 g, 92% yield) as a white solid. MS Calcd.: 404; MS Found: 405 [M+H]+.

Step 3: Synthesis of 4-(allyloxy)-3,5-bis(benzyloxy)benzoic acid (B3). To a solution of compound B2 (2.98 g, 7.38 mmol) in THF/H2O (30 mL/10 mL) was added LiOH·H2O (0.62 g, 14.75 mmol). The reaction mixture was stirred at 50° C. overnight. The reaction mixture was adjusted to pH=5 with 1N HCl solution and extracted with EA (50 mL×2). The combined organic layers were washed with brine (30 mL×2), dried over Na2SO4, filtered and concentrated to give compound B3 (2.4 g, 83% yield) as a white solid. MS Calcd.: 390; MS Found: 391 [M+H]+.

Step 4: Synthesis of (2R,3R)-5,7-bis(benzyloxy)-2-(3,4,5-tris(benzyloxy)phenyl)chroman-3-yl 4-(allyloxy)-3,5-bis(benzyloxy)benzoate (1). To a mixture of compound SM1 (1.0 g, 1.32 mmol) in DCM (30 mL) was added compound B3 (774 mg, 1.98 mmol), EDCI (757 mg, 3.96 mmol) and DMAP (32 mg, 0.26 mmol) at ice-water bath. The solution was stirred at room temperature overnight. The reaction mixture was diluted with H2O (50 mL) and extracted with DCM (30 mL×2). The combined organic layers were washed with brine (30 mL×2), dried over Na2SO4, filtered and concentrated. The residue was purified by flash chromatography on reverse phase silica gel (ACN/H2O=5%-95%, 254 nm, 40 min) to give compound 1 (1.2 g, 81% yield) as yellow oil. MS Calcd.: 1128. MS Found: 1129 [M+H]+.

Step 5: Synthesis of (2R,3R)-5,7-bis(benzyloxy)-2-(3,4,5-tris(benzyloxy)phenyl)chroman-3-yl 3,5-bis(benzyloxy)-4-hydroxybenzoate (2). A mixture of compound 1 (7.0 g, 6.2 mmol) in THF (50 mL) was added NaBH4 (352 mg, 9.3 mmol) at ice-water bath. After 5 minutes Pd(pph3)4 (358 mg, 0.31 mmol) was added. The mixture was stirred at room temperature for 5 hours. The reaction mixture was concentrated and the residue was purified by flash chromatography on silica gel (PE/EA=1/1-DCM/MeOH=10/1) to give compound 2 (4.2 g, 62% yield) as yellow oil. MS Calcd.: 1088. MS Found: 1089 [M+H]+.

Step 6: Synthesis of (2R,3R)-5,7-bis(benzyloxy)-2-(3,4,5-tris(benzyloxy)phenyl)chroman-3-yl 3,5-bis(benzyloxy)-4-((3-methylbutanoyl)oxy)benzoate (4A-1). To a mixture of compound 2 (300 mg, 0.28 mmol) in DCM (30 mL) was added 3-methylbutanoic acid (34 mg, 0.34 mmol), EDCI (161 mg, 0.84 mmol) and DMAP (68 mg, 0.56 mmol) at 0° C. The reaction mixture was stirred at room temperature for 2 hours. The reaction mixture was diluted with water (30 mL) and extracted with DCM (20 mL×2). The combined organic layers were dried over Na2SO4 and filtered. The filtrate was concentrated to give a crude product, which was purified by flash chromatography on reverse phase silica gel (ACN/H2O=5%-95%, 254 nm, 40 min) to give compound 4A-1 (312 mg, 97% yield) as yellow oil. MS Calcd.: 1172. MS Found: 1173 [M+H]+.

Step 7: Synthesis of Compound 73. To a mixture of compound 4A-1 (312 mg, 0.27 mmol) in 20 mL of EA was added Pd(OH)2 (10% wt., 32 mg). The mixture was stirred at room temperature under H2 atmosphere (15 PSI) overnight. The reaction mixture was filtered and concentrated. The residue was purified by pre-UPLC to give (2R,3R)-5,7-dihydroxy-2-(3,4,5-trihydroxyphenyl)chroman-3-yl 3,5-dihydroxy-4-((3-methylbutanoyl)oxy)benzoate (65 mg, 45% yield) as a yellow solid. 1H NMR (400 MHZ, CD3OD) δ: 7.25-6.95 (m, 2H), 6.48 (s, 2H), 5.95-5.94 (m, 2H), 5.56 (d, J=13.6 Hz, 1H), 4.97 (s, 1H), 3.03-2.96 (m, 1H), 2.85 (d, J=17.6 Hz, 1H), 2.46 (d, J=6.8 Hz, 2H), 2.22-2.15 (m, 1H), 1.05 (d, J=6.8 Hz, 6H). MS Calcd.: 542; MS Found: 543 [M+H]+.

### Compound 74

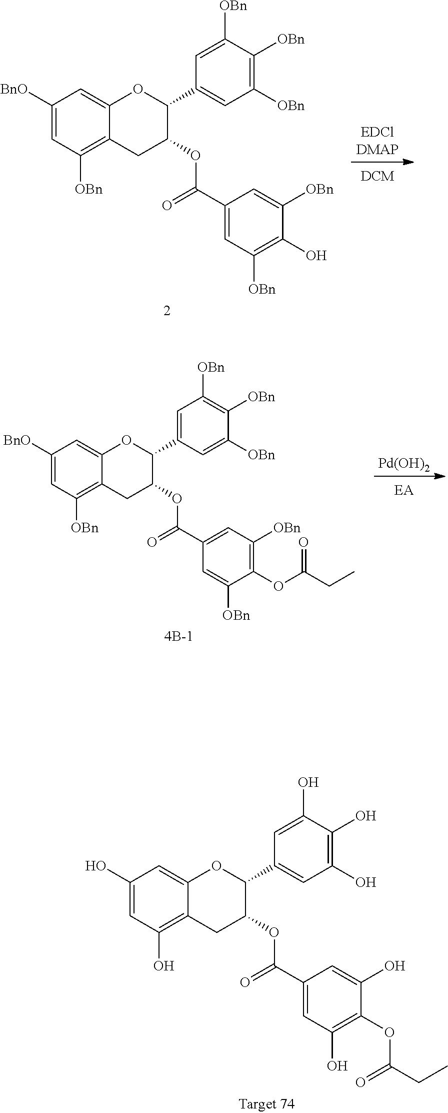

Step 1: Synthesis of (2R,3R)-5,7-bis(benzyloxy)-2-(3,4,5-tris(benzyloxy)phenyl)chroman-3-yl 3,5-bis(benzyloxy)-4-(propionyloxy)benzoate(4B-1). To a mixture of compound 2 (see Compound 73 for preparation, 300 mg, 0.28 mmol) and DIEA (106.9 mg, 0.83 mmol) in THF (6 mL) was added propinyl chloride (76.1 mg, 0.83 mmol) at 0° C. The reaction mixture was stirred at room temperature for 3 hours. The reaction mixture was diluted with water (30 mL) and extracted with EA (20 mL×2). The combined organic layers were dried over Na2SO4 and filtered. The filtrate was concentrated to give a crude product, which was purified by flash chromatography on reverse phase silica gel (ACN/H2O=5%-95%, 254 nm, 40 min) to give compound 4B-1 (280 mg, 88% yield) as a yellow solid. MS Calcd.: 1144. MS Found: 1145 [M+H]+.

Step 2. Synthesis of Compound 74. To a mixture of compound 4B-1 (350 mg, 0.31 mmol) in 70 mL of EA was added Pd(OH)2 (10% wt., 70 mg). The mixture was stirred at room temperature under H2 atmosphere (15 PSI) overnight. The reaction mixture was filtered and concentrated. The residue was purified by pre-UPLC to give (2R,3R)-5,7-dihydroxy-2-(3,4,5-trihydroxyphenyl)chroman-3-yl 3,5-dihydroxy-4-(propionyloxy)benzoate (90 mg, 57.3% yield) as a white solid. 1H NMR (400 MHZ, DMSO-d6) δ: 9.86-9.70 (m, 2H), 9.3 (s, 1H), 9.05 (s, 1H), 8.71 (s, 2H), 7.98 (s, 1H), 7.18-6.87 (m, 2H), 6.41 (s, 2H), 5.96-5.95 (m, 1H), 5.85-5.84 (m, 1H), 5.41 (d, J=24.4 Hz, 1H), 4.99 (s, 1H), 2.99-2.94 (m, 1H), 2.72-2.68 (m, 1H), 2.62-2.5 (m, 2H), 1.14-1.1 (m, 3H). MS Calcd.: 514. MS Found: 515 [M+H]+.

### Compound 75

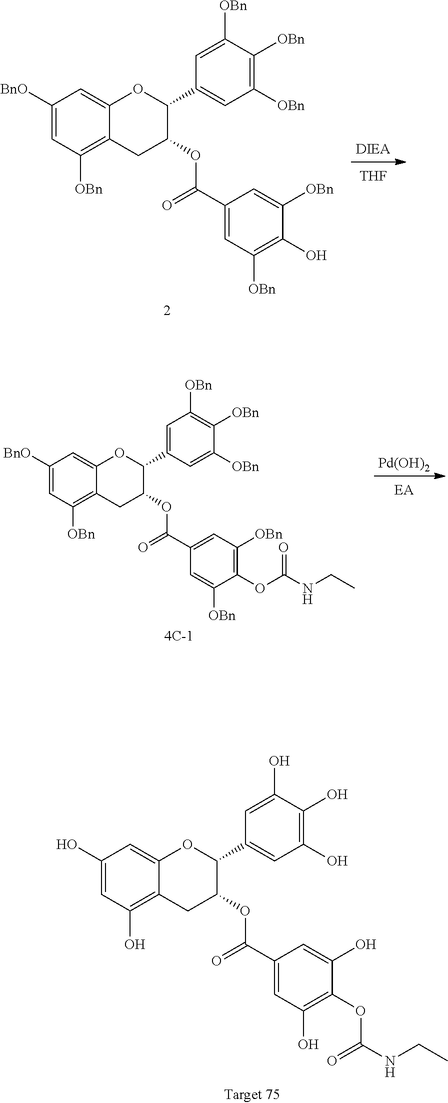 [SMILES: O=C(O[C@@H]1Cc2c(OCc3ccccc3)cc(OCc3ccccc3)cc2O[C@@H]1c1cc(OCc2ccccc2)c(OCc2ccccc2)c(OCc2ccccc2)c1)c1cc(OCc2ccccc2)c(O)c(OCc2ccccc2)c1 ; CCNC(=O)Oc1c(OCc2ccccc2)cc(C(=O)O[C@@H]2Cc3c(OCc4ccccc4)cc(OCc4ccccc4)cc3O[C@@H]2c2cc(OCc3ccccc3)c(OCc3ccccc3)c(OCc3ccccc3)c2)cc1OCc1ccccc1 ; "Pd(OH)2" ; "EA" ; "THF" ; CCNC(=O)Oc1c(O)cc(C(=O)O[C@@H]2Cc3c(O)cc(O)cc3O[C@@H]2c2cc(O)c(O)c(O)c2)cc1O]

Step 1: Synthesis of (2R,3R)-5,7-bis(benzyloxy)-2-(3,4,5-tris(benzyloxy)phenyl)chroman-3-yl 3,5-bis(benzyloxy)-4-((ethylcarbamoyl)oxy)benzoate (4C-1). To a mixture of compound 2 (see Compound 73 for preparation, 700 mg, 0.64 mmol) in THF (30 mL) was added DIEA (249.5 mg, 1.93 mmol), Ethyl isocyante (136 mg, 1.93 mmol) at 0° C. The reaction mixture was stirred at room temperature for 3 hours. The reaction mixture was diluted with water (30 mL) and extracted with EA (20 mL×2). The combined organic layers were dried over Na2SO4 and filtered. The filtrate was concentrated to give a crude product, which was purified by flash chromatography on reverse phase silica gel (ACN/H2O=5%-95%, 254 nm, 40 min) to give compound 4C-1 (460 mg, 61.7% yield) as a white solid. MS Calcd.: 1159. MS Found: 1160 [M+H]+.

Step 2: Synthesis of Compound 75. To a mixture of compound 4C-1 (450 mg, 0.388 mmol) in 100 mL of EA was added Pd(OH)2 (10% wt., 100 mg). The mixture was stirred at room temperature under H2 atmosphere (15 PSI) overnight. The reaction mixture was filtered and concentrated. The residue was purified by pre-UPLC to give (2R,3R)-5,7-dihydroxy-2-(3,4,5-trihydroxyphenyl)chroman-3-yl4-((ethylcarbamoyl)oxy)-3,5-dihydroxybenzoate (65 mg, 31.7% yield) as a white solid. 1H NMR (400 MHZ, CD3OD) δ: 6.99 (s, 2H), 6.53 (s, 2H), 6.00 (s, 2H), 5.61 (s, 1H), 5.01 (s, 1H), 3.28-3.21 (m, 2H), 3.08-3.01 (m, 1H), 2.90 (d, J=23.2 Hz, 1H), 1.28-1.19 (m, 3H). MS Calcd.: 529. MS Found: 530 [M+H].

### Compound 76

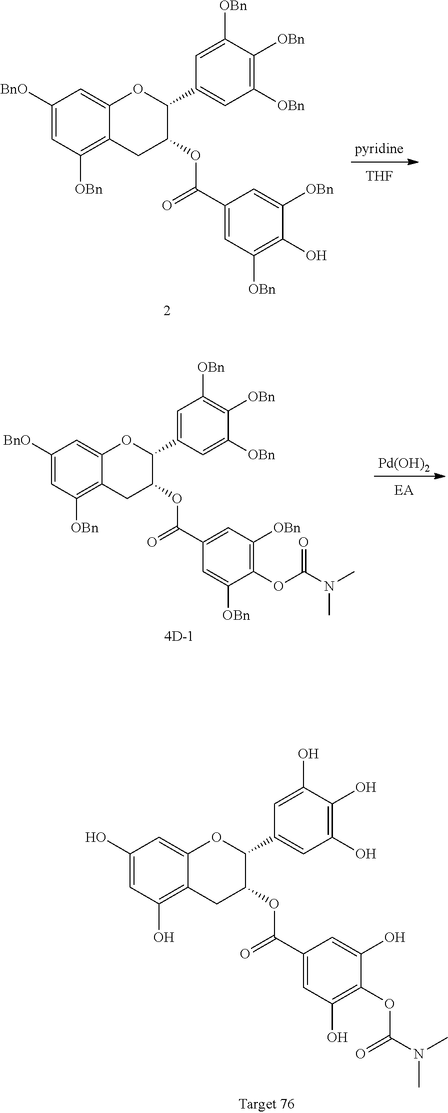 [SMILES: O=C(O[C@@H]1Cc2c(OCc3ccccc3)cc(OCc3ccccc3)cc2O[C@@H]1c1cc(OCc2ccccc2)c(OCc2ccccc2)c(OCc2ccccc2)c1)c1cc(OCc2ccccc2)c(O)c(OCc2ccccc2)c1 ; CN(C)C(=O)Oc1c(OCc2ccccc2)cc(C(=O)O[C@@H]2Cc3c(OCc4ccccc4)cc(OCc4ccccc4)cc3O[C@@H]2c2cc(OCc3ccccc3)c(OCc3ccccc3)c(OCc3ccccc3)c2)cc1OCc1ccccc1 ; "Pd(OH)2" ; "EA" ; "THF" ; CN(C)C(=O)Oc1c(O)cc(C(=O)O[C@@H]2Cc3c(O)cc(O)cc3O[C@@H]2c2cc(O)c(O)c(O)c2)cc1O]

Step 1: Synthesis of (2R,3R)-5,7-bis(benzyloxy)-2-(3,4,5-tris(benzyloxy)phenyl)chroman-3-yl 3,5-bis(benzyloxy)-4-((dimethylcarbamoyl)oxy)benzoate (4D-1). To a mixture of compound 2 (see Compound 73 for preparation, 2.9 g, 2.66 mmol) in THF (50 mL) was added pyridine (841 mg, 10.64 mmol) and Bis(trichloromethyl)Carbonate (316 mg, 1.06 mmol) at ice-water bath. After 10 minute a solution of dimethylamine in THF (2.0 M, 4.0 mL, 7.99 mmol) was added at 0° C. The reaction mixture was stirred at room temperature for 1 hour. The reaction mixture was diluted with water (30 mL) and extracted with DCM (30 mL×2). The combined organic layers were dried over Na2SO4 and filtered. The filtrate was concentrated to give a crude product which was purified by flash chromatography on reverse phase silica gel (ACN/H2O=5%-95%, 254 nm, 40 min) to give compound 4D-1 (2.16 g, 70% yield) as yellow oil. MS Calcd.: 1159. MS Found: 1160 [M+H]+.

Step 2: Synthesis of Compound 76. To a mixture of compound 4D-1 (2.16 g, 1.86 mmol) in EA (40 mL) was added Pd(OH)2 (10% wt., 200 mg). The mixture was stirred at room temperature under H2 of balloon overnight. The reaction mixture was filtered and concentrated. The residue was purified by pre-UPLC to give (2R,3R)-5,7-dihydroxy-2-(3,4,5-trihydroxyphenyl)chroman-3-yl 4-((dimethylcarbamoyl)oxy)-3,5-dihydroxybenzoate (80 mg, 8% yield) as a white solid. 1H NMR (400 MHZ, CD3OD) δ: 7.76 (d, J=1.6 Hz, 1H), 7.13 (d, J=2.0 Hz, 1H), 6.51 (s, 2H), 6.98 (s, 2H), 5.54 (s, 1H), 4.99 (s, 1H), 3.14 (s, 3H), 3.03-2.98 (m, 4H), 2.89-2.84 (m, 1H). MS Calcd.: 529. MS Found: 530 [M+H]+.

### Compound 77

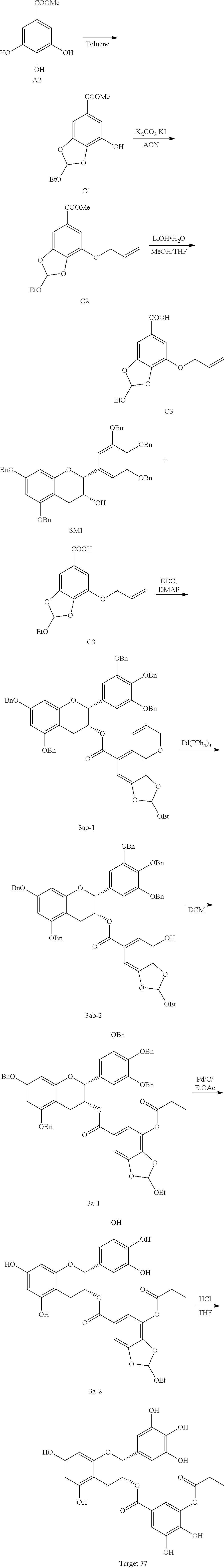

Step 1: Synthesis of methyl 2-ethoxy-7-hydroxybenzo[d][1,3]dioxole-5-carboxylate (C1). To a solution of compound A2 (7.2 g, 39.1 mmol) in Toluene (50 mL) was added triethoxymethane (17.38 g, 117.3 mmol) and Amberlyst. 15(H) (2.0 g). The reaction mixture was stirred at 120° C. overnight. The reaction mixture was filtered and washed with toluene (30 mL). The filtrate was concentrated to give compound C1 (6.7 g, 71% yield) as yellow oil which was used to the next step without further purification. MS Calcd.: 240; MS Found: 241 [M+H]+

Step 2: Synthesis of methyl 7-(allyloxy)-2-ethoxybenzo[d][1,3]dioxole-5-carboxylate (C2). To a solution of compound C1 (6.7 g, 27.9 mmol) in ACN (50 mL) was added K2CO3 (11.55 g, 83.7 mmol), KI (4.63 g, 27.9 mmol) and 3-bromoprop-1-ene (4.35 g, 36.3 mmol). The reaction mixture was stirred at room temperature overnight. The reaction mixture was concentrated and the residue was purified by flash chromatography on silica gel (PE/EA=10/1-5/1) to give compound C2 (6.8 g, 87% yield) as yellow oil. MS Calcd.: 280; MS Found: 281 [M+H]+.

Step 3: Synthesis of 7-(allyloxy)-2-ethoxybenzo[d][1,3]dioxole-5-carboxylic acid(C3). To a solution of compound C2 (9.1 g, 32.5 mmol) in THF/MeOH (20 mL/20 mL) was added LiOH·H2O (2.05 g, 48.7 mmol). The reaction mixture was stirred at 60° C. overnight. The reaction mixture was adjusted to pH=5 with 1N HCl solution and extracted with EA (30 mL×2). The combined organic layers were washed with brine (30 mL×2), dried over Na2SO4, filtered and concentrated to give compound C3 (8.21 g, 95% yield) as a yellow solid. MS Calcd.: 266; MS Found: 267 [M+H]+.

Step 4: Synthesis of (2R,3R)-5,7-bis(benzyloxy)-2-(3,4,5-tris(benzyloxy)phenyl)chroman-3-yl 7-(allyloxy)-2-ethoxybenzo[d][1,3]dioxole-5-carboxylate (3ab-1). To a mixture of compound SM1 (3.0 g, 3.96 mmol) in DCM (30 mL) was added compound C3 (1.27 g, 4.76 mmol), EDCI (2.28 g, 11.88 mmol) and DMAP (480 mg, 3.96 mmol) at ice-water bath. The solution was stirred at room temperature for 2 hours. The reaction mixture was diluted with H2O (50 mL) and extracted with DCM (30 mL×2). The combined organic layers were washed with brine (30 mL×2), dried over Na2SO4, filtered and concentrated. The residue was purified by flash chromatography on reverse phase silica gel (ACN/H2O=5%-95%, 254 nm, 30 min) to give compound 3ab-1 (1.5 g, 38% yield) as yellow oil. MS Calcd.: 1004. MS Found: 1005 [M+H]+.

Step 5: Synthesis of (2R,3R)-5,7-bis(benzyloxy)-2-(3,4,5-tris(benzyloxy)phenyl)chroman-3-yl 2-ethoxy-7-hydroxybenzo[d][1,3]dioxole-5-carboxylate (3ab-2). A mixture of compound 3ab-1 (1.5 g, 1.49 mmol) in THF (30 mL) was added NaBH4 (85 mg, 2.24 mmol) at ice-water bath. After stirring for 5 minute Pd(pph3)4 (86 mg, 0.07 mmol) was added. The mixture was stirred at room temperature under N2 overnight. The reaction mixture was concentrated and the residue was purified by flash chromatography on silica gel (PE/EA=1/1-DCM/MeOH=10/1) to give compound 3ab-2 (1.2 g, 83% yield) as a yellow solid. MS Calcd.: 964. MS Found: 965 [M+H]+.

Step 6: Synthesis of (2R,3R)-5,7-bis(benzyloxy)-2-(3,4,5-tris(benzyloxy)phenyl)chroman-3-yl 2-ethoxy-7-(propionyloxy)benzo[d][1,3]dioxole-5-carboxylate(3a-1). To a mixture of compound 3ab-2 (1.2 g, 1.24 mmol) in DCM (30 mL) was added propionic acid (110 mg, 1.49 mmol), EDCI (713 mg, 3.72 mmol) and DMAP (152 mg, 1.24 mmol) at 0° C. The reaction mixture was stirred at room temperature overnight. The reaction mixture was diluted with water (30 mL) and extracted with DCM (20 mL×2). The combined organic layers were dried over Na2SO4 and filtered. The filtrate was concentrated to give the crude product which was purified by flash chromatography on reverse phase silica gel (ACN/H2O=5%-95%, 254 nm, 40 min) to give compound 3a-1 (1.08 g, 85% yield) as yellow oil. MS Calcd.: 1020. MS Found: 1021 [M+H]+.

Step 7: Synthesis of (2R,3R)-5,7-dihydroxy-2-(3,4,5-trihydroxyphenyl)chroman-3-yl 2-ethoxy-7-(propionyloxy)benzo[d][1,3]dioxole-5-carboxylate (3a-2). To a mixture of compound 3a-1 (1.08 g, 1.06 mmol) in 20 mL of EA was added Pd(OH)2 (10% wt., 110 mg). The mixture was stirred at room temperature under H2 atmosphere (15 PSI) overnight. The reaction mixture was filtered and concentrated to give compound 3a-2 (0.52 g, 87% yield) as a yellow solid. MS Calcd.: 570. MS Found: 571 [M+H]+.

Step 8: Synthesis of Compound 77. To a mixture of compound 3a-2 (0.74 g, 1.3 mmol) in THF (30 mL) was added 2N HCl solution (5 mL) at 0° C. The reaction mixture was stirred at room temperature overnight. The reaction mixture was diluted with water (20 mL) and extracted with EA (20 mL×2). The combined organic layers were dried over Na2SO4, filtered and concentrated. The residue was purified by pre-UPLC to give (2R,3R)-5,7-dihydroxy-2-(3,4,5-trihydroxyphenyl)chroman-3-yl 3,4-dihydroxy-5-(propionyloxy)benzoate (80 mg, 12% yield) as a white solid. 1H NMR (400 MHZ, CD3OD) δ: 7.27-6.98 (m, 2H), 6.51 (s, 2H), 5.99-5.97 (m, 2H), 5.58 (d, J=16.8 Hz, 1H), 5.00 (s, 1H), 3.05-2.99 (m, 1H), 2.87 (d, J=19.2 Hz, 1H), 2.67-2.61 (m, 2H), 1.25-1.21 (m, 3H). MS Calcd.: 514. MS Found: 515 [M+H]+.

### Compound 78

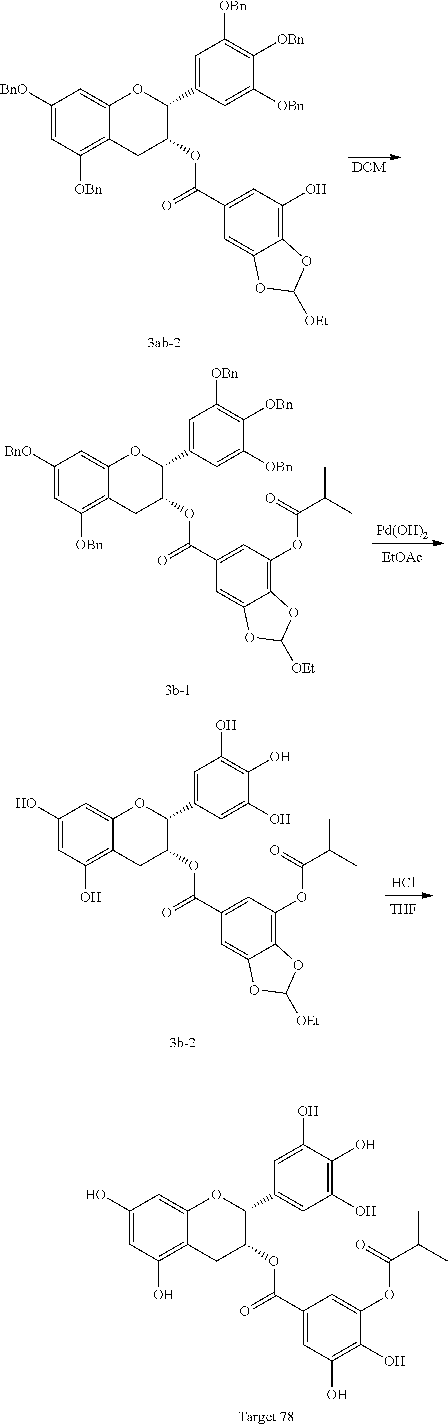

Step 1: Synthesis of (2R,3R)-5,7-bis(benzyloxy)-2-(3,4,5-tris(benzyloxy)phenyl)chroman-3-yl 2-ethoxy-7-(isobutyryloxy)benzo[d][1,3]dioxole-5-carboxylate (3b-1). To a mixture of compound 3ab-2 (300 mg, 0.31 mmol) in DCM (20 mL) was added isobutyric acid (41 mg, 0.47 mmol), EDCI (178 mg, 0.93 mmol) and DMAP (38 mg, 0.31 mmol) at 0° C. The reaction mixture was stirred at room temperature overnight. The reaction mixture was diluted with water (30 mL) and extracted with DCM (20 mL×2). The combined organic layer was dried over Na2SO4 and filtered. The filtrate was concentrated to give the crude product which was purified by flash chromatography on reverse phase silica gel (ACN/H2O=5%-95%, 254 nm, 40 min) to give compound 3b-1 (285 mg, 89% yield) as a white solid. MS Calcd.: 1034. MS Found: 1035 [M+H]+.

Step 2: Synthesis of (2R,3R)-5,7-dihydroxy-2-(3,4,5-trihydroxyphenyl)chroman-3-yl 2-ethoxy-7-(isobutyryloxy)benzo[d][1,3]dioxole-5-carboxylate (3b-2). To a mixture of compound 3b-1 (285 g, 0.28 mmol) in 20 mL of EA was added Pd(OH)2 (10% wt., 30 mg). The mixture was stirred at room temperature under H2 atmosphere (15 PSI) overnight. The reaction mixture was filtered and concentrated to give compound 3b-2 (160 mg, 100% yield) as a yellow solid which was used to the next step without further purification. MS Calcd.: 584. MS Found: 585 [M+H]+.

Step 3: Synthesis of Compound 78. To a mixture of compound 3b-2 (160 mg, 0.27 mmol) in THF (15 mL) was added 2N HCl solution (3 mL) at 0° C. The reaction mixture was stirred at room temperature overnight. The reaction mixture was diluted with water (20 mL) and extracted with EA (20 mL×2). The combined organic layers were dried over Na2SO4, filtered and concentrated. The residue was purified by pre-UPLC to give (2R,3R)-5,7-dihydroxy-2-(3,4,5-trihydroxyphenyl)chroman-3-yl 3,4-dihydroxy-5-(isobutyryloxy)benzoate (31 mg, 21% yield) as a white solid. 1H NMR (400 MHZ, CD3OD) δ: 7.15-6.85 (m, 2H), 6.38 (s, 2H), 6.85 (d, J=4.4 Hz, 2H), 5.46 (d, J=14.8 Hz, 1H), 4.88 (s, 1H), 2.92-2.86 (m, 1H), 2.77-2.70 (m, 2H), 1.20-1.17 (m, 6H). MS Calcd.: 528. MS Found: 529 [M+H]+.

### Compound 79

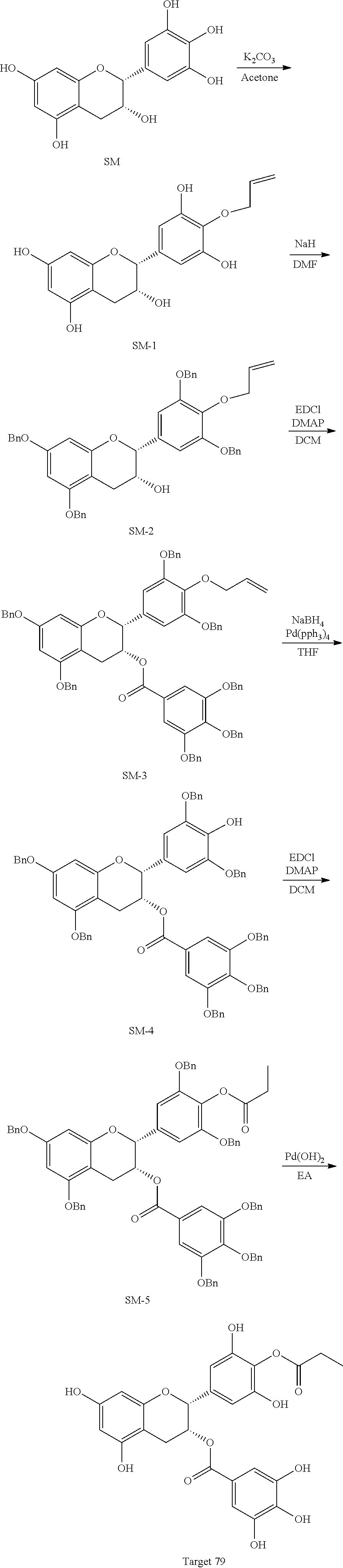

Step 1: Synthesis of (2R,3R)-2-(4-(allyloxy)-3,5-dihydroxyphenyl)chroman-3,5,7-triol (SM-1). To a solution of compound SM1 (300 mg, 0.98 mmol) in Acetone (20 mL) was added K2CO3 (270 mg, 1.96 mmol) at 0° C. The mixture was stirred at 0° C. for 30 minute. Compound 3-bromoprop-1-ene (142 mg, 1.18 mmol) was added at 0° C. and the solution was stirred at 55° C. for 3 hours. The reaction mixture was concentrated. The residue was purified by flash chromatography on reverse phase silica gel (ACN/H2O=5%-95%, 254 nm, 30 min) to give compound SM-1 (102 mg, 30% yield) as a yellow solid. 1H NMR (400 MHZ, CD3OD): 6.53 (s, 2H), 6.17-6.08 (m, 1H), 5.95-5.92 (m, 2H), 5.32-5.27 (m, 1H), 5.18-5.15 (m, 1H), 4.77 (s, 1H), 4.59-4.52 (m, 2H), 4.19-4.18 (m, 1H), 2.88-2.83 (m, 1H), 2.77-2.71 (m, 1H). MS Calcd.: 346. MS Found: 347 [M+H]+.

Step 2: Synthesis of (2R,3R)-2-(4-(allyloxy)-3,5-bis(benzyloxy)phenyl)-5,7-bis(benzyloxy)chroman-3-ol (SM-2). To a solution of compound SM-1 (6.7 g, 19.35 mmol) in DMF (80 mL) was added NaH (3.25 mg, 81.25 mmol, 60% wt. in mineral oil) at 0° C. The mixture was stirred at 0° C. for 20 minutes. BnCl (1028 g, 81.25 mmol) was added at 0° C. and the solution was stirred at room temperature overnight. The reaction mixture was diluted with H2O (50 mL) and extracted with EA (50 mL×2). The combined organic layers were washed with brine (30 mL×2), dried over Na2SO4, filtered and concentrated. The residue was purified by flash chromatography on silica gel (PE/EA=2/1) to give the crude product. The residue was further purify by flash chromatography on reverse phase silica gel (ACN/H2O=5%-95%, 254 nm, 45 min) to give compound SM-2 (4.2 g, 31% yield) as yellow oil. MS Calcd.: 706; MS Found: 707 [M+H]+

Step 3: Synthesis of (2R,3R)-2-(4-(allyloxy)-3,5-bis(benzyloxy)phenyl)-5,7-bis(benzyloxy)chroman-3-yl 3,4,5-tris(benzyloxy)benzoate (SM-3). To a solution of compound SM-2 (4.2 g, 5.94 mmol) in DCM (50 mL) was added 3,4,5-tris(benzyloxy)benzoic acid (3.40 g, 7.72 mmol), EDCI (3.42 g, 17.82 mmol) and DMAP (725 mg, 5.94 mmol) at 0° C. The reaction mixture was stirred at room temperature overnight. The reaction mixture was diluted with water (30 mL) and extracted with DCM (20 mL×2). The combined organic layers were dried over Na2SO4 and filtered. The filtrate was concentrated to give the crude product which was purified by flash chromatography on reverse phase silica gel (ACN/H2O=5%-95%, 254 nm, 40 min) to give compound SM-3 (5.8 g, 86% yield) as yellow oil. MS Calcd.: 1128; MS Found: 1145 [M+H]++17.

Step 4: Synthesis of (2R,3R)-5,7-bis(benzyloxy)-2-(3,5-bis(benzyloxy)-4-hydroxyphenyl)chroman-3-yl 3,4,5-tris(benzyloxy)benzoate (SM-4). A mixture of compound SM-3 (5.8 g, 5.14 mmol) in THF (50 mL) was added NaBH4 (293 mg, 7.70 mmol) at ice-water bath. After stirring for 5 minutes, Pd(pph3)4 (594 mg, 0.514 mmol) was added. The mixture was stirred at room temperature overnight. The reaction mixture was diluted with water (30 mL) and extracted with DCM (30 mL×2). The combined organic layers were washed with brine (30 ml×2), dried over Na2SO4, filtered and concentrated to give compound SM-4 (5.31 g, 95% yield) as yellow oil. MS Calcd.: 1088. MS Found: 1105 [M+H]++17.

Step 5: Synthesis of (2R,3R)-5,7-bis(benzyloxy)-2-(3,5-bis(benzyloxy)-4-(propionyloxy)phenyl)chroman-3-yl 3,4,5-tris(benzyloxy)benzoate (SM-5). To a mixture of compound SM-4 (800 mg, 0.73 mmol) in DCM (30 mL) was added propionic acid (65 mg, 0.44 mmol), EDCI (420 mg, 2.19 mmol) and DMAP (89 mg, 0.73 mmol) at 0° C. The reaction mixture was stirred at room temperature overnight. The reaction mixture was diluted with water (30 mL) and extracted with DCM (20 mL×2). The combined organic layers were dried over Na2SO4 and filtered. The filtrate was concentrated to give a crude product, which was purified by flash chromatography on reverse phase silica gel (ACN/H2O=5%-95%, 254 nm, 40 min) to give compound SM-5 (680 mg, 88% yield) as yellow oil. MS Calcd.: 1044. MS Found: 1162 [M+H]++17.

Step 6: Synthesis of Compound 79. To a mixture of compound SM-5 (680 mg, 0.59 mmol) in EA (20 mL) was added Pd(OH)2 (10% wt., 60 mg). The mixture was stirred at room temperature under H2 of balloon overnight. The reaction mixture was filtered and concentrated. The residue was purified by pre-UPLC to give (2R,3R)-2-(3,5-dihydroxy-4-(propionyloxy)phenyl)-5,7-dihydroxychroman-3-yl 3,4,5-trihydroxybenzoate (65 mg, 21% yield) as a white solid. 1H NMR (400 MHZ, CD3OD) o: 6.84 (s, 2H), 6.47 (s, 2H), 5.86 (s, 2H), 5.45 (s, 1H), 492 (s, 1H), 3.12-3.08 (m, 1H), 2.92-2.87 (m, 2H), 2.78-2.73 (m, 2H), 1.04 (t, J=3.6 Hz, 3H). MS Calcd.: 514.4. MS Found: 515.4 [M+H]+.

### Compound 80

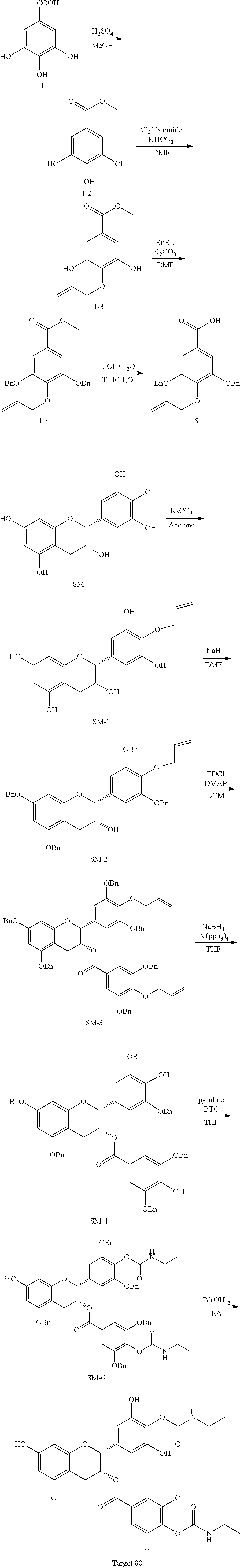

Step 1: Synthesis of methyl 3,4,5-trihydroxybenzoate (1-2). To a solution of compound 1-1 (20 g, 0.12 mol) in MeOH (200 mL) was added H2SO4 (6 mL) at 0° C. The mixture was stirred at 80° C. overnight. The reaction mixture was neutralized with Na2CO3 solution at 0° C. and extracted with EA (100 mL×3). The combined organic layers were washed with brine (100 mL×2), dried over Na2SO4, filtered and concentrated to give compound 1-2 (15 g, 70% yield) as a yellow solid. MS Calcd.: 184. MS Found: 185 [M+H]+.

Step 2: Synthesis of methyl 4-(allyloxy)-3,5-dihydroxybenzoate (1-3). To a solution of compound 1-2 (2.92 g, 15.87 mmol) in DMF (30 mL) was added 3-bromoprop-1-ene (1.92 g, 15.87 mmol), NaHCO3(5.33 g, 63.46 mmol) and KI (2.63 g, 15.87 mmol). The reaction mixture was stirred at room temperature overnight. The reaction mixture was diluted with H2O (50 mL) and extracted with EA (50 mL×2). The combined organic layers were washed with brine (30 mL×2), dried over Na2SO4, filtered and concentrated. The residue was purified by flash chromatography on silica gel (PE/EA=5/1-1/1) to give compound 1-3 (2.4 g, 67% yield) as yellow oil. MS Calcd.: 224. MS Found: 225 [M+H]+.

Step 3: Synthesis of methyl 4-(allyloxy)-3,5-bis(benzyloxy)benzoate (1-4). To a solution of compound 1-3 (1.8 g, 8.03 mmol) in DMF (30 mL) was added K2CO3 (2.22 g, 16.06 mmol) and BnBr (4.12 g, 24.1 mmol) at ice-water bath. The reaction mixture was stirred at 60° C. overnight. The reaction mixture was diluted with H2O (30 mL) and extracted with EA (50 mL×2). The combined organic layers were washed with brine (30 mL×2), dried over Na2SO4, filtered and concentrated. The residue was purified by flash chromatography on silica gel (PE/EA=5/1-3/1) to give compound 1-4 (2.98 g, 92% yield) as a white solid. MS Calcd.: 404; MS Found: 405 [M+H]+.

Step 4: Synthesis of 4-(allyloxy)-3,5-bis(benzyloxy)benzoic acid (1-5). To a solution of compound 1-4 (2.98 g, 7.38 mmol) in THF/H2O (30 mL/10 mL) was added LiOH·H2O (0.62 g, 14.75 mmol). The reaction mixture was stirred at 50° C. overnight. The reaction mixture was adjusted to pH=5 with 1N HCl solution and extracted with EA (50 mL×2). The combined organic layers were washed with brine (30 mL×2), dried over Na2SO4, filtered and concentrated to give compound 1-5 (2.4 g, 83% yield) as a white solid. MS Calcd.: 390; MS Found: 391 [M+H]+.

Step 5: Synthesis of (2R,3R)-2-(4-(allyloxy)-3,5-dihydroxyphenyl)chroman-3,5,7-triol (SM-1). To a solution of compound SM1 (300 mg, 0.98 mmol) in Acetone (20 mL) was added K2CO3 (270 mg, 1.96 mmol) at 0° C. The mixture was stirred at 0° C. for 30 minutes. Then 3-bromoprop-1-ene (142 mg, 1.18 mmol) was added at 0° C. and the solution was stirred at 55° C. for 3 hours. The reaction mixture was concentrated. The residue was purified by flash chromatography on reverse phase silica gel (ACN/H2O=5%-95%, 254 nm, 30 min) to give compound SM-1 (102 mg, 30% yield) as a yellow solid. 1H NMR (400 MHZ, CD3OD) δ: 6.53 (s, 2H), 6.17-6.08 (m, 1H), 5.95-5.92 (m, 2H), 5.32-5.27 (m, 1H), 5.18-5.15 (m, 1H), 4.77 (s, 1H), 4.59-4.52 (m, 2H), 4.19-4.18 (m, 1H), 2.88-2.83 (m, 1H), 2.77-2.71 (m, 1H). MS Calcd.: 346; MS Found: 347 [M+H]+.

Step 6: Synthesis of (2R,3R)-2-(4-(allyloxy)-3,5-bis(benzyloxy)phenyl)-5,7-bis(benzyloxy)chroman-3-ol (SM-2). To a solution of compound SM-1 (6.7 g, 19.35 mmol) in DMF (80 mL) was added NaH (3.25 mg, 81.25 mmol, 60% wt. in mineral oil) at 0° C. The mixture was stirred at 0° C. for 20 minutes. BnCl (1028 g, 81.25 mmol) was added at 0° C. and the solution was stirred at room temperature overnight. The reaction mixture was diluted with H2O (50 mL) and extracted with EA (50 mL×2). The combined organic layers were washed with brine (30 mL×2), dried over Na2SO4, filtered and concentrated. The residue was purified by flash chromatography on silica gel (PE/EA=2/1) to give the crude product. The residue was further purify by flash chromatography on reverse phase silica gel (ACN/H2O=5%-95%, 254 nm, 45 min) to give compound SM-2 (4.2 g, 31% yield) as yellow oil. MS Calcd.: 706; MS Found: 707 [M+H]+

Step 7: Synthesis of (2R,3R)-2-(4-(allyloxy)-3,5-bis(benzyloxy)phenyl)-5,7-bis(benzyloxy)chroman-3-yl 4-(allyloxy)-3,5-bis(benzyloxy)benzoate (SM-3). To a solution of compound SM-2 (2.31 g, 3.27 mmol) in DCM (50 mL) was added 3,4,5-tris(benzyloxy)benzoic acid (1.40 g, 3.59 mmol), EDCI (1.88 g, 9.81 mmol) and DMAP (399 mg, 3.27 mmol) at 0° C. The reaction mixture was stirred at room temperature overnight. The reaction mixture was diluted with water (30 mL) and extracted with DCM (20 mL×2). The combined organic layers were dried over Na2SO4 and filtered. The filtrate was concentrated to give the crude product which was purified by flash chromatography on reverse phase silica gel (ACN/H2O=5%-95%, 254 nm, 40 min) to give compound SM-3 (1.72 g, 49% yield) as yellow oil. MS Calcd.: 1078. MS Found: 1079 [M+H]+.

Step 8: Synthesis of (2R,3R)-5,7-bis(benzyloxy)-2-(3,5-bis(benzyloxy)-4-hydroxyphenyl)chroman-3-yl 3,5-bis(benzyloxy)-4-hydroxybenzoate (SM-4). A mixture of compound SM-3 (1.72 g, 1.59 mmol) in THF (50 mL) was added NaBH4 (90 mg, 2.38 mmol) at ice-water bath. After 5 minutes, Pd(pph3)4 (184 mg, 0.16 mmol) was added. The mixture was stirred at room temperature overnight. The reaction mixture was diluted with water (30 mL) and extracted with DCM (30 mL×2). The combined organic layers were washed with brine (30 ml×2), dried over Na2SO4, filtered and concentrated to give compound SM-4 (5.31 g, 95% yield) as yellow oil. MS Calcd.: 998. MS Found: 999 [M+H]+.

Step 9: Synthesis of (2R,3R)-5,7-bis(benzyloxy)-2-(3,5-bis(benzyloxy)-4-((ethylcarbamoyl)oxy)phenyl)chroman-3-yl 3,5-bis(benzyloxy)-4-((ethylcarbamoyl)oxy)benzoate (SM-6). To a mixture of compound SM-4 (1.0 g, 10 mmol) in THF (30 mL) was added pyridine (3.16 g, 40 mmol) and Bis(trichloromethyl)Carbonate (1.19 g, 4.0 mmol) at ice-water bath. After 10 minutes, ethanamine (676 mg, 15 mmol) was added at 0° C. The reaction mixture was stirred at room temperature for 1 hour. The reaction mixture was diluted with water (30 mL) and extracted with DCM (20 mL×2). The combined organic layers were dried over Na2SO4 and filtered. The filtrate was concentrated to give the crude product which was purified by flash chromatography on reverse phase silica gel (ACN/H2O=5%-95%, 254 nm, 40 min) to give compound SM-6 (0.732 g, 64% yield) as yellow oil. MS Calcd.: 1141; MS Found: 1158 [M+H]++17.

Step 10: Synthesis of Compound 80. To a mixture of compound SM-6 (530 mg, 0.46 mmol) in EA (20 mL) was added Pd(OH)2 (10% wt., 53 mg). The mixture was stirred at room temperature under H2 of balloon overnight. The reaction mixture was filtered and concentrated. The residue was purified by pre-UPLC to give (2R,3R)-2-(4-((ethylcarbamoyl)oxy)-3,5-dihydroxyphenyl)-5,7-dihydroxychroman-3-yl 4-((ethylcarbamoyl)oxy)-3,5-dihydroxybenzoate (50 mg, 18% yield) as a white solid. 1H NMR (400 MHZ, CD3OD) δ: 7.13-6.84 (m, 2H), 6.44 (d, J=2.8 Hz, 2H), 5.87-5.85 (m, 2H), 5.47 (d, J=16 Hz, 1H), 4.92 (s, 1H), 3.12-3.07 (m, 4H), 2.93-2.88 (m, 1H), 2 . . . 80-2.74 (m, 1H), 1.08-1.05 (m, 6H). MS Calcd.: 600. MS Found: 601 [M+H]+

### Compound 81

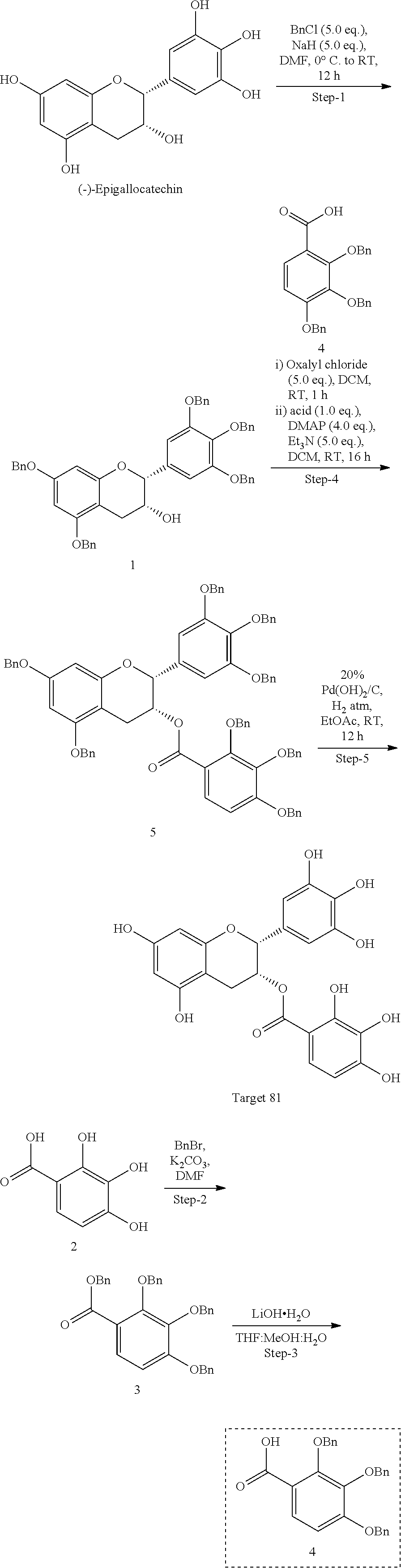

Step 1: Synthesis of (2R,3R)-5,7-bis(benzyloxy)-2-(3,4,5-tris(benzyloxy)phenyl)chroman-3-ol (1). To a stirred solution of EGC (5.0 g, 16.33 mmol, 1.0 eq.) in dry DMF (50 mL) was added NaH (60%. wt in mineral oil) (3.266 g, 81.69 mmol, 5.0 eq.) at 0° C. and stirred for 0.5 h. To this was added BnCl (9.4 mL, 81.69 mmol, 5.0 eq.) drop wise at 0° C. The suspension was allowed to stir at RT for 12 h. After complete consumption of the starting material, the reaction mixture was quenched with sat. aq. Na₂SO₄ (2 mL) and filtered through pad of celite. The celite pad was washed with EtOAc (100 mL). The combined solvents were sequentially washed with H₂O (100 mL) and brine (30 mL). The organic layer was dried over Na₂SO₄, filtered and concentrated. The obtained residue was purified by flash chromatography on silica gel (PE/EA=6/1) to give 1 (6.20 g, 50% yield) as a pale yellow solid. ¹H NMR (400 MHZ, DMSO-d₆): δ 7.50-7.19 (m, 25H), 6.94 (s, 2H), 6.34 (d, J=2.4 Hz, 1H), 6.17 (d, J=2.0 Hz, 1H), 5.20-5.01 (m, 8H), 5.00-4.88 (m, 9H), 4.84 (d, J=4.4 Hz, 1H), 4.15 (d, J=3.6 Hz, 1H), 2.88-2.62 (m, 2H).

Step 2: Synthesis of benzyl 2,3,4-tris(benzyloxy)benzoate (3). To a solution of 2, 3, 4-trihydroxybenzoic acid (2.5 g, 14.70 mmol, 1.0 eq.) in DMF (25 mL) was added K₂CO₃ (12.176 g, 88.23 mmol, 6.0 eq.) and BnBr (10.5 mL, 88.23 mmol, 6.0 eq.) at 0° C. The mixture was stirred at RT for 16 h. After complete consumption of the starting material, reaction mixture was filtered through pad of celite. The celite pad was washed with EtOAc (50 mL). The combined solvents were sequentially washed with H₂O (50 mL) and brine (30 mL). The organic layer was dried over Na₂SO₄, filtered and concentrated. The obtained residue was purified by flash chromatography on silica gel (PE/EA=6/1) to give 3 (5.5 g, 70.5% yield) as a white solid. ¹HNMR (400 MHZ, DMSO-d₆): δ 7.58 (d, J=8.8 Hz, 1H), 7.49 (d, J=6.8 Hz, 2H), 7.42-7.26 (m, 18), 7.08 (d, J=9.2 Hz, 1H), 5.27 (s, 2H), 5.23 (s, 2H), 4.97 (s, 4H).

Step 3: Synthesis of (2,3,4-tris(benzyloxy)benzoic acid (4). To a solution of Benzyl 2,3,4-tris(benzyloxy) benzoate (4.3 g, 8.11 mmol, 1.0 eq.) in THF:MeOH:H₂O (1:1:1) (45 mL) was added LiOH H₂O (1.703 g, 40.56 mmol, 5.0 eq.). The solution was stirred at RT for 4 h. The reaction mixture was concentrated to remove THF. Then the mixture was diluted with H₂O (50 mL) and extracted with Et₂O (2×30 mL). The aqueous phase was adjusted to pH<3 with 1 N HCl. Then the obtained solid was filtered through sintered funnel and dried to give the 2, 3, 4-tris(benzyloxy)benzoic acid (4) (2.5 g, 70.2% yield) as a white solid. ¹HNMR (400 MHZ, DMSO-d₆); δ 12.65 (s, 1H), 7.56-7.27 (m, 15H), 7.05 (d, J=8.8 Hz, 1H), 5.23 (s, 2H), 5.00 (s, 2H), 4.97 (s, 2H).

Step 4: Synthesis of (2R,3R)-5,7-bis(benzyloxy)-2-(3,4,5-tris(benzyloxy)phenyl)chroman-3-yl 2,3,4-tris(benzyloxy)benzoate (5). To a solution of carboxylic acid (4) (0.873 g, 1.98 mmol, 1.0 eq.) in CH₂Cl₂ (10 mL) was added (COCl)₂ (0.86 mL, 9.92 mmol, 5.0 eq.) and 2 drops of dry DMF at 0° C. The mixture was stirred at RT for 1 h. Solvent was evaporated from the reaction mixture under reduced pressure in the presence of organ gas to give acid chloride. To this was added a mixture of 1 (1.5 g, 1.98 mmol, 1.0 eq.), DMAP (0.966 g, 7.92 mmol, 4.0 eq.) and TEA (1.1 mL, 7.92 mmol, 4.0 eq.) dissolved in CH₂Cl₂ (20 mL) at 0° C. The resulting mixture was stirred at RT for 16 h. The reaction mixture was diluted with H₂O (30 mL) and extracted with CH₂Cl₂ (2×50 mL). The combined organic layers were washed with brine (30 mL), dried over Na₂SO₄, filtered and concentrated. The residue was purified by flash chromatography on silica gel (PE/EA=7/1) to give 5 (1.56 g, 66.7% yield) as a white solid. ¹H NMR (400 MHZ, DMSO-d₆): δ 7.44-7.17 (m, 40H), 7.11 (d, J=7.2 Hz, 2H), 7.02 (dd, J=10.4, 5.6 Hz, 2H), 6.40 (d, J=2.0 Hz, 1H),), 6.23 (d, J=2.0 Hz, 1H), 5.68 (s, 1H), 5.27 (s, 1H), 5.17-4.64 (m, 16H), 3.17 (d, J=12.0, 1H), 2.95 (d, J=16.8 Hz, 1H).

Step 5: Synthesis of (2R,3R)-5,7-dihydroxy-2-(3,4,5-trihydroxyphenyl)chroman-3-yl 2-fluoro-3,4,5-trihydroxybenzoate (Target-81). To a solution of 5 (1.55 g, 1.31 mmol, 1.0 eq.) in THF (18 mL) and MeOH (12 mL) was added Pd(OH)₂/C (20% wt., 0.184 g). The mixture was stirred at room temperature under H₂ atmosphere for overnight. The reaction mixture was filtered and the filtrate was concentrated. The residue was purified by prep-HPLC to give Target-81 (0.334 g, 55.4% yield) as pale pink color solid. ¹H NMR (400 MHZ, DMSO-d₆) δ 10.21 (bs, 2H), 9.32 (bs, 2H), 9.09 (bs, 2H), 8.75 (bs, 2H), 7.01 (d, J=8.8 Hz, 1H), 6.40 (s, 2H), 6.33 (d, J=8.8 Hz, 1H), 5.92 (d, J=2.0 Hz, 1H), 5.82 (d, J=2.0 Hz, 1H), 5.39 (s, 1H), 5.00 (s, 1H), 2.97 (dd, J=16.8, 3.6 Hz, 1H), 2.74 (d, J=16.8 Hz, 1H)

### Example 2. In Vitro Activity

Compounds of Table 1, above, were examined in vitro for activity against DYRK1A, DYRK1B, DYRK2 via an ELISA assay.

### General Assay Procedure

DYRK1A assay. Substrate, HT-PRD (Proline rich domain, residues 746-864 of dynamin 1a, prepared as N-terminal tagged 6×His fusion protein), was diluted in dilution buffer (25 mM Tris-HCl, pH 7.4 and 100 mM NaCl) to a concentration of 2 ng/μl or higher and used to coat a 96-well plate (BD Falcon #353072) with 100 μl per well (200 ng/well unless otherwise indicated) at 4° C. overnight. Unbound materials were washed away with dilution buffer and wells were blocked with 150 μl blocking buffer (2% BSA, 1×PBS, and 0.25% Tween 20) at room temperature for 60 min. After blocking, wells were washed extensively with dilution buffer before subjecting to phosphorylation. DYRK1A phosphorylation was performed in wells with 100 μl reaction mix containing 25 mM HEPES, pH7.4, 100 mM NaCl, 5 mM MgCl₂, 100 UM ATP (Sigma-Aldrich Chemicals), inhibitor if needed, and 5 ng HT-497 (6×His tagged rat truncated DYRK1A isoform X1 containing residues 1-497). Reactions were initiated by adding HT-497 and continued for 30 min (unless otherwise indicated) at 30° C. At the end point, wells were washed with ˜350 μl dilution buffer three times to terminate the reaction. A set of inhibition experiments typically consisted of a no-inhibitor control plus a series of eight inhibitor concentrations in the range of 0.000625 UM-100 μM (final) depending on the strength of inhibitor. Each point was run in quadruplicate with DMSO present in all assays at 0.2% final concentration. DMSO, up to 5%, did not affect the potency of compound 3 and harmine. HT-PRD phosphorylation was subsequently determined by the sandwich antibody staining protocol, first with 100 μl mouse mAb 3D3 specific for detecting DYRK1A-phosphorylated PRD (60 min at room temperature) then with 100 μl alkaline phosphatase (AP)-linked anti-mouse secondary antibody (60 min at room temperature), followed by colorimetric reaction with 100 μl PNPP solution (PNPP substrate tablet, Thermo Fisher). The extent of AP reaction was monitored at A=405 nm until OD for the no-inhibitor controls reached between 1-2. The readings were then recorded for subsequent IC₅₀ calculation. The DYRK1A assay protocol can also support DYRK1B and DYRK2 phosphorylation reactions in the enzyme concentration-dependent manner; therefore, the method was adapted for measuring the activity of candidate compounds against DYRK1B and DYRK2. For DYRK2, the reactions were performed as described above with 6 ng HT-DYRK2 (6×His tagged full-length human DYRK2 isoform 1). For DYRK1B, the assays were also similarly conducted but with 70 ng GST-DYRK1B (glutathione S-transferase tagged full length human DYRK1B isoform p65) and an extended kinase reaction time of 60 min.

Kinase Verification. Kinase preparations were verified by the following immunological and biochemical criteria to ensure the identity of each kinase before use. (1) Immunoreactivity only with the cognate antibody and (2) Sensitivity to 50 nM inhibitor AZ-191. With IC₅₀ of 88 nM, 17 nM, and 1890 nM for DYRK1A, DYRK1B, and DYRK2 (Anne L. Ashford, David Oxley, Jason Kettle, Kevin Hudson, Sylvie Guichard, Simon J. Cook, Pamela A. Lochhead; A novel DYRK1B inhibitor AZ191 demonstrates that DYRK1B acts independently of GSK3β to phosphorylate cyclin D1 at Thr286, not Thr288. Biochem J 1 Jan. 2014; 457 (1): 43-56. doi: https://doi.org/10.1042/BJ20130461), respectively, 50 nM AZ-191 sensitivity can be used to rapidly differentiate these kinases.

Dilution factors for both mAb 3D3 and secondary antibody were pre-determined for each batch of antibody to ensure that neither antibody was limiting in the assay. A stock to be determined was serially diluted (from 1000 to 256,000-fold) and each dilution was used together with a non-limiting concentration of the other antibody to assess the level of HT-PRD phosphorylated under standard ELISA reaction conditions without inhibitor. OD 405 readings were normalized to the 1000-fold dilution and plotted against the dilutions of the testing antibody. Dilutions in the normalized OD 405 plateau can be used for the assay. 1:2000 dilutions were routinely used for Baker Abx resin purified 3D3 stock (˜1.5 mg/ml) and 1:2000 dilutions of commercial AP-conjugated secondary antibody (Jackson ImmunoResearch #115-055-146) for the assay.

Data transformation, calculation, plotting, curve fitting, and IC 50 calculation were performed in KaleidaGraph (http://www.synergy.com/wordpress_650164087; Mac version 4.1). Data was corrected for background (readings from wells with only PNPP) before subsequent manipulations. To determine IC 60, the residual DYRK1A activity was first calculated as the ratio to the no-inhibitor control in that set. The resulting residual activity was then plotted against its corresponding inhibitor concentrations in semi-log graph and the plot was fit to the sigmoidal equation, y=a+(b−a)/(1+(x/c)^{d}), for IC₅₀ calculation. Results, shown in Table 2, below, are presented as mean±standard error of mean.

### Results

TABLE 2
DYRK1A Activity

| Compound | DYRK1A IC₅₀ |
|---|---|
| 3 | ** |
| 4 | ** |
| 5 | ** |
| 6 | ** |
| 7 | ** |
| 8 | ** |
| 9 | ** |
| 10 | * |
| 11 | ** |
| 12 | ** |
| 13 | * |
| 14 | ** |
| 15 | * |
| 16 | * |
| 17 | * |
| 18 | ** |
| 19 | ** |
| 20 | * |
| 21 | * |
| 22 | * |
| 23 | * |
| 24 | * |
| 25 | * |
| 26 | * |
| 27 | ** |
| 28 | * |
| 29 | * |
| 30 | * |
| 31 | ** |
| 32 | ** |
| 33 | ** |
| 34 | ** |
| 35 | * |
| 36 | * |
| 37 | * |
| 38 | ** |
| 39 | ** |
| 40 | *** |
| 41 | ** |
| 42 | ** |
| 43 | * |
| 44 | * |
| 45 | ** |
| 46 | *** |
| 47 | *** |
| 48 | ** |
| 49 | ** |
| 50 | ** |
| 51 | *** |
| 52 | ** |
| 53 | * |
| 55 | *** |
| 59 | ** |
| 60 | ** |
| 61 | ** |
| 62 | ** |
| 63 | ** |
| 64 | ** |
| 65 | ** |
| 66 | ** |
| 68 | *** |
| 69 | ** |
| 70 | ** |
| 71 | ** |
| 72 | ** |

(* IC50 > 1000 nM, ** IC50 > 100 nM, *** IC50 < 100 nM)

Compounds of Table 1, above, were examined in vitro for activity against DYRK1B, via an ELISA assay.

### Results

TABLE 3
DYRK1B Activity

| Compound | DYRK1B IC₅₀ |
|---|---|
| 3 | ** |
| 4 | ** |
| 40 | *** |
| 51 | *** |
| 55 | ** |
| 68 | *** |

(* IC50 > 1000 nM, ** IC50 > 100 nM, *** IC50 < 100 nM)

### Example 3. In vivo Efficacy of Compound 40

Compound 40 of Table 1 was examined for application in neurodegenerative disorders using a MOG₃₅₋₅₅-induced murine model of chronic progressive Experimental Autoimmune Encephalomyelitis (EAE), an inflammation model predictive for multiple sclerosis (MS).

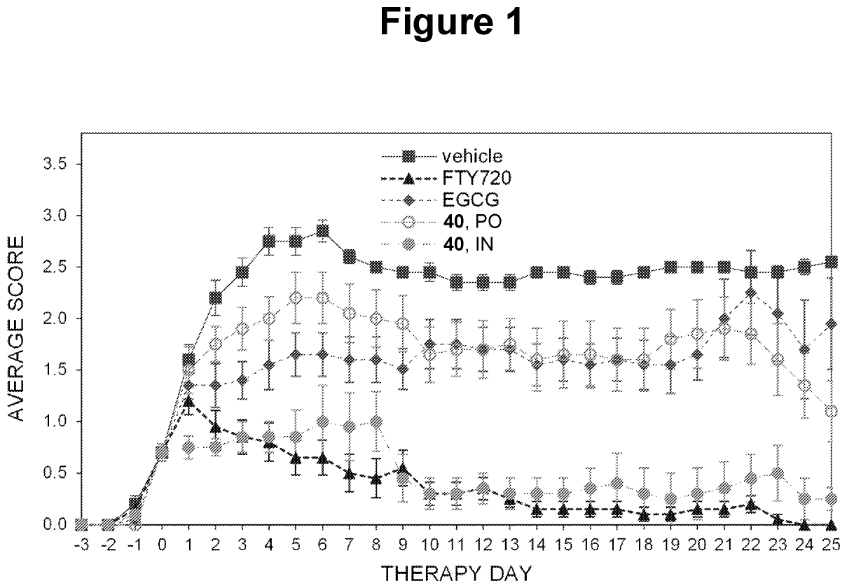

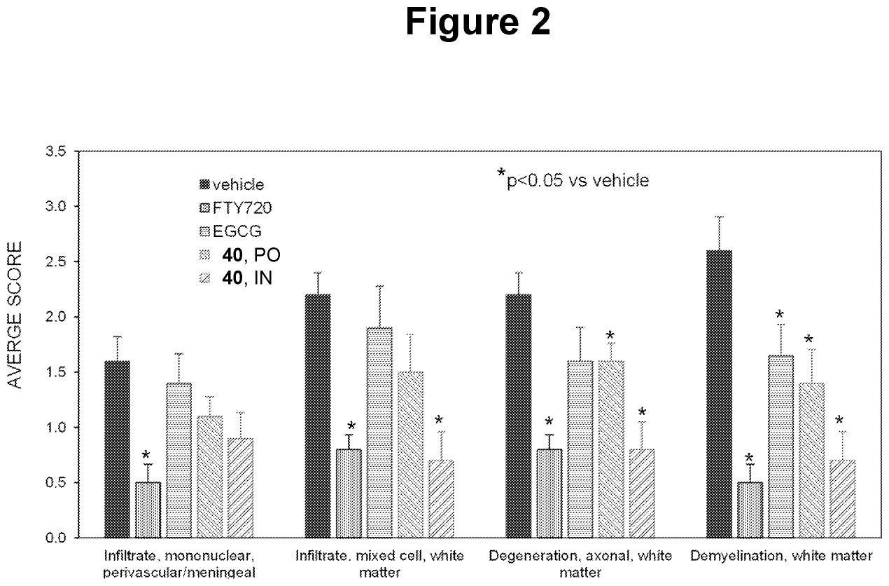

Compound 40 was tested both PO and IN, and compared against two reference compounds, namely compound 3 and Fingolimod (FTY720). As shown in FIG. 1, twice daily intranasal administration of compound 40 (15 mg/kg) resulted in a reversal of the disease similar to that observed with FTY720. This treatment regimen also significantly reduced brain IFNγ (90%) and IL-17 (79%). Despite literature reports, no significant activity was observed for compound 3 in this model. Histopathology analysis of a section of the spinal cord showed that mice treated intranasally with compound 40 exhibited significant (p<0.001) reductions in lesion severity scores when compared to the other treatment groups and is similar to FTY720 (FIG. 2). Staining with hematoxylin and eosin revealed that compound 40 led to significant spinal cord inflammation reductions compared to the vehicle control group.

Desirably, compound 40 also preserved axons and myelin during chronic EAE, as reflected by the staining of neurofilaments assessed by Bielschowsky silver staining and myelin determined by LFB staining. Without being bound by theory, the present inventors note that compound 40 could have a peripheral immunomodulation effect and an overall neuroprotective effect, which could have advantageous implications for the treatment of neurodegenerative disorders like Alzheimer's disease and Multiple Sclerosis.

### Example 3. PK Characterization of Compound 68

Pharmacokinetic studies were conducted for compound 68 as part of a stand-alone study in adult male C57BL/6 mice (n=3 per time point; 7-time points) using 1) a single IV dose (5 mg/kg), 2) a single PO dose (30 mg/kg), and 3) an intranasal, IN dose of 30 mg/kg. The IV/PO study's absolute oral bioavailability was 4%, whereas the intranasal bioavailability was >100%. In another separate study, adult male mice C57BL/6 mice were administered compound 68 by way of the intranasal route at 10 mg/kg along with other compounds in a cassette dosing study. It was concluded that exposure of compound 68 (Cmax and AUC) increased from 10 to 30 mg/kg and emphasized the greater bioavailability of compound 68 when administered IN versus PO. Results are shown in Table 3, below.

TABLE 3
Pharmacokinetic properties of Compound 68

| | | i.v. | intranasal | intranasal | oral |
|---|---|---|---|---|---|
| | unit | (5 mg/kg) | (10 mg/kg) | (30 mg/kg) | (30 mg/kg) |
| T_{1/2} | H | 1.04 | 1.06 | 1.97 | 1.27 |
| Cₘₐₓ | ng/mL | 257 | 489 | 3143 | 41 |
| AUC₀₋ₜ | ng h/mL | 334 | 702 | 4950 | 78 |
| AUC_{0-inf.} | ng h/mL | 348 | 716 | 4988 | 92 |
| V_{Z} (IV) | L/kg | 22 | 41.7 | 17.1 | 602 |
| V_{Z}/F (IN, PO) | | | | | |
| CL (IV) | mL/min/kg | 240 | 240 | 842 | 100 |
| CL/F (IN, PO) | | | | | |
| F | % | NA | >100 | >100 | 4 |

Whole-brain homogenate analyses showed that compound 68 was measurable for 4 hours after dosing only when administered intranasally (e.g., 120 ng/g @ 1 hr post-dose) while, when given IV or PO, the drug concentration was below the lower limit of quantitation at all 7 time-points (LLOQ=approx. 5 ng/ml plasma/brain). The apparent volume of distribution (Vz) was high after IV and IN dosing at 10 mg/kg but even higher after PO dosing. The plasma clearance values were high and exceeded hepatic blood flow for all three dosing regimens. Overall, when compound 68 was administered intranasally and 10 and 30 mg/kg, it showed exceptionally good bioavailability as assessed by plasma Cmax and AUC values and was far superior to PO administration.

### Example 4. Safety Profile of Compound 68

Compound 68 was screened (c=10 UM) in the Eurofins' safetyscreen87® panel. The drug showed a very clean profile; among the 87 different targets tested, significant activity was only observed against the following enzymes: COX1 (IC₅₀=1200 nM), COX2 (IC₅₀=182 nM), and PDE3A (IC₅₀=380 nM). These targets are not considered problematic since they have broad anti-inflammatory activity in the brain, potentially beneficial in Alzheimer's disease and other neurodegenerative disorders. Notable is the potent activity against COX2, similar to the known inhibitor Rofecoxib (Vioxx®, IC₅₀=260 nM). The use of the proprietary nose-to-brain delivery technology will minimize any safety concerns due to peripheral off-target activity. No activity (@ c=10 μM) was observed against the Potassium channel hERG and other ion channels, minimizing the risk of cardiovascular toxicity.

To assess potential drug-drug interaction (DDI) with compound 68, following FDA guidance, compound 68 was tested against 12 transporter proteins (Eurofins panels): compound 68 is not a substrate of any of them, including the critical transporters BCRP and PgP. Compound 68 was also tested for its inhibitor activity against the 8 major CYP enzymes in human liver microsomes. Also, in this case, no inhibition of the CYP enzymes (@ c=10 μM) was observed, further minimizing the potential for DDI.

### Example 5. Genotoxicity of Compound 68

A mini-Ames assay was conducted with compound 68 using 4 strains of Salmonella typhimurium (TA98, TA100, TA97a, and TA1535). The compound dissolved in DMSO was evaluated at 5 concentrations up to 100M in the absence or presence of metabolic activation (rat liver S9). No evidence of cytotoxicity or mutagenicity was noted in any strain over the tested dose range with or without metabolic activation. Thus, compound 68 is not mutagenic and cytotoxic in bacteria at concentrations up to 100 μM.

### Example 6. In Vitro ADME Profile of Compound 68

The metabolic stability of compound 68 was measured across five species. The results indicated that the compound is anticipated to have a low-medium hepatic extraction ratio (EH) in vivo. Half-life in human, dog, and rats is very similar and higher than in monkey and mice.

### Example 7. In Vivo Efficacy of Compound 68

Compound 68 of Table 1 was examined for anti-inflammatory activity using a lipopolysaccharide (LPS)-induced TNF-α model.

### Study Protocol

12-week-old male C57BL/6 mice were used. All mice were housed in a room that was automatically maintained at 21-25° C. and relative humidity (45%-65%) with a controlled light-dark cycle. The ip injection (750 μg/kg) of LPS or vehicle (saline) was administered daily for 5 days 30 min. after the treatment of Compound 68. Compound 68 was given intranasally (3, 10, and 30 mg/kg) and orally (30 mg/kg).

On day 5, one hour after LPS treatment, all animals were euthanized via CO₂. The plasma, hippocampus and cortex were collected and immediately stored at liquid nitrogen and transferred to −80° C. Tissues were used for ELISA and Western Blot analysis to determine levels of TNF-α and pTau.

### Results

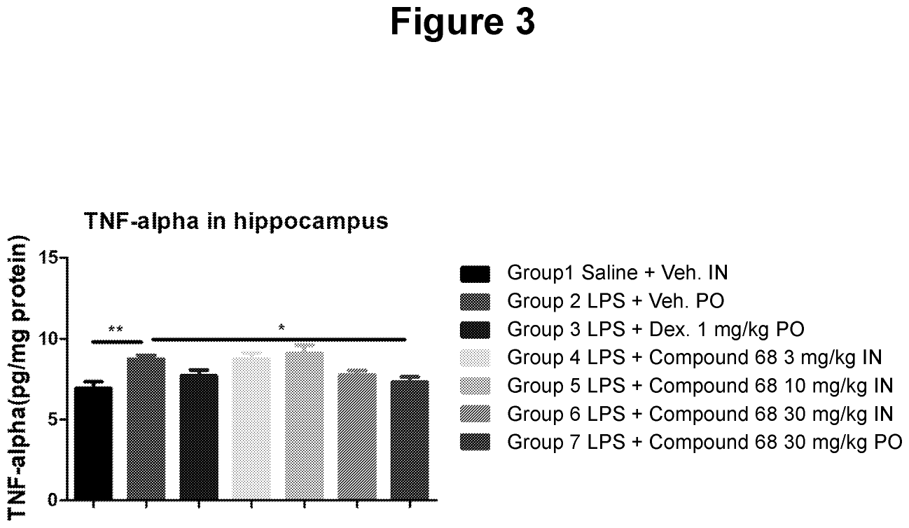

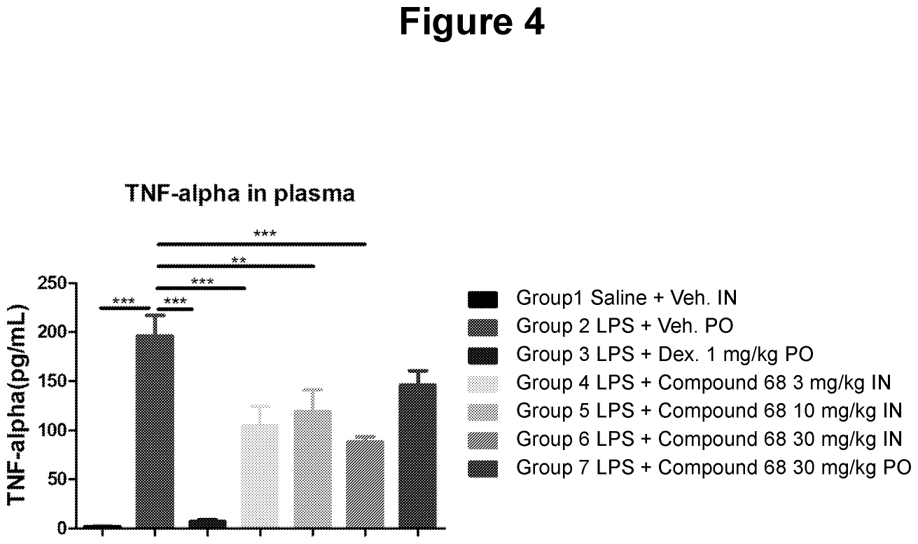

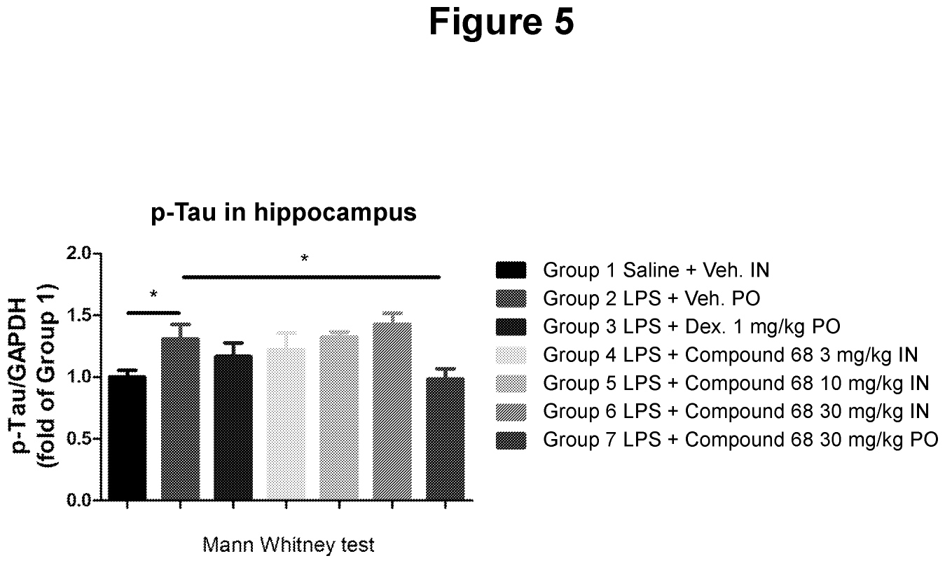

TNF-α levels in collected hippocampus tissue, determined via ELISA assay, are shown in FIG. 3. TNF-α levels in collected plasma, determined via ELISA assay, are shown in FIG. 4. And pTau levels in collected hippocampus, determined via Western Blot (AT8 antibody), are shown in FIG. 5. As shown in FIGS. 3-5, Compound 68 can desirably inhibit lipopolysaccharide-induced expression of TNF-alpha and/or pTau.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A compound of Formula I 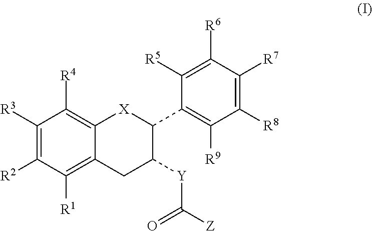
or a pharmaceutically acceptable salt thereof, wherein
R¹, R², R³, and R⁴ are each independently hydrogen, halogen, —NO₂, —CN, C₁-C₁₀ alkyl, C₁-C₁₀ haloalkyl, —NH₂, —NH(C₁-C₁₀ alkyl), —N(C₁-C₁₀ alkyl)₂, —OH, C₁-C₁₀ alkoxy, C₁-C₁₀ haloalkoxy, —SH, hydroxy(C₁-C₁₀ alkyl), alkoxy(C₁-C₁₀ alkyl), amino(C₁-C₁₀ alkyl), —CONH₂, —CONH(C₁-C₁₀ alkyl), —CON(C₁-C₁₀ alkyl)₂, —OC(O)NH₂, —OC(O)NH(C₁-C₁₀ alkyl), —OC(O)N(C₁-C₁₀ alkyl)₂, —CO₂H, —CO₂(C₁-C₁₀ alkyl), —CHO, —CO(C₁-C₁₀ alkyl), —OC(O)(C₁-C₁₀ alkyl), —S(O)₀₋₂(C₁-C₁₀ alkyl), or —NH(S(O)₀₋₂(C₁-C₁₀ alkyl));
R⁵ and R⁹ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or —SH;
R⁷ is hydrogen, halogen, —NO₂, —CN, C₁-C₁₀ alkyl, C₁-C₁₀ haloalkyl, —NH₂, —NH(C₁-C₁₀ alkyl), —N(C₁-C₁₀ alkyl)₂, —OH, C₁-C₁₀ alkoxy, C₁-C₁₀ haloalkoxy, —SH, hydroxy(C₁-C₁₀ alkyl), alkoxy(C₁-C₁₀ alkyl), amino(C₁-C₁₀ alkyl), —CONH₂, —CONH(C₁-C₁₀ alkyl), —CON(C₁-C₁₀ alkyl)₂, —CO₂H, —CO₂(C₁-C₁₀ alkyl), —CHO, —CO(C₁-C₁₀ alkyl), —S(O)₀₋₂(C₁-C₁₀ alkyl), or —NH(S(O)₀₋₂(C₁-C₁₀ alkyl));
R⁶ and R⁸ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or —SH;
X is O or C;
Y is O or NH; and
Z is 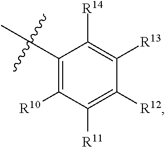
wherein
R¹⁰ and R¹⁴ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or —SH;
R¹² is hydrogen, halogen, —NO₂, —CN, C₁-C₁₀ alkyl, C₁-C₁₀ haloalkyl, —NH₂, —NH(C₁-C₁₀ alkyl), —N(C₁-C₁₀ alkyl)₂, —OH, C₁-C₁₀ alkoxy, C₁-C₁₀ haloalkoxy, —SH, hydroxy(C₁-C₁₀ alkyl), alkoxy(C₁-C₁₀ alkyl), amino(C₁-C₁₀ alkyl), —CONH₂, —CONH(C₁-C₁₀ alkyl), —CON(C₁-C₁₀ alkyl)₂, —CO₂H, —CO₂(C₁-C₁₀ alkyl), —CHO, —CO(C₁-C₁₀ alkyl), —S(O)₀₋₂(C₁-C₁₀ alkyl), or —NH(S(O)₀₋₂(C₁-C₁₀ alkyl);
R¹¹ and R¹³ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or —SH;
or Z is 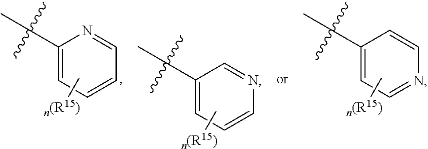
wherein
n is 0-4; and
each R¹⁵ is independently C₁-C₆ alkyl, C₁-C₆ haloalkyl, —NH₂, —NH(C₁-C₆ alkyl), —N(C₁-C₆ alkyl)₂, —OH, C₁-C₆ alkoxy, or C₁-C₆ haloalkoxy;
or Z is 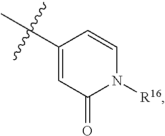
wherein
R¹⁶ is hydrogen, C₁-C₆ alkyl, C₁-C₆ haloalkyl, —OH, C₁-C₆ alkoxy, or C₁-C₆ haloalkoxy;
or Z is 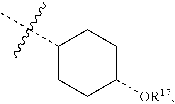
wherein
R¹⁷ is hydrogen, C₁-C₆ alkyl, or C₁-C₆ haloalkyl;
wherein the compound is not
(2R,3R)-2-(3,5-dihydroxy-4-methoxyphenyl)-5,7-dihydroxychroman-3-yl 3,4,5-trihydroxybenzoate;
(2R,3R)-5,7-dihydroxy-2-(3,4,5-trihydroxyphenyl)chroman-3-yl 3,5-dihydroxy-4-methoxybenzoate;
(2R,3R)-5,7-dihydroxy-2-(3,4,5-trihydroxyphenyl)chroman-3-yl 3,4,5-trihydroxybenzoate;
(2S,3R)-5,7-dihydroxy-2-(3,4,5-trihydroxyphenyl)chroman-3-yl 3,4,5-trihydroxybenzoate;
(2R,3S)-5,7-dihydroxy-2-(3,4,5-trihydroxyphenyl)chroman-3-yl 3,4,5-trihydroxybenzoate;
(2R,3R)-2-(3,4-dihydroxyphenyl)-5,7-dihydroxychroman-3-yl 3,4,5-trihydroxybenzoate;
(2S,3R)-2-(3,4-dihydroxyphenyl)-5,7-dihydroxychroman-3-yl 3,4,5-trihydroxybenzoate;
(2R,3R)-5,7-dihydroxy-2-(3,4,5-trihydroxyphenyl)chroman-3-yl 3,4-dihydroxy-5-methoxybenzoate;
(2R,3S)-5,7-dihydroxy-2-(3,4,5-trihydroxyphenyl)chroman-3-yl 3,4-difluorobenzoate; or
(2S,3R)-5,7-dihydroxy-2-(3,4,5-trihydroxyphenyl)chroman-3-yl 2,3,4-trihydroxybenzoate.

2. The compound of claim 1, wherein the compound is of Formula I-A

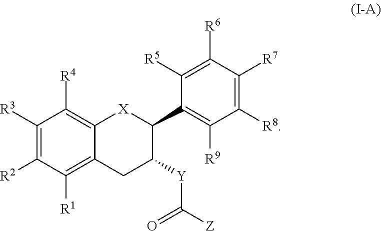

3. The compound of claim 1, wherein the compound is of Formula I-B

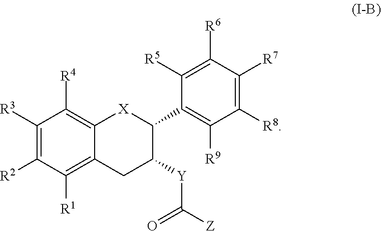

4. The compound of claim 1, wherein X is O, and/or wherein Y is O.

5. (canceled)

6. The compound of claim 1, wherein R¹, R², R³, and R⁴ are each independently hydrogen, —OH, C₁-C₁₀ alkoxy, —OC(O)(C₁-C₁₀ alkyl), or —OC(O)NH(C₁-C₁₀ alkyl).

7-8. (canceled)

9. The compound of claim 1, wherein R⁵ and R⁹ are each independently hydrogen, —F, or —OH.

10-11. (canceled)

12. The compound of claim 1, wherein R⁷ is —OH, C₁-C₁₀ alkoxy, —CONH₂, —CONH(C₁-C₁₀ alkyl), —CO(C₁-C₁₀ alkyl), or —NH(S(O)₀₋₂(C₁-C₁₀ alkyl)).

13-14. (canceled)

15. The compound of claim 1, wherein R⁶ and R⁸ are each independently hydrogen, —OH, C₁-C₆ alkoxy, or C₁-C₆ haloalkoxy.

16. (canceled)

17. The compound of claim 1, wherein
R⁵ and R⁹ are each independently hydrogen or —F;
R⁷ is —OH;
R⁶ and R⁸ are each independently hydrogen or —OH;
at least one of R⁵ and R⁹ is hydrogen; and
at least one of R⁶ and R⁸ is —OH.

18. The compound of claim 1, wherein
Z is 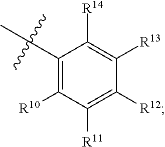
R¹⁰ and R¹⁴ are each independently hydrogen, —F, or —OH;
R¹² is —OH, C₁-C₁₀ alkoxy, —CONH₂, —CONH(C₁-C₁₀ alkyl), —CO(C₁-C₁₀ alkyl), or —NH(S(O)₀₋₂(C₁-C₁₀ alkyl)); and
R¹¹ and R¹³ are each independently hydrogen, —OH, C₁-C₆ alkoxy, or C₁-C₆ haloalkoxy.

19. The compound of claim 18, wherein at least one of R¹⁰ and R¹⁴ is F; and/or wherein R¹³ and R¹⁴ are each hydrogen; and/or wherein R¹⁰ and R¹¹ are each —OH; and/or wherein R¹² is —OH; and/or wherein at least one of R¹¹ and R¹³ is —OH, C₁-C₆ alkoxy, or C₁-C₆ haloalkoxy.

20-23. (canceled)

24. The compound of claim 1, wherein the compound is of Formula II, Formula II-A, or Formula II-B, Formula III, Formula III-A, or Formula III-B

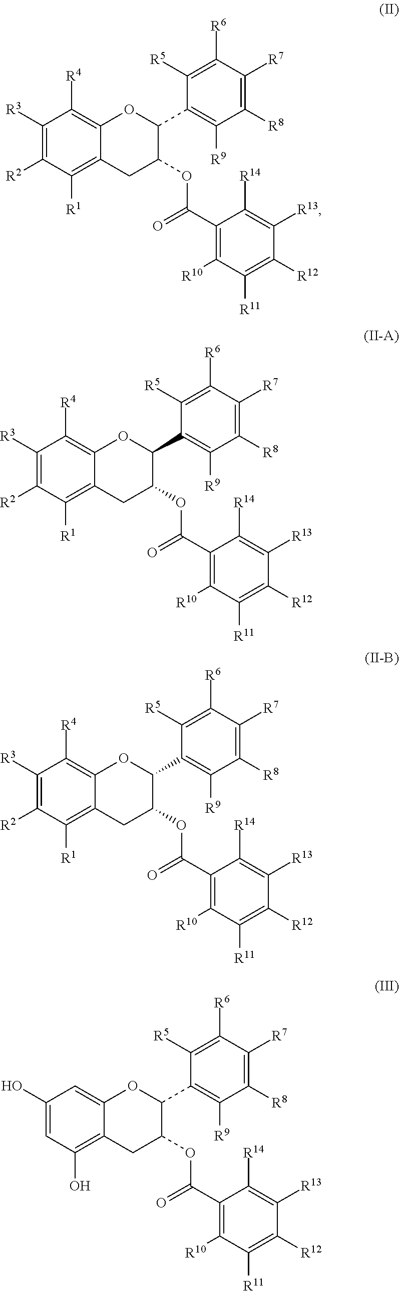

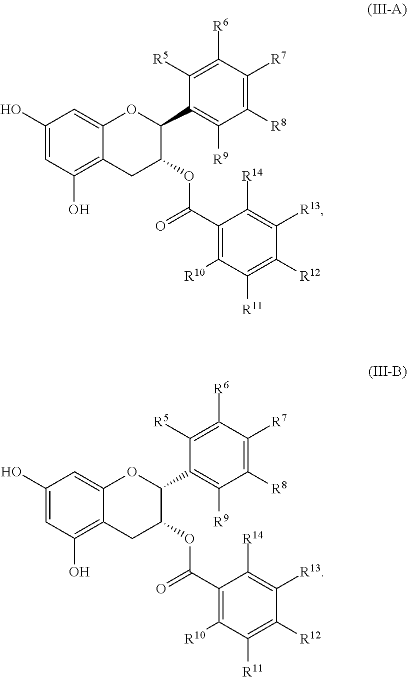

25-29. (canceled)

30. The compound of claim 24, wherein at least one of R⁵, R⁹, R¹⁰, and R¹⁴ is not hydrogen; or wherein R⁵, R⁹, R¹⁰, and R¹⁴ are each independently hydrogen, halogen, or —OH.

31. (canceled)

32. The compound of claim 24, wherein at least one of R⁵, R⁹, R¹⁰, and R¹⁴ is F.

33. The compound of claim 24, wherein
R⁵, R⁹, R¹⁰, and R¹⁴ are each independently hydrogen or —F, and one or two of R⁵, R⁹, R¹⁰, and R¹⁴ are F;
optionally wherein R⁷ and R¹² are each independently —OH, C₁-C₁₀ alkoxy, —CONH₂, —CONH(C₁-C₁₀ alkyl), —CO(C₁-C₁₀ alkyl), or —NH(S(O)₀₋₂(C₁-C₁₀ alkyl).

34-36. (canceled)

37. The compound of claim 24, wherein R⁶, R⁸, R¹¹, and R¹³ are each independently hydrogen, —OH, C₁-C₆ alkoxy, or C₁-C₆ haloalkoxy, and/or wherein at least one of R⁶, R⁸, R¹¹, and R¹³ is C₁-C₆ alkoxy or C₁-C₆ haloalkoxy; and/or R⁷, R⁸, R¹¹, and R¹² are ach —OH and R¹³ is —OH, C₁-C₆ alkoxy, or C₁-C₆ haloalkoxy; and/or wherein at least one of R⁵, R⁹, R¹⁰, and R¹⁴ is —F, positioned para to a substituent selected from —OH, C₁-C₆ alkoxy, or C₁-C₆ haloalkoxy, and/or R¹³ is —OH, C₁-C₆ alkoxy, or C₁-C₆ haloalkoxy and R¹⁰ is —F.

38-41. (canceled)

42. The compound of claim 24, wherein
(a) R¹, R³, R⁷, R⁸, R¹¹, and R¹² are each —OH;
R², R⁴, and R⁹ are each hydrogen;
R⁶ and R¹³ are each —OH, C₁-C₆ alkoxy, or C₁-C₆ haloalkoxy; and
at least one of R⁵, R¹⁰, and R¹⁴ is —F,
optionally wherein R⁵ and R¹⁴ are each hydrogen and R¹⁰ is —F, or
(b) R¹, R³, R⁷, and R¹² are each —OH;
R², R⁴, and R⁹ ach hydrogen;
R⁵, R¹³, and R¹⁴ are each independently hydrogen or —F;
R⁶ and R⁸ are each independently hydrogen or —OH; and
R¹⁰ and R¹¹ are each independently hydrogen, —OH, C₁-C₆ alkoxy, or C₁-C₆ haloalkoxy;
optionally wherein R¹⁰ and R¹¹ are each independently —OH, C₁-C₆ alkoxy, or C₁-C₆ haloalkoxy; or
(c) R¹, R³, R⁶, R⁷, R⁸, R¹⁰, R¹¹, and R¹² are each —OH, and R², R⁴, R⁵, R⁹, R¹³, and R¹⁴ are each independently hydrogen or —F.

43-46. (canceled)

47. The compound of claim 1, wherein
(a) Z is 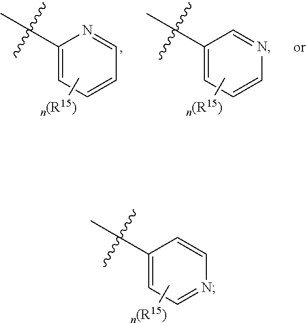
n is 0-2; and
each R¹⁵ is independently —NH₂, —OH, or C₁-C₆ alkoxy; or
(b) Z is 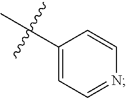 [SMILES: *c1ccncc1]
or
(c) Z is 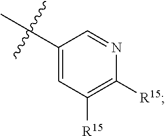
and
each R¹⁵ is independently —NH₂ or —OH; or
(d) Z is 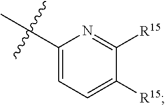
and
each R¹⁵ is independently —NH₂ or —OH;
(e) Z is 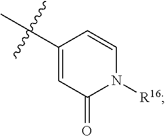
and
R¹⁶ is hydrogen or —OH; or
(f) Z is

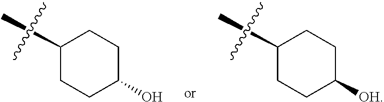

48-52. (canceled)

53. The compound of claim 1, wherein the compound is
N-((2R,3S)-5,7-dihydroxy-2-(3,4,5-trihydroxyphenyl)chroman-3-yl)-3,4,5-trihydroxybenzamide;
N-((2R,3S)-5,7-dihydroxy-2-(3,4,5-trihydroxyphenyl)chroman-3-yl)-3,4-dihydroxy-5-methoxybenzamide;
(2R,3R)-5,7-dihydroxy-2-(3,4,5-trihydroxyphenyl)chroman-3-yl 3,4-dihydroxybenzoate;
(2R,3R)-5,7-dihydroxy-2-(3,4,5-trihydroxyphenyl)chroman-3-yl 3-((ethylcarbamoyl)oxy)-4,5-dihydroxybenzoate;
(2R,3R)-5,7-dihydroxy-2-(3,4,5-trihydroxyphenyl)chroman-3-yl 4-((ethylcarbamoyl)oxy)-3,5-dihydroxybenzoate;
(2R,3R)-5,7-dihydroxy-2-(3,4,5-trihydroxyphenyl)chroman-3-yl 3,5-dihydroxy-4-(isobutyryloxy)benzoate;
N-((2R,3S)-5,7-dihydroxy-2-(3,4,5-trihydroxyphenyl)chroman-3-yl)-3,4-dihydroxybenzamide;
N-((2R,3S)-5,7-dihydroxy-2-(3,4,5-trihydroxyphenyl)chroman-3-yl)-4-hydroxybenzamide;
N-((2R,3S)-5,7-dihydroxy-2-(3,4,5-trihydroxyphenyl)chroman-3-yl)-3,4-difluorobenzamide;
(2R,3S)-5,7-dihydroxy-2-(3,4,5-trihydroxyphenyl)chroman-3-yl 3,4-dihydroxy-5-methoxybenzoate;
(2R,3S)-5,7-dihydroxy-2-(3,4,5-trihydroxyphenyl)chroman-3-yl 3,4-dihydroxybenzoate;
(2R,3S)-5,7-dihydroxy-2-(3,4,5-trihydroxyphenyl)chroman-3-yl 4-hydroxybenzoate;
(2R,3R)-5,7-dihydroxy-2-(3,4,5-trihydroxyphenyl)chroman-3-yl 4-hydroxybenzoate;
(2R,3S)-5,7-dihydroxy-2-(3,4,5-trihydroxyphenyl)chroman-3-yl isonicotinate;
(2R,3R)-5,7-dihydroxy-2-(3,4,5-trihydroxyphenyl)chroman-3-yl isonicotinate;
(2R,3R)-5,7-dihydroxy-2-(3,4,5-trihydroxyphenyl)chroman-3-yl 3,4-difluorobenzoate;
(2R,3R)-5,7-dihydroxy-2-(3,4,5-trihydroxyphenyl)chroman-3-yl 1-hydroxy-2-oxo-1,2-dihydropyridine-4-carboxylate;
(2R,3S)-5,7-dihydroxy-2-(3,4,5-trihydroxyphenyl)chroman-3-yl 5,6-dihydroxynicotinate;
(2R,3S)-2-(3,4-dihydroxyphenyl)-5,7-dihydroxychroman-3-yl 3,4-dihydroxybenzoate;
(2R,3S)-2-(3,4-dihydroxyphenyl)-5,7-dihydroxychroman-3-yl 6-amino-5-hydroxynicotinate;
(2R,3S)-2-(3,4-dihydroxyphenyl)-5,7-dihydroxychroman-3-yl 3-hydroxy-4-(methylsulfonamido)benzoate;
(2S,3R)-2-(3,4-dihydroxyphenyl)-5,7-dihydroxychroman-3-yl 3,4-dihydroxybenzoate;
(2S,3R)-5-hydroxy-2-(3,4,5-trihydroxyphenyl)chroman-3-yl 3,4,5-trihydroxybenzoate;
(2R,3S)-5-hydroxy-3-(3,4,5-trihydroxyphenyl)-1,2,3,4-tetrahydronaphthalen-2-yl 3,4,5-trihydroxybenzoate;
(2S,3R)-5,7-dihydroxy-2-(3,4,5-trihydroxyphenyl)chroman-3-yl 3,4-dihydroxybenzoate;
(2S,3R)-5,7-dihydroxy-2-(3,4,5-trihydroxyphenyl)chroman-3-yl 4-amino-3-hydroxybenzoate;
(2S,3R)-5,7-dihydroxy-2-(3,4,5-trihydroxyphenyl)chroman-3-yl 6-amino-5-hydroxynicotinate;
(2S,3R)-5,7-dihydroxy-2-(3,4,5-trihydroxyphenyl)chroman-3-yl 3-hydroxy-4-(methylsulfonamido)benzoate;
(2S,3R)-2-(3,4,5-trihydroxyphenyl)chroman-3-yl 3,4,5-trihydroxybenzoate;
(2S,3R)-5,7-dihydroxy-2-(3,4,5-trihydroxyphenyl)chroman-3-yl 2-fluoro-3,4,5-trihydroxybenzoate;
(2S,3R)-5,7-dihydroxy-2-(3,4,5-trihydroxyphenyl)chroman-3-yl 2-fluoro-4,5-dihydroxybenzoate;
(2S,3R)-5,7-dihydroxy-2-(3,4,5-trihydroxyphenyl)chroman-3-yl 2,4,5-trihydroxybenzoate;
(2S,3R)-5,7-dihydroxy-2-(3,4,5-trihydroxyphenyl)chroman-3-yl (1s,4S)-4-hydroxycyclohexane-1-carboxylate;
(2S,3R)-5,7-dihydroxy-2-(3,4,5-trihydroxyphenyl)chroman-3-yl (1r,4R)-4-hydroxycyclohexane-1-carboxylate;
(2S,3R)-2-(2-fluoro-4,5-dihydroxyphenyl)-5,7-dihydroxychroman-3-yl 3,4,5-trihydroxybenzoate;
(2S,3R)-2-(2-fluoro-3,4,5-trihydroxyphenyl)-5,7-dihydroxychroman-3-yl 3,4,5-trihydroxybenzoate;
(2S,3R)-2-(2-fluoro-3,4,5-trihydroxyphenyl)-5,7-dihydroxychroman-3-yl 2-fluoro-3,4,5-trihydroxybenzoate;
(2S,3R)-2-(2-fluoro-4,5-dihydroxyphenyl)-5,7-dihydroxychroman-3-yl 2-fluoro-3,4,5-trihydroxybenzoate;
(2S,3R)-2-(3,4-dihydroxy-5-methoxyphenyl)-5,7-dihydroxychroman-3-yl 2-fluoro-3,4,5-trihydroxybenzoate;
(2S,3R)-5,7-dihydroxy-2-(3,4,5-trihydroxyphenyl)chroman-3-yl 2-fluoro-4,5-dihydroxy-3-methoxybenzoate;
(2S,3R)-5,7-dihydroxy-2-(3,4,5-trihydroxyphenyl)chroman-3-yl 2,6-difluoro-3,4,5-trihydroxybenzoate;
(2S,3R)-2-(4,5-dihydroxy-2-methylphenyl)-5,7-dihydroxychroman-3-yl 2-fluoro-3,4,5-trihydroxybenzoate;
(2R,3S)-3-(3,4,5-trihydroxyphenyl)-1,2,3,4-tetrahydronaphthalen-2-yl 3,4,5-trihydroxybenzoate;
(2R,3S)-3-(3,4,5-trihydroxyphenyl)-1,2,3,4-tetrahydronaphthalen-2-yl 2-fluoro-3,4,5-trihydroxybenzoate;
(2S,3R)-2-(3,4-dihydroxyphenyl)-5,7-dihydroxychroman-3-yl 2-fluoro-3,4,5-trihydroxybenzoate;
(2R,3R)-2-(3,4-dihydroxyphenyl)-5,7-dihydroxychroman-3-yl 2-fluoro-3,4,5-trihydroxybenzoate;
(2S,3R)-5,7-dihydroxy-2-(3,4,5-trihydroxyphenyl)chroman-3-yl 5,6-dihydroxypicolinate;
(2R,3R)-5,7-dihydroxy-2-(3,4,5-trihydroxyphenyl)chroman-3-yl 2-fluoro-3,4,5-trihydroxybenzoate;
(2S,3R)-5,7-dihydroxy-2-(3,4,5-trihydroxyphenyl)chroman-3-yl 2,6-difluoro-3,4-dihydroxy-5-methoxybenzoate;
(2S,3R)-5,7-dihydroxy-2-(3,4,5-trihydroxyphenyl)chroman-3-yl 3-(difluoromethoxy)-4,5-dihydroxybenzoate;
(2S,3R)-5,7-dihydroxy-2-(3,4,5-trihydroxyphenyl)chroman-3-yl 2,6-difluoro-3,4-dihydroxy-5-isopropoxybenzoate;
(2S,3R)-5,7-dihydroxy-2-(3,4,5-trihydroxyphenyl)chroman-3-yl 4,5-dihydroxy-2-(trifluoromethyl)benzoate;
(2S,3R)-5,7-dihydroxy-2-(3,4,5-trihydroxyphenyl)chroman-3-yl 3,4-dihydroxy-2-methylbenzoate;
(2S,3R)-2-(3,4-dihydroxyphenyl)-5,7-dihydroxychroman-3-yl 2,6-difluoro-3,4-dihydroxy-5-methoxybenzoate;
(2S,3R)-5,7-dihydroxy-2-(3,4,5-trihydroxyphenyl)chroman-3-yl 5-(difluoromethoxy)-2-fluoro-3,4-dihydroxybenzoate;
(2S,3R)-5,7-dihydroxy-2-(3,4,5-trihydroxyphenyl)chroman-3-yl 3-fluoro-4,5-dihydroxybenzoate;
(2S,3R)-2-(4-((ethylcarbamoyl)oxy)-3,5-dihydroxyphenyl)-5,7-dihydroxychroman-3-yl 2-fluoro-3,4,5-trihydroxybenzoate;
(2S,3R)-5,7-dihydroxy-2-(3,4,5-trihydroxyphenyl)chroman-3-yl 2-fluoro-3,4-dihydroxy-5-methoxybenzoate;
(2R,3R)-5-hydroxy-7-(propionyloxy)-2-(3,4,5-trihydroxyphenyl)chroman-3-yl 3,4,5-trihydroxybenzoate;
(2R,3R)-7-((ethylcarbamoyl)oxy)-5-hydroxy-2-(3,4,5-trihydroxyphenyl)chroman-3-yl
3,4,5-trihydroxybenzoate;
(2R,3R)-7-(hexanoyloxy)-5-hydroxy-2-(3,4,5-trihydroxyphenyl)chroman-3-yl 3,4,5-trihydroxybenzoate;
(2R,3R)-2-(4-((ethylcarbamoyl)oxy)-3,5-dihydroxyphenyl)-5,7-dihydroxychroman-3-yl
3,4,5-trihydroxybenzoate;
(2R,3R)-5,7-dihydroxy-2-(3,4,5-trihydroxyphenyl)chroman-3-yl 3,5-dihydroxy-4-((3-methylbutanoyl)oxy)benzoate;
(2R,3R)-5,7-dihydroxy-2-(3,4,5-trihydroxyphenyl)chroman-3-yl 3,5-dihydroxy-4-(propionyloxy)benzoate;
(2R,3R)-5,7-dihydroxy-2-(3,4,5-trihydroxyphenyl)chroman-3-yl 4-((ethylcarbamoyl)oxy)-3,5-dihydroxybenzoate;
(2R,3R)-5,7-dihydroxy-2-(3,4,5-trihydroxyphenyl)chroman-3-yl 4-((dimethylcarbamoyl)oxy)-3,5-dihydroxybenzoate;
(2R,3R)-5,7-dihydroxy-2-(3,4,5-trihydroxyphenyl)chroman-3-yl 3,4-dihydroxy-5-(propionyloxy)benzoate;
(2R,3R)-5,7-dihydroxy-2-(3,4,5-trihydroxyphenyl)chroman-3-yl 3,4-dihydroxy-5-(isobutyryloxy)benzoate;
(2R,3R)-2-(3,5-dihydroxy-4-(propionyloxy)phenyl)-5,7-dihydroxychroman-3-yl 3,4,5-trihydroxybenzoate;
(2R,3R)-2-(4-((ethylcarbamoyl)oxy)-3,5-dihydroxyphenyl)-5,7-dihydroxychroman-3-yl 4-((ethylcarbamoyl)oxy)-3,5-dihydroxybenzoate; or
(2R,3R)-5,7-dihydroxy-2-(3,4,5-trihydroxyphenyl)chroman-3-yl 2-fluoro-3,4,5-trihydroxybenzoate.

54. The compound of claim 1, wherein the compound is (2S,3R)-5,7-dihydroxy-2-(3,4,5-trihydroxyphenyl)chroman-3-yl 2-fluoro-3,4,5-trihydroxybenzoate;
(2S,3R)-5,7-dihydroxy-2-(3,4,5-trihydroxyphenyl)chroman-3-yl 2-fluoro-4,5-dihydroxybenzoate;
(2S,3R)-2-(2-fluoro-3,4,5-trihydroxyphenyl)-5,7-dihydroxychroman-3-yl 3,4,5-trihydroxybenzoate;
(2S,3R)-2-(2-fluoro-3,4,5-trihydroxyphenyl)-5,7-dihydroxychroman-3-yl 2-fluoro-3,4,5-trihydroxybenzoate;
(2S,3R)-2-(2-fluoro-4,5-dihydroxyphenyl)-5,7-dihydroxychroman-3-yl 2-fluoro-3,4,5-trihydroxybenzoate;
(2S,3R)-5,7-dihydroxy-2-(3,4,5-trihydroxyphenyl)chroman-3-yl 2-fluoro-4,5-dihydroxy-3-methoxybenzoate;
(2S,3R)-5,7-dihydroxy-2-(3,4,5-trihydroxyphenyl)chroman-3-yl 2,6-difluoro-3,4,5-trihydroxybenzoate;
(2S,3R)-2-(3,4-dihydroxyphenyl)-5,7-dihydroxychroman-3-yl 2-fluoro-3,4,5-trihydroxybenzoate;
(2S,3R)-5,7-dihydroxy-2-(3,4,5-trihydroxyphenyl)chroman-3-yl 2,6-difluoro-3,4-dihydroxy-5-methoxybenzoate;
(2S,3R)-5,7-dihydroxy-2-(3,4,5-trihydroxyphenyl)chroman-3-yl 2-fluoro-3,4-dihydroxy-5-methoxybenzoate; or
(2R,3R)-5,7-dihydroxy-2-(3,4,5-trihydroxyphenyl)chroman-3-yl 2-fluoro-3,4,5-trihydroxybenzoate.

55. The compound of claim 1, wherein the compound is (2S,3R)-5,7-dihydroxy-2-(3,4,5-trihydroxyphenyl)chroman-3-yl 2-fluoro-3,4-dihydroxy-5-methoxybenzoate, or where the compound is (2R,3R)-5,7-dihydroxy-2-(3,4,5-trihydroxyphenyl)chroman-3-yl 2-fluoro-3,4,5-trihydroxybenzoate.

56. (canceled)

57. A pharmaceutical composition comprising one or more compounds according to claim 1 and a pharmaceutically acceptable carrier, excipient, adjuvant, and/or diluent.

58. An intranasal pharmaceutical composition comprising one or more compounds according to claim 1, present in a combined amount of 1-40 wt. %, and one or more of
a permeation enhancer, present in an amount of 0.1-20 wt. %;
a chelator/anti-oxidant, present in an amount of 0.1-20 wt. %;
a humectant; present in an amount of 1-30 wt. %; and
a preservative, present in an amount of 0.03-2 wt. %;
wherein the pH of the composition is 4.0-6.5.

59-69. (canceled)

70. A method for treating or limiting oxidative stress, inflammation, a central nervous system disorder, a tumor, a diabetes, obesity, a systemic disorder, a neurological disorder, a viral infection, nonalcoholic fatty liver disease (NAFLD), or uveitis, comprising administering to a subject in need thereof a therapeutically effective amount of one or more compounds of Formula I 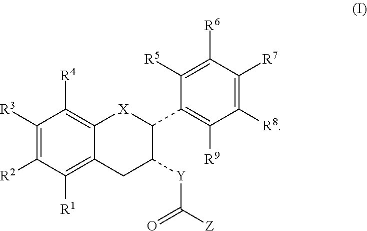
or a pharmaceutically acceptable salt thereof, wherein
R¹, R², R³, and R⁴ are each independently hydrogen, halogen, —NO₂, —CN, C₁-C₁₀ alkyl, C₁-C₁₀ haloalkyl, —NH₂, —NH(C₁-C₁₀ alkyl), —N(C₁-C₁₀ alkyl)₂, —OH, C₁-C₁₀ alkoxy, C₁-C₁₀ haloalkoxy, —SH, hydroxy(C₁-C₁₀ alkyl), alkoxy(C₁-C₁₀ alkyl), amino(C₁-C₁₀ alkyl), —CONH₂, —CONH(C₁-C₁₀ alkyl), —CON(C₁-C₁₀ alkyl)₂, —OC(O)NH₂, —OC(O)NH(C₁-C₁₀ alkyl), —OC(O)N(C₁-C₁₀ alkyl)₂, —CO₂H, —CO₂(C₁-C₁₀ alkyl), —CHO, —CO(C₁-C₁₀ alkyl), —OC(O)(C₁-C₁₀ alkyl), —S(O)₀₋₂(C₁-C₁₀ alkyl), or —NH(S(O)₀₋₂(C₁-C₁₀ alkyl));
R⁵ and R⁹ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or —SH;
R⁷ is hydrogen, halogen, —NO₂, —CN, C₁-C₁₀ alkyl, C₁-C₁₀ haloalkyl, —NH₂, —NH(C₁-C₁₀ alkyl), —N(C₁-C₁₀ alkyl)₂, —OH, C₁-C₁₀ alkoxy, C₁-C₁₀ haloalkoxy, —SH, hydroxy(C₁-C₁₀ alkyl), alkoxy(C₁-C₁₀ alkyl), amino(C₁-C₁₀ alkyl), —CONH₂, —CONH(C₁-C₁₀ alkyl), —CON(C₁-C₁₀ alkyl)₂, —CO₂H, —CO₂(C₁-C₁₀ alkyl), —CHO, —CO(C₁-C₁₀ alkyl), —S(O)₀₋₂(C₁-C₁₀ alkyl), or —NH(S(O)₀₋₂(C₁-C₁₀ alkyl));
R⁶ and R⁸ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or —SH;
X is O or C;
Y is O or NH; and
Z is 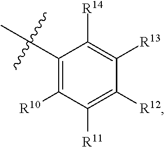
wherein
R¹⁰ and R¹⁴ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or —SH;
R¹² is hydrogen, halogen, —NO₂, —CN, C₁-C₁₀ alkyl, C₁-C₁₀ haloalkyl, —NH₂, —NH(C₁-C₁₀ alkyl), —N(C₁-C₁₀ alkyl)₂, —OH, C₁-C₁₀ alkoxy, C₁-C₁₀ haloalkoxy, —SH, hydroxy(C₁-C₁₀ alkyl), alkoxy(C₁-C₁₀ alkyl), amino(C₁-C₁₀ alkyl), —CONH₂, —CONH(C₁-C₁₀ alkyl), —CON(C₁-C₁₀ alkyl)₂, —CO₂H, —CO₂(C₁-C₁₀ alkyl), —CHO, —CO(C₁-C₁₀ alkyl), —S(O)₀₋₂(C₁-C₁₀ alkyl), or —NH(S(O)₀₋₂(C₁-C₁₀ alkyl));
R¹¹ and R¹³ are each independently hydrogen, halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or —SH;
or Z is 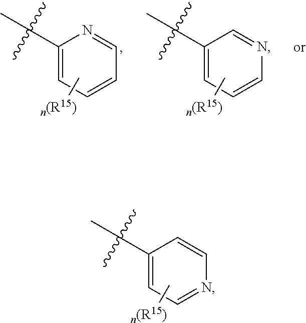
wherein
n is 0-4; and
each R¹⁵ is independently C₁-C₆ alkyl, C₁-C₆ haloalkyl, —NH₂, —NH(C₁-C₆ alkyl), —N(C₁-C₆ alkyl)₂, —OH, C₁-C₆ alkoxy, or C₁-C₆ haloalkoxy;
or Z is 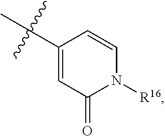
wherein
R¹⁶ is hydrogen, C₁-C₆ alkyl, C₁-C₆ haloalkyl, —OH, C₁-C₆ alkoxy, or C₁-C₆ haloalkoxy;
or Z is 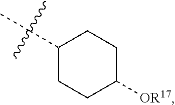
wherein
R¹⁷ is hydrogen, C₁-C₆ alkyl, or C₁-C₆ haloalkyl.

71-94. (canceled)

95. A method of preparing gallocatechin from epigallocatechin, comprising
(a) contacting epigallocatechin with an aqueous buffer system at a first temperature greater than 50° C. for a first period of time to provide a crude reaction mixture comprising gallocatechin;
(b) cooling the crude reaction mixture to a second temperature lower than the first temperature to provide a precipitated crude material comprising gallocatechin;
(c) separating the precipitated crude material from the crude reaction mixture; and then
(d) recrystallizing the separated crude material in an aqueous solvent to provide a purified material comprising gallocatechin.

96-109. (canceled)
